(19) United States
(12) Patent Application Publication
(10) Pub. No.: US 2023/0121952 A1
(43) Pub. Date: Apr. 20, 2023

(54) COMPOUNDS

(71) Applicant: INFLAZOME LIMITED, Dublin 2 (IE)

(72) Inventors: Matthew COOPER, Cambridge (GB); David MILLER, Cambridge (GB); Angus MACLEOD, Cambridge (GB); Stephen THOM, Nottingham (GB); Jonathan SHANNON, Nottingham (GB); Celia Amparo INCERTI-PRADILLOS, Nottingham (GB); Thomas ALANINE, Nottingham (GB); Shawn JOHNSTONE, Québec (CA); Juliette SABBATANI, Québec (CA)

(21) Appl. No.: 17/800,184

(22) PCT Filed: Feb. 16, 2021
(86) PCT No.: PCT/EP2021/053741

§ 371 Date: Aug. 16, 2022

(30) Foreign Application Priority Data
Feb. 18, 2020 (GB) 2002216.6
Mar. 31, 2020 (GB) 2004686.8

(51) Int. Cl. C07D 515/22; A61P 35/00

(52) U.S. Cl. CPC C07D 515/22; A61P 35/00

(57) ABSTRACT

The present invention relates to macrocyclic compounds, such as macrocyclic sulfonyl triazoles. The present invention further relates to associated salts, solvates, prodrugs and pharmaceutical compositions, and to the use of such compounds in the treatment and prevention of medical disorders and diseases, most especially by NLRP3 inhibition.

### FIELD OF THE INVENTION

The present invention relates to macrocyclic compounds, such as macrocyclic sulfonyl triazoles. The present invention further relates to associated salts, solvates, prodrugs and pharmaceutical compositions, and to the use of such compounds in the treatment and prevention of medical disorders and diseases, most especially by NLRP3 inhibition.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1).

Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3−/− mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al., J Pharmacol Exp Ther, 299: 187-197, 2001). CRIDs are a class of diarylsulfonylurea-containing compounds that inhibit the post-translational processing of IL-1R. Post-translational processing of IL-1R is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59(5), 1691-1710, 2016; and WO 2016/131098 A1, WO 2017/129897 A1, WO 2017/140778 A1, WO 2017/184623 A1, WO 2017/184624 A1, WO 2018/015445 A1, WO 2018/136890 A1, WO 2018/215818 A1, WO 2019/008025 A1, WO 2019/008029 A1, WO 2019/034686 A1, WO 2019/034688 A1, WO 2019/034690 A1, WO 2019/034692 A1, WO 2019/034693 A1, WO 2019/034696 A1, WO 2019/034697 A1, WO 2019/043610 A1, WO 2019/092170 A1, WO 2019/092171 A1, WO 2019/092172 A1, WO 2019/166619 A1, WO 2019/166621 A1 and WO 2019/166623 A1). Certain sulfoximine-containing compounds are also disclosed as inhibitors of NLRP3 (WO 2018/225018 A1, WO 2019/023145 A1, WO 2019/023147 A1, and WO 2019/068772 A1).

In addition, certain heterocyclic sulfonyl compounds, such as sulfonyl triazoles, are disclosed as inhibitors of NLRP3 (see WO 2019/211463 A1).

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

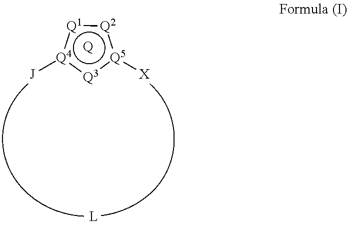

wherein:
J is —SO—, —SO₂—, —SO(═NH)— or —SO(═NR^{j})—;
Q¹ and Q² are each independently selected from O, S, N, NH, NR^{q}, CH, CHal or CR^{qq}, provided that at least one of Q¹ and Q² is selected from N, NH and NR^{q};
Q³ is selected from O, S, N, NH and NR^{q}; and
Q⁴ and Q⁵ are each independently selected from C and N, provided that at least one of Q⁴ and Q⁵ is C;
such that ring Q is a 5-membered heteroaryl ring;
X is —O—, —NH—, —NR^{x}—, —CH₂—, —CH(Hal)-, —C(Hal)₂-, —CH(R^{xx})—, —C(Hal)(R^{xx})— or —C(R^{xx})₂—;
L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N, O and S in its carbon skeleton;
-J-, ring Q, —X— and -L- together form a ring, such that the minimum single ring size that encompasses all or part of each of -J-, ring Q, —X— and -L- is from 8 to 30 atoms;
each R^{j}, R^{q} and R^{x} is independently selected from a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton;
each R^{qq} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton;
each R^{xx} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton, or any two R^{xx} may, together with the carbon atom to which they are attached, form a saturated or unsaturated cyclic group, wherein the cyclic group may optionally be substituted; and
each Hal is independently selected from F, Cl, Br or I.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms.

More typically, a cyclic group is a 3- to 7-membered monocyclic group, which means it contains from 3 to 7 ring atoms.

As used herein, where it is stated that a monovalent cyclic group is monocyclic, it is to be understood that the monovalent cyclic group is not substituted with a divalent bridging substituent (e.g. —O—, —S—, —NH—, —N(R^{β})—, —N(O)(R^{β})—, —N⁺(R^{β})₂— or —R^{α}—) so as to form a bridged, fused or spiro substituent. However, unless stated otherwise, a substituted monovalent monocyclic group may be substituted with one or more further monovalent cyclic groups. Similarly, where it is stated that a monovalent cyclic group is bicyclic, it is to be understood that the monovalent cyclic group including any bridged, fused or spiro divalent bridging substituents attached to the monovalent cyclic group, but excluding any monovalent cyclic substituents, is bicyclic.

Likewise, where it is stated that a divalent cyclic group is monocyclic, it is to be understood that while one or more bridged, fused or spiro ring structures may be formed via the two positions of attachment of the divalent cyclic group to the remainder of the molecule, the divalent cyclic group is not substituted at other positions with a divalent bridging substituent (e.g. —O—, —S—, —NH—, —N(R^{β})—, —N(O)(R^{β})—, —N⁺(R^{β})₂— or —R^{α}—) so as to form a further bridged, fused or spiro substituent. However, unless stated otherwise, a substituted divalent monocyclic group may be substituted with one or more further monovalent cyclic groups. Similarly, where it is stated that a divalent cyclic group is bicyclic, it is to be understood that the divalent cyclic group including any bridged, fused or spiro divalent bridging substituents attached to the cyclic group, but excluding any monovalent cyclic substituents or any structures formed via the two positons of attachment of the divalent cyclic group to the remainder of the molecule, is bicyclic.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

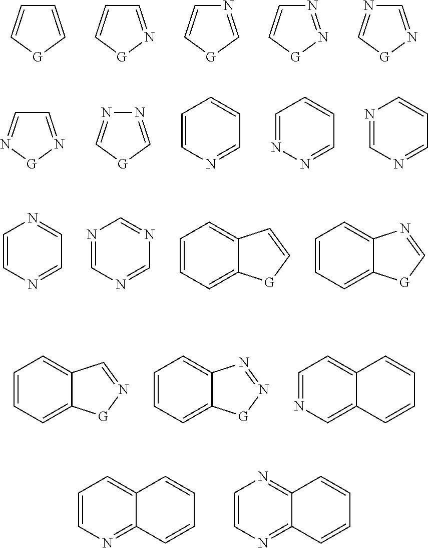

wherein G=O, S or NH. Particular examples of 5- or 6-membered heteroaryl groups include furanyl, thiophenyl, pyrrolyl, imidazolyl, pyrazolyl, oxazolyl, isoxazolyl, thiazolyl, isothiazolyl, triazolyl, furazanyl, oxadiazolyl, thiadiazolyl, tetrazolyl, pyridinyl, pyridazinyl, pyrimidinyl, pyrazinyl and triazinyl groups.

Unless stated otherwise, where a cyclic group or moiety is stated to be non-aromatic, such as a cycloalkyl, cycloalkenyl or non-aromatic heterocyclic group, it is to be understood that the group or moiety, excluding any ring systems which are part of or formed by substituents, is non-aromatic. Similarly, where a cyclic group or moiety is stated to be aromatic, such as an aryl or a heteroaryl group, it is to be understood that the group or moiety, excluding any ring systems which are part of or formed by substituents, is aromatic. A cyclic group or moiety is considered non-aromatic, when it does not have any tautomers that are aromatic. When a cyclic group or moiety has a tautomer that is aromatic, it is considered aromatic, even if it has tautomers that are not aromatic.

By way of example, the following are considered aromatic heterocyclic groups, because they have an aromatic tautomer:

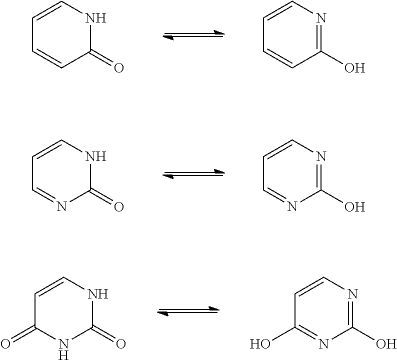 [SMILES: O=c1cccc[nH]1 ; Oc1ccccn1 ; O=c1nccc[nH]1 ; Oc1ncccn1 ; O=c1cc[nH]c(=O)[nH]1 ; Oc1ccnc(O)n1]

For the avoidance of doubt, the term “non-aromatic heterocyclic group” does not exclude heterocyclic groups or moieties which may possess aromatic character only by virtue of mesomeric charge separation.

For example, the following is considered a non-aromatic heterocyclic group, because it does not have an aromatic tautomer:

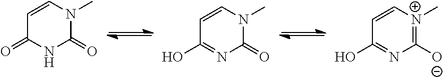

because the last shown structure is not taken into consideration because of mesomeric charge separation.

For the avoidance of doubt, where is stated that a bicyclic or polycyclic group is “saturated” it is to be understood that all of the ring systems within the bicyclic or polycyclic group (excluding any ring systems which are part of or formed by optional substituents) are saturated.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety, such as L:

(i) each hydrogen atom may optionally be replaced by a monovalent substituent independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —CORP; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(═NOR^{β})R^{β}; —C(N₂)R^{β}; —R^{α}—C(═NH)R^{β}; —R^{α}—C(═NH)NH₂; R^{α}—C(═NH)NHR^{β}; —R^{α}—C(═NH)N(R^{β})₂; —R^{α}—C(═NR^{β})R^{β}; —R^{α}—C(═NR^{β})NHR^{β}; R^{α}—C(═NR^{β})N(R^{β})₂; —R^{α}—C(═NOH)R^{β}; —R^{α}—C(═NOR^{β})R^{β}; —R^{α}—C(N₂)R^{β}; —NH—CHO; —NR^{β}—CHO; —NH—COR^{β}; —NR^{β}—COR^{β}; —NH—COOR^{β}; —NR^{β}—COOR^{β}; —NH—C(═NH)R^{β}; NR^{β}—C(═NH)R^{β}; —NH—C(═NH)NH₂; —NR^{β}—C(═NH)NH₂; —NH—C(═NH)NHR^{β}; NR^{β}—C(═NH)NHR^{β}; —NH—C(═NH)N(R^{β})₂; —NR^{β}—C(═NH)N(R^{β})₂; —NH—C(═NR^{β})R^{β}; NR^{β}—C(═NR^{β})R^{β}; —NH—C(═NR^{β})NHR^{β}; —NR^{β}—C(═NR^{β})NHR^{β}; —NH—C(═NR^{β})N(R^{β})₂; NR^{β}—C(═NR^{β})N(R^{β})₂; —NH—C(═NOH)R^{β}; —NR^{β}—C(═NOH)R^{β}; —NH—C(═NOR^{β})R^{β}; NR^{β}—C(═NOR^{β})R^{β}; —CONH₂; —CONHR^{β}; —CON(R^{β})₂; —NH—CONH₂; —NR^{β}—CONH₂; —NH—CONHR^{β}; —NR^{β}—CONHR^{β}; —NH—CON(R^{β})₂; —NR^{β}—CON(R^{β})₂; —R^{α}—NH—CHO; R^{α}—NR^{β}—CHO; —R^{α}—NH—COR^{β}; —R^{α}—NR^{β}—COR^{β}; —R^{α}—NH—COOR^{β}; —R^{α}—NR^{β}—COOR^{β}; R^{α}—NH—C(═NH)R^{β}; —R^{α}—NR^{β}—C(═NH)R^{β}; —R^{α}—NH—C(═NH)NH₂; —R^{α}—NR^{β}—C(═NH)NH₂; R^{α}—NH—C(═NH)NHR^{β}; —R^{α}—NR^{β}—C(═NH)NHR^{β}; —R^{α}—NH—C(═NH)N(R^{β})₂; R^{α}—NR^{β}—C(═NH)N(R^{β})₂; —R^{α}—NH—C(═NR^{β})R^{β}; —R^{α}—NR^{β}—C(═NR^{β})R^{β}; R^{α}—NH—C(═NR^{β})NHR^{β}; —R^{α}—NR^{β}—C(═NR^{β})NHR^{β}; —R^{α}—NH—C(═NR^{β})N(R^{β})₂; R^{α}—NR^{β}—C(═NR^{β})N(R^{β})₂; —R^{α}—NH—C(═NOH)R^{β}; —R^{α}—NR^{β}—C(═NOH)R^{β}; R^{α}—NH—C(═NOR^{β})R^{β}; —R^{α}—NR^{β}—C(═NOR^{β})R^{β}; —R^{α}—CONH₂; —R^{α}—CONHR^{β}; —R^{α}—CON(R^{β})₂; —R^{α}—NH—CONH₂; —R^{α}—NR^{β}—CONH₂; —R^{α}—NH—CONHR^{β}; R^{α}—NR^{β}—CONHR^{β}; —R^{α}—NH—CON(R^{β})₂; —R^{α}—NR^{β}—CON(R^{β})₂; —O—R^{α}—OH; —O—R^{α}—OR^{β}; O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or

(ii) any two hydrogen atoms attached to the same carbon or nitrogen atom may optionally be replaced by a π-bonded substituent independently selected from oxo (═O), ═S, ═NH or ═NR^{β}; and/or

(iii) any sulfur atom may optionally be substituted with one or two π-bonded substituents independently selected from oxo (═O), ═NH or ═NR^{β}; and/or

(iv) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R³ groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R³ may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —CO(C₃-C₇ cycloalkyl), —CO(C₃-C₇ halocycloalkyl), —COO(C₁-C₄ alkyl), —COO(C₁-C₄ haloalkyl), —COO(C₃-C₇ cycloalkyl), —COO(C₃-C₇ halocycloalkyl), halo, —OH, —NH₂, —CN, —C≡CH, oxo (═O), phenyl, halophenyl, or optionally halo-substituted 4- to 6-membered heterocyclic group.

Typically, the compounds of the present invention comprise at most one quaternary ammonium group such as —N⁺(R^{β})₃ or —N⁺(R^{β})₂—.

Where reference is made to a —R^{α}—C(N₂)R^{β} group, what is intended is:

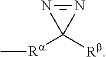

Typically a substituted group comprises 1, 2, 3 or 4 substituents, more typically 1, 2 or 3 substituents, more typically 1 or 2 substituents, and more typically 1 substituent.

Unless stated otherwise, any optional substituent is only attached to the group or moiety which is optionally substituted. For example, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. L¹) must only be attached to the specified group or moiety and may not be attached to a second group or moiety (e.g. L²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

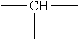

is replaced by

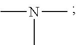

—CH₂— is replaced by —NH—, —O— or —S—; or
—CH₃ is replaced by —NH₂, —OH or —SH;
—CH═ is replaced by —N═;
CH₂═ is replaced by NH═, O═ or S═; or
CH≡ is replaced by N≡;

provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group, what is intended is that:
—CH₂— is replaced by

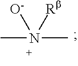

or
—CH₂— is replaced by

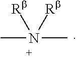

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

For the avoidance of doubt, where it is stated that a compound or a group, such as R¹, R² or L, contains from x to y atoms other than hydrogen or halogen, it is to be understood that the compound or group as a whole, including any optional substituents, contains from x to y atoms other than hydrogen or halogen. Such a compound or group may contain any number of hydrogen or halogen atoms. Similarly, where it is stated that a compound or a group, such as R¹, R² or L, contains from x to y atoms other than hydrogen, it is to be understood that the compound or group as a whole, including any optional substituents, contains from x to y atoms other than hydrogen. Such a compound or group may contain any number of hydrogen atoms.

Unless stated otherwise, any reference to a compound or group is to be considered a reference to all tautomers of that compound or group. For example, any reference to a compound of formula (I) wherein Q¹ and Q² are both N, Q³ is NH, and Q⁴ and Q⁵ are both C, is to be understood to encompass the tautomeric forms (a), (b) and (c) shown below:

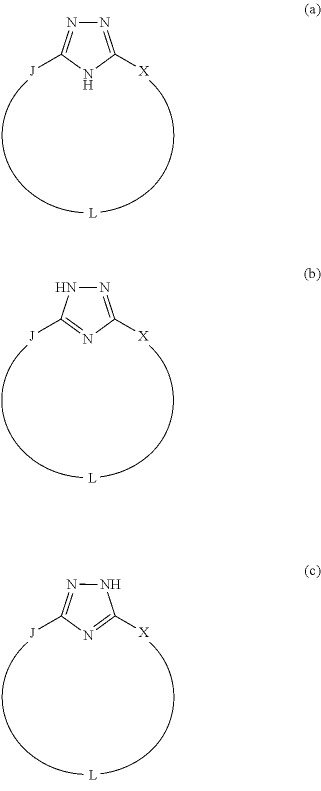

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

As stated, J is —SO—, —SO₂—, —SO(═NH)— or —SO(═NR^{j})—.

In one embodiment, J is —SO₂—, —SO(═NH)— or —SO(═NR^{j})—.

As stated, R^{j} is selected from a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton.

In one embodiment, R^{j} is selected from —CN or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

More typically, R^{j} is selected from —CN or a C₁-C₄ alkyl, C₁-C₄ fluoroalkyl, C₃-C₄ cycloalkyl group or C₃-C₄ fluorocycloalkyl group. For example, R^{j} may be selected from —CN, or a methyl, ethyl, n-propyl, isopropyl or cyclopropyl group, wherein any methyl, ethyl, n-propyl, isopropyl or cyclopropyl group may optionally be substituted with one or more fluoro groups.

Yet more typically, R^{j} is —CN.

In one embodiment, J is —SO—, —SO₂—, —SO(═NH)— or —SO(═NCN)—. More typically, J is —SO—, —SO₂— or —SO(═NH)—. More typically still, J is —SO₂— or —SO(═NH)—. Most typically, J is —SO₂—.

As stated:
Q¹ and Q² are each independently selected from O, S, N, NH, NR^{q}, CH, CHal or CR^{qq}, provided that at least one of Q¹ and Q² is selected from N, NH and NR^{q};
Q³ is selected from O, S, N, NH and NR^{q}; and
Q⁴ and Q⁵ are each independently selected from C and N, provided that at least one of Q⁴ and Q⁵ is C;
such that ring Q is a 5-membered heteroaryl ring.

As will be understood, since at least one of Q¹ and Q² is selected from N, NH and NR^{q}, and at least one of Q⁴ and Q⁵ is C, the 5-membered heteroaryl ring structure of ring Q must contain at least one carbon atom and at least one nitrogen atom. Typically, the 5-membered heteroaryl ring structure of ring Q contains at least two carbon atoms and at least one nitrogen atom. More typically, the 5-membered heteroaryl ring structure of ring Q contains at least two carbon atoms and at least two nitrogen atoms.

As stated, each Hal is independently selected from F, Cl, Br or I. In one embodiment, each Hal is independently selected from F, Cl or Br. More typically, each Hal is independently selected from F or C1. Most typically, each Hal is F.

As stated, each R^{q} is independently selected from a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton.

In one embodiment, each R^{q} is independently selected from a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In a further embodiment, each R^{q} is independently selected from a C₁-C₄ alkyl, C₁-C₄ fluoroalkyl, C₃-C₄ cycloalkyl group or C₃-C₄ fluorocycloalkyl group. For example, each R^{q} may independently be selected from a methyl, ethyl, n-propyl, isopropyl or cyclopropyl group, wherein any methyl, ethyl, n-propyl, isopropyl or cyclopropyl group may optionally be substituted with one or more fluoro groups.

In yet another embodiment, each R^{q} is independently selected from a methyl group, wherein the methyl group may optionally be substituted with one or more fluoro groups.

As stated, each R^{qq} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton.

In one embodiment, each R^{qq} is independently selected from —CN, —OH, —NH₂, or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In a further embodiment, each R^{qq} is independently selected from —OH, —NH₂, or a C₁-C₄ alkyl, C₁-C₄ fluoroalkyl, C₃-C₄ cycloalkyl group or C₃-C₄ fluorocycloalkyl group.

In yet another embodiment, each R^{qq} is independently selected from a methyl group, wherein the methyl group may optionally be substituted with one or more fluoro groups.

In one embodiment, Q¹ and Q² are each independently selected from O, S, N, NH, CH or CF, provided that at least one of Q¹ and Q² is selected from N or NH.

In another embodiment, Q¹ and Q² are each independently selected from N, NH and NR^{q}.

Most typically, Q¹ and Q² are both N.

In one embodiment, Q³ is selected from O, S, N and NH.

In another embodiment, Q³ is selected from N, NH and NR^{q}.

Most typically, Q³ is NH.

In one embodiment, Q⁴ is C or N and Q⁵ is C. Most typically, Q⁴ and Q⁵ are both C.

As stated, X is —O—, —NH—, —NR^{x}—, —CH₂—, —CH(Hal)-, —C(Hal)₂-, —CH(R^{xx})—, —C(Hal)(R^{xx})— or —C(R^{xx})₂—, wherein:
R^{x} is independently selected from a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton;
each R^{xx} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton, or any two R^{xx} may, together with the carbon atom to which they are attached, form a saturated or unsaturated cyclic group, wherein the cyclic group may optionally be substituted; and
each Hal is independently selected from F, Cl, Br or I.

In one embodiment, R^{x} is independently selected from a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In a further embodiment, R^{x} is independently selected from a C₁-C₄ alkyl, C₁-C₄ fluoroalkyl, C₃-C₄ cycloalkyl group or C₃-C₄ fluorocycloalkyl group. For example, each R^{x} may independently be selected from a methyl, ethyl, n-propyl, isopropyl or cyclopropyl group, wherein any methyl, ethyl, n-propyl, isopropyl or cyclopropyl group may optionally be substituted with one or more fluoro groups.

In yet another embodiment, R^{x} is a methyl group, wherein the methyl group may optionally be substituted with one or more fluoro groups.

In one embodiment, each R^{xx} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton. Typically in such an embodiment, each R^{xx} is independently selected from —CN, —OH, —NH₂, or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In another embodiment, two R^{xx}, together with the carbon atom to which they are attached, form a saturated or unsaturated cyclic group, wherein the cyclic group may optionally be substituted. As will be understood, such a cyclic group is attached to the ring formed by -J-, ring Q, —X— and -L- as a spiro-group. Typically in such an embodiment, two R^{xx}, together with the carbon atom to which they are attached, form a 3- to 7-membered saturated cyclic group, wherein the saturated cyclic group may optionally include one or two ring heteroatoms independently selected from N and O in its carbon skeleton, and wherein the saturated cyclic group may optionally be substituted with one or more groups independently selected from fluoro, chloro, —CN, —OH, —NH₂ and oxo (═O).

In a further embodiment, each R^{xx} is independently selected from a C₁-C₄ alkyl, C₁-C₄ fluoroalkyl, C₃-C₄ cycloalkyl group or C₃-C₄ fluorocycloalkyl group, or two R^{xx} may, together with the carbon atom to which they are attached, form a 3- or 4-membered cycloalkyl group, or form an oxetanyl group, wherein the 3- or 4-membered cycloalkyl group or the oxetanyl group may optionally be fluoro-substituted. For example, each R^{xx} may independently be selected from a methyl, ethyl, n-propyl, isopropyl or cyclopropyl group, or two R^{xx} may, together with the carbon atom to which they are attached, form a cyclopropyl group, wherein any methyl, ethyl, n-propyl, isopropyl or cyclopropyl group may optionally be substituted with one or more fluoro groups.

In a yet another embodiment, each R^{xx} is a methyl group, wherein the methyl group may optionally be substituted with one or more fluoro groups.

Typically, where X is —O—, —NH— or —NR^{x}—, Q⁵ is C.

In one embodiment, X is —O—, —NH—, —NR^{x}—, —CH₂—, —CH(F)—, —CH(Cl)— or —CH(R^{xx})—, wherein typically:
R^{x} is a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O); and
R^{xx} is selected from —CN, —OH, —NH₂, or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In another embodiment, X is —O—, —NH— or —NR^{x}—. Typically in such an embodiment, R^{x} is a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In a further embodiment, X is —NH— or —NR^{x}—. Typically in such an embodiment, R^{x} is a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

In a typical embodiment, X is —O— or —NH—. Most typically, in accordance with any of the above embodiments, X is —NH—.

As stated, L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N, O and S in its carbon skeleton.

In one embodiment, L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N and O in its carbon skeleton.

Typically the hydrocarbylene group of L includes at least one cyclic group.

Typically L, including any optional substituents, contains in total from 4 to 50 carbon, nitrogen, oxygen and sulfur atoms. More typically L, including any optional substituents, contains in total from 10 to 40 carbon, nitrogen, oxygen and sulfur atoms.

More typically still L, including any optional substituents, contains in total from 20 to 35 carbon, nitrogen, oxygen and sulfur atoms.

As stated, -J-, ring Q, —X— and -L- together form a ring, such that the minimum single ring size that encompasses all or part of each of -J-, ring Q, —X— and -L- is from 8 to 30 atoms. Typically, the minimum single ring size that encompasses all or part of each of -J-, ring Q, —X— and -L- is from 12 to 24 atoms. More typically, the minimum single ring size that encompasses all or part of each of -J-, ring Q, —X— and -L- is from 14 to 20 atoms.

As will be understood, the compounds of the invention may be bicyclic ring systems, or may be tricyclic or polycyclic ring systems, for example due to the presence of cyclic groups within -L-. However, the compounds of formula (I) must meet the criteria that -J-, ring Q, —X— and -L- together form a ring, such that the minimum single ring size that encompasses all or part of each of -J-, ring Q, —X— and -L- is from 8 to 30 atoms. It will be appreciated that for bicyclic, tricyclic or polycyclic ring systems, alternate single ring sizes that encompass all or part of each of -J-, ring Q, —X— and -L- may be identified; it is the smallest of these possible alternate single ring sizes that is relevant for determining the minimum ring size. By way of example, consider the bicyclic structure (A) below:

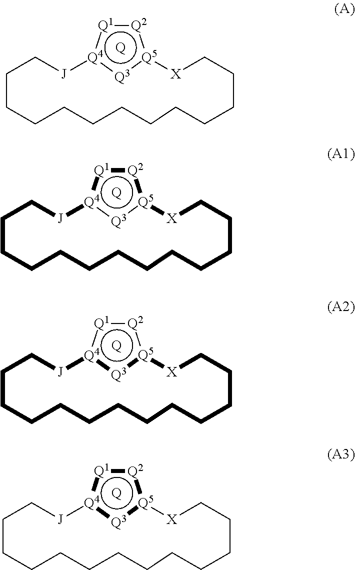

Three single ring sizes within the bicyclic structure may be identified, namely a 19-atom ring illustrated in bold in structure (A1), a 18-atom ring illustrated in bold in structure (A2), and a 5-atom ring illustrated in bold in structure (A3). Of these three single ring sizes, only the two rings illustrated in bold in (A1) and (A2) encompass all or part of each of -J-, ring Q, —X— and -L-. Of these two rings, the ring illustrated in bold in structure (A2) is the smallest. Hence for structure (A), the minimum single ring size that encompasses all or part of each of -J-, ring Q, —X— and -L- is 18 atoms.

In one embodiment of the first aspect of the invention, the compound has the formula (Ia):

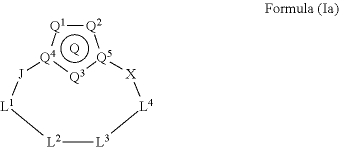

wherein:
J, Q¹, Q², Q³, Q⁴, Q⁵, ring Q and X are as previously defined;
-J-, ring Q, —X—, -L¹-, -L²-, -L³- and -L⁴- together form a ring, such that the minimum single ring size that encompasses all or part of each of -J-, ring Q, —X—, -L¹-, -L²-, -L³- and -L⁴- is from 8 to 30 atoms;
L¹ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents;
L² is an alkylene, alkenylene or alkynylene group, wherein the alkylene, alkenylene or alkynylene group may be straight-chained or branched, or be or include one or more cyclic groups, wherein one or more carbon atoms in the backbone of the alkylene, alkenylene or alkynylene group may optionally be replaced by one or more heteroatoms independently selected from N, O and S, and wherein the alkylene, alkenylene or alkynylene group may optionally be substituted with one or more monovalent substituents, and/or one or more π-bonded substituents;
L³ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents; and
L⁴ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents.

For the avoidance of doubt:
where L¹ is a divalent 3- to 7-membered monocyclic group, a divalent 5- to 12-membered bicyclic group, or a divalent 7- to 18-membered tricyclic group, a ring atom of the monocyclic, bicyclic or tricyclic group is directly attached to the sulfur atom of J, and the same or a different ring atom of the monocyclic, bicyclic or tricyclic group is directly attached to L²;
where L³ is a divalent 3- to 7-membered monocyclic group, a divalent 5- to 12-membered bicyclic group, or a divalent 7- to 18-membered tricyclic group, a ring atom of the monocyclic, bicyclic or tricyclic group of L³ is directly attached to a ring atom of the monocyclic, bicyclic or tricyclic group of L⁴, and the same or a different ring atom of the monocyclic, bicyclic or tricyclic group of L³ is directly attached to L²; and
a ring atom of the divalent 3- to 7-membered monocyclic group, divalent 5- to 12-membered bicyclic group, or divalent 7- to 18-membered tricyclic group of L⁴ is directly attached to the oxygen, carbon or nitrogen atom of X that is in turn directly attached to Q⁵, and the same or a different ring atom of the monocyclic, bicyclic or tricyclic group of L⁴ is either (i) directly attached to a ring atom of the divalent 3- to 7-membered monocyclic group, divalent 5- to 12-membered bicyclic group, or divalent 7- to 18-membered tricyclic group of L³, or (ii), where L³ is a bond, directly attached to L².

Where L¹ is a cyclic group, such as a divalent 3- to 7-membered monocyclic group, a divalent 5- to 12-membered bicyclic group, or a divalent 7- to 18-membered tricyclic group, the ring atom of the cyclic group that is directly attached to the sulfur atom of J may be a nitrogen or a carbon atom.

Typically, the ring atom of the divalent 3- to 7-membered monocyclic group, divalent 5- to 12-membered bicyclic group, or divalent 7- to 18-membered tricyclic group of L⁴ that is directly attached to the oxygen, carbon or nitrogen atom of X is a carbon atom.

As stated, -J-, ring Q, —X—, -L¹-, -L²-, -L³- and -L⁴- together form a ring, such that the minimum single ring size that encompasses all or part of each of -J-, ring Q, —X—, -L¹-, -L²-, -L³- and -L⁴- is from 8 to 30 atoms. Typically, the minimum single ring size that encompasses all or part of each of -J-, ring Q, —X—, -L¹-, -L²-, -L³- and -L⁴- is from 12 to 24 atoms. More typically, the minimum single ring size that encompasses all or part of each of -J-, ring Q, —X—, -L¹-, -L²-, -L³- and -L⁴- is from 14 to 20 atoms.

As stated, L¹ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or r-bonded substituents. More typically, L¹ is a bond, a divalent 3- to 7-membered monocyclic group, a divalent 7- to 11-membered bicyclic group, or a divalent 9- to 16-membered tricyclic group, any of which may optionally be substituted with one or more monovalent substituents and/or n-bonded substituents.

In one embodiment, L¹ is a bond.

In one embodiment, where L¹ is a bond, the atom of L² that is directly attached to the sulfur atom of J is a nitrogen or a carbon atom.

In another embodiment, L¹ is a divalent 3- to 7-membered monocyclic group, a divalent 5- to 12-membered bicyclic group, or a divalent 7- to 18-membered tricyclic group, any of which may optionally be substituted with one or more monovalent substituents and/or n-bonded substituents. More typically in such an embodiment, L¹ is a divalent 3- to 7-membered monocyclic group, a divalent 7- to 11-membered bicyclic group, or a divalent 9- to 16-membered tricyclic group, any of which may optionally be substituted with one or more monovalent substituents and/or n-bonded substituents.

In one embodiment, L¹ is a divalent 3- to 7-membered monocyclic group, or a divalent 5- to 12-membered bicyclic group, either of which may optionally be substituted with one or more monovalent substituents and/or n-bonded substituents. Typically in such an embodiment, L¹ is a divalent 3- to 7-membered monocyclic group, or a divalent 7- to 11-membered bicyclic group, either of which may optionally be substituted with one or more monovalent substituents and/or n-bonded substituents.

In one aspect of such an embodiment, L¹ is a divalent phenyl, naphthalene, 5- or 6-membered monocyclic heteroaryl, or 8- to 10-membered bicyclic heteroaryl group, any of which may optionally be substituted with one or more monovalent substituents. More typically in such an embodiment, L¹ is a divalent phenyl, or 5- or 6-membered monocyclic heteroaryl group, any of which may optionally be substituted with one or more monovalent substituents.

In another aspect of such an embodiment, L¹ is a divalent fused 7- to 11-membered bicyclic group, wherein a first ring in the bicyclic structure is aromatic and a second ring in the bicyclic structure is non-aromatic, wherein the first ring may optionally be substituted with one or more monovalent substituents, and wherein the second ring may optionally be substituted with one or more monovalent substituents and/or π-bonded substituents. Typically in such an embodiment, the first ring is a 5- or 6-membered ring and the second ring is a 5- or 6-membered ring.

In yet another aspect of such an embodiment, L¹ is a divalent saturated 3- to 7-membered monocyclic group, or a divalent saturated 5- to 12-membered bicyclic group, any of which may optionally be substituted with one or more monovalent substituents and/or π-bonded substituents. Typically in such an aspect, L¹ is a divalent saturated 3- to 7-membered monocyclic group, or a divalent saturated 7- to 11-membered bicyclic group, any of which may optionally be substituted with one or more monovalent substituents and/or π-bonded substituents. For example, L¹ may be a 3- to 7-membered monocyclic cycloalkylene group, a divalent saturated 4- to 7-membered monocyclic heterocyclic group, a 7- to 11-membered bicyclic cycloalkylene group, or a divalent saturated 7- to 11-membered bicyclic heterocyclic group, any of which may optionally be substituted with one or more monovalent substituents and/or π-bonded substituents.

In one embodiment, L¹ is a divalent saturated 3- to 7-membered monocyclic group, which may optionally be substituted with one or more monovalent substituents and/or r-bonded substituents. In one aspect of such an embodiment, L¹ is a divalent saturated 4- to 7-membered monocyclic heterocyclic group, which may optionally be substituted with one or more monovalent substituents and/or π-bonded substituents.

In another embodiment, L¹ is a divalent saturated 7- to 11-membered fused bicyclic group, which may optionally be substituted with one or more monovalent substituents and/or π-bonded substituents. In one aspect of such an embodiment, L¹ is a divalent saturated 7- to 11-membered fused bicyclic heterocyclic group, which may optionally be substituted with one or more monovalent substituents and/or π-bonded substituents.

In yet another embodiment, L¹ is a divalent 5- to 12-membered spiro bicyclic group, which may optionally be substituted with one or more monovalent substituents and/or r-bonded substituents. For example, L¹ may be a divalent saturated 7- to 11-membered spiro bicyclic group, which may optionally be substituted with one or more monovalent substituents and/or π-bonded substituents. In one aspect of such an embodiment, L¹ is a divalent saturated 7- to 11-membered spiro bicyclic heterocyclic group, which may optionally be substituted with one or more monovalent substituents and/or π-bonded substituents.

In a further embodiment, L¹ is a divalent 5- to 12-membered bridged bicyclic group, which may optionally be substituted with one or more monovalent substituents and/or r-bonded substituents. For example, L¹ may be a divalent saturated 7- to 11-membered bridged bicyclic group, which may optionally be substituted with one or more monovalent substituents and/or π-bonded substituents. In one aspect of such an embodiment, L¹ is a divalent saturated 7- to 11-membered bridged bicyclic heterocyclic group, which may optionally be substituted with one or more monovalent substituents and/or π-bonded substituents.

As stated, L² is an alkylene, alkenylene or alkynylene group, wherein the alkylene, alkenylene or alkynylene group may be straight-chained or branched, or be or include one or more cyclic groups, wherein one or more carbon atoms in the backbone of the alkylene, alkenylene or alkynylene group may optionally be replaced by one or more heteroatoms independently selected from N, O and S, and wherein the alkylene, alkenylene or alkynylene group may optionally be substituted with one or more monovalent substituents, and/or one or more π-bonded substituents.

In one embodiment, L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N, O and S, and wherein the alkylene or alkenylene group may optionally be substituted with one or more monovalent substituents, and/or one or more π-bonded substituents.

In another embodiment, L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more monovalent substituents, and/or one or more π-bonded substituents.

In a further embodiment, L² is an alkylene or alkenylene group, wherein the alkylene or alkenylene group may be straight-chained or branched, or include a single cyclic group, wherein one or more carbon atoms in the backbone of the alkylene or alkenylene group may optionally be replaced by one or more heteroatoms independently selected from N and O, and wherein the alkylene or alkenylene group may optionally be substituted with one or more monovalent substituents, and/or one or more π-bonded substituents.

In another embodiment, L² is an alkylene or alkenylene group, wherein the alkylene or alkenylene group is straight-chained or branched, wherein one or more carbon atoms in the backbone of the alkylene or alkenylene group may optionally be replaced by one or more heteroatoms independently selected from N and O, and wherein the alkylene or alkenylene group may optionally be substituted with one or more monovalent substituents, and/or one or more π-bonded substituents.

In one embodiment, L² is an alkylene group, wherein the alkylene group may be straight-chained or branched, or include a single cyclic group, wherein the alkylene group optionally includes one, two or three heteroatoms independently selected from O and N in its carbon skeleton, and wherein the alkylene group may optionally be substituted with one or more monovalent substituents, and/or one or more π-bonded substituents. Typically in such an embodiment, the single cyclic group (where present) is monocyclic.

In another embodiment, L² is a straight-chained alkylene group, wherein the straight-chained alkylene group optionally includes one or two heteroatoms independently selected from O and N in its carbon skeleton, and wherein the straight-chained alkylene group may optionally be substituted with one or more monovalent substituents, and/or one or more π-bonded substituents.

Typically, any alkylene, alkenylene or alkynylene group of L² includes at least one heteroatom independently selected from O and N in its carbon skeleton. In one embodiment, the atom of L² that is directly attached to L³ is O or N. In a further embodiment, the atom of L² that is directly attached to L³ is O.

Typically L², including any optional substituents, contains in total from 1 to 20 carbon, nitrogen, oxygen and sulfur atoms. More typically L², including any optional substituents, contains in total from 2 to 15 carbon, nitrogen, oxygen and sulfur atoms. More typically still L², including any optional substituents, contains in total from 2 to 10 carbon, nitrogen, oxygen and sulfur atoms. Yet more typically L², including any optional substituents, contains in total from 3 to 7 carbon, nitrogen, oxygen and sulfur atoms.

Typically, L² has a chain length of from 1 to 15 atoms. More typically, L² has a chain length of from 2 to 12 atoms. More typically still, L² has a chain length of from 2 to 8 atoms. Yet more typically, L² has a chain length of from 3 to 6 atoms. As will be understood, the “chain length” of L² refers to the number of atoms of L² that are bonded to each other in a continuous chain between L¹ and L³, as measured by the shortest route. By way of example, structure (C) has a chain length of 3 atoms, whereas structure (D) has a chain length of 5 atoms:

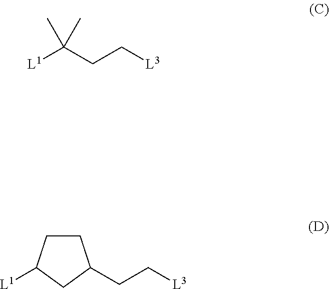

As stated, L³ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or r-bonded substituents. More typically, L³ is a bond, a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, L³ is a bond.

In another embodiment, L³ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. More typically in such an embodiment, L³ is a divalent 3- to 7-membered monocyclic group, a divalent 7- to 11-membered bicyclic group, or a divalent 9- to 16-membered tricyclic group, any of which may optionally be substituted with one or more monovalent substituents and/or π-bonded substituents.

In one embodiment, L³ is a divalent 3- to 7-membered monocyclic group, or a divalent 7- to 11-membered bicyclic group, either of which may optionally be substituted with one or more monovalent substituents and/or n-bonded substituents. In one aspect of such an embodiment, L³ is a divalent phenyl, naphthalene, 5- or 6-membered monocyclic heteroaryl, or 8- to 10-membered bicyclic heteroaryl group, any of which may optionally be substituted with one or more monovalent substituents. For example, L³ may be a divalent phenyl or 5- or 6-membered monocyclic heteroaryl group, any of which may optionally be substituted with one or more monovalent substituents. More typically, L³ is a divalent phenyl or 6-membered monocyclic heteroaryl group, such as a divalent phenyl or divalent pyridinyl group, any of which may optionally be substituted with one or more monovalent substituents. Most typically, L³ is a divalent pyridinyl group, which may optionally be substituted with one or more monovalent substituents.

As stated, L⁴ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents. More typically, L⁴ is a divalent 3- to 7-membered monocyclic group, a divalent 7- to 11-membered bicyclic group, or a divalent 9- to 16-membered tricyclic group, any of which may optionally be substituted with one or more monovalent substituents and/or n-bonded substituents.

In one embodiment, the ring of the divalent monocyclic, bicyclic or tricyclic group of L⁴ that is directly attached to X is aromatic. For example, L⁴ may be selected from:

(i) a divalent phenyl or 5- or 6-membered heteroaryl group, wherein the divalent phenyl or 5- or 6-membered heteroaryl group may optionally be substituted with one or more monovalent substituents; or

(ii) a divalent 7- to 11-membered fused bicyclic group, wherein a first ring in the bicyclic structure is aromatic, and a second ring in the bicyclic structure is aromatic or non-aromatic, wherein X is directly attached to a ring atom of the first ring, wherein L³ is directly attached to a ring atom of either the first or the second ring, and wherein the divalent 7- to 11-membered fused bicyclic group may optionally be substituted with one or more monovalent substituents and/or n-bonded substituents; or

(iii) a divalent 9- to 16-membered fused tricyclic group, wherein a first ring in the tricyclic structure is aromatic, a second ring in the tricyclic structure is aromatic or non-aromatic, and a third ring in the tricyclic structure is aromatic or non-aromatic, wherein X is directly attached to a ring atom of the first ring, wherein L³ is directly attached to a ring atom of any of the first, second or third rings, and wherein the divalent 9- to 16-membered fused tricyclic group may optionally be substituted with one or more monovalent substituents and/or π-bonded substituents.

In one embodiment, L⁴ is a divalent 3- to 7-membered monocyclic group, or a divalent 7- to 11-membered fused bicyclic group, either of which may optionally be substituted with one or more monovalent substituents and/or π-bonded substituents. Typically in such an embodiment, L⁴ is a divalent 5- or 6-membered monocyclic group, or a divalent 8- to 10-membered fused bicyclic group, either of which may optionally be substituted with one or more monovalent substituents and/or π-bonded substituents. For example, L⁴ may be a phenyl or 5- or 6-membered heteroaryl group, optionally wherein a 5- or 6-membered cyclic group is fused to the phenyl or 5- or 6-membered heteroaryl group, wherein X is directly attached to a ring atom of the phenyl or 5- or 6-membered heteroaryl group, wherein L³ is directly attached to a ring atom of any of the phenyl, 5-or 6-membered heteroaryl or fused 5- or 6-membered cyclic groups, wherein the phenyl or 5- or 6-membered heteroaryl group may optionally be further substituted with one or more monovalent substituents, and wherein the fused 5- or 6-membered cyclic group may optionally be substituted with one or more monovalent substituents and/or π-bonded substituents.

In one aspect of such an embodiment, X and L³ are directly attached to the same ring of L⁴. For example, L⁴ may be a phenyl or 5- or 6-membered heteroaryl group, optionally wherein a 5- or 6-membered cyclic group is fused to the phenyl or 5- or 6-membered heteroaryl group, wherein X is directly attached to a first ring atom of the phenyl or 5-or 6-membered heteroaryl group, wherein L³ is directly attached to a second ring atom of the phenyl or 5- or 6-membered heteroaryl group, wherein the phenyl or 5- or 6-membered heteroaryl group may optionally be further substituted with one or more monovalent substituents, and wherein the fused 5- or 6-membered cyclic group may optionally be substituted with one or more monovalent substituents and/or π-bonded substituents. Typically, L⁴ is a phenyl or a 6-membered heteroaryl group, optionally wherein a 5- or 6-membered cyclic group is fused to the phenyl or 6-membered heteroaryl group, wherein X is directly attached to a first ring atom of the phenyl or 6-membered heteroaryl group, wherein L³ is directly attached to a second ring atom of the phenyl or 6-membered heteroaryl group, wherein the phenyl or 6-membered heteroaryl group may optionally be further substituted with one or more monovalent substituents, and wherein the fused 5- or 6-membered cyclic group may optionally be substituted with one or more monovalent substituents and/or π-bonded substituents. Typically, where X and L³ are directly attached to the same ring, the ring atom of L⁴ that is directly attached to L³ is at the α-position relative to the ring atom of L⁴ that is directly attached to X. Typically in such an embodiment, the ring to which X and L³ are directly attached is further substituted at the α′-position, typically wherein the substituent at the α′-position comprises at least one carbon atom and/or forms part of a ring structure that is ortho-fused to the ring to which X and L³ are directly attached across the α′,β′ positions. For example, L⁴ may be a divalent phenyl or 5- or 6-membered heteroaryl group (typically a phenyl or a 6-membered heteroaryl group), wherein the ring atom of L⁴ that is directly attached to L³ is at the α-position relative to the ring atom of L⁴ that is directly attached to X, wherein either:
(i) a 5- or 6-membered cyclic group is fused to the divalent phenyl or 5- or 6-membered heteroaryl group across the α′,β′ positions, wherein the fused 5- or 6-membered cyclic group may optionally be substituted with one or more monovalent substituents and/or n-bonded substituents; or
(ii) the divalent phenyl or 5- or 6-membered heteroaryl group is substituted at the α′-position with a monovalent substituent comprising at least one carbon atom;

and wherein the divalent phenyl or 5- or 6-membered heteroaryl group may optionally be further substituted with one or more monovalent substituents.

As used herein, the nomenclature α, β, α′,β′ refers to the position of the atoms of a cyclic group, such as L⁴, relative to the specified point of attachment of the cyclic group to the remainder of the molecule. For example, where L⁴ is a divalent 2,3-dihydro-1H-indenyl moiety attached to X at the 4-position and to L³ at the 5-position, the α, β, α′ and β′ positions relative to the ring atom of L⁴ that is directly attached to X are as follows:

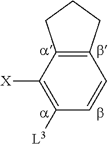

For the avoidance of doubt, where it is stated that a cyclic group, such as a phenyl or a heteroaryl group, is substituted at the α and/or α′ positions, it is to be understood that one or more hydrogen atoms at the α and/or α′ positions respectively are replaced by one or more substituents, such as any optional substituent as defined herein. Unless stated otherwise, the term “substituted” does not include the replacement of one or more ring carbon atoms by one or more ring heteroatoms.

Typically, where X and L³ are directly attached to the same ring of L⁴, L³ is a divalent 3- to 7-membered monocyclic group, or a divalent 7- to 11-membered bicyclic group, either of which may optionally be substituted with one or more monovalent substituents and/or π-bonded substituents. For example, where X and L³ are directly attached to the same ring of L⁴, L³ may be a divalent phenyl, naphthalene, 5- or 6-membered monocyclic heteroaryl, or 8- to 10-membered bicyclic heteroaryl group, any of which may optionally be substituted with one or more monovalent substituents.

Typically in such an embodiment, where L³ is a divalent 7- to 11-membered bicyclic group such as a naphthalene or 8- to 10-membered bicyclic heteroaryl group, L² and L⁴ are directly attached to the same ring of L³.

More typically, where X and L³ are directly attached to the same ring of L⁴, L³ is a divalent phenyl or 5- or 6-membered monocyclic heteroaryl group, any of which may optionally be substituted with one or more monovalent substituents. For example, L³ may be a divalent phenyl or 6-membered monocyclic heteroaryl group, such as a divalent phenyl or divalent pyridinyl group, any of which may optionally be substituted with one or more monovalent substituents. Most typically, L³ is a divalent pyridinyl group, which may optionally be substituted with one or more monovalent substituents.

In another embodiment, L⁴ is a divalent 7- to 11-membered fused bicyclic group, or a divalent 9- to 16-membered fused tricyclic group, either of which may optionally be substituted with one or more monovalent substituents and/or π-bonded substituents. Typically in such an embodiment, L⁴ is a divalent 8- to 10-membered fused bicyclic group or a divalent 11- to 14-membered fused tricyclic group, either of which may optionally be substituted with one or more monovalent substituents and/or π-bonded substituents. For example, L⁴ may be a phenyl or 5- or 6-membered heteroaryl group, wherein a first 5- or 6-membered cyclic group is fused to the phenyl or 5- or 6-membered heteroaryl group, optionally wherein a second 5- or 6-membered cyclic group is fused to the phenyl or 5- or 6-membered heteroaryl group, wherein X is directly attached to a ring atom of the phenyl or 5- or 6-membered heteroaryl group, wherein L³ is directly attached to a ring atom of any of the phenyl, 5- or 6-membered heteroaryl or fused 5- or 6-membered cyclic groups, wherein the phenyl or 5- or 6-membered heteroaryl group may optionally be further substituted with one or more monovalent substituents, and wherein the fused 5- or 6-membered cyclic groups may optionally be substituted with one or more monovalent substituents and/or π-bonded substituents.

In one aspect of such an embodiment, X and L³ are directly attached to different rings within the bicyclic or tricyclic group. For example, L⁴ may be a phenyl or 5- or 6-membered heteroaryl group, wherein a ring atom of the phenyl or 5- or 6-membered heteroaryl group is directly attached to X, wherein a first 5- or 6-membered cyclic group is fused to the phenyl or 5- or 6-membered heteroaryl group, wherein a ring atom of the first fused 5- or 6-membered cyclic group is directly attached to L³, wherein optionally a second 5- or 6-membered cyclic group is fused to the phenyl or 5- or 6-membered heteroaryl group, wherein the phenyl or 5- or 6-membered heteroaryl group may optionally be further substituted with one or more monovalent substituents, and wherein either fused 5- or 6-membered cyclic group may optionally be substituted with one or more monovalent substituents and/or π-bonded substituents.

Typically, where X and L³ are directly attached to different rings within the divalent bicyclic or tricyclic group of L⁴, L³ is a bond, such that X and L² are directly attached to different rings within the bicyclic or tricyclic group. Typically, X is directly attached to a ring atom of a first ring of the bicyclic or tricyclic group, a second ring of the bicyclic or tricyclic group is ortho-fused to the first ring across the α,β positions of the first ring, relative to the ring atom of the first ring that is directly attached to X, and L³ (or L² where L³ is a bond) is directly attached to a ring atom of the second ring that is not also a ring atom of the first ring. For example, L⁴ may be a phenyl or 5- or 6-membered heteroaryl group (typically a phenyl or a 6-membered heteroaryl group), wherein a ring atom of the phenyl or 5- or 6-membered heteroaryl group is directly attached to X, wherein a first 5- or 6-membered cyclic group is fused to the phenyl or 5- or 6-membered heteroaryl group across the α,β positions of the phenyl or 5- or 6-membered heteroaryl group, relative to the ring atom that is directly attached to X, wherein a ring atom of the first fused 5- or 6-membered cyclic group is directly attached to L², wherein either
(i) a second 5- or 6-membered cyclic group is fused to the phenyl or 5- or 6-membered heteroaryl group across the α′,β′ positions; or
(ii) the phenyl or 5- or 6-membered heteroaryl group is substituted at the α′-position with a monovalent substituent comprising at least one carbon atom;

wherein the phenyl or 5- or 6-membered heteroaryl group may optionally be further substituted with one or two monovalent substituents, and wherein either fused 5- or 6-membered cyclic group may optionally be substituted with one or more monovalent substituents and/or π-bonded substituents.

Where L¹, L², L³ or L⁴ is substituted with one or more monovalent substituents, the monovalent substituents may be independently selected from any monovalent substituent as discussed above. In one embodiment, where L¹, L², L³ or L⁴ is substituted with one or more monovalent substituents, each monovalent substituent is independently selected from a halo, C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, wherein:
each R¹¹ is independently selected from a bond, or a C₁-C₄ alkylene group, wherein the C₁-C₄ alkylene group may be straight-chained or branched, or be or include a C₃-C₄ cycloalkylene group, and wherein the C₁-C₄ alkylene group may optionally be substituted with one or more halo groups;
each R¹² is independently selected from a 3- to 6-membered cyclic group, wherein the 3- to 6-membered cyclic group may optionally be substituted with one or more halo groups and/or one, two or three substituents independently selected from —CN, —R¹⁴, —OH, —OR¹⁴, —NH₂, —NHR¹⁴ and —N(R¹⁴)₂;
each R¹³ is independently selected from hydrogen or a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups and/or one, two or three substituents independently selected from —CN, —R¹⁴, —OH, —OR¹⁴, —NH₂, —NHR¹⁴ and —N(R¹⁴)₂, or any two R¹³ attached to the same nitrogen atom may together form a C₂-C₅ alkylene or C₂-C₅ haloalkylene group; and
each R¹⁴ is independently selected from a C₁-C₄ alkyl or C₁-C₄ haloalkyl group.

Where L¹, L², L³ or L⁴ is substituted with one or more π-bonded substituents, the π-bonded substituents may be independently selected from any π-bonded substituent as discussed above. In one embodiment, where L¹, L², L³ or L⁴ is substituted with one or more π-bonded substituents, each π-bonded substituent is independently selected from ═O or ═NR¹³, wherein R¹³ is as defined above.

In one embodiment of the first aspect of the invention, the compound has the formula (Ib):

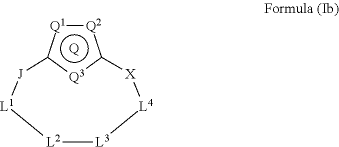

wherein:
J is —SO—, —SO₂—, —SO(═NH)— or —SO(═NR^{j})—;
Q¹ and Q² are each independently selected from N, NH and NR^{q};
Q³ is selected from O, S, N and NH;
ring Q is aromatic;
X is —O—, —NH—, —NR^{x}—, —CH₂—, —CH(F)—, —CH(Cl)— or —CH(R^{xx})—;
L¹, L², L³ and L⁴ are as previously defined;
-J-, ring Q, —X—, -L¹-, -L²-, -L³- and -L⁴- together form a ring, such that the minimum single ring size that encompasses all or part of each of -J-, ring Q, —X—, -L¹-, -L²-, -L³- and -L⁴- is from 8 to 30 atoms;
R^{j} is selected from —CN or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O);
each R^{q} and R^{x} is independently selected from a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O); and
R^{xx} is selected from —CN, —OH, —NH₂, or a saturated C₁-C₆ hydrocarbyl group, wherein the saturated C₁-C₆ hydrocarbyl group may be straight-chained or branched, or be or include a cyclic group, wherein the saturated C₁-C₆ hydrocarbyl group may optionally include one or two heteroatoms independently selected from N and O in its carbon skeleton, and wherein the saturated C₁-C₆ hydrocarbyl group may optionally be substituted with one or more groups independently selected from fluoro, chloro, —CN, —OH, —NH₂ and oxo (═O).

Typically, where the compound has the formula (Ib), J is —SO—, —SO₂—, —SO(═NH)— or —SO(═NCN)—.

Typically, the minimum single ring size that encompasses all or part of each of -J-, ring Q, —X—, -L¹-, -L²-, -L³- and -L⁴- is from 12 to 24 atoms. More typically, the minimum single ring size that encompasses all or part of each of -J-, ring Q, —X—, -L¹-, -L²-, -L³-and -L⁴- is from 14 to 20 atoms.

In another embodiment of the first aspect of the invention, the compound has the formula (Ic):

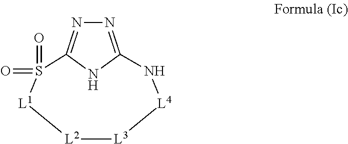

wherein L¹, L², L³ and L⁴ are as previously defined, and wherein the minimum single ring size that encompasses all or part of each of -L¹-, -L²-, -L³-, -L⁴- and

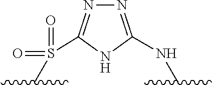 [SMILES: *Nc1nnc(S(*)(=O)=O)[nH]1]

is from 8 to 30 atoms. Typically, the minimum single ring size that encompasses all or part of each of -L¹-, -L²-, -L³-, -L⁴- and

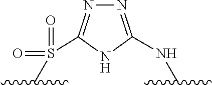 [SMILES: *Nc1nnc(S(*)(=O)=O)[nH]1]

is from 12 to 24 atoms. More typically, the minimum single ring size that encompasses all or part of each of -L¹-, -L²-, -L³-, -L⁴- and

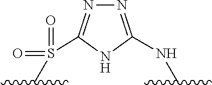 [SMILES: *Nc1nnc(S(*)(=O)=O)[nH]1]

is from 14 to 20 atoms.

In another embodiment of the first aspect of the invention, the compound has the formula (Ic′):

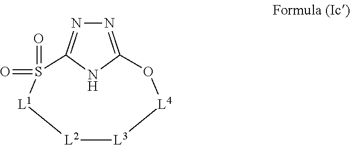

wherein L¹, L², L³ and L⁴ are as previously defined, and wherein the minimum single ring size that encompasses all or part of each of -L¹-, -L²-, -L³-, -L⁴- and

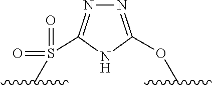 [SMILES: *Oc1nnc(S(*)(=O)=O)[nH]1]

is from 8 to 30 atoms. Typically, the minimum single ring size that encompasses all or part of each of -L¹-, -L²-, -L³-, -L⁴- and

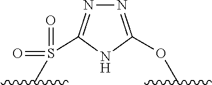 [SMILES: *Oc1nnc(S(*)(=O)=O)[nH]1]

is from 12 to 24 atoms. More typically, the minimum single ring size that encompasses all or part of each of -L¹-, -L²-, -L³-, -L⁴- and

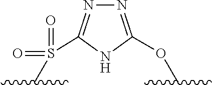 [SMILES: *Oc1nnc(S(*)(=O)=O)[nH]1]

is from 14 to 20 atoms.

In a first exemplary embodiment, where the compound has the formula (Ib) or, more typically, (Ic) or (Ic′):
L¹ is a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L};
L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups and/or one or more oxo (═O) groups;
L³ is a divalent phenyl or 5- or 6-membered heteroaryl group, wherein the divalent phenyl or 5- or 6-membered heteroaryl group may optionally be substituted with one or more halo groups and/or one or more substituents R^{L};
L⁴ is a divalent phenyl or 5- or 6-membered heteroaryl group, wherein the divalent phenyl or 5- or 6-membered heteroaryl group may optionally be substituted with one or more halo groups and/or one or more substituents R^{L};
the ring atom of L⁴ that is directly attached to L³ is at the α-position relative to the ring atom of L⁴ that is directly attached to X (or, where the compound has the formula (Ic) or (Ic′), relative to the ring atom of L⁴ that is directly attached to the nitrogen atom of the —NH— group or the oxygen atom of the —O— group respectively);
each R^{L} is independently selected from a C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, and/or any two R^{L} attached to the same divalent phenyl or 5- or 6-membered heteroaryl group of L³ or L⁴ may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group; and
R¹¹, R¹² and R¹³ are as previously defined.

In one aspect of the first exemplary embodiment, L¹ is a divalent phenyl, or 5- or 6-membered heteroaryl group, wherein the divalent phenyl or 5- or 6-membered heteroaryl group may optionally be substituted with one or more halo groups and/or one or more substituents R^{L}. Typically, where L¹ is a divalent phenyl, or 5- or 6-membered heteroaryl group, it is unsubstituted or substituted with one or more halo groups and/or one or two substituents R^{L}.

In another aspect of the first exemplary embodiment, L¹ is a divalent phenyl, or 5- or 6-membered heteroaryl group, wherein the divalent phenyl or 5- or 6-membered heteroaryl group is ortho-fused to a 5- or 6-membered cyclic group, wherein the divalent phenyl or 5- or 6-membered heteroaryl group may optionally be further substituted with one or two substituents independently selected from halo groups and R^{L}, and wherein the fused 5- or 6-membered cyclic group may optionally be substituted with one or more halo groups and/or one or more oxo (═O) groups and/or one or more substituents R^{L}. Typically, the divalent phenyl or 5- or 6-membered heteroaryl group is unsubstituted or substituted with one or two halo groups and/or a single substituent R^{L}. Typically, the fused 5- or 6-membered cyclic group is unsubstituted or substituted with one or more halo groups and/or one or two substituents independently selected from oxo (═O) groups and R^{L}. In one embodiment, the fused 5- or 6-membered cyclic group is non-aromatic, such as a fused non-aromatic 5- or 6-membered heterocyclic group. In another embodiment, the fused 5- or 6-membered cyclic group is aromatic, such as a fused heteroaryl group.

Typically, where L¹ is a divalent phenyl, or 5- or 6-membered heteroaryl group, e.g. in accordance with either of the above two aspects of the first exemplary embodiment, the ring atom of L that is directly attached to L² is at the α- or β-position relative to the ring atom of L that is directly attached to the sulfur atom of J (or to the sulfur atom of the —SO₂— group where the compound has the formula (Ic) or (Ic′)). More typically, where L¹ is a divalent phenyl or 5- or 6-membered heteroaryl group, the ring atom of L¹ that is directly attached to L² is at the β-position relative to the ring atom of L that is directly attached to the sulfur atom of J (or to the sulfur atom of the —SO₂— group).

In yet another aspect of the first exemplary embodiment, L¹ is a divalent saturated 4- to 7-membered monocyclic heterocyclic group, wherein the divalent saturated 4- to 7-membered monocyclic heterocyclic group may optionally be substituted with one or more halo groups and/or one or more oxo (═O) groups and/or one or more substituents R^{L}. For example, L¹ may be a divalent saturated 4- to 7-membered monocyclic heterocyclic group, wherein the divalent saturated 4- to 7-membered monocyclic heterocyclic group includes one or two heteroatoms selected from nitrogen and oxygen in its ring structure, and wherein the divalent saturated 4- to 7-membered monocyclic heterocyclic group may optionally be substituted with one or more halo groups and/or one or more oxo (═O) groups and/or one or more substituents R^{L}.

Typically, the divalent saturated 4- to 7-membered monocyclic heterocyclic group includes at least one nitrogen atom in its ring structure. For example, L¹ may be selected from a divalent azetidinyl, pyrrolidinyl, piperidinyl, piperazinyl or morpholinyl group, any of which may optionally be substituted with one or more halo groups and/or one or more oxo (═O) groups and/or one or more substituents R^{L}. Typically, where L¹ is a divalent saturated 4- to 7-membered monocyclic heterocyclic group, such as a divalent azetidinyl, pyrrolidinyl, piperidinyl, piperazinyl or morpholinyl group, it is unsubstituted or substituted with one or more fluoro groups and/or one or more chloro groups and/or one or two oxo (═O) groups and/or one or two substituents R^{L}. In one embodiment, where the divalent saturated 4- to 7-membered monocyclic heterocyclic group includes at least one nitrogen atom in its ring structure, the ring atom of L that is directly attached to the sulfur atom of J (or to the sulfur atom of the —SO₂— group where the compound has the formula (Ic) or (Ic′)) is a nitrogen atom. Typically, where L¹ is a divalent saturated 4- to 7-membered monocyclic heterocyclic group, the ring atom of L¹ that is directly attached to L² is at the α-, β- or γ-position relative to the ring atom of L¹ that is directly attached to the sulfur atom of J (or to the sulfur atom of the —SO₂— group where the compound has the formula (Ic) or (Ic′)). More typically in such an aspect, the ring atom of L¹ that is directly attached to L² is at the β- or γ-position relative to the ring atom of L¹ that is directly attached to the sulfur atom of J (or to the sulfur atom of the —SO₂— group). In one embodiment, where L¹ is a divalent saturated 4- to 7-membered monocyclic heterocyclic group, the ring atom of L¹ that is directly attached to L² is at the γ-position relative to the ring atom of L¹ that is directly attached to the sulfur atom of J (or to the sulfur atom of the —SO₂— group).

In a further aspect of the first exemplary embodiment, L¹ is a divalent saturated 7- to 11-membered spiro bicyclic heterocyclic group, wherein the divalent saturated 7- to 11-membered spiro bicyclic heterocyclic group may optionally be substituted with one or more halo groups and/or one or more oxo (═O) groups and/or one or more substituents R^{L}. Typically, where L¹ is a divalent saturated 7- to 11-membered spiro bicyclic heterocyclic group, it is unsubstituted or substituted with one or more fluoro groups and/or one or more chloro groups and/or one or two oxo (═O) groups and/or one or two substituents R^{L}. In one embodiment, the divalent saturated 7- to 11-membered spiro bicyclic heterocyclic group includes one, two or three heteroatoms independently selected from nitrogen and oxygen in its bicyclic ring structure. Typically, the divalent saturated 7- to 11-membered spiro bicyclic heterocyclic group includes at least one nitrogen atom in its bicyclic ring structure. In one embodiment, where the divalent saturated 7- to 11-membered spiro bicyclic heterocyclic group includes at least one nitrogen atom in its bicyclic ring structure, the ring atom of L¹ that is directly attached to the sulfur atom of J (or to the sulfur atom of the —SO₂— group where the compound has the formula (Ic) or (Ic′)) is a nitrogen atom. In a further embodiment, a first ring in the divalent saturated 7- to 11-membered spiro bicyclic heterocyclic group is a 4- to 6-membered ring, and a second ring in the divalent saturated 7- to 11-membered spiro bicyclic heterocyclic group is a 4- to 6-membered ring. Typically in such an embodiment, a ring atom of the first ring is directly attached to the sulfur atom of J (or to the sulfur atom of the —SO₂— group where the compound has the formula (Ic) or (Ic′)), and a ring atom of the second ring is directly attached to L². In one aspect of such an embodiment, both the first ring and the second ring of the divalent saturated 7- to 11-membered spiro bicyclic heterocyclic group are heterocyclic. For example, L¹ may be a divalent saturated 7- to 11-membered spiro bicyclic heterocyclic group, wherein a first ring in the bicyclic group is a 4- to 6-membered ring containing at least one nitrogen atom in its ring structure, and a second ring in the bicyclic group is a 4- to 6-membered ring containing at least one nitrogen atom in its ring structure, wherein a ring atom of the first ring is directly attached to the sulfur atom of J (or to the sulfur atom of the —SO₂— group where the compound has the formula (Ic) or (Ic′)), wherein a ring atom of the second ring is directly attached to L², and wherein the divalent saturated 7- to 11-membered spiro bicyclic heterocyclic group may optionally be substituted with one or more halo groups and/or one or more oxo (═O) groups and/or one or more substituents R^{L}. Typically, where a first ring in the bicyclic group is a 4- to 6-membered ring containing at least one nitrogen atom in its ring structure, a nitrogen ring atom of the first ring is directly attached to the sulfur atom of J (or to the sulfur atom of the —SO₂— group where the compound has the formula (Ic) or (Ic′)). Typically, where a second ring in the bicyclic group is a 4- to 6-membered ring containing at least one nitrogen atom in its ring structure, a nitrogen ring atom of the second ring is directly attached to L².

In another aspect of the first exemplary embodiment, L¹ is a divalent saturated 7- to 11-membered bridged bicyclic heterocyclic group, wherein the divalent saturated 7- to 11-membered bridged bicyclic heterocyclic group may optionally be substituted with one or more halo groups and/or one or more oxo (═O) groups and/or one or more substituents R^{L}. Typically, where L¹ is a divalent saturated 7- to 11-membered bridged bicyclic heterocyclic group, it is unsubstituted or substituted with one or more fluoro groups and/or one or more chloro groups and/or one or two oxo (═O) groups and/or one or two substituents R^{L}. In one embodiment, the divalent saturated 7- to 11-membered bridged bicyclic heterocyclic group includes one, two or three heteroatoms independently selected from nitrogen and oxygen in its bicyclic ring structure. Typically, the divalent saturated 7- to 11-membered bridged bicyclic heterocyclic group includes at least one nitrogen atom in its bicyclic ring structure. More typically, the divalent saturated 7- to 11-membered bridged bicyclic heterocyclic group includes one or two heteroatoms independently selected from nitrogen and oxygen in its bicyclic ring structure, wherein at least one heteroatom is a nitrogen atom. In one embodiment, where the divalent saturated 7- to 11-membered bridged bicyclic heterocyclic group includes at least one nitrogen atom in its bicyclic ring structure, the ring atom of L that is directly attached to the sulfur atom of J (or to the sulfur atom of the —SO₂— group where the compound has the formula (Ic) or (Ic′)) is a nitrogen atom. In another embodiment, where the divalent saturated 7- to 11-membered bridged bicyclic heterocyclic group includes at least one nitrogen atom in its bicyclic ring structure, the ring atom of L that is directly attached to L² is a nitrogen atom. In a further embodiment, the divalent saturated 7- to 11-membered bridged bicyclic heterocyclic group includes a first and a second nitrogen atom in its bicyclic ring structure, such that the first nitrogen atom is directly attached to the sulfur atom of J (or to the sulfur atom of the —SO₂— group where the compound has the formula (Ic) or (Ic′)), and the second nitrogen atom is directly attached to L².

In one aspect of the first exemplary embodiment, L² contains in total from 2 to 15 carbon, nitrogen and oxygen atoms. Typically, L² contains in total from 2 to 10 carbon, nitrogen and oxygen atoms. More typically, L² contains in total from 3 to 7 carbon, nitrogen and oxygen atoms. Typically, L² has a chain length of from 2 to 12 atoms. More typically, L² has a chain length of from 2 to 8 atoms. Yet more typically, L² has a chain length of from 3 to 6 atoms.

In another aspect of the first exemplary embodiment, L² is an alkylene or alkenylene group, wherein the alkylene or alkenylene group may be straight-chained or branched, or be or include a single monocyclic group, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups and/or one or more oxo (═O) groups. In one embodiment, the alkylene or alkenylene group of L² is straight-chained or branched. Typically, L² includes at least one heteroatom independently selected from O and N in its carbon skeleton. More typically, L² includes one, two or three heteroatoms independently selected from O and N in its carbon skeleton. Typically, the atom of L² that is directly attached to L³ is O or N. More typically, the atom of L² that is directly attached to L³ is O.

Typically, where L³ is a divalent phenyl, or 5- or 6-membered heteroaryl group, it is unsubstituted or substituted with one or more halo groups and/or one or two substituents R^{L}. More typically, L³ is a divalent phenyl or 6-membered heteroaryl group, wherein the divalent phenyl or 6-membered heteroaryl group is unsubstituted or substituted with one or more halo groups and/or one or two substituents R^{L}.

In one embodiment, where L³ is a divalent phenyl or 5- or 6-membered heteroaryl group, the ring atom of L³ that is directly attached to L² is at the α- or β-position relative to the ring atom of L³ that is directly attached to L⁴. In a further embodiment, where L³ is a divalent phenyl or 5- or 6-membered heteroaryl group, the ring atom of L³ that is directly attached to L² is at the β-position relative to the ring atom of L³ that is directly attached to L⁴.

As stated, in accordance with the first exemplary embodiment, L⁴ is a divalent phenyl or 5- or 6-membered heteroaryl group, wherein the divalent phenyl or 5- or 6-membered heteroaryl group may optionally be substituted with one or more halo groups and/or one or more substituents R^{L}. Typically, L⁴ is a divalent phenyl or 6-membered heteroaryl group, wherein the divalent phenyl or 6-membered heteroaryl group may optionally be substituted with one or more halo groups and/or one or more substituents R^{L}.

In one aspect of the first exemplary embodiment, the divalent phenyl or 5- or 6-membered heteroaryl group of L⁴ is substituted at the α′-position, relative to the ring atom of L⁴ that is directly attached to X (or, where the compound has the formula (Ic), relative to the ring atom of L⁴ that is directly attached to the nitrogen atom of the —NH— group; or, where the compound has the formula (Ic′), relative to the ring atom of L⁴ that is directly attached to the oxygen atom of the —O— group), with a substituent R^{L}, wherein R^{L} is as defined above. Typically, the substituent at the α′-position is selected from a C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³ or —R¹¹—CON(R¹³)₂ group, wherein R¹¹, R¹² and R¹³ are as previously defined. More typically, the substituent at the α′-position is selected from a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups.

In a further aspect of the first exemplary embodiment, the divalent phenyl or 5- or 6-membered heteroaryl group of L⁴ is ortho-fused to a 5- or 6-membered cyclic group across the α′,β′-positions, relative to the ring atom of L⁴ that is directly attached to X (or, where the compound has the formula (Ic), relative to the ring atom of L⁴ that is directly attached to the nitrogen atom of the —NH— group; or, where the compound has the formula (Ic′), relative to the ring atom of L⁴ that is directly attached to the oxygen atom of the —O— group), wherein the ortho-fused 5- or 6-membered cyclic group is optionally substituted with one or more halo groups, and/or one or two oxo (═O) groups, and/or one, two or three substituents independently selected from a C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group, wherein R¹¹, R¹² and R¹³ are as previously defined. Typically, the ortho-fused 5- or 6-membered cyclic group is unsubstituted or substituted with one or more halo groups and/or a single oxo (═O) group and/or one, two or three substituents independently selected from a —OH, —CN, —NH₂, C₁-C₄ alkyl, C₁-C₄ haloalkyl, —O(C₁-C₄ alkyl) or —O(C₁-C₄ haloalkyl) group. More typically, the ortho-fused 5- or 6-membered cyclic group is unsubstituted or substituted with one or more halo groups.

As will be understood, in either of the above two aspects of the first exemplary embodiment, the divalent phenyl or 5- or 6-membered heteroaryl group of L⁴ may optionally be further substituted with one or more halo groups and/or one or more further substituents R^{L}. Typically, the divalent phenyl or 5- or 6-membered heteroaryl group of L⁴ may optionally be further substituted with one or more halo groups and/or one or two further substituents R^{L}. More typically, the 5- or 6-membered heteroaryl group of L⁴ may optionally be further substituted with one or more halo groups and/or one or two methyl and/or halomethyl groups.

In a second exemplary embodiment of the first aspect of the invention, the compound has the formula (Id):

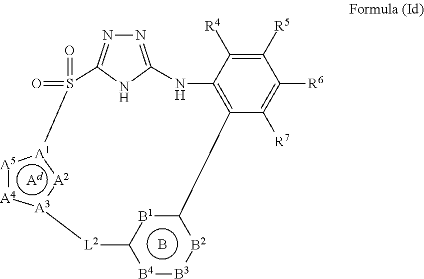

wherein:
A¹ and A³ are each independently selected from C and N, and A², A⁴ and A⁵ are each independently selected from N, CH, CY¹, CR^{A}, NH and NR^{A}, such that ring A^{d} is a 5-membered heteroaryl ring containing one, two or three nitrogen atoms in its ring structure;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₅ alkylene or C₁-C₅ fluoroalkylene group may optionally be replaced by a single oxygen atom;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

In a third exemplary embodiment of the first aspect of the invention, the compound has the formula (Id′):

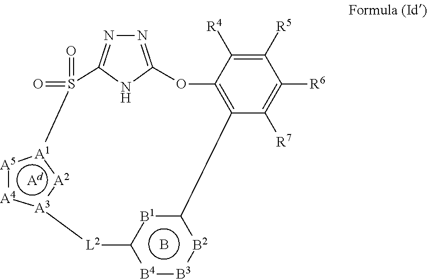

wherein A^{d}, A¹, A², A³, A⁴, A⁵, L², B, B¹, B², B³, B⁴, R⁴, R⁵, R⁶ and R⁷ are as defined in relation to the second exemplary embodiment.

In one embodiment of the second or third exemplary embodiment, ring A^{d} is a 5-membered heteroaryl ring containing two or three nitrogen atoms in its ring structure.

In one embodiment, A¹ is C.

In a further embodiment, A¹ is C, A³ is independently selected from C and N, and A², A⁴ and A⁵ are each independently selected from N, CH, CY¹, CR^{A}, NH and NR^{A}, such that ring A^{d} is a 5-membered heteroaryl ring containing two nitrogen atoms in its ring structure. For example, ring A^{d} may be a pyrazole ring. In one aspect of such an embodiment, A³ is N.

In one embodiment, at least one of A², A⁴ and A⁵ is selected from N, CH, CY¹ and NH. Typically, at least two of A², A⁴ and A⁵ are selected from N, CH, CY¹ and NH. In a further embodiment, A², A⁴ and A⁵ are each independently selected from N, CH, CY¹ and N—H.

In one embodiment of the second or third exemplary embodiment of the first aspect of the invention, each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally fluoro-substituted, and wherein each R^{A} contains, in total, from 1 to 6 carbon, nitrogen and oxygen atoms. Typically in such an embodiment, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{A} contains, in total, from 1 to 4 carbon, nitrogen and oxygen atoms.

In a fourth exemplary embodiment of the first aspect of the invention, the compound has the formula (Ie):

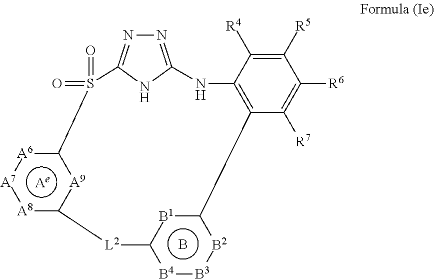

wherein:
A⁶, A⁷, A⁸ and A⁹ are each independently selected from N, CH, CY¹ and CR^{A}, such that ring A^{e} is a 6-membered aryl ring or a 6-membered heteroaryl ring containing one, two or three nitrogen atoms in its ring structure;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₅ alkylene or C₁-C₅ fluoroalkylene group may optionally be replaced by a single oxygen atom;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

In a fifth exemplary embodiment of the first aspect of the invention, the compound has the formula (Ie′):

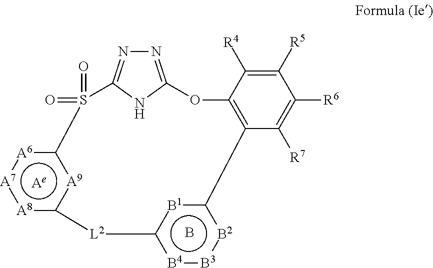

wherein A^{e}, A⁶, A⁷, A⁸, A⁹, L², B, B¹, B², B³, B⁴, R⁴, R⁵, R⁶ and R⁷ are as defined in relation to the fourth exemplary embodiment.

In one embodiment of the fourth or fifth exemplary embodiment, ring A^{e} is a 6-membered aryl ring or a 6-membered heteroaryl ring containing one or two nitrogen atoms in its ring structure. In a further embodiment, ring A^{e} is a 6-membered aryl ring or a 6-membered heteroaryl ring containing a single nitrogen atom in its ring structure.

In one embodiment, at least one of A⁶, A⁷, A⁸ and A⁹ is selected from N, CH and CY¹. Typically, at least two of A⁶, A⁷, A⁸ and A⁹ are independently selected from N, CH and CY¹. More typically, at least three of A⁶, A⁷, A⁸ and A⁹ are independently selected from N, CH and CY¹. In a further embodiment, A⁶, A⁷, A⁸ and A⁹ are each independently selected from N, CH and CY¹.

In one embodiment, A⁶, A⁷, A⁸ and A⁹ are each independently selected from CH, CY¹ and CR^{A}, such that ring A^{e} is a 6-membered aryl ring. Typically in such an embodiment, at least one of A⁶, A⁷, A⁸ and A⁹ is selected from CH and CY¹. More typically, at least two of A⁶, A⁷, A⁸ and A⁹ are independently selected from CH and CY¹. More typically still, at least three of A⁶, A⁷, A⁸ and A⁹ are independently selected from CH and CY¹. In a further embodiment, A⁶, A⁷, A⁸ and A⁹ are each independently selected from CH and CY¹.

In one embodiment of the fourth or fifth exemplary embodiment of the first aspect of the invention, each R^{A} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally fluoro-substituted, and wherein each R^{A} contains, in total, from 1 to 6 carbon, nitrogen and oxygen atoms. Typically in such an embodiment, each R^{A} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{A} contains, in total, from 1 to 4 carbon, nitrogen and oxygen atoms.

In a sixth exemplary embodiment of the first aspect of the invention, the compound has the formula (If):

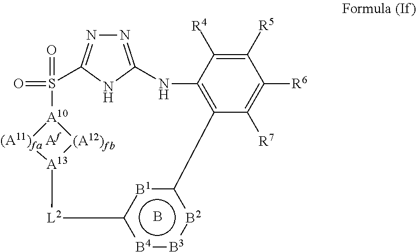

wherein:
A¹⁰ and A¹³ are each independently selected from N, CH, CY² and CR^{AA}, and each A¹¹ and A¹² is independently selected from O, NH, NR^{AAA}, C═O, CH₂, CH(Y²), CH(R^{AA}), C(Y²)₂, C(Y²)(R^{AA}) and C(R^{AA})₂, such that ring A^{f} contains one or two atoms independently selected from oxygen and nitrogen in its ring structure;
fa is 1, 2 or 3, and fb is 1, 2 or 3, provided that fa+fb≤5;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1}₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₅ alkylene or C₁-C₅ fluoroalkylene group may optionally be replaced by a single oxygen atom;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

In a seventh exemplary embodiment of the first aspect of the invention, the compound has the formula (If):

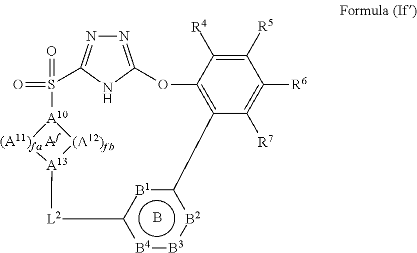

wherein A^{f}, A¹⁰, A¹¹, A¹², A¹³, fa, fb, L², B, B¹, B², B³, B⁴, R⁴, R⁵, R⁶ and R⁷ are as defined in relation to the sixth exemplary embodiment.

As will be understood, since ring A^{f} of the sixth or seventh exemplary embodiment of the first aspect of the invention contains one or two atoms independently selected from oxygen and nitrogen in its ring structure, all other atoms within the ring structure of ring A^{f} will be carbon atoms. Typically, each ring carbon atom of ring R^{f} is directly attached to at least one other ring carbon atom of ring R^{f}. Typically, each ring nitrogen or oxygen atom of ring R^{f} is directly attached to two ring carbon atoms of ring R^{f}.

Typically, in accordance with the sixth or seventh exemplary embodiment of the first aspect of the invention, each Y² is F or Cl. More typically, each Y² is F.

Typically, 2≤fa+fb≤4. More typically, 3≤fa+fb≤4.

In one embodiment of the sixth or seventh exemplary embodiment of the first aspect of the invention, A¹⁰ is independently selected from N, CH and CY², A¹³ is independently selected from N, CH, CY² and CR^{AA}, any two A¹¹ and/or A¹² are each independently selected from O, NH, NR^{AAA}, C═O, CH₂, CH(Y²), CH(R^{AA}), C(Y²)₂, C(Y²)(R^{AA}) and C(R^{AA})₂, and each remaining A¹¹ and A¹² is independently selected from O, NH, CH₂, CH(Y²), and C(Y²)₂.

In another embodiment, A¹⁰ and A¹³ are each independently selected from N, CH and CY², any two A¹¹ and/or A¹² are each independently selected from O, NH, NR^{AAA}, C═O, CH₂, CH(Y²), CH(R^{AA}), C(Y²)₂, C(Y²)(R^{AA}) and C(R^{AA})₂, and each remaining A¹ and A¹² is independently selected from O, NH, CH₂, CH(Y²), and C(Y²)₂.

In a further embodiment of the sixth or seventh exemplary embodiment of the first aspect of the invention, A¹⁰ is independently selected from N, CH and CF, A¹³ is independently selected from N, CH, CF and CR^{AA}, one A¹¹ or A¹² is independently selected from O, NH, NR^{AAA}, C═O, CH₂, CHF, CH(R^{AA}), CF₂, CF(R^{AA}) and C(R^{AA})₂, and each remaining A¹ and A¹² is independently selected from O, NH, CH₂, CHF, and CF₂.

In yet another embodiment, A¹⁰ and A¹³ are each independently selected from N, CH and CF, one A¹ or A¹² is independently selected from O, NH, NR^{AAA}, C═O, CH₂, CHF, CH(R^{AA}), CF₂, CF(R^{AA}) and C(R^{AA})₂, and each remaining A¹¹ and A¹² is independently selected from O, NH, CH₂, CHF, and CF₂.

In one embodiment of the sixth or seventh exemplary embodiment of the first aspect of the invention, A¹⁰ and A¹³ are each independently selected from N, CH, CY² and CR^{AA}, and each A¹ and A¹² is independently selected from NH, NR^{AAA}, C═O, CH₂, CH(Y²), CH(R^{AA}), C(Y²)₂, C(Y²)(R^{AA}) and C(R^{AA})₂, such that ring A^{f} contains a single nitrogen atom in its ring structure.

Typically in such an embodiment, A¹⁰ and A¹³ are each independently selected from N, CH and CY², any two A¹¹ and/or A¹² are each independently selected from NH, NR^{AAA}, C═O, CH₂, CH(Y²), CH(R^{AA}), C(Y²)₂, C(Y²)(R^{AA}) and C(R^{AA})₂, and each remaining A¹¹ and A¹² is independently selected from NH, CH₂, CH(Y²), and C(Y²)₂. Equally typically in such an embodiment, A¹⁰ is independently selected from N, CH and CY², A¹³ is independently selected from N, CH, CY² and CR^{AA}, one A¹ or A¹² is independently selected from NH, NR^{AAA}, C═O, CH₂, CH(Y²), CH(R^{AA}), C(Y²)₂, C(Y²)(R^{AA}) and C(R^{AA})₂, and each remaining A¹¹ and A¹² is independently selected from NH, CH₂, CH(Y²), and C(Y²)₂. In one aspect of such an embodiment, A¹⁰ is N. More typically, either:

(i) A¹⁰ is N, A¹³ is independently selected from CH and CF, one A¹¹ or A¹² is independently selected from C═O, CH₂, CHF, CH(R^{AA}), CF₂, CF(R^{AA}) and C(R^{AA})₂, and each remaining A¹¹ and A¹² is independently selected from CH₂, CHF, and CF₂; or

(ii) A¹⁰ is N, A¹³ is independently selected from CH, CF and CR^{AA}, and each A¹¹ and A¹² is independently selected from CH₂, CHF, and CF₂.

In one embodiment of the sixth or seventh exemplary embodiment of the first aspect of the invention, each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally fluoro-substituted, and wherein each R^{AA} contains, in total, from 1 to 6 carbon, nitrogen and oxygen atoms. Typically in such an embodiment, each R^{AA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{AA} contains, in total, from 1 to 4 carbon, nitrogen and oxygen atoms. More typically in such an embodiment, each R^{AA} is independently selected from —OMe, —OEt, —OiPr, —OnPr, —CH₂OMe, —CH₂CH₂OMe, —CH₂OEt, —NHMe, —NMe₂, —NHEt, —N(Me)Et, —NHiPr, —NHnPr, —CH₂NH₂, —CH₂NHMe, —CH₂NHEt, —CH₂NMe₂, —CH₂CH₂NH₂, —CH₂CH₂NHMe,

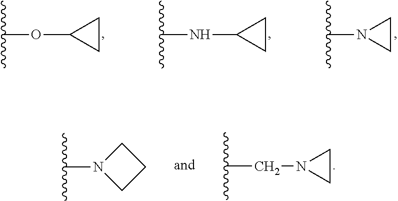

Yet more typically in such an embodiment, each R^{AA} is independently selected from —NMe₂, —N(Me)Et, —CH₂NMe₂,

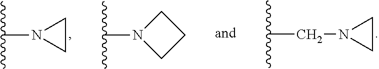

In another embodiment of the fourth exemplary embodiment of the first aspect of the invention, each R^{AAA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{AAA} contains, in total, from 1 to 6 carbon, nitrogen and oxygen atoms. Typically in such an embodiment, each R^{AAA} is independently selected from a C₁-C₄ alkyl, C₁-C₄ fluoroalkyl, C₃-C₄ cycloalkyl or C₃-C₄ fluorocycloalkyl group.

In an eighth exemplary embodiment of the first aspect of the invention, the compound has the formula (Ig):

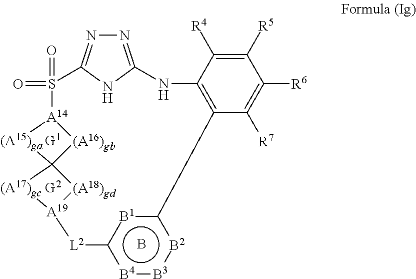

wherein:
A¹⁴ and A¹⁹ are each independently selected from N, CH, CY² and CR^{AA}, and each A¹⁵, A¹⁶, A¹⁷ and A¹⁸ is independently selected from O, NH, NR^{AAA}, C═O, CH₂, CH(Y²), CH(R^{AA}), C(Y²)₂, C(Y²)(R^{AA}) and C(R^{AA})₂, such that ring G¹ contains zero, one or two atoms independently selected from oxygen and nitrogen in its ring structure, and ring G² contains zero, one or two atoms independently selected from oxygen and nitrogen in its ring structure;
ga is 0, 1, 2, 3 or 4 and gb is 0, 1, 2, 3 or 4 provided that 1≤ga+gb≤5;
gc is 0, 1, 2, 3 or 4 and gd is 0, 1, 2, 3 or 4 provided that 1≤gc+gd≤5;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₅ alkylene or C₁-C₅ fluoroalkylene group may optionally be replaced by a single oxygen atom;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

In a ninth exemplary embodiment of the first aspect of the invention, the compound has the formula (Ig′):

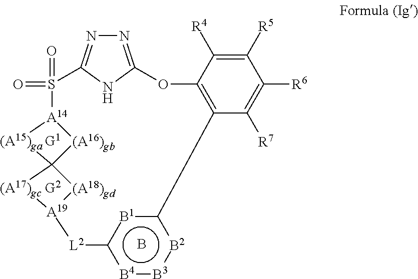

wherein G¹, G², A¹⁴, A¹⁵, A¹⁶, A¹⁷, A¹⁸, A¹⁹, ga, gb, gc, gd, L², B, B¹, B², B³, B⁴, R⁴, R⁵, R⁶ and R⁷ are as defined in relation to the eighth exemplary embodiment.

As will be understood, for either of the eighth or ninth exemplary embodiments, where ga is 0, A¹⁴ is directly attached to the spiro carbon atom common to rings G¹ and G², i.e. for the eighth exemplary embodiment where ga is 0 the compound has the formula (Iga):

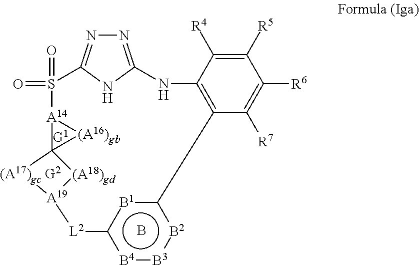

wherein all remaining groups are as defined in relation to Formula (Ig).

Likewise, for either of the eighth or ninth exemplary embodiments, where gb is 0, A¹⁴ is directly attached to the spiro carbon atom common to rings G¹ and G², where gc is 0, A¹⁹ is directly attached to the spiro carbon atom common to rings G¹ and G², and where gd is 0, A¹⁹ is directly attached to the spiro carbon atom common to rings G¹ and G².

As will be understood, where ring G¹ contains zero oxygen or nitrogen atoms in its ring structure, all ring atoms within the ring structure of ring G¹ will be carbon atoms.

Where ring G¹ contains one or two atoms selected from oxygen and nitrogen in its ring structure, all other atoms within the ring structure of ring G¹ will be carbon atoms.

Likewise, where ring G² contains zero oxygen or nitrogen atoms in its ring structure, all ring atoms within the ring structure of ring G² will be carbon atoms. Where ring G² contains one or two atoms selected from oxygen and nitrogen in its ring structure, all other atoms within the ring structure of ring G² will be carbon atoms.

Typically, each ring carbon atom of ring G¹ is directly attached to at least one other ring carbon atom of ring G¹, and each ring carbon atom of ring G² is directly attached to at least one other ring carbon atom of ring G². Typically, each ring nitrogen or oxygen atom of ring G¹ is directly attached to two ring carbon atoms of ring G¹, and each ring nitrogen or oxygen atom of ring G² is directly attached to two ring carbon atoms of ring G².

Typically, in accordance with the eighth or ninth exemplary embodiment of the first aspect of the invention, each Y² is F or Cl. More typically, each Y² is F.

In one embodiment, ring G¹ contains one or two atoms selected from oxygen and nitrogen in its ring structure. In such an embodiment, 2≤ga+gb≤5.

In another embodiment, ring G² contains one or two atoms selected from oxygen and nitrogen in its ring structure. In such an embodiment, 2≤gc+gd≤5.

In yet another embodiment, one of rings G¹ and G² contains a single nitrogen atom in its ring structure, and the other of rings G¹ and G² contains a single nitrogen atom, two nitrogen atoms or a nitrogen and an oxygen atom in its ring structure. In one aspect of such an embodiment, A¹⁴ is N and A¹⁹ is N.

Typically, 2≤ga+gb≤4, and 2≤gc+gd≤4.

In one embodiment, A¹⁴ and A¹⁹ are each independently selected from N, CH and CY², any two A¹⁵, A¹⁶, A¹⁷ and/or A¹⁸ are each independently selected from O, NH, NR^{AAA}, C═O, CH₂, CH(Y²), CH(R^{AA}), C(Y²)₂, C(Y²)(R^{AA}) and C(R^{AA})₂, and each remaining A¹⁵, A¹⁶, A¹⁷ and A¹⁸ is independently selected from O, NH, CH₂, CH(Y²), and C(Y²)₂.

In a further embodiment, A¹⁴ and A¹⁹ are each independently selected from N, CH and CF, one A¹⁵, A¹⁶, A¹⁷ or A¹⁸ is independently selected from O, NH, NR^{AAA}, C═O, CH₂, CHF, CH(R^{AA}), CF₂, CF(R^{AA}) and C(R^{AA})₂, and each remaining A¹⁵, A¹⁶, A¹⁷ and A¹⁸ is independently selected from O, NH, CH₂, CHF, and CF₂.

In one embodiment, A¹⁴ and A¹⁹ are each independently selected from N, CH, CY² and CR^{AA}, each A¹⁵ and A¹⁶ is independently selected from NH, NR^{AAA}, C═O, CH₂, CH(Y²), CH(R^{AA}), C(Y²)₂, C(Y²)(R^{AA}) and C(R^{AA})₂, and each A¹⁷ and A¹⁸ is independently selected from O, NH, NR^{AAA}, C═O, CH₂, CH(Y²), CH(R^{AA}), C(Y²)₂, C(Y²)(R^{AA}) and C(R^{AA})₂, such that ring G¹ contains a single nitrogen atom in its ring structure, and ring G² contains a single nitrogen atom, two nitrogen atoms or a nitrogen and an oxygen atom in its ring structure. Typically in such an embodiment, A¹⁴ and A¹⁹ are each independently selected from N, CH and CY², one A¹⁵ or A¹⁶ is independently selected from NH, NR^{AAA}, C═O, CH₂, CH(Y²), CH(R^{AA}), C(Y²)₂, C(Y²)(R^{AA}) and C(R^{AA})₂, each remaining A¹⁵ and A¹⁶ is independently selected from NH, CH₂, CH(Y²), and C(Y²)₂, one A¹⁷ or A¹⁸ is independently selected from O, NH, NR^{AAA}, C═O, CH₂, CH(Y²), CH(R^{AA}), C(Y²)₂, C(Y²)(R^{AA}) and C(R^{AA})₂, and each remaining A¹⁷ and A¹⁸ is independently selected from O, NH, CH₂, CH(Y²), and C(Y²)₂. In one embodiment, A¹⁴ is N and A¹⁹ is N. More typically, A¹⁴ is N, A¹⁹ is N, one A¹⁵ or A¹⁶ is independently selected from C═O, CH₂, CHF, CH(R^{AA}), CF₂, CF(R^{AA}) and C(R^{AA})₂, each remaining A¹⁵ and A¹⁶ is independently selected from CH₂, CHF, and CF₂, one A¹⁷ or A¹⁸ is independently selected from C═O, CH₂, CHF, CH(R^{AA}), CF₂, CF(R^{AA}) and C(R^{AA})₂, and each remaining A¹⁷ and A¹⁸ is independently selected from O, CH₂, CHF, and CF₂, such that ring G¹ contains a single nitrogen atom in its ring structure, and ring G² contains a single nitrogen atom and optionally a single oxygen atom in its ring structure.

In another embodiment, A¹⁴ and A¹⁹ are each independently selected from N, CH, CY² and CR^{AA}, and each A¹⁵, A¹⁶, A¹⁷ and A¹⁸ is independently selected from NH, NR^{AAA}, C═O, CH₂, CH(Y²), CH(R^{AA}), C(Y²)₂, C(Y²)(R^{AA}) and C(R^{AA})₂, such that ring G¹ contains a single nitrogen atom in its ring structure, and ring G² contains a single nitrogen atom in its ring structure. Typically in such an embodiment, A¹⁴ and A¹⁹ are each independently selected from N, CH and CY², any two A¹⁵, A¹⁶, A¹⁷ and/or A¹⁸ are each independently selected from NH, NR^{AAA}, C═O, CH₂, CH(Y²), CH(R^{AA}), C(Y²)₂, C(Y²)(R^{AA}) and C(R^{AA})₂, and each remaining A¹⁵, A¹⁶, A¹⁷ and A¹⁸ is independently selected from NH, CH₂, CH(Y²), and C(Y²)₂. In one embodiment, A¹⁴ is N and A¹⁹ is N.

More typically, A¹⁴ is N, A¹⁹ is N, one A¹⁵, A¹⁶, A¹⁷ or A¹⁸ is independently selected from C═O, CH₂, CHF, CH(R^{AA}), CF₂, CF(R^{AA}) and C(R^{AA})₂, and each remaining A¹⁵, A¹⁶, A¹⁷ and A¹⁸ is independently selected from CH₂, CHF, and CF₂.

In one embodiment, A¹⁴ is N, A¹⁹ is N, each A¹⁵ and A¹⁶ is CH₂, one A¹⁷ or A¹⁸ is selected from O and CH₂, and each remaining A¹⁷ and A¹⁸ is CH₂.

In another embodiment, A¹⁴ is N, A¹⁹ is N, and each A¹⁵, A¹⁶, A¹⁷ and A¹⁸ is CH₂.

In one embodiment of the eighth or ninth exemplary embodiment of the first aspect of the invention, each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally fluoro-substituted, and wherein each R^{AA} contains, in total, from 1 to 6 carbon, nitrogen and oxygen atoms. Typically in such an embodiment, each R^{AA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{AA} contains, in total, from 1 to 4 carbon, nitrogen and oxygen atoms.

In another embodiment of the eighth or ninth exemplary embodiment of the first aspect of the invention, each R^{AAA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{AAA} contains, in total, from 1 to 6 carbon, nitrogen and oxygen atoms. Typically in such an embodiment, each R^{AAA} is independently selected from a C₁-C₄ alkyl, C₁-C₄ fluoroalkyl, C₃-C₄ cycloalkyl or C₃-C₄ fluorocycloalkyl group.

In a tenth exemplary embodiment of the first aspect of the invention, the compound has the formula (Ih):

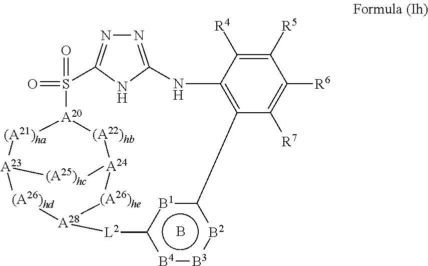

wherein:
each A²⁰, A²³, A²⁴ and A²⁸ is independently selected from N, CH, CY² and CR^{AA}, and each A²¹, A²², A²⁵, A²⁶ and A²⁷ is independently selected from O, NH, NR^{AAA}, C═O, CH₂, CH(Y²), CH(R^{AA}), C(Y²)₂, C(Y²)(R^{AA}) and C(R^{AA})₂, such that the divalent bridged bicyclic group defined by A²⁰, A²¹, A²², A²³, A²⁴, A²⁵, A²⁶, A²⁷ and A²⁸ contains zero, one, two or three atoms independently selected from oxygen and nitrogen in its ring structure;
ha is 0, 1 or 2;
hb is 0, 1 or 2;
he is 1, 2 or 3;
hd is 0, 1 or 2;
he is 0, 1 or 2;
1≤ha+hb+hc+hd+he≤7;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₅ alkylene or C₁-C₅ fluoroalkylene group may optionally be replaced by a single oxygen atom;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

In an eleventh exemplary embodiment of the first aspect of the invention, the compound has the formula (Ih′):

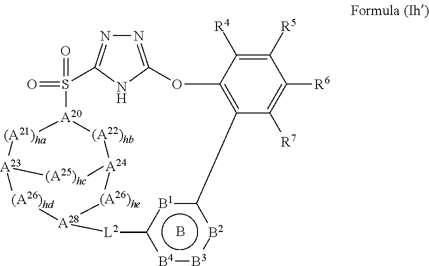

wherein A²⁰, A²¹, A²², A²³, A²⁴, A²⁵, A²⁶, A²⁷, A²⁸, ha, hb, he, hd, he, L², B, B¹, B², B³, B⁴, R⁴, R⁵, R⁶ and R⁷ are as defined in relation to the tenth exemplary embodiment.

As will be understood, for either of the tenth or eleventh exemplary embodiments, where ha is 0, A²⁰ is directly attached to A²³. Similarly, where hb is 0, A²⁰ is directly attached to A²⁴, where hd is 0, A²³ is directly attached to A²⁸, and where he is 0, A²⁴ is directly attached to A²⁸.

As will be understood, where the divalent bridged bicyclic group defined by A²⁰, A²¹, A²², A²³, A²⁴, A²⁵, A²⁶, A²⁷ and A²⁸ contains zero oxygen or nitrogen atoms in its ring structure, all ring atoms within the ring structure of the divalent bridged bicyclic group will be carbon atoms. Where the divalent bridged bicyclic group defined by A²⁰, A²¹, A²², A²³, A²⁴, A²⁵, A²⁶, A²⁷ and A²⁸ contains one, two or three atoms independently selected from oxygen and nitrogen in its ring structure, all other atoms within the ring structure of the divalent bridged bicyclic group will be carbon atoms. Typically, each ring carbon atom of the divalent bridged bicyclic group is directly attached to at least one other ring carbon atom of the divalent bridged bicyclic group. Typically, each ring nitrogen or oxygen atom of the divalent bridged bicyclic group is directly attached to at least two ring carbon atoms of the divalent bridged bicyclic group. Typically, where A²³ is N, the nitrogen atom of A²³ is directly attached to three ring carbon atoms of the divalent bridged bicyclic group. Typically, where A²⁴ is N, the nitrogen atom of A²⁴ is directly attached to three ring carbon atoms of the divalent bridged bicyclic group.

Typically, in accordance with the tenth or eleventh exemplary embodiment of the first aspect of the invention, each Y² is F or Cl. More typically, each Y² is F.

In one embodiment, the divalent bridged bicyclic group defined by A²⁰, A²¹, A²², A²³, A²⁴, A²⁵, A²⁶, A²⁷ and A²⁸ contains one or two atoms independently selected from oxygen and nitrogen in its ring structure.

Typically, ha is 0 or 1, hb is 0 or 1, he is 1 or 2, hd is 0 or 1, and he is 0 or 1.

Typically, 0≤ha+hb≤2. More typically, ha+hb=1.

Typically, 0≤hd+he≤2. More typically, hd+he=1.

Typically, 2≤ha+hb+he+hd+he≤5. More typically, 3≤ha+hb+he+hd+he≤4.

In one embodiment of the tenth or eleventh exemplary embodiment of the first aspect of the invention, each A²⁰, A²³, A²⁴ and A²⁸ is independently selected from N, CH and CY², any two A²¹, A²², A²⁵, A²⁶ and A²⁷ are independently selected from O, NH, NR^{AAA}, C═O, CH₂, CH(Y²), CH(R^{AA}), C(Y²)₂, C(Y²)(R^{AA}) and C(R^{AA})₂, and each remaining A²¹, A²², A²⁵, A²⁶ and A²⁷ is independently selected from O, NH, CH₂, CH(Y²) and C(Y²)₂.

In a further embodiment, each A²⁰, A²³, A²⁴ and A²⁸ is independently selected from N, CH and CF, one A²¹, A²², A²⁵, A²⁶ or A²⁷ is independently selected from O, NH, NR^{AAA}, C═O, CH₂, CHF, CH(R^{AA}), CF₂, CF(R^{AA}) and C(R^{AA})₂, and each remaining A²¹, A²², A²⁵, A²⁶ and A²⁷ is independently selected from O, NH, CH₂, CHF and CF₂.

In another embodiment, each A²⁰, A²³, A²⁴ and A²⁸ is independently selected from N, CH, CY² and CR^{AA}, and each A²¹, A²², A²⁵, A²⁶ and A²⁷ is independently selected from NH, NR^{AAA}, C═O, CH₂, CH(Y²), CH(R^{AA}), C(Y²)₂, C(Y²)(R^{AA}) and C(R^{AA})₂, such that the divalent bridged bicyclic group defined by A²⁰, A²¹, A²², A²³, A²⁴, A²⁵, A²⁶, A²⁷ and A²⁸ contains one or two nitrogen atoms in its ring structure. Typically in such an embodiment, each A²⁰, A²³, A²⁴ and A²⁸ is independently selected from N, CH and CY², any two A²¹, A²², A²⁵, A²⁶ and A²⁷ are independently selected from NH, NR^{AAA}, C═O, CH₂, CH(Y²), CH(R^{AA}), C(Y²)₂, C(Y²)(R^{AA}) and C(R^{AA})₂, and each remaining A²¹, A²², A²⁵, A²⁶ and A²⁷ is independently selected from NH, CH₂, CH(Y²) and C(Y²)₂. In one embodiment, A²⁰ is N and A²⁸ is N. More typically, A²⁰ is N, A²⁸ is N, each A²³ and A²⁴ is independently selected from CH and CF, one A²¹, A²², A²⁵, A²⁶ or A²⁷ is independently selected from C═O, CH₂, CHF, CH(R^{AA}), CF₂, CF(R^{AA}) and C(R^{AA})₂, and each remaining A²¹, A²², A²⁵, A²⁶ and A²⁷ is independently selected from CH₂, CHF and CF₂.

In one embodiment, A²⁰ is N, A²⁸ is N, each A²³ and A²⁴ is CH, and each A²¹, A²², A²⁵, A²⁶ and A²⁷ is CH₂. Typically in such an embodiment, ha is 0 or 1, hb is 0 or 1, he is 1 or 2, hd is 0 or 1, he is 0 or 1, and 2≤ha+hb+he+hd+he≤5. More typically in such an embodiment, ha is 0 or 1, hb is 0 or 1, he is 1 or 2, hd is 0 or 1, he is 0 or 1, ha+hb=1, and hd+he=1.

In one embodiment of the tenth or eleventh exemplary embodiment of the first aspect of the invention, each R^{AA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally fluoro-substituted, and wherein each R^{AA} contains, in total, from 1 to 6 carbon, nitrogen and oxygen atoms. Typically in such an embodiment, each R^{AA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally fluoro-substituted, and wherein each R^{AA} contains, in total, from 1 to 4 carbon, nitrogen and oxygen atoms.

In another embodiment of the tenth or eleventh exemplary embodiment of the first aspect of the invention, each R^{AAA} is independently selected from a saturated hydrocarbyl group, wherein the saturated hydrocarbyl group is straight-chained or branched, or is or includes a cyclic group, wherein the saturated hydrocarbyl group optionally includes a single heteroatom O or N in its carbon skeleton, wherein the saturated hydrocarbyl group is optionally substituted with one or more fluoro groups and/or a single oxo (═O) group, and wherein each R^{AAA} contains, in total, from 1 to 6 carbon, nitrogen and oxygen atoms. Typically in such an embodiment, each R^{AAA} is independently selected from a C₁-C₄ alkyl, C₁-C₄ fluoroalkyl, C₃-C₄ cycloalkyl or C₃-C₄ fluorocycloalkyl group.

In one embodiment of any of the second to eleventh exemplary embodiments of the first aspect of the invention, ring B is a 6-membered aryl ring or a 6-membered heteroaryl ring containing one or two nitrogen atoms in its ring structure.

In a further embodiment, ring B is a 6-membered aryl ring or a 6-membered heteroaryl ring containing a single nitrogen atom in its ring structure. In one example of such an embodiment, B¹, B² and B³ are each independently selected from CH, CY¹ and CR^{B}, and B⁴ is selected from N, CH, CY¹ and CR^{B}.

In one embodiment, two of B¹, B², B³ and B⁴ are each independently selected from N, CH, CY¹ and CR^{B}, and the remaining two of B¹, B², B³ and B⁴ are each independently selected from N, CH and CY¹. Typically, one of B¹, B², B³ and B⁴ is selected from N, CH, CY¹ and CR^{B}, and the remaining three of B¹, B², B³ and B⁴ are each independently selected from N, CH and CY¹. In one aspect of such an embodiment, each R^{B} where present is independently selected from a methyl or fluoromethyl group.

In a further embodiment, B¹, B², B³ and B⁴ are each independently selected from N, CH and CY¹. In one example of such an embodiment, B¹, B² and B³ are each independently selected from CH and CY¹, and B⁴ is selected from N, CH and CY¹. Most typically, B¹, B² and B³ are each CH, and B⁴ is N.

In one embodiment, each Y¹ is F.

In one embodiment of any of the second to eleventh exemplary embodiments of the first aspect of the invention, L² is a straight-chained alkylene group, wherein the straight-chained alkylene group includes one or two heteroatoms independently selected from O and N in its carbon skeleton, wherein L² has a chain length of from 2 to 8 atoms (more typically from 3 to 6 atoms), and wherein L² may optionally be substituted with one or more fluoro groups and/or one oxo (═O) group and/or with one, two, three or four groups R^{L2}, wherein each R^{L2} is independently selected from a C₁-C₃ alkyl or C₁-C₃ fluoroalkyl group, or wherein any two R^{L2} may together form a straight-chained C₁-C₅ alkylene or a straight-chained C₁-C₅ fluoroalkylene group, wherein one carbon atom in the backbone of the C₁-C₅ alkylene or C₁-C₅ fluoroalkylene group may optionally be replaced by a single oxygen atom. In one aspect of such an embodiment, the atom of L² that is directly attached to ring B is O.

In another embodiment of any of the second to eleventh exemplary embodiments of the first aspect of the invention, L² is a straight-chained alkylene group, wherein the straight-chained alkylene group includes one or two heteroatoms independently selected from O and N in its carbon skeleton, wherein L² has a chain length of from 2 to 8 atoms (more typically from 3 to 6 atoms), and wherein L² may optionally be substituted with one or more fluoro groups and/or one oxo (═O) group and/or with one, two, three or four groups R^{L2}, wherein each R^{L2} is independently selected from a methyl or fluoromethyl group, or wherein any two R^{L2} may together form a straight-chained C₁-C₃ alkylene or a straight-chained C₁-C₃ fluoroalkylene group such that L² contains a single 3-6 membered monocyclic ring. In one aspect of such an embodiment, the atom of L² that is directly attached to ring B is O.

In yet another embodiment, L² is a straight-chained alkylene group, wherein the straight-chained alkylene group includes one or two heteroatoms independently selected from O and N in its carbon skeleton, wherein L² has a chain length of from 2 to 8 atoms, and wherein L² may optionally be substituted with one or more fluoro groups and/or one oxo (═O) group and/or with one, two, three or four groups R^{L2}, wherein each R^{L2} is independently selected from a methyl or fluoromethyl group, or wherein any two R^{L2} attached to the same carbon atom may together with the carbon atom to which they are attached form a cyclopropyl group, wherein the cyclopropyl group may optionally be fluoro-substituted. In one aspect of such an embodiment, the atom of L² that is directly attached to ring B is O.

In one embodiment of any of the second to eleventh exemplary embodiments of the first aspect of the invention, R⁴ is selected from a C₃-C₄ alkyl, C₃-C₄ fluoroalkyl, C₃-C₅ cycloalkyl or C₃-C₅ fluorocycloalkyl group, and R⁵ is hydrogen, F, or a methyl or fluoromethyl group. Typically in such an embodiment, R⁵ is hydrogen or F.

In another embodiment, R⁴ and R⁵ together form a divalent group selected from —CH₂CH₂CH₂—, —CH₂CH₂O— and —OCH₂CH₂—, wherein the divalent group formed by R⁴ and R⁵ may optionally be fluoro-substituted.

In one embodiment of any of the second to eleventh exemplary embodiments of the first aspect of the invention, R⁶ and R⁷ are each independently selected from hydrogen, F, or a methyl or fluoromethyl group. Typically in such an embodiment, R⁶ is hydrogen or F and R⁷ is hydrogen, F, or a methyl or fluoromethyl group.

In one embodiment of the first aspect of the invention, any compound of formula (I), (Ia), (Ib), (Ic), (Ic′), (Id), (Id′), (Ie), (Ie′), (If), (If), (Ig), (Ig′), (Iga), (Ih) or (Ih′) contains from 10 to 80 atoms other than hydrogen or halogen. More typically, any compound of formula (I), (Ia), (Ib), (Ic), (Ic′), (Id), (Id′), (Ie), (Ie′), (If), (If), (Ig), (Ig′), (Iga), (Ih) or (Ih′) contains from 15 to 60 atoms other than hydrogen or halogen. Yet more typically, any compound of formula (I), (Ia), (Ib), (Ic), (Ic′), (Id), (Id′), (Ie), (Ie′), (If), (If), (Ig), (Ig′), (Iga), (Ih) or (Ih′) contains from 20 to 50 atoms other than hydrogen or halogen. More typically still, any compound of formula (I), (Ia), (Ib), (Ic), (Ic′), (Id), (Id′), (Ie), (Ie′), (If), (If), (Ig), (Ig′), (Iga), (Ih) or (Ih′) contains from 25 to 40 atoms other than hydrogen or halogen.

In one aspect of any of the above embodiments, the compound of formula (I), (Ia), (Ib), (Ic), (Ic′), (Id), (Id′), (Ie), (Ie′), (If), (If), (Ig), (Ig′), (Iga), (Ih) or (Ih′) has a molecular weight of from 250 to 2000 Da. Typically, the compound of formula (I), (Ia), (Ib), (Ic), (Ic′), (Id), (Id′), (Ie), (Ie′), (If), (If), (Ig), (Ig′), (Iga), (Ih) or (Ih′) has a molecular weight of from 275 to 900 Da. More typically, the compound of formula (I), (Ia), (Ib), (Ic), (Ic′), (Id), (Id′), (Ie), (Ie′), (If), (If), (Ig), (Ig′), (Iga), (Ih) or (Ih′) has a molecular weight of from 300 to 600 Da.

A second aspect of the invention provides a compound selected from the group consisting of:

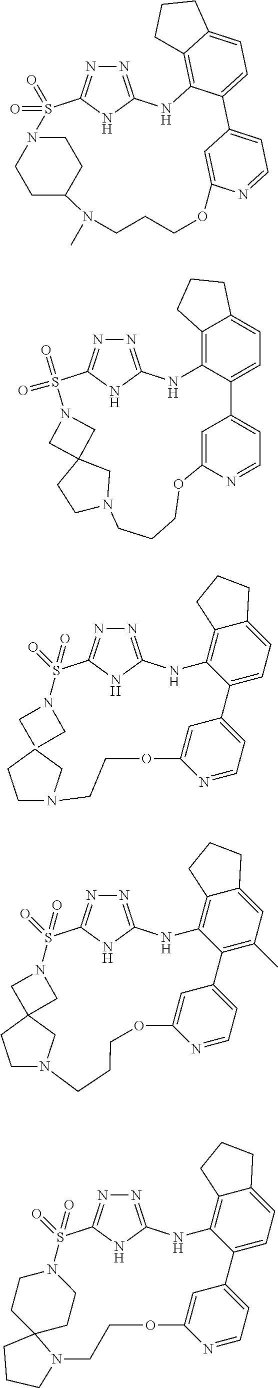 [SMILES: CN1CCCOc2cc(ccn2)-c2ccc3c(c2Nc2nnc([nH]2)S(=O)(=O)N2CCC1CC2)CCC3 ; O=S1(=O)c2nnc([nH]2)Nc2c(ccc3c2CCC3)-c2ccnc(c2)OCCCN2CCC3(C2)CN1C3 ; O=S(=O)(c1nnc(Nc2c(-c3ccnc(OCCN4CCCC4)c3)ccc3c2CCC3)[nH]1)N1CCC1 ; Cc1cc2c(c3c1-c1ccnc(c1)OCCCN1CCC4(C1)CN(C4)S(=O)(=O)c1nnc([nH]1)N3)CCC2 ; O=S1(=O)c2nnc([nH]2)Nc2c(ccc3c2CCC3)-c2ccnc(c2)OCCN2CCCC23CCN1CC3]

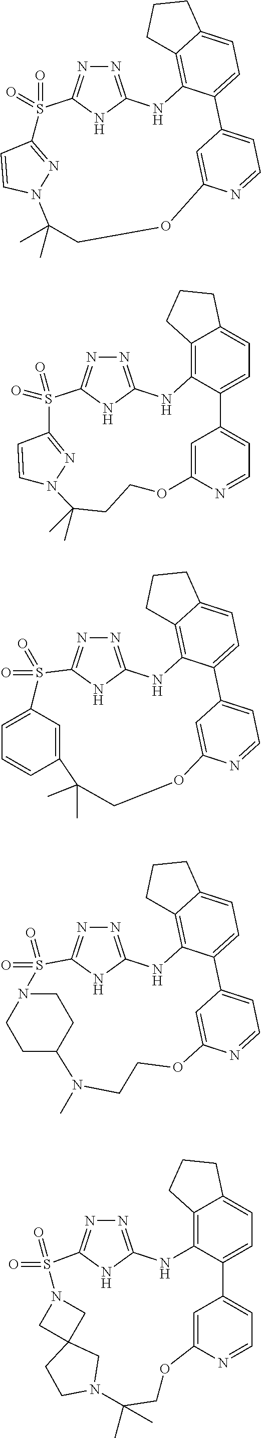 [SMILES: CC1(C)COc2cc(ccn2)-c2ccc3c(c2Nc2nnc([nH]2)S(=O)(=O)c2ccn1n2)CCC3 ; CC1(C)CCOc2cc(ccn2)-c2ccc3c(c2Nc2nnc([nH]2)S(=O)(=O)c2ccn1n2)CCC3 ; CC1(C)COc2cc(ccn2)-c2ccc3c(c2Nc2nnc([nH]2)S(=O)(=O)c2cccc1c2)CCC3 ; CN1CCOc2cc(ccn2)-c2ccc3c(c2Nc2nnc([nH]2)S(=O)(=O)N2CCC1CC2)CCC3 ; CC1(C)COc2cc(ccn2)-c2ccc3c(c2Nc2nnc([nH]2)S(=O)(=O)N2CC4(CCN1C4)C2)CCC3]

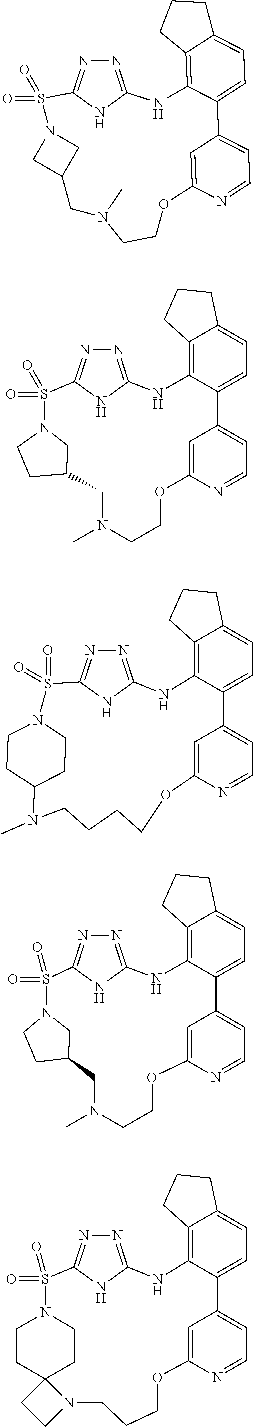

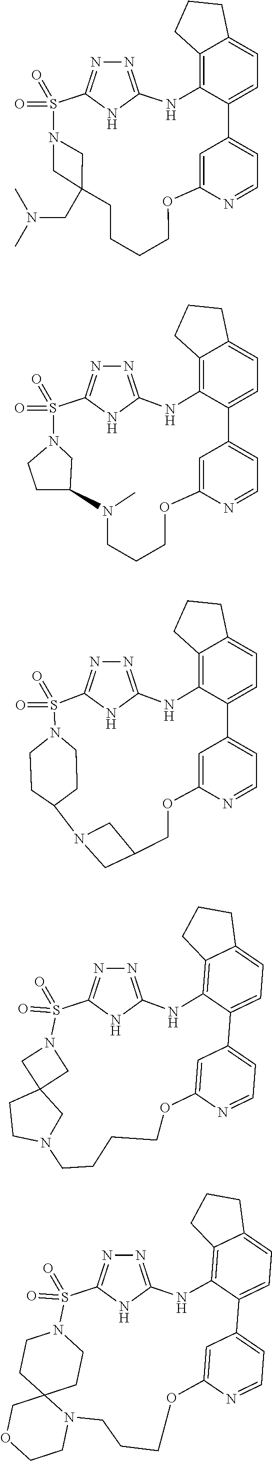 [SMILES: CN(C)CC12CCCCOc3cc(ccn3)-c3ccc4c(c3Nc3nnc([nH]3)S(=O)(=O)N(C1)C2)CCC4 ; CN1CCCOc2cc(ccn2)-c2ccc3c(c2Nc2nnc([nH]2)S(=O)(=O)N2CC[C@H]1C2)CCC3 ; O=S1(=O)c2nnc([nH]2)Nc2c(ccc3c2CCC3)-c2ccnc(c2)OCC2CN(C2)C2CCN1CC2 ; O=S1(=O)c2nnc([nH]2)Nc2c(ccc3c2CCC3)-c2ccnc(c2)OCCCCN2CCC3(C2)CN1C3 ; O=S1(=O)c2nnc([nH]2)Nc2c(ccc3c2CCC3)-c2ccnc(c2)OCCCN2CCOCC23CCN1CC3]

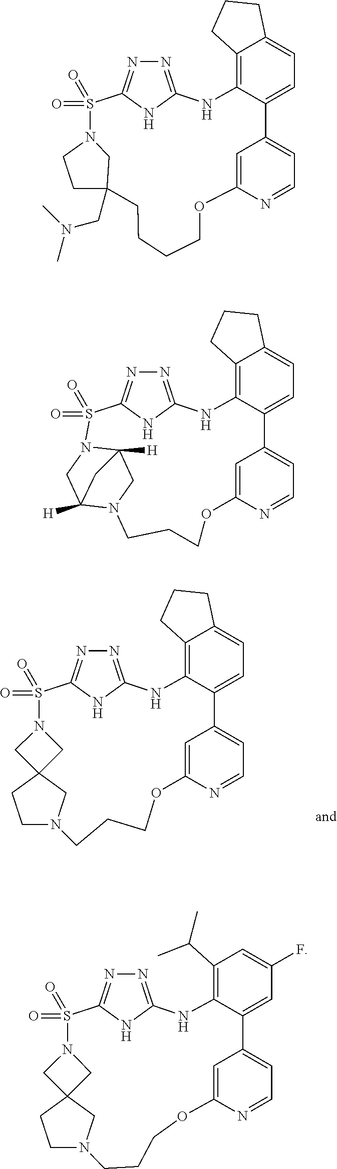 [SMILES: CN(C)CC12CCCCOc3cc(ccn3)-c3ccc4c(c3Nc3nnc([nH]3)S(=O)(=O)N(CC1)C2)CCC4 ; O=S1(=O)c2nnc([nH]2)Nc2c(ccc3c2CCC3)-c2ccnc(c2)OCCCN2C[C@@H]3C[C@H]2CN31 ; O=S1(=O)c2nnc([nH]2)Nc2c(ccc3c2CCC3)-c2ccnc(c2)OCCCN2CCC3(C2)CN1C3 ; CC(C)c1cc(F)cc2c1Nc1nnc([nH]1)S(=O)(=O)N1CC3(CCN(CCCOc4cc-2ccn4)C3)C1]

A third aspect of the invention provides a pharmaceutically acceptable salt, solvate or prodrug of any compound of the first or second aspect of the invention.

The compounds of the present invention can be used both, in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptom, the amelioration or palliation of a condition/symptom, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers.

Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL-1β and IL-18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing an individual as having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

Genetic diseases in which a role for NLRP3 has been suggested include sickle cell disease (Vogel et al., Blood, 130(Suppl 1): 2234, 2017), and Valosin Containing Protein disease (Nalbandian et al., Inflammation, 40(1): 21-41, 2017).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur J Immunol, 40: 595-653, 2010). In particular, NLRP3 mutations have been found to be responsible for a set of rare autoinflammatory diseases known as CAPS (Ozaki et al., J Inflammation Research, 8: 15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type 1 diabetes (T1D), psoriasis, rheumatoid arthritis (RA), Behcet's disease, Schnitzler's syndrome, macrophage activation syndrome, Coeliac disease (Masters, Clin Immunol, 147(3): 223-228, 2013; Braddock et al., Nat Rev Drug Disc, 3: 1-10, 2004; Inoue et al., Immunology, 139: 11-18, 2013; Coll et al., Nat Med, 21(3): 248-55, 2015; Scott et al., Clin Exp Rheumatol, 34(1): 88-93, 2016; Pontillo et al., Autoimmunity, 43(8): 583-589, 2010; and Guo et al., Clin Exp Immunol, 194(2): 231-243, 2018), systemic lupus erythematosus (Lu et al., J Immunol, 198(3): 1119-29, 2017) including lupus nephritis (Zhao et al., Arthritis and Rheumatism, 65(12): 3176-3185, 2013), multiple sclerosis (Xu et al., J Cell Biochem, 120(4): 5160-5168, 2019), and systemic sclerosis (Artlett et al., Arthritis Rheum, 63(11): 3563-74, 2011).

NLRP3 has also been shown to play a role in a number of respiratory and lung diseases including chronic obstructive pulmonary disorder (COPD), asthma (including steroid-resistant asthma and eosinophilic asthma), bronchitis, asbestosis, volcanic ash induced inflammation, and silicosis (Cassel et al., Proceedings of the National Academy of Sciences, 105(26): 9035-9040, 2008; Chen et al., ERJ Open Research, 4: 00130-2017, 2018; Chen et al., Toxicological Sciences, 170(2): 462-475, 2019; Damby et al., Front Immun, 8: 2000, 2018; De Nardo et al., Am J Pathol, 184: 42-54, 2014; Lv et al., J Biol Chem, 293(48): 18454, 2018; and Kim et al., Am J Respir Crit Care Med, 196(3): 283-97, 2017).

NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014; Cheng et al., Autophagy, 1-13, 2020; Couturier et al., J Neuroinflamm, 13: 20, 2016; and Dempsey et al., Brain Behav Immun, 61: 306-316, 2017), intracranial aneurysms (Zhang et al., J Stroke & Cerebrovascular Dis, 24(5): 972-979, 2015), intracerebral haemorrhages (ICH) (Ren et al., Stroke, 49(1): 184-192, 2018), cerebral ischemia-reperfusion injuries (Fauzia et al., Front Pharmacol, 9: 1034, 2018; Hong et al., Neural Plasticity, 2018: 8, 2018; Ye et al., Experimental Neurology, 292: 46-55, 2017), general anesthesia neuroinflammation (Fan et al., Front Cell Neurosci, 12: 426, 2018), sepsis-associated encephalopathy (SAE) (Fu et al., Inflammation, 42(1): 306-318, 2019), perioperative neurocognitive disorders including postoperative cognitive dysfunction (POCD) (Fan et al., Front Cell Neurosci, 12: 426, 2018; and Fu et al., International Immunopharmacology, 82: 106317, 2020), early brain injury (subarachnoid haemorrhage SAH) (Luo et al., Brain Res Bull, 146: 320-326, 2019), and traumatic brain injury (Ismael et al., J Neurotrauma, 35(11): 1294-1303, 2018; and Chen et al., Brain Research, 1710: 163-172, 2019).

NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (NASH) (Mridha et al., J Hepatol, 66(5): 1037-46, 2017).

A role for NLRP3 via IL-1β has also been suggested in atherosclerosis (Chen et al., Journal of the American Heart Association, 6(9): e006347, 2017; and Chen et al., Biochem Biophys Res Commun, 495(1): 382-387, 2018), myocardial infarction (van Hout et al., Eur Heart J, 38(11): 828-36, 2017), cardiovascular disease (Janoudi et al., European Heart Journal, 37(25): 1959-1967, 2016), cardiac hypertrophy and fibrosis (Gan et al., Biochim Biophys Acta, 1864(1): 1-10, 2018), heart failure (Sano et al., J Am Coll Cardiol, 71(8): 875-66, 2018), aortic aneurysm and dissection (Wu et al., Arterioscler Thromb Vase Biol, 37(4): 694-706, 2017), cardiac injury induced by metabolic dysfunction (Pavillard et al., Oncotarget, 8(59): 99740-99756, 2017; and Zhang et al., Biochimica et Biophysica Acta, 1863(6): 1556-1567, 2017), atrial fibrillation (Yao et al., Circulation, 138(20): 2227-2242, 2018), hypertension (Gan et al., Biochim Biophys Acta, 1864(1): 1-10, 2018), and other cardiovascular events (Ridker et al., N Engl J Med, doi: 10.1056/NEJMoa1707914, 2017).

Other diseases, disorders and conditions in which NLRP3 has been shown to be involved include:
ocular diseases such as both wet and dry age-related macular degeneration (Doyle et al., Nature Medicine, 18: 791-798, 2012; and Tarallo et al., Cell, 149(4): 847-59, 2012), diabetic retinopathy (Loukovaara et al., Acta Ophthalmol, 95(8): 803-808, 2017) and optic nerve damage (Puyang et al., Sci Rep, 6: 20998, 2016 Feb. 19);
liver diseases including non-alcoholic steatohepatitis (NASH) (Henao-Meija et al., Nature, 482: 179-185, 2012), ischemia reperfusion injury of the liver (Yu et al., Transplantation, 103(2): 353-362, 2019), fulminant hepatitis (Pourcet et al., Gastroenterology, 154(5): 1449-1464, e20, 2018), liver fibrosis (Zhang et al., Parasit Vectors, 12(1): 29, 2019), and liver failure including acute liver failure (Wang et al., Hepatol Res, 48(3): E194-E202, 2018);
kidney diseases including nephrocalcinosis (Anders et al., Kidney Int, 93(3): 656-669, 2018), kidney fibrosis including chronic crystal nephropathy (Ludwig-Portugall et al., Kidney Int, 90(3): 525-39, 2016), obesity related glomerulopathy (Zhao et al., Mediators of Inflammation, article 3172647, 2019), acute kidney injury (Zhang et al., Diabetes, Metabolic Syndrome and Obesity: Targets and Therapy, 12: 1297-1309, 2019), and renal hypertension (Krishnan et al., Br J Pharmacol, 173(4): 752-65, 2016;
Krishnan et al., Cardiovasc Res, 115(4): 776-787, 2019; Dinh et al., Aging, 9(6): 1595-1606, 2017);
conditions associated with diabetes including diabetic encephalopathy (Zhai et al., Molecules, 23(3): 522, 2018), diabetic retinopathy (Zhang et al., Cell Death Dis, 8(7): e2941, 2017), diabetic nephropathy (also called diabetic kidney disease) (Chen et al., BMC Complementary and Alternative Medicine, 18: 192, 2018), and diabetic hypoadiponectinemia (Zhang et al., Biochimica et Biophysica Acta (BBA)—Molecular Basis of Disease, 1863(6): 1556-1567, 2017);
inflammatory reactions in the lung and skin (Primiano et al., J Immunol, 197(6): 2421-33, 2016) including lung ischemia-reperfusion injury (Xu et al., Biochemical and Biophysical Research Communications, 503(4): 3031-3037, 2018), epithelial to mesenchymal transition (EMT) (Li et al., Experimental Cell Research, 362(2): 489-497, 2018), contact hypersensitivity (such as bullous pemphigoid (Fang et al., J Dermatol Sci, 83(2): 116-23, 2016)), atopic dermatitis (Niebuhr et al., Allergy, 69(8): 1058-67, 2014), Hidradenitis suppurativa (Alikhan et al., J Am Acad Dermatol, 60(4): 539-61, 2009), acne vulgaris (Qin et al., J Invest Dermatol, 134(2): 381-88, 2014), and sarcoidosis (Jager et al., Am J Respir Crit Care Med, 191: A5816, 2015);
inflammatory reactions in the joints (Braddock et al., Nat Rev Drug Disc, 3: 1-10, 2004) and osteoarthritis (Jin et al., PNAS, 108(36): 14867-14872, 2011);
conditions associated with arthritis including arthritic fever (Verma, Linköping University Medical Dissertations, No. 1250, 2011);
amyotrophic lateral sclerosis (Gugliandolo et al., Inflammation, 41(1): 93-103, 2018);
cystic fibrosis (Iannitti et al., Nat Commun, 7: 10791, 2016);
stroke (Walsh et al., Nature Reviews, 15: 84-97, 2014; Ye et al., Experimental Neurology, 292: 46-55, 2017);
headaches including migraine (He et al., Journal of Neuroinflammation, 16: 78, 2019);
chronic kidney disease (Granata et al., PLoS One, 10(3): e0122272, 2015);
Sjögren's syndrome (Vakrakou et al., Journal of Autoimmunity, 91: 23-33, 2018);
graft-versus-host disease (Takahashi et al., Scientific Reports, 7: 13097, 2017);
sickle cell disease (Vogel et al., Blood, 130(Suppl 1): 2234, 2017); and
colitis and inflammatory bowel diseases including ulcerative colitis and Crohn's disease (Braddock et al., Nat Rev Drug Disc, 3: 1-10, 2004; Neudecker et al., J Exp Med, 214(6): 1737-52, 2017; Wu et al., Mediators Inflamm, 2018: 3048532, 2018; and
Lazaridis et al., Dig Dis Sci, 62(9): 2348-56, 2017), and sepsis (intestinal epithelial disruption) (Zhang et al., Dig Dis Sci, 63(1): 81-91, 2018).

Genetic ablation of NLRP3 has been shown to protect from HSD (high sugar diet), HFD (high fat diet) and HSFD-induced obesity (Pavillard et al., Oncotarget, 8(59): 99740-99756, 2017).

The NLRP3 inflammasome has been found to be activated in response to oxidative stress, sunburn (Hasegawa et al., Biochemical and Biophysical Research Communications, 477(3): 329-335, 2016), and UVB irradiation (Schroder et al., Science, 327: 296-300, 2010).

NLRP3 has also been shown to be involved in inflammatory hyperalgesia (Dolunay et al., Inflammation, 40: 366-386, 2017), wound healing (Ito et al., Exp Dermatol, 27(1): 80-86, 2018), burn healing (Chakraborty et al., Exp Dermatol, 27(1): 71-79, 2018), pain including allodynia, multiple sclerosis-associated neuropathic pain (Khan et al., Inflammopharmacology, 26(1): 77-86, 2018), chronic pelvic pain (Zhang et al., Prostate, 79(12): 1439-1449, 2019) and cancer-induced bone pain (Chen et al., Pharmacological Research, 147: 104339, 2019), and intra-amniotic inflammation/infection associated with preterm birth (Faro et al., Biol Reprod, 100(5): 1290-1305, 2019; and Gomez-Lopez et al., Biol Reprod, 100(5): 1306-1318, 2019).

The inflammasome, and NLRP3 specifically, has also been proposed as a target for modulation by various pathogens including bacterial pathogens such as Staphylococcus aureus, including methicillin-resistant Staphylococcus aureus (MRSA) (Cohen et al., Cell Reports, 22(9): 2431-2441, 2018; and Robinson et al., JCI Insight, 3(7): e97470, 2018), Mycobacterium tuberculosis (TB) (Subbarao et al., Scientific Reports, 10: 3709, 2020), Bacillus cereus (Mathur et al., Nat Microbiol, 4: 362-374, 2019), Salmonella typhimurium (Diamond et al., Sci Rep, 7(1): 6861, 2017), and group A Streptococcus (LaRock et al., Science Immunology, 1(2): eaah3539, 2016); viruses such as DNA viruses (Amsler et al., Future Virol, 8(4): 357-370, 2013), influenza A virus (Coates et al., Front Immunol, 8: 782, 2017), chikungunya, Ross river virus, and alpha viruses (Chen et al., Nat Microbiol, 2(10): 1435-1445, 2017); fungal pathogens such as Candida albicans (Tucey et al., mSphere, 1(3), pii: e00074-16, 2016); and other pathogens such as T. gondii (Gov et al., J Immunol, 199(8): 2855-2864, 2017), helminth worms (Alhallaf et al., Cell Reports, 23(4): 1085-1098, 2018), leishmania (Novais et al., PLoS Pathogens, 13(2): e1006196, 2017), and Plasmodium (Strangward et al., PNAS, 115(28): 7404-7409, 2018). NLRP3 has been shown to be required for the efficient control of viral, bacterial, fungal, and helminth pathogen infections (Strowig et al., Nature, 481: 278-286, 2012). NLRP3 activity has also been associated with increased susceptibility to viral infection such as by the human immunodeficiency virus (HIV) (Pontillo et al., J Aquir Immune Defic Syndr, 54(3): 236-240, 2010). An increased risk for early mortality amongst patients co-infected with HIV and Mycobacterium tuberculosis (TB) has also been associated with NLRP3 activity (Ravimohan et al., Open Forum Infectious Diseases, 5(5): ofy075, 2018).

NLRP3 has been implicated in the pathogenesis of many cancers (Menu et al., Clinical and Experimental Immunology, 166: 1-15, 2011; and Masters, Clin Immunol, 147(3): 223-228, 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al., Lancet, S0140-6736(17)32247-X, 2017). Inhibition of the NLRP3 inflammasome or IL-1β has also been shown to inhibit the proliferation and migration of lung cancer cells in vitro (Wang et al., Oncol Rep, 35(4): 2053-64, 2016), and NLRP3 has been shown to suppress NK cell-mediated control of carcinogenesis and metastases (Chow et al., Cancer Res, 72(22): 5721-32, 2012). A role for the NLRP3 inflammasome has been suggested in myelodysplastic syndromes (Basiorka et al., Blood, 128(25): 2960-2975, 2016) and also in the carcinogenesis of various other cancers including glioma (Li et al., Am J Cancer Res, 5(1): 442-449, 2015), colon cancer (Allen et al., J Exp Med, 207(5): 1045-56, 2010), melanoma (Dunn et al., Cancer Lett, 314(1): 24-33, 2012), breast cancer (Guo et al., Scientific Reports, 6: 36107, 2016), inflammation-induced tumours (Allen et al., J Exp Med, 207(5): 1045-56, 2010; and Hu et al., PNAS, 107(50): 21635-40, 2010), multiple myeloma (Li et al., Hematology, 21(3): 144-51, 2016), and squamous cell carcinoma of the head and neck (Huang et al., J Exp Clin Cancer Res, 36(1): 116, 2017; and Chen et al., Cellular and Molecular Life Sciences, 75: 2045-2058, 2018). Activation of the NLRP3 inflammasome has also been shown to mediate chemoresistance of tumour cells to 5-fluorouracil (Feng et al., J Exp Clin Cancer Res, 36(1): 81, 2017), and activation of the NLRP3 inflammasome in peripheral nerves contributes to chemotherapy-induced neuropathic pain (Jia et al., Mol Pain, 13: 1-11, 2017).

Accordingly, any of the diseases, disorders or conditions listed above may be treated or prevented in accordance with the fifth, sixth, seventh, eighth, ninth or tenth aspect of the present invention. Particular examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease including paediatric Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behcet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler's syndrome, macrophage activation syndrome, Blau syndrome, vitiligo or vulvodynia;

(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, squamous cell carcinoma of the head and neck, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;

(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus (including MRSA), Helicobacter pylori, Bacillus anthracis, Bacillus cereus, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, Uropathogenic Escherichia coli (UPEC) or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from Schistosoma, roundworms, tapeworms or flukes), prion infections, and co-infections with any of the aforementioned (e.g. with HIV and Mycobacterium tuberculosis);

(v) central nervous system diseases such as Parkinson's disease, Alzheimer's disease, dementia, motor neuron disease, Huntington's disease, cerebral malaria, brain injury from pneumococcal meningitis, intracranial aneurysms, intracerebral haemorrhages, sepsis-associated encephalopathy, perioperative neurocognitive disorder, postoperative cognitive dysfunction, early brain injury, traumatic brain injury, cerebral ischemia-reperfusion injury, stroke, general anesthesia neuroinflammation and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, cardiac hypertrophy and fibrosis, embolism, aneurysms including abdominal aortic aneurysm, metabolism induced cardiac injury, and pericarditis including Dressler's syndrome;

(viii) respiratory diseases including chronic obstructive pulmonary disorder (COPD), asthma such as allergic asthma, eosinophilic asthma, and steroid-resistant asthma, asbestosis, silicosis, volcanic ash induced inflammation, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4, alcoholic fatty liver disease (AFLD), alcoholic steatohepatitis (ASH), ischemia reperfusion injury of the liver, fulminant hepatitis, liver fibrosis, and liver failure including acute liver failure;

(x) renal diseases including chronic kidney disease, oxalate nephropathy, nephrocalcinosis, glomerulonephritis, diabetic nephropathy, obesity related glomerulopathy, kidney fibrosis including chronic crystal nephropathy, acute renal failure, acute kidney injury, and renal hypertension;

(xi) ocular diseases including those of the ocular epithelium, age-related macular degeneration (AMD) (dry and wet), Sjögren's syndrome, uveitis, corneal infection, diabetic retinopathy, optic nerve damage, dry eye, and glaucoma;

(xii) skin diseases including dermatitis such as contact dermatitis and atopic dermatitis, contact hypersensitivity, psoriasis, sunburn, skin lesions, hidradenitis suppurativa (HS), other cyst-causing skin diseases, pyoderma gangrenosum, and acne vulgaris including acne conglobata;

(xiii) lymphatic conditions such as lymphangitis and Castleman's disease;

(xiv) psychological disorders such as depression and psychological stress;

(xv) graft versus host disease;

(xvi) pain such as pelvic pain, hyperalgesia, allodynia including mechanical allodynia, neuropathic pain including multiple sclerosis-associated neuropathic pain, and cancer-induced bone pain;

(xvii) conditions associated with diabetes including diabetic encephalopathy, diabetic retinopathy, diabetic nephropathy, diabetic vascular endothelial dysfunction, and diabetic hypoadiponectinemia;

(xviii) conditions associated with arthritis including arthritic fever;

(xix) headache including cluster headaches, idiopathic intracranial hypertension, migraine, low pressure headaches (e.g. post-lumbar puncture), Short-Lasting Unilateral Neuralgiform Headache With Conjunctival Injection and Tearing (SUNCT), and tension-type headaches;

(xx) wounds and burns, including skin wounds and skin burns; and

(xxi) any disease where an individual has been determined to carry a germline or somatic non-silent mutation in NLRP3.

In one embodiment, the disease, disorder or condition is selected from:

(i) inflammation;

(ii) an auto-immune disease;

(iii) cancer;

(iv) an infection;

(v) a central nervous system disease;

(vi) a metabolic disease;

(vii) a cardiovascular disease;

(viii) a respiratory disease;

(ix) a liver disease;

(x) a renal disease;

(xi) an ocular disease;

(xii) a skin disease;

(xiii) a lymphatic condition;

(xiv) a psychological disorder;

(xv) graft versus host disease;

(xvi) allodynia;

(xvii) a condition associated with diabetes; and

(xviii) any disease where an individual has been determined to carry a germline or somatic non-silent mutation in NLRP3

In another embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(ii) atopic dermatitis;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) sickle cell disease;

(xxx) systemic juvenile idiopathic arthritis;

(xxxi) systemic lupus erythematosus;

(xxxii) traumatic brain injury;

(xxxiii) transient ischemic attack;

(xxxiv) ulcerative colitis; or

(xxxv) Valosin Containing Protein disease.

In another embodiment of the fifth, sixth, seventh, eighth, ninth or tenth aspect of the present invention, the treatment or prevention comprises a reduction in susceptibility to viral infection. For instance, the treatment or prevention may comprise a reduction in susceptibility to HIV infection.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis;

(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;

(vi) a vascular condition such as atherosclerosis, Behcet's disease, vasculitides, or Wegener's granulomatosis;

(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, Legionella, Lyme disease, influenza A, Epstein-Barr virus infection, viral encephalitis/aseptic meningitis, or pelvic inflammatory disease;

(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;

(xv) a cancer, including those cancers listed above;

(xvi) a burn, wound, trauma, haemorrhage or stroke;

(xvii) radiation exposure;

(xviii) a metabolic disease such as type 2 diabetes (T2D), atherosclerosis, obesity, gout or pseudo-gout; and/or

(xix) pain such as inflammatory hyperalgesia, pelvic pain, allodynia, neuropathic pain, or cancer-induced bone pain.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention.

Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

As will be understood, where the further active agent is a small chemical entity, any reference to a specific small chemical entity below is to be understood to encompass all salt, hydrate, solvate, polymorphic and prodrug forms of the specific small chemical entity. Similarly, where the further active agent is a biologic such as a monoclonal antibody, any reference to a specific biologic below is to be understood to encompass all biosimilars thereof.

In some embodiments, the one or more chemotherapeutic agents are selected from abiraterone acetate, altretamine, amsacrine, anhydrovinblastine, auristatin, azacitidine, 5-azacytidine, azathioprine, adriamycin, bexarotene, bicalutamide, BMS 184476, bleomycin, bortezomib, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cladribine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, ixazomib, lenalidomide, lenalidomide-dexamethasone, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, revlimid, rhizoxin, sertenef, streptozocin, stramustine phosphate, tretinoin, tasonermin, taxol, topotecan, tamoxifen, teniposide, taxane, tegafur/uracil, thalidomide, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), Type I interferon ligands such as interferon alpha and interferon beta, Type I interferon mimetics, Type II interferon ligands such as interferon gamma, Type II interferon mimetics, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), cytokines (including interleukins, such as interleukin-1, interleukin-2, interleukin-5, interleukin-10, interleukin-12, and interleukin-33), interleukin-1 ligands and mimetics (such as rilonacept, anakinra, and anakinra-dexamethasone), interleukin-2 ligands and mimetics, interleukin-5 ligands and mimetics, interleukin-10 ligands and mimetics, interleukin-12 ligands and mimetics, and/or interleukin-33 ligands and mimetics.

In some embodiments, the one or more antibodies may comprise one or more monoclonal antibodies. In some embodiments, the one or more antibodies are anti-TNFα and/or anti-IL-6 antibodies, in particular anti-TNFα and/or anti-IL-6 monoclonal antibodies. In some embodiments, the one or more antibodies are selected from abatacept, abciximab, adalimumab, alemtuzumab, atezolizumab, atlizumab, avelumab, basiliximab, belimumab, benralizumab, bevacizumab, bretuximab vedotin, brodalumab, canakinumab, cetuximab, ceertolizumab pegol, daclizumab, denosumab, dupilumab, durvalumab, eculizumab, efalizumab, elotuzumab, gemtuzumab, golimumab, guselkumab, ibritumomab tiuxetan, infliximab, ipilimumab, ixekizumab, mepolizumab, muromonab-CD3, natalizumab, nivolumab, ofatumumab, omalizumab, palivizumab, panitumuab, pembrolizumab, ranibizumab, reslizumab, risankizumab, rituximab, sarilumab, secukinumab, siltuximab, tildrakizumab, tocilizumab, tositumomab, trastuzumab, and/or ustekinumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from thalidomide, lenalidomide, endostatin, angiogenin inhibitors, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides (CDNs), such as c-di-AMP, c-di-GMP, and cGAMP, and/or modified cyclic di-nucleotides that may include one or more of the following modification features: 2′-O/3′-0 linkage, phosphorothioate linkage, adenine and/or guanine analogue, and/or 2′-OH modification (e.g. protection of the 2′-OH with a methyl group or replacement of the 2′-OH by —F or —N₃). In some embodiments, the one or more STING agonists are selected from BMS-986301, MK-1454, ADU-S100, a diABZI, 3′3′-cGAMP, and/or 2′3′-cGAMP.

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCBo24360, galunisertib, ulocuplumab, BKT140, bavituximab, CC-90002, bevacizumab, and/or MNRP1685A.

In some embodiments, the one or more immunomodulatory agents may comprise a complement pathway modulator. Complement pathway modulators modulate the complement activation pathway. Complement pathway modulators may act to block action of the C3 and/or C3a and/or C3aR1 receptor, or may act to block action of the C5 and/or C5a and/or C5aR₁ receptor. In some embodiments, the complement pathway modulator is a C5 complement pathway modulator and may be selected from eculizumab, ravulizumab (ALXN1210), ABP959, RA101495, tesidolumab (LFG316), zimura, crovalimab (R07112689), pozelimab (REGN3918), GNR-045, SOBI005, and/or coversin. In some embodiments, the complement pathway modulator is a C5a complement pathway modulator and may be selected from cemdisiran (ALN-CC5), IFX-1, IFX-2, IFX-3, and/or olendalizumab (ALXN1007). In some embodiments, the complement pathway modulator is a C5aR1 complement pathway modulator and may be selected from ALS-205, MOR-210/TJ210, DF2593A, DF3016A, DF2593A, avacopan (CCX168), and/or IPH5401.

In some embodiments, the one or more immunomodulatory agents may comprise an anti-TNFα agent. In some embodiments, the anti-TNFα agent may be an antibody or an antigen-binding fragment thereof, a fusion protein, a soluble TNFα receptor (e.g. a soluble TNFR1 or soluble TNFR2), an inhibitory nucleic acid, or a small molecule TNFα antagonist. In some embodiments, the inhibitory nucleic acid may be a ribozyme, a small hairpin RNA, a small interfering RNA, an antisense nucleic acid, or an aptamer.

In some embodiments, the anti-TNFα agent is selected from adalimumab, certolizumab pegol, etanercept, golimumab, infliximab, CDP571, and biosimilars thereof (such as adalimumab-adbm, adalimumab-adaz, adalimumab-atto, etanercept-szzs, infliximab-abda and infliximab-dyyb).

In some embodiments, the one or more immunomodulatory agents may comprise azithromycin, clarithromycin, erythromycin, levofloxacin and/or roxithromycin.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents; anti-inflammatory agents (including non-steroidal anti-inflammatory agents; small molecule anti-inflammatory agents (such as colchicine); and anti-inflammatory biologics that target for example TNF, IL-5, IL-6, IL-17 or IL-33); JAK inhibitors; phosphodiesterase inhibitors; CAR T therapies; anti-psoriatic agents (including anthralin and its derivatives); vitamins and vitamin-derivatives (including retinoinds, and VDR receptor ligands); steroids; corticosteroids; glucocorticoids (such as dexamethasone, prednisone and triamcinolone acetonide); ion channel blockers (including potassium channel blockers); immune system regulators (including cyclosporin, FK 506, and glucocorticoids); lutenizing hormone releasing hormone agonists (such as leuprolidine, goserelin, triptorelin, histrelin, bicalutamide, flutamide and/or nilutamide); hormones (including estrogen); and/or uric acid lowering agents (such as allopurinol).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disease, disorder or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES—COMPOUND SYNTHESIS

All solvents, reagents and compounds were purchased and used without further purification unless stated otherwise.

### Abbreviations

2-MeTHF 2-methyltetrahydrofuran
Ac acetyl
AcCl acetyl chloride
Ac₂O acetic anhydride
AcOH acetic acid
app apparent
aq aqueous
B₂Pin₂ bis(pinacolato)diboron, also called 4,4,4′,4′,5,5,5′,5′-octamethyl-2,2′-bi(1,3,2-dioxaborolane)
Boc tert-butyloxycarbonyl
br broad
Cbz carboxybenzyl
CDI 1,1-carbonyl-diimidazole
conc concentrated
d doublet
DABCO 1,4-diazabicyclo[2.2.2]octane
DBU 1,8-diazabicyclo[5.4.0]undec-7-ene
DCE 1,2-dichloroethane, also called ethylene dichloride
DCM dichloromethane
dd double doublet
ddd double double doublet
DIAD diisopropyl azodicarboxylate
DIPEA, DIEA N,N-diisopropylethylamine, also called Hunig's base
DIBAL diisobutylaluminum hydride
DMA dimethylacetamide
DMAP 4-dimethylaminopyridine, also called N,N-dimethylpyridin-4-amine
DME dimethoxyethane
DMEDA N,N-dimethyl-1,2-ethanediamine
DMF N,N-dimethylformamide
DMF-DMA N,N-dimethylformamide dimethyl acetal
DMSO dimethyl sulfoxide
EDC 1-ethyl-3-(3-dimethylaminopropyl)carbodiimide
eq or equiv equivalent
(ES+) electrospray ionization, positive mode
Et ethyl
EtOAc ethyl acetate
EtOH ethanol
Ex example
FC flash column chromatography on silica gel
h hour(s)
HATU 1-[bis(dimethylamino)methylene]-1H-1,2,3-triazolo[4,5-b]pyridinium 3-oxid hexafluorophosphate
HPLC high performance liquid chromatography
Hz hertz
Int intermediate
KOAc potassium acetate
KO^{t}Bu potassium tert-butoxide
LC liquid chromatography
LHMDS lithium bis(trimethylsilyl)amide
m multiplet
m-CPBA 3-chloroperoxybenzoic acid
Me methyl
MeCN acetonitrile
MeOH methanol
(M+H)⁺ protonated molecular ion
MHz megahertz
min minute(s)
MS mass spectrometry
Ms mesyl, also called methanesulfonyl
MsCl mesyl chloride, also called methanesulfonyl chloride
MTBE methyl tert-butyl ether, also called tert-butyl methyl ether
m/z mass-to-charge ratio
NaOMe sodium methoxide
NaO^{t}Bu sodium tert-butoxide
NBS 1-bromopyrrolidine-2,5-dione, also called N-bromosuccinimide
NCS 1-chloropyrrolidine-2,5-dione, also called N-chlorosuccinimide
NHMDS sodium bis(trimethylsilyl)amide
NMP N-methylpyrrolidine
NMR nuclear magnetic resonance (spectroscopy)
p pentuplet
Pd₂(dba)₃ tris(dibenzylideneacetone) dipalladium(0)
PdCl₂(dppf) [1,1′-bis(diphenylphosphino)ferrocene] dichloropalladium(II), also called Pd(dppf)Cl₂
PE petroleum ether
Ph phenyl
PMB p-methoxybenzyl, also called 4-methoxybenzyl
prep-HPLC preparative high performance liquid chromatography
prep-TLC preparative thin layer chromatography
PTSA p-toluenesulfonic acid
q quartet
quant. quantitative
RP reversed phase
RT room temperature
s singlet
sat saturated
SCX solid supported cation exchange (resin)
SEM 2-(trimethylsilyl)ethoxymethyl
sept septuplet
SPhos 2-Dicyclohexylphosphino-2′,6′-dimethoxybiphenyl
SPhos-Pd-G3 (2-dicyclohexylphosphino-2′,6′-dimethoxybiphenyl) [2-(2′-amino-1,1′-biphenyl)]palladium(II) methanesulfonate
t triplet
TBDMS tert-butyl dimethylsilyl
tBu tert-butyl
T3P propylphosphonic anhydride
TBME tert-butyl methyl ether, also called methyl tert-butyl ether
TEA triethylamine
Tf triflyl, also called trifluoromethanesulfonyl
TFA 2,2,2-trifluoroacetic acid
TFAA trifluoroacetic anhydride
THF tetrahydrofuran
TLC thin layer chromatography
TMS trimethylsilyl
wt % weight percent or percent by weight
XantPhos 4,5-bis(diphenylphosphino)-9,9-dimethylxanthene
Xphos 2-dicyclohexylphosphino-2′,4′,6′-triisopropylbiphenyl
XPhos-Pd-G3 (2-dicyclohexylphosphino-2′,4′,6′-triisopropyl-1,1′-biphenyl)[2-(2′-amino-1,1′-biphenyl)]palladium(II) methanesulfonate

Experimental Methods

Nuclear Magnetic Resonance

Spectra were measured at 298 K, unless indicated otherwise, and were referenced relative to the solvent resonance. The chemical shifts are reported in parts per million.

LC-MS

LC-MS Methods: Using Agilent 1100 & DAD detector. Mobile Phase A: 0.1% HCOOH in water (v/v); B: acetonitrile. Column: EVO C18 3.0×50 mm, 5 μm.

Preparative Reversed Phase HPLC General Methods

Neutral prep-HPLC (x-y % MeCN in water): C18 column, eluting with a H₂O-MeCN gradient using UV detection at 214 and 254 nm.

Basic prep-HPLC (x-y % MeCN in water): C18 column, eluting with a 10 mM NH₄HCO₃-MeCN gradient using UV detection at 214 and 254 nm.

Acidic prep-HPLC (x-y % MeCN in water): C18 column, eluting with a water (0.1% formic acid)-MeCN (0.1% formic acid) gradient using UV detection at 214 and 254 nm.

### Synthesis of Intermediates

### Intermediate A1: 2-[(3,5-dibromo-1,2,4-triazol-1-yl)methoxy]ethyl-trimethyl-silane

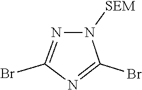 [SMILES: C[Si](C)(C)CCOCn1nc(Br)nc1Br]

2-(Trimethylsilyl)ethoxymethyl chloride (20.7 mL, 0.117 mol) was added to a mixture of 3,5-dibromo-4H-1,2,4-triazole (25.0 g, 0.110 mol), and K₂CO₃ (22.8 g, 0.165 mol) in MeCN (250.0 mL). The mixture was stirred at 22° C. for 4 h. The mixture was filtered, and the filtrate was concentrated. The product was purified by FC (0-50% EtOAc/hexanes) to provide the title compound as an oil (32.2 g, 82%).

¹H NMR (500 MHz, CDCl₃) δ 5.43 (s, 2H), 3.71-3.58 (m, 2H), 1.00-0.87 (m, 2H), −0.02 (s, 9H).

### Intermediate A2: methyl 3-[[5-[[5-(2-fluoro-4-pyridyl)indan-4-yl]amino]-1-(2-trimethylsilylethoxymethyl)-1,2,4-triazol-3-yl]sulfanyl]propanoate

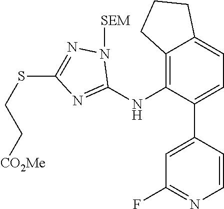 [SMILES: COC(=O)CCSc1nc(Nc2c(-c3ccnc(F)c3)ccc3c2CCC3)n(COCC[Si](C)(C)C)n1]

### Step A: N-indan-4-ylacetamide

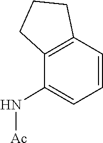 [SMILES: CC(=O)Nc1cccc2c1CCC2]

Acetic anhydride (12.8 mL, 134 mmol) was added to a mixture of indan-4-amine (14.4 mL, 116 mmol) and TEA (21.4 mL, 152 mmol) in DCM (225 mL) at 0° C. The mixture was stirred at 22° C. for 1 h and diluted with aq. HCl (1M, 50.0 mL). The aqueous phase was extracted with DCM (3×50.0 mL), and the combined organic layers were washed with sat. aq. NaHCO₃ (50.0 mL), dried (Na₂SO₄), filtered, and concentrated to provide the title compound as a solid (20.1 g, 99%).

LCMS m/z 176.49 (M+H)⁺ (ES⁺).

¹H NMR (400 MHz, CDCl₃) δ 7.71 (d, J=8.0 Hz, 1H), 7.14 (t, J=7.7 Hz, 1H), 7.02 (d, J=7.4 Hz, 2H), 2.94 (t, J=7.5 Hz, 2H), 2.80 (t, J=7.4 Hz, 2H), 2.18 (s, 3H), 2.09 (dd, J=14.9, 7.3 Hz, 2H).

### Step B: N-(5-bromoindan-4-yl)acetamide

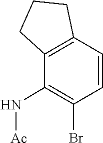 [SMILES: CC(=O)Nc1c(Br)ccc2c1CCC2]

Pd(OAc)₂ (98.0%, 1.27 g, 5.53 mmol) was added to a mixture of N-indan-4-ylacetamide (94.0%, 20.6 g, 111 mmol) and PTSA (98.5%, 11.7 g, 60.8 mmol) in toluene (250 mL). The mixture was stirred at 22° C. for 5 min, and NBS (99.0%, 21.9 g, 122 mmol) was added. The mixture was stirred at 22° C. for 18 h and then diluted with sat. aq. Na₂S203 (200 mL), and EtOAc (500 mL). The organic phase was washed with sat aq. NaHCO₃ (250 mL) and stirred with Na₂SO₄ and activated carbon (3 g) for 1 h. The mixture was filtered on Celite washing with DCM (200 mL). The filtrate was concentrated to 200 mL. The mixture was diluted with hexanes (500 mL) and then filtered. The solid was washed with hexanes (200 mL) and dried to provide the title compound as a solid (24.1 g, 86%).

LCMS m/z 255 (M+H)⁺ (ES⁺).

¹H NMR (400 MHz, CDCl₃) δ 7.34 (d, J=7.8 Hz, 1H), 7.03 (brs, 1H), 6.99 (d, J=8.0 Hz, 1H), 2.89 (dt, J=15.2, 7.4 Hz, 4H), 2.22 (s, 3H), 2.12-2.01 (m, 2H).

### Step C: (5-bromoindan-4-yl)ammonium chloride

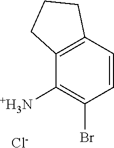 [SMILES: [NH3+]c1c(Br)ccc2c1CCC2 ; [Cl-]]

HCl (12.0 M, 158 mL, 1.90 mol) was added to a mixture of N-(5-bromoindan-4-yl)-acetamide (24.1 g, 94.8 mmol) in water (158 mL). The mixture was stirred at 100° C. for 18 h and filtered. The solid was washed with water (300 mL) and EtOAc (100 mL) and dried to provide the title compound as a solid (17 g, 72%).

LCMS m/Z 214.1 (M-Cl)⁺ (ES⁺).

¹H NMR (400 MHz, DMSO-d₆) δ 7.14 (d, J=7.9 Hz, 1H), 6.48 (d, J=7.9 Hz, 1H), 6.01 (s, 3H), 2.80-2.75 (m, 2H), 2.75-2.70 (m, 2H), 1.99 (p, J=7.5 Hz, 2H).

### Step D: 5-(2-fluoro-4-pyridyl)indan-4-amine

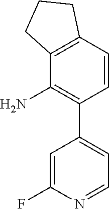 [SMILES: Nc1c(-c2ccnc(F)c2)ccc2c1CCC2]

Pd(dppf)Cl₂-DCM (99.0%, 1.78 g, 2.16 mmol) was added to a degassed mixture of (5-bromoindan-4-yl)ammonium chloride (10.4 g, 41.7 mmol), (2-fluoro-4-pyridyl)boronic acid (98.0%, 7.34 g, 51.1 mmol), and K₂CO₃ (99.0%, 18.0 g, 129 mmol) in 1,4-dioxane and water (5:1, 158.5 mL) at 22° C. under N₂. The mixture was stirred at 80° C. for 3 h, and activated carbon was added (2.00 g). The mixture was stirred at 20° C. for 30 min, then filtered on Celite, washing with EtOAc (200 mL). The filtrate was diluted with EtOAc (300 mL) and sat. aq. NaHCO₃ (300 mL). The organic phase was washed with brine (300 mL) and stirred with Na₂SO₄ and activated carbon (2.00 g) for 10 min. The mixture was filtered on celite, washing with EtOAc (100 mL), and the filtrate was concentrated. The residue was filtered on silica gel, washing with 10% EtOAc in hexanes (1.00 L). The filtrate was concentrated to provide the title compound as a solid (10.9 g, 70%).

LCMS m/z 229.4 (M+H)⁺ (ES⁺).

¹H NMR (400 MHz, CDCl₃) δ 8.24 (d, J=5.1 Hz, 1H), 7.32 (ddd, J=5.1, 2.0, 1.4 Hz, 1H), 7.06 (d, J=0.8 Hz, 1H), 6.97 (d, J=7.6 Hz, 1H), 6.78 (d, J=7.6 Hz, 1H), 3.78 (s, 2H), 2.97 (t, J=7.6 Hz, 2H), 2.77 (t, J=7.4 Hz, 2H), 2.27-2.07 (m, 2H).

¹⁹F NMR (376 MHz, CDCl₃) δ −67.83 (s)

### Step E: 5-bromo-N-[5-(2-fluoro-4-pyridyl)indan-4-yl]-2-(2-trimethylsilylethoxy-methyl)-1,2,4-triazol-3-amine

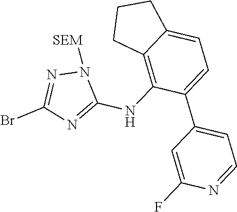 [SMILES: C[Si](C)(C)CCOCn1nc(Br)nc1Nc1c(-c2ccnc(F)c2)ccc2c1CCC2]

LHMDS (1.00 M, 150 mL, 150 mmol) was added over 15 min to a mixture of 5-(2-fluoro-4-pyridyl)indan-4-amine (10.9 g, 47.8 mmol) and 2-[(3,5-dibromo-1,2,4-triazol-1-yl)methoxy]ethyl-trimethyl-silane (Intermediate A1) (41.5 g, 116 mmol) in 2-MeTHF (50.0 mL) at 0° C. The mixture was stirred at 0° C. for 3 h and at 20° C. for 1 h. The mixture was diluted with EtOAc (500 mL) and sat. aq. NH₄Cl (400 mL). The organic phase was washed with sat. aq. NaHCO₃ (300 mL) and brine (300 mL). The organic phase was mixed with Na₂SO₄ and activated carbon. The mixture was filtered on Celite, and the filtrate was concentrated. The residue was filtered on a silica pad, washing with hexanes (250 mL). A precipitate formed. The suspension was filtered, and the solid was rinsed with hexanes (100 mL) and dried to provide a solid. The silica pad was re-washed with 5% EtOAc in hexanes (500 mL). The filtrate was concentrated, diluted with hexanes (300 mL), filtered, and dried to provide a solid. The silica pad was re-washed again with 10% EtOAc in hexanes (1.00 L), and the filtrate was concentrated and dried to provide a solid. All solid materials were combined to provide the title compound as a solid (10.5 g, 44%).

LCMS m/z 504.3 (M+H)⁺ (ES⁺).

¹H NMR (400 MHz, CDCl₃) δ 8.20 (d, J=5.1 Hz, 1H), 7.22 (d, J=7.8 Hz, 1H), 7.20-7.16 (m, 1H), 7.12 (d, J=7.6 Hz, 1H), 6.94 (s, 1H), 6.18 (s, 1H), 5.26 (s, 2H), 3.56-3.47 (m, 2H), 3.02 (t, J=7.5 Hz, 2H), 2.78 (t, J=7.4 Hz, 2H), 2.22-2.07 (m, 2H), 0.79-0.70 (m, 2H), 0.01 (s, 9H).

¹⁹F NMR (376 MHz, CDCl₃) δ −67.39 (s).

### Step F: methyl 3-[[5-[[5-(2-fluoro-4-pyridyl)indan-4-yl]amino]-1-(2-trimethylsilyl-ethoxymethyl)-1,2,4-triazol-3-yl]sulfanyl]propanoate

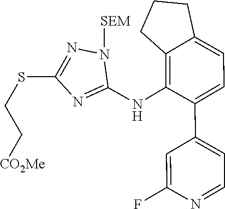 [SMILES: COC(=O)CCSc1nc(Nc2c(-c3ccnc(F)c3)ccc3c2CCC3)n(COCC[Si](C)(C)C)n1]

DIPEA (2.51 mL, 14.7 mmol) was added to a mixture of 5-bromo-N-[5-(2-fluoro-4-pyridyl)indan-4-yl]-2-(2-trimethylsilylethoxymethyl)-1,2,4-triazol-3-amine (3.70 g, 7.33 mmol), Pd₂(dba)₃ (0.672 g, 0.733 mmol), Xantphos (0.424 g, 0.733 mmol), and methyl 3-sulfanylpropanoate (1.62 mL, 14.7 mmol) in dioxane (50.0 mL) at 22° C. under N₂. The mixture was stirred at 100° C. for 16 h and diluted with water (100 mL). The aqueous phase was extracted with EtOAc (3×100 mL), and the combined organic phases were dried (Na₂SO₄), filtered, and concentrated. The product was purified by FC (0-50% EtOAc/hexanes) to provide the title compound as an oil (3.20 g, 80%).

LCMS m/z 566.4 (M+Na)⁺ (ES⁺).

¹H NMR (400 MHz, CDCl₃) δ 8.18 (d, J=5.2 Hz, 1H), 7.20 (dd, J=4.6, 2.6 Hz, 2H), 7.11 (d, J=7.7 Hz, 1H), 6.95 (s, 1H), 6.09 (s, 1H), 5.23 (s, 2H), 3.69 (s, 3H), 3.55-3.44 (m, 2H), 3.22 (t, J=7.3 Hz, 2H), 3.00 (t, J=7.4 Hz, 2H), 2.76 (td, J=7.3, 3.1 Hz, 4H), 2.11 (p, J=7.5 Hz, 2H), 0.81-0.63 (m, 2H), −0.00 (s, 9H).

¹⁹F NMR (471 MHz, CDCl₃) δ −67.52 (s).

### Intermediate A3: sodium 5-[[5-(2-fluoro-4-pyridyl)indan-4-yl]amino]-1-(2-trimethylsilylethoxymethyl)-1,2,4-triazole-3-sulfinate

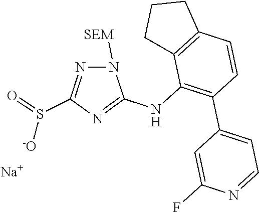

### Step A: methyl 3-[[5-[[5-(2-fluoro-4-pyridyl)indan-4-yl]amino]-1-(2-trimethylsilyl-ethoxymethyl)-1,2,4-triazol-3-yl]sulfonyl]propanoate

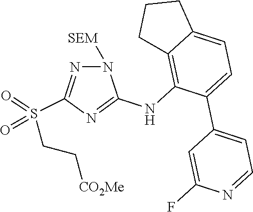 [SMILES: COC(=O)CCS(=O)(=O)c1nc(Nc2c(-c3ccnc(F)c3)ccc3c2CCC3)n(COCC[Si](C)(C)C)n1]

m-CPBA (3.30 g, 14.7 mmol) was added to a mixture of methyl 3-[[5-[[5-(2-fluoro-4-pyridyl)indan-4-yl]amino]-1-(2-trimethylsilylethoxymethyl)-1,2,4-triazol-3-yl]-sulfanyl]propanoate (Intermediate A2) (3.20 g, 5.89 mmol) in DCM (60.0 mL) at 0° C. under N₂. The mixture was stirred at 22° C. for 4 h and diluted with Na₂S₂O₃ (aq. sat., 50 mL). The aqueous phase was extracted with CHCl₃ (3×100 mL), and the combined organic phases were dried (Na₂SO₄), filtered and concentrated. The product was purified by FC (0-100% EtOAc/hexanes) to provide the title compound as a solid (3.01 g, 88%).

LCMS m/z 576.1 (M+H)⁺ (ES⁺).

¹H NMR (500 MHz, CDCl₃) δ 8.19 (d, J=5.1 Hz, 1H), 7.24 (s, 1H), 7.20 (dt, J=5.1, 1.5 Hz, 1H), 7.14 (d, J=7.7 Hz, 1H), 6.94 (s, 1H), 6.49 (s, 1H), 5.39 (s, 2H), 3.71 (s, 3H), 3.64-3.43 (m, 4H), 3.02 (t, J=7.4 Hz, 2H), 2.79 (ddd, J=21.2, 11.4, 5.7 Hz, 4H), 2.13 (dq, J=14.8, 7.5 Hz, 2H), 0.91-0.72 (m, 2H), 0.01 (s, 9H).

¹⁹F NMR (376 MHz, CDCl₃) δ −67.40 (s).

### Step B: sodium 5-[[5-(2-fluoro-4-pyridyl)indan-4-yl]amino]-1-(2-trimethylsilylethoxy-methyl)-1,2,4-triazole-3-sulfinate

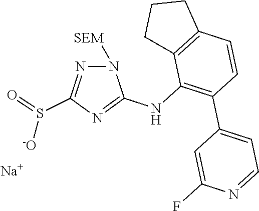

Sodium 2-methylbutan-2-olate (1.00 M in THF, 12.5 mL, 12.5 mmol) was added to a mixture of methyl 3-[[5-[[5-(2-fluoro-4-pyridyl)indan-4-yl]amino]-1-(2-trimethylsilyl-ethoxymethyl)-1,2,4-triazol-3-yl]sulfonyl]propanoate (79%, 2.78 g, 3.81 mmol) in THF (50.0 mL) at 0° C. The mixture was stirred at 0° C. for 1 h. Dowex MAC-3 Hydrogen form (7.11 g, 22.9 mmol) was added, and the mixture was stirred at 22° C. for 10 min. The mixture was filtered, washing with Et₂O (20 mL). The filtrate was concentrated. The residue was diluted with hexanes (30.0 mL) and concentrated four times. The product was dried to provide the title compound as a solid (2.01 g, 82%).

LCMS m/z 490.4 (M-Na+2H)⁺ (ES⁺).

¹H NMR (400 MHz, DMSO-d₆) δ 8.27 (d, J=5.3 Hz, 1H), 8.18 (d, J=5.2 Hz, 1H), 7.32 (d, J=5.2 Hz, 1H), 7.24 (s, 2H), 7.12 (s, 1H), 5.25 (s, 2H), 3.56-3.42 (m, 2H), 2.95 (t, J=7.5 Hz, 2H), 2.61 (t, J=7.3 Hz, 2H), 2.00 (dd, J=14.6, 7.2 Hz, 2H), 0.91-0.74 (m, 2H), −0.03 (s, 9H).

### Intermediate A4: ammonium 5-[[5-(2-fluoro-4-pyridyl)indan-4-yl]amino]-1-(2-trimethylsilylethoxymethyl)-1,2,4-triazole-3-sulfinate

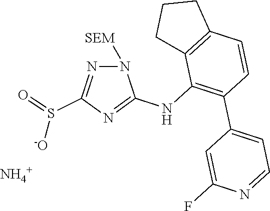

tBuONa (361 mg, 3.75 mmol) was added to a mixture of methyl 3-[[5-[[5-(2-fluoro-4-pyridyl)indan-4-yl]amino]-1-(2-trimethylsilylethoxymethyl)-1,2,4-triazol-3-yl]-sulfonyl]propanoate (Intermediate A3, Step A) (1.80 g, 3.13 mmol) in THF (30.0 mL) at 0° C. under N₂. The mixture was stirred at 0° C. for 2 h and diluted with aq. NH₄Cl (1.00 mL). The mixture was concentrated. The product was purified by basic prep HPLC (0-100% MeCN in water) to provide the title compound as a solid (506 mg, 32%).

LCMS m/z 490.3 (M-NH₄+2H)⁺ (ES⁺).

¹H NMR (400 MHz, DMSO-d₆) δ 8.23 (s, 1H), 8.18 (d, J=5.2 Hz, 1H), 7.31 (d, J=4.4 Hz, 1H), 7.24 (br, 2H), 7.14 (br, 4H), 7.11 (s, 1H), 5.23 (s, 2H), 3.55-3.45 (m, 2H), 2.95 (t, J=7.3 Hz, 2H), 2.61 (t, J=7.2 Hz, 2H), 2.04-1.93 (m, 2H), 0.85-0.76 (m, 2H), −0.03 (s, 9H).

¹⁹F NMR (376 MHz, DMSO-d6) δ −69.20 (s).

### Intermediate A5: 3-hydroxypropyl-methyl-piperidin-1-ium-4-yl-ammonium dichloride

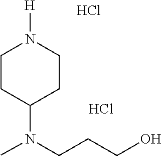

### Step A: tert-butyl4-[3-hydroxypropyl(methyl)amino]piperidine-1-carboxylate

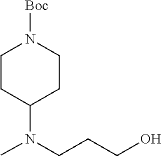 [SMILES: CN(CCCO)C1CCN(C(=O)OC(C)(C)C)CC1]

tert-Butyl 4-(methylamino)piperidine-1-carboxylate (2.50 g, 11.7 mmol) was added to a mixture of 3-bromopropan-1-ol (1.12 ml, 12.8 mmol) and DIPEA (3.00 mL, 17.5 mmol) in MeCN (50.0 ml) at 22° C. under N₂. The mixture was stirred at 60° C. for 16 h and concentrated. The product was purified by neutral prep HPLC (0-40% MeCN in water) to provide the title compound as an oil (790 mg, 25%).

¹H NMR (400 MHz, CDCl₃) δ 4.18 (s, 2H), 3.92-3.72 (m, 2H), 2.74-2.68 (m, 2H), 2.67-2.63 (m, 2H), 2.59 (tt, J=11.9, 3.7 Hz, 1H), 2.28 (s, 3H), 1.70 (td, J=11.5, 5.7 Hz, 4H), 1.61 (s, 1H), 1.45 (s, 9H), 1.49-1.36 (m, 2H).

### Step B: 3-hydroxypropyl-methyl-piperidin-1-ium-4-yl-ammonium dichloride

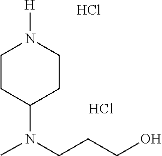

4 M HCl in dioxane (7.25 ml, 29.2 mmol) was added to a mixture of tert-butyl 4-[3-hydroxypropyl(methyl)amino]piperidine-1-carboxylate (790 mg, 2.90 mmol) in MeOH (3.5 ml) at 22° C. under N₂. The mixture was stirred at 22° C. for 4 h and diluted with Et₂O (20.0 ml). The mixture was filtered washing with Et₂O (2×20 mL). The filtrate was concentrated to provide the title compound as a solid (600 mg, 84%).

¹H NMR (500 MHz, DMSO-d₆) δ 10.94 (s, 1H), 9.22 (s, 1H), 9.03 (d, J=9.8 Hz, 1H), 3.76-3.44 (m, 4H), 3.40 (m, 2H), 3.23-3.11 (m, 1H), 3.11-3.00 (m, 1H), 2.92 (s, 2H), 2.68 (d, J=4.9 Hz, 3H), 2.25 (m, 1H), 2.17 (m, 1H), 2.03-1.78 (m, 4H).

### Intermediate A6: 5-bromo-6-methyl-4-nitro-indane

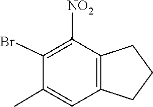

### Step A: N-indan-5-ylacetamide

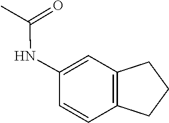

TEA (11.8 mL, 84.5 mmol) was added to a mixture of indan-5-amine (7.50 g, 56.3 mmol) in DCM (125 mL) at 0° C. The mixture was stirred for 20 min, and acetyl chloride (12.1 mL, 169 mmol) was added. The mixture was stirred for 30 min at 0° C., and at 22° C. for 16 h. The mixture was diluted with MeOH (20.0 mL, and water (30.0 mL). The aqueous phase was extracted with DCM (3×40.0 mL), and the combined organic phases were dried (Na₂SO₄) and concentrated. The residue was purified by FC (20-60% EtOAc/hexanes) to provide the title compound as a solid (9.0 g, 91%).

LCMS m/z 176.2 (M+H)⁺ (ES⁺).

¹H NMR (500 MHz, CDCl₃) δ 7.44 (s, 1H), 7.18-7.10 (m, 3H), 2.86 (dd, J=17.7, 7.5 Hz, 4H), 2.14 (s, 3H), 2.05 (p, J=7.4 Hz, 2H).

### Step B: N-(6-bromoindan-5-yl)acetamide

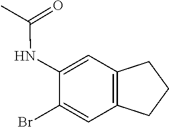

Bromine (3.29 mL, 64.2 mmol) was a e to a mixture of N-indan-5-ylacetamide (9.00 g, 51.4 mmol) in acetic acid (175 mL) at 0° C. The mixture was stirred at 22° C. for 1.5 h. The mixture was diluted with water, and the precipitate was filtered, washed with water (50.0 mL), and dried to provide the title compound as a solid (13.0 g, 99%).

LCMS m/z 255.2 (M+H)⁺ (ES⁺).

¹H NMR (400 MHz, CDCl₃) δ 8.11 (s, 1H), 7.37 (s, 1H), 2.86 (q, J=7.4 Hz, 5H), 2.22 (s, 3H), 2.06 (dd, J=14.9, 7.4 Hz, 2H).

### Step C: N-(6-bromo-4-nitro-indan-5-yl)acetamide

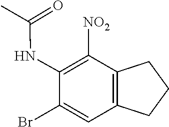 [SMILES: CC(=O)Nc1c(Br)cc2c(c1[N+](=O)[O-])CCC2]

Fuming HNO₃ (4.96 ml, 108 mmol) was added to a mixture of N-(6-bromoindan-5-yl)acetamide (13.7 g, 53.9 mmol) in TFA (60.0 mL) at 0° C. under N₂. The mixture was stirred at 0° C. for 45 min and poured into ice (200 mL). The aqueous phase was extracted with DCM (4×150 mL), and the combined organic phases were washed with brine (200 mL), dried (Na₂SO₄), filtered, and concentrated to provide the title compound as a solid (16.1 g, 99%).

LCMS m/z 300.0 (M+H)⁺ (ES⁺).

¹H NMR (400 MHz, CDCl₃) δ 7.65 (s, 1H), 7.38 (s, 1H), 3.09 (t, J=7.5 Hz, 2H), 2.99 (t, J=7.6 Hz, 2H), 2.21 (s, 3H), 2.18-2.13 (m, 2H).

### Step D: N-(6-methyl-4-nitro-indan-5-yl)acetamide

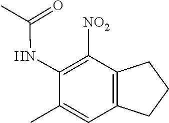 [SMILES: CC(=O)Nc1c(C)cc2c(c1[N+](=O)[O-])CCC2]

1,1′-Bis(diphenylphosphino)ferrocenedichloropalladium(II) (2.89 g, 3.53 mmol) was added to a mixture of N-(6-bromo-4-nitro-indan-5-yl)acetamide (10.6 g, 35.3 mmol), methylboronic acid (6.35 g, 163 mmol), and K₂CO₃ (22.5 g, 163 mmol) in 1,4-dioxane (250 mL) and water (70.0 mL) at 22° C. under N₂. The mixture was stirred at 80° C. for 16 h and poured into ice. The aqueous phase was extracted with EtOAc (4×200 mL), and the combined organic phases were washed with brine (300 mL), dried (Na₂SO₄), filtered, and concentrated. The product was purified by FC (0-2% MeOH/DCM) to provide the title compound as a solid (4.47 g, 54%).

LCMS m/z 235.3 (M+H)⁺ (ES⁺).

¹H NMR (400 MHz, CDCl₃) δ 7.74 (s, 1H), 7.31 (s, 1H), 3.09 (t, J=7.5 Hz, 2H), 2.94 (t, J=7.5 Hz, 2H), 2.26 (s, 3H), 2.18 (s, 3H), 2.12 (p, J=7.6 Hz, 2H).

### Step E: 6-methyl-4-nitro-indan-5-amine

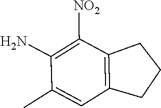

HCl (6M in water, 134 mL, 804 mmol) was added to a mixture of N-(6-methyl-4-nitro-indan-5-yl)acetamide (6.06 g, 25.9 mmol) at 22° C. under N₂. The mixture was stirred at 100° C. for 16 h and poured into a mixture of ice (140 mL) and NaOH (36.2 g, 905 mmol). The aqueous phase was extracted with EtOAc (3×250 mL), and the combined organic phases were washed with brine (200 mL), dried (Na₂SO₄), filtered, and concentrated to provide the title compound as a solid (4.82 g, 97%).

LCMS m/z 193.2 (M+H)⁺ (ES⁺).

¹H NMR (400 MHz, CDCl₃) δ 7.14 (s, 1H), 5.80 (s, 2H), 3.29 (t, J=7.5 Hz, 2H), 2.81 (dd, J=11.5, 4.3 Hz, 2H), 2.19 (t, J=2.8 Hz, 3H), 2.09-1.99 (m, 2H).

### Step F: 5-bromo-6-methyl-4-nitro-indane

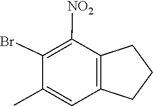

Isoamyl nitrite (0.240 ml, 1.79 mmol) was added to a mixture of 6-methyl-4-nitro-indan-5-amine (0.150 g, 0.780 mmol), cupric bromide (1.74 mg, 0.0078 mmol) and copper(I) bromide (134 mg, 0.936 mmol) in MeCN (2.60 ml) at 0° C. under N₂. The mixture was stirred at 60° C. for 3 h and diluted with water (20.0 mL). The aqueous phase was extracted with EtOAc (3×20.0 mL), and the combined organic phases were washed with brine (10.0 mL), dried (Na₂SO₄), filtered, and concentrated. The product was purified by FC (0-100% EtOAc/hexanes) to provide the title compound as a solid (120 mg, 60%).

¹H NMR (500 MHz, DMSO-d₆) δ 7.49 (s, 1H), 2.90 (dt, J=10.4, 7.5 Hz, 4H), 2.40 (s, 3H), 2.08 (p, J=7.6 Hz, 2H).

### Intermediate A7: 3-(2,6-diazaspiro[3.4]octan-6-yl)propan-1-ol

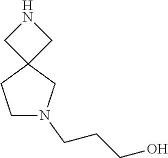 [SMILES: OCCCN1CCC2(CNC2)C1]

### Step A: tert-butyl 7-[3-[tert-butyl(dimethyl)silyl]oxypropyl]-2,7-diazaspiro[3.4]octane-2-carboxylate

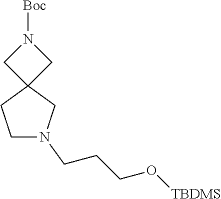

Sodium triacetoxyborohydride (699 mg, 3.30 mmol) was added to a mixture of tert-butyl 2,7-diazaspiro[3.4]octane-2-carboxylate (500 mg, 2.36 mmol) and 3-[tert-butyl(dimethyl)silyl]oxypropanal (444 mg, 2.36 mmol) in DCE (10.0 ml) at 22° C. under N₂. The mixture was stirred at 22° C. for 4 h and diluted with aq. NaHCO₃ (20.0 mL). The aqueous phase was extracted with EtOAc (3×30.0 mL), and the combined organic phases were washed with brine (30.0 mL), dried (Na₂SO₄), filtered, and concentrated. The product was purified by FC (0-10% MeOH/DCM) to provide the title compound as an oil (241 mg, 26.6%).

LCMS m/z 384.6 (M)⁺ (ES⁺).

¹H NMR (500 MHz, CDCl₃) δ 3.86 (d, J=8.5 Hz, 2H), 3.82 (d, J=8.6 Hz, 2H), 3.65 (t, J=6.3 Hz, 2H), 2.73 (s, 2H), 2.59 (s, 2H), 2.51 (s, 2H), 2.05 (t, J=6.9 Hz, 2H), 1.80-1.63 (m, 2H), 1.43 (s, 9H), 0.88 (s, 9H), 0.04 (s, 6H).

### Step B: 3-(2,6-diazaspiro[3.4]octan-6-yl)propan-1-ol

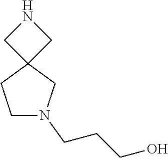 [SMILES: OCCCN1CCC2(CNC2)C1]

A mixture of TFA (5.89 ml, 79.3 mmol) and tert-butyl 7-[3-[tert-butyl(dimethyl)silyl]-oxypropyl]-2,7-diazaspiro[3.4]octane-2-carboxylate (610 mg, 1.59 mmol) was stirred at 22° C. for 1 h and diluted with water (50.0 ml). The aqueous phase was washed with Et₂O (2×50 mL) and concentrated. The residue was passed through an Amberlite IRA-402(OH) ion exchange column eluting with MeOH, and concentrated to provide the title compound as an oil (270 mg, 98%).

¹H NMR (500 MHz, CDCl₃) δ 3.85-3.76 (m, 2H), 3.55 (s, 4H), 2.81 (s, 2H), 2.72-2.62 (m, 2H), 2.59 (t, J=7.1 Hz, 2H), 2.02 (t, J=7.1 Hz, 2H), 1.71 (dt, J=10.6, 5.6 Hz, 2H).

Two exchangeable protons not observed.

### Intermediate A8: 2-(1,8-diazoniaspiro[4.5]decan-1-yl)ethanol di-(2,2,2-trifluoroacetate)

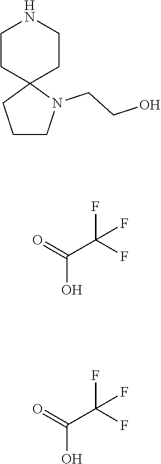 [SMILES: OCCN1CCCC12CCNCC2 ; O=C(O)C(F)(F)F ; O=C(O)C(F)(F)F]

### Step A: tert-butyl 1-[2-[tert-butyl(dimethyl)silyl]oxyethyl]-1,8-diazaspiro[4.5]decane-8-carboxylate

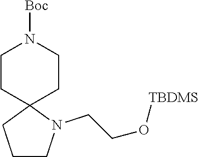

Cs₂CO₃ (793 mg, 2.43 mmol) was added to a mixture of tert-butyl 1,8-diazaspiro[4.5]-decane-8-carboxylate (450 mg, 1.87 mmol) and 2-bromoethoxy-tert-butyl-dimethylsilane (422 uL, 1.97 mmol) in DMF (7.50 mL) at 22° C. The mixture was stirred at 45° C. for 19 h and diluted with sat. aq. NH₄Cl (50 mL) and water (50 mL). The aqueous phase was extracted with EtOAc (3×50 mL, and the combined organic phases were dried (Na₂SO₄), filtered, and concentrated. The product was purified by neutral prep HPLC (0-1000% MeCN in water) to provide the title compound as an oil (370 mg, 49%).

LCMS m/z 399.5 (M)⁺ (ES⁺).

¹H NMR (400 MHz, CDCl₃) δ 4.10 (s, 2H), 3.68 (dt, J=13.7, 7.0 Hz, 2H), 2.84 (dd, J=15.2, 9.0 Hz, 2H), 2.71 (s, 2H), 2.56 (t, J=7.0 Hz, 2H), 1.98-1.69 (m, 4H), 1.69-1.49 (m, 2H), 1.45 (d, J=1.0 Hz, 9H), 1.39-1.15 (m, 2H), 0.94 (t, J=31.1 Hz, 9H), 0.06 (dd, J=4.9, 1.6 Hz, 6H).

### Step B: 2-(1,8-diazoniaspiro[4.5]decan-1-yl)ethanol di-(2,2,2-trifluoroacetate)

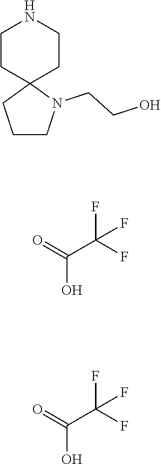 [SMILES: OCCN1CCCC12CCNCC2 ; O=C(O)C(F)(F)F ; O=C(O)C(F)(F)F]

Synthesized according to the procedure outlined for 3-(2,6-diazaspiro[3.4]octan-6-yl)-propan-1-ol (Intermediate A7, Step B), from TFA (3.73 ml, 50.2 mmol) and tert-butyl 1-[2-[tert-butyl(dimethyl)silyl]oxyethyl]-1,8-diazaspiro[4.5]decane-8-carboxylate (400 mg, 1.00 mmol) for 30 min at 22° C., to provide the title compound as an oil (288 mg, 69%).

LCMS m/z 186.2 (M-2TFA+2H)⁺ (ES⁺).

¹H NMR (400 MHz, DMSO-d₆) δ 4.27 (s, 1H), 3.42 (t, J=6.8 Hz, 2H), 3.36 (bs, 3H), 2.87 (d, J=11.9 Hz, 2H), 2.73 (t, J=6.8 Hz, 2H), 2.56-2.29 (m, 4H), 1.73-1.55 (m, 4H), 1.40 (td, J=12.5, 4.4 Hz, 2H), 1.14 (d, J=10.7 Hz, 2H).

¹⁹F NMR (376 MHz, DMSO-d₆) δ −73.49 (d, J=3.3 Hz).

### Intermediate A9: 2-(2,6-diazaspiro[3.4]octan-6-yl)ethanol di-(2,2,2-trifluoroacetic acid)

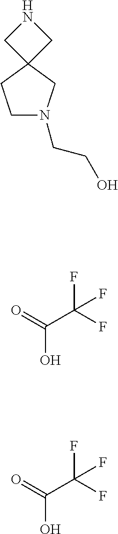 [SMILES: OCCN1CCC2(CNC2)C1 ; O=C(O)C(F)(F)F ; O=C(O)C(F)(F)F]

### Step A: tert-butyl 7-[2-[tert-butyl(dimethyl)silyl]oxyethyl]-2,7-diazaspiro[3-4]octane-2-carboxylate

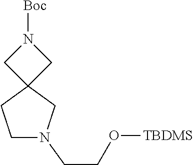

Synthesized according to the procedure outlined for tert-butyl 1-[2-[tert-butyl-(dimethyl)silyl]oxyethyl]-1,8-diazaspiro[4.5]decane-8-carboxylate (Intermediate A8, Step A), from Cs₂CO₃ (146 mg, 0.447 mmol), tert-butyl 2,7-diazaspiro[3.4]octane-2-carboxylate (73.0 mg, 0.344 mmol) and 2-bromoethoxy-tert-butyl-dimethyl-silane (0.104 mL, 0.481 mmol) in DMF (1.00 mL) at 22° C. for 16 h, to provide the title compound as an oil (98.0 mg, 77%).

LCMS m/z 371.4 (M+H)⁺ (ES⁺).

¹H NMR (400 MHz, DMSO-d₆) δ 3.72 (s, 4H), 3.64 (t, J=6.3 Hz, 2H), 2.67 (s, 2H), 2.54-2.44 (m, 4H), 1.93 (t, J=7.1 Hz, 2H), 1.36 (s, 9H), 0.85 (d, J=2.9 Hz, 9H), 0.03 (d, J=3.2 Hz, 6H).

### Step B: 2-(2,6-diazaspiro[3.4]octan-6-yl)ethanol di-(2,2,2-trifluoroacetic acid)

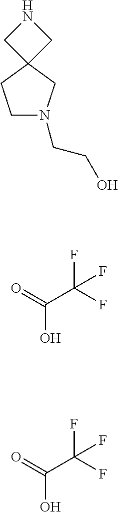 [SMILES: OCCN1CCC2(CNC2)C1 ; O=C(O)C(F)(F)F ; O=C(O)C(F)(F)F]

Synthesized according to the procedure outlined for 3-(2,6-diazaspiro[3.4]octan-6-yl)-propan-1-ol (Intermediate A7, Step B), from TFA (0.982 ml, 13.2 mmol) and tert-butyl 7-[2-[tert-butyl(dimethyl)silyl]oxyethyl]-2,7-diazaspiro[3.4]octane-2-carboxylate (98.0 mg, 0.264 mmol) for 4 h at 22° C., to provide the title compound as an oil (49 mg, 48%).

LCMS m/z 157.1 (M-2TFA+H)⁺ (ES⁺).

¹H NMR (400 MHz, DMSO-d₆) δ 7.32 (br, 3H), 4.76 (br, 1H), 3.92 (d, J=10.3 Hz, 2H), 3.89 (d, J=10.1 Hz, 2H), 3.53 (t, J=5.6 Hz, 2H), 3.06-2.96 (m, 2H), 2.80-2.72 (m, 2H), 2.72-2.65 (m, 2H), 2.10 (t, J=5.9 Hz, 2H).

¹⁹F NMR (376 MHz, DMSO-d₆) δ −73.53 (s).

### Intermediate A10: sodium 5-[[5-(2-fluoro-4-pyridyl)-6-methyl-indan-4-yl]amino]-1-(2-trimethylsilylethoxymethyl)-1,2,4-triazole-3-sulfinate

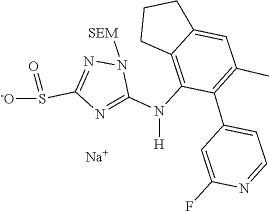

### Step A: 5-bromo-6-methyl-indan-4-amine

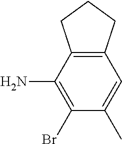

Zn dust (7.92 g, 119 mmol) was added to a mixture of 5-bromo-6-methyl-4-nitro-indane (Intermediate A6) (4.66 g, 18.2 mmol) and NH₄Cl (6.44 g, 119 mmol) in 1,4-dioxane and water (3:1, 120 mL) at 0° C. under N₂. The mixture was stirred at 22° C. for 2 h and filtered through a pad of Celite washing with EtOAc (250 mL). The filtrate was concentrated. The product was purified by FC (50-100% EtOAc/hexanes) to provide the title compound as a solid (3.81 g, 93%).

LCMS m/z 226.7 (M+H)⁺ (ES⁺).

¹H NMR (400 MHz, CDCl₃) δ 6.60 (d, J=0.5 Hz, 1H), 4.01 (s, 2H), 2.84 (t, J=7.5 Hz, 2H), 2.73 (t, J=7.4 Hz, 2H), 2.35 (d, J=0.5 Hz, 3H), 2.15-2.07 (m, 2H).

### Step B: 5-(2-fluoro-4-pyridyl)-6-methyl-indan-4-amine

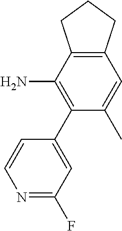 [SMILES: Cc1cc2c(c(N)c1-c1ccnc(F)c1)CCC2]

(2-Fluoro-4-pyridyl)boronic acid (1.79 g, 12.5 mmol) was added to a mixture of 5-bromo-6-methyl-indan-4-amine (2.82 g, 12.5 mmol), Pd₂(dba)₃ (760 mg, 1.28 mmol), S-Phos (1.08 g, 2.58 mmol) and K₃PO₄ (8.29 g, 38.3 mmol) in toluene (30.0 mL) at 22° C. under N₂. The mixture was stirred at 100° C. for 1 h and (2-fluoro-4-pyridyl)boronic acid (1.82 g, 12.7 mmol) was added. The mixture was stirred at 100° C. for 1 h and (2-fluoro-4-pyridyl)boronic acid (1.75 g, 12.2 mmol) was added. The mixture was stirred at 100° C. for 1 h, and activated charcoal (1 g) was added. The mixture was stirred at 22° C. for 10 min, filtered over Celite washing with EtOAc (400 mL), and the filtrate was concentrated. The product was purified by FC (0-30% EtOAc/hexanes) to provide the title compound as a solid (1.57 g, 46%).

LCMS m/z 242.9 (M+H)⁺ (ES⁺).

¹H NMR (400 MHz, DMSO-d₆) δ 8.29 (d, J=5.1 Hz, 1H), 7.14 (ddd, J=5.1, 2.3, 1.3 Hz, 1H), 6.97 (s, 1H), 6.46 (s, 1H), 4.30 (s, 2H), 2.78 (t, J=7.5 Hz, 2H), 2.65 (t, J=7.3 Hz, 2H), 2.04-1.94 (m, 2H), 1.88 (s, 3H).

¹⁹F NMR (376 MHz, DMSO-d₆) δ −68.62 (s).

### Step C: 5-bromo-N-[5-(2-fluoro-4-pyridyl)-6-methyl-indan-4-yl]-2-(2-trimethylsilylethoxymethyl)-1,2,4-triazol-3-amine

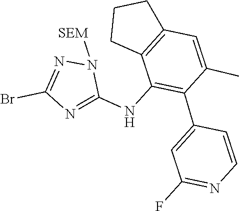 [SMILES: Cc1cc2c(c(Nc3nc(Br)nn3COCC[Si](C)(C)C)c1-c1ccnc(F)c1)CCC2]

Synthesized according to the procedure outlined for 5-bromo-N-[5-(2-fluoro-4-pyridyl)indan-4-yl]-2-(2-trimethylsilylethoxymethyl)-1,2,4-triazol-3-amine (Intermediate A2, Step E) from LHMDS (1.00 M in THF, 11.0 mL, 11.0 mmol), 5-(2-fluoro-4-pyridyl)-6-methyl-indan-4-amine (1.03 g, 3.81 mmol) and 2-[(3,5-dibromo-1,2,4-triazol-1-yl)methoxy]ethyl-trimethyl-silane (Intermediate A1) (3.33 g, 9.32 mmol) in THF (7.50 mL) at 23° C. for 1 h. The product was further purified by FC (0-30% EtOAc/hexanes) to provide the title compound as a solid (1.16 g, 59%).

LCMS m/z 518.8 (M−H)⁻ (ES⁻).

¹H NMR (400 MHz, DMSO-d₆) δ 8.48 (s, 1H), 8.19 (d, J=5.1 Hz, 1H), 7.17 (s, 1H), 7.10-7.04 (m, 1H), 6.92 (s, 1H), 5.15 (s, 2H), 3.38-3.30 (m, 2H), 2.93 (t, J=7.4 Hz, 2H), 2.64 (t, J=7.3 Hz, 2H), 2.04 (s, 3H), 2.02-1.95 (m, 2H), 0.78-0.70 (m, 2H), −0.05 (s, 9H).

¹⁹F NMR (376 MHz, DMSO-d₆) δ −69.20 (s).

### Step D: methyl 3-[[5-[[5-(2-fluoro-4-pyridyl)-6-methyl-indan-4-yl]amino]-1-(2-trimethylsilylethoxymethyl)-1,2,4-triazol-3-yl]sulfanyl]propanoate

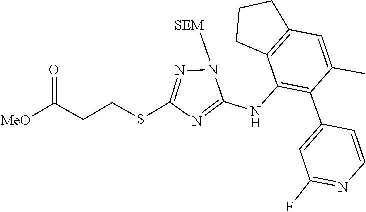 [SMILES: COC(=O)CCSc1nc(Nc2c3c(cc(C)c2-c2ccnc(F)c2)CCC3)n(COCC[Si](C)(C)C)n1]

Synthesized according to the procedure outlined for methyl 3-[[5-[[5-(2-fluoro-4-pyridyl)indan-4-yl]amino]-1-(2-trimethylsilylethoxymethyl)-1,2,4-triazol-3-yl]-sulfanyl]propanoate (Intermediate A2, Step F), from DIPEA (1.00 mL, 5.78 mmol), Pd₂(dba)₃ (273 mg, 0.289 mmol), Xantphos (178 mg, 0.301 mmol), 5-bromo-N-[5-(2-fluoro-4-pyridyl)-6-methyl-indan-4-yl]-2-(2-trimethylsilylethoxymethyl)-1,2,4-triazol-3-amine (1.55 g, 2.99 mmol), and methyl 3-sulfanylpropanoate (660 μL, 5.84 mmol) in dioxane (10.0 mL) at 100° C. for 17 h, to provide the title compound as an oil (1.28 g, 69%).

LCMS m/z 556.9 (M−H)⁻ (ES⁻).

¹H NMR (400 MHz, DMSO-d₆) δ 8.20 (s, 1H), 8.17 (d, J=5.1 Hz, 1H), 7.13 (s, 1H), 7.07 (ddd, J=5.1, 2.1, 1.3 Hz, 1H), 6.91 (s, 1H), 5.12 (s, 2H), 3.59 (s, 3H), 3.36-3.29 (m, 2H), 3.08 (t, J=7.0 Hz, 2H), 2.91 (t, J=7.4 Hz, 2H), 2.69 (t, J=7.0 Hz, 2H), 2.62 (t, J=7.4 Hz, 2H), 2.03 (s, 3H), 2.02-1.93 (m, 2H), 0.78-0.70 (m, 2H), −0.06 (s, 9H).

¹⁹F NMR (376 MHz, DMSO-d₆) δ −69.27 (s).

### Step E: methyl 3-[[5-[[5-(2-fluoro-4-pyridyl)-6-methyl-indan-4-yl]amino]-1-(2-trimethylsilylethoxymethyl)-1,2,4-triazol-3-yl]sulfonyl]propanoate

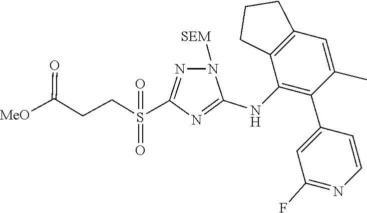 [SMILES: COC(=O)CCS(=O)(=O)c1nc(Nc2c3c(cc(C)c2-c2ccnc(F)c2)CCC3)n(COCC[Si](C)(C)C)n1]

Synthesized according to the procedure outlined for methyl 3-[[5-[[5-(2-fluoro-4-pyridyl)indan-4-yl]amino]-1-(2-trimethylsilylethoxymethyl)-1,2,4-triazol-3-yl]-sulfonyl]propanoate (Intermediate A3, Step A), from m-CPBA (77%, 1.44 g, 6.43 mmol) and methyl 3-[[5-[[5-(2-fluoro-4-pyridyl)-6-methyl-indan-4-yl]amino]-1-(2-trimethylsilylethoxymethyl)-1,2,4-triazol-3-yl]sulfanyl]propanoate (1.28 g, 2.18 mmol) in DCM (10.0 mL) at 22° C. for 1.5 h, except that the FC used 0-50% EtOAc/hexanes, to provide the title compound as a solid (90%, 1.07 g, 75%).

LCMS m/z 588.5 (M−H)⁻ (ES⁻).

¹H NMR (400 MHz, DMSO-d₆) δ 8.77 (s, 1H), 8.16 (d, J=5.1 Hz, 1H), 7.20 (s, 1H), 7.10 (ddd, J=5.1, 2.1, 1.3 Hz, 1H), 6.92 (s, 1H), 5.31 (s, 2H), 3.60 (s, 3H), 3.54 (t, J=7.2 Hz, 2H), 3.37-3.30 (m, 2H), 2.94 (t, J=7.4 Hz, 2H), 2.70-2.61 (m, 4H), 2.05 (s, 3H), 2.03-1.95 (m, 2H), 0.79-0.71 (m, 2H), −0.05 (s, 9H).

¹⁹F NMR (376 MHz, DMSO-d₆) δ −69.04 (s).

### Step F: sodium 5-[[5-(2-fluoro-4-pyridyl)-6-methyl-indan-4-yl]amino]-1-(2-trimethylsilylethoxymethyl)-1,2,4-triazole-3-sulfinate

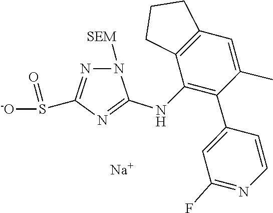

Synthesized according to the procedure outlined for sodium 5-[[5-(2-fluoro-4-pyridyl)indan-4-yl]amino]-1-(2-trimethylsilylethoxymethyl)-1,2,4-triazole-3-sulfinate (Intermediate A3, Step B), from sodium 2-methylbutan-2-olate (1.40 M in THF, 1.74 mL, 2.44 mmol) and methyl 3-[[5-[[5-(2-fluoro-4-pyridyl)-6-methyl-indan-4-yl]amino]-1-(2-trimethylsilylethoxymethyl)-1,2,4-triazol-3-yl]sulfonyl]propanoate (0.959 g, 1.63 mmol) in THF (25 mL) at 0° C. for 90 min, to afford the title compound as a solid (475 mg, 56%).

LCMS m/504.7 (M-Na+2H)⁺ (ES⁺).

¹H NMR (400 MHz, DMSO-d₆) δ 8.17 (d, J=5.1 Hz, 1H), 7.72 (s, 1H), 7.13-7.06 (m, 2H), 6.95 (s, 1H), 5.12 (s, 2H), 3.38-3.32 (m, 2H), 2.91 (t, J=7.2 Hz, 2H), 2.58 (s, 2H), 2.03 (s, 3H), 2.01-1.90 (m, 2H), 0.83-0.66 (m, 2H), −0.02-−0.06 (m, 9H).

¹⁹F NMR (376 MHz, DMSO-d₆) δ −69.16 (s).

### Intermediate A11: 2-(3-iodopyrazol-1-yl)-2-methyl-propan-1-ol

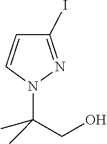

### Step A: tert-butyl 2-(3-iodopyrazol-1-yl)-2-methyl-propanoate

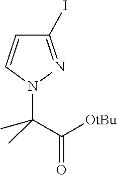 [SMILES: CC(C)(C)OC(=O)C(C)(C)n1ccc(I)n1]

tert-Butyl 2-bromo-2-methyl-propanoate (7.51 mL, 39.5 mmol) was added to a mixture of 3-iodo-1H-pyrazole (6.48 g, 33.4 mmol) and NHMDS (1.0 M in THF, 7.51 mL, 39.5 mmol) in DMF (50.0 mL) at 0° C. under N₂. The mixture was stirred at 55° C. for 16 h. The mixture was cooled to 22° C. and concentrated. The product was purified by FC (0-30% EtOAc/hexanes) to provide the title compound as an oil (8.04 g, 72%).

¹H NMR (500 MHz, CDCl₃) δ 7.37 (d, J=2.4 Hz, 1H), 6.43 (d, J=2.4 Hz, 1H), 1.79 (s, 6H), 1.39 (s, 9H).

### Step B: 2-(3-iodopyrazol-1-yl)-2-methyl-propan-1-ol

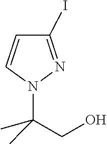

DIBAL (1M in THF, 71.7 mL, 71.7 mmol) was added to a mixture of tert-butyl 2-(3-iodopyrazol-1-yl)-2-methyl-propanoate (8.04 g, 23.9 mmol) in THF (250 mL) at −78° C. under N₂. The mixture was stirred at −78° C. for 2 h and at 22° C. for 2 h, and diluted with aq. NaOH (2.5 M, 20.0 mL). The mixture was stirred for 30 min at 22° C. and filtered. The filtrate was concentrated, and the product was purified by FC (0-50% EtOAc/hexanes) to provide the title compound as an oil (3.95 g, 62%).

LCMS m/z 267.1 (M+H)⁺ (ES⁺).

¹H NMR (400 MHz, CDCl₃) δ 7.35 (d, J=2.4 Hz, 1H), 6.42 (d, J=2.4 Hz, 1H), 3.79 (s, 2H), 2.73 (s, 1H), 1.53 (s, 6H).

### Intermediate A12: 3-(3-iodopyrazol-1-yl)-3-methyl-butan-1-ol

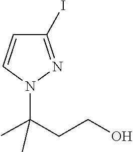

### Step A: ethyl 3-(3-iodopyrazol-1-yl)-3-methyl-butanoate

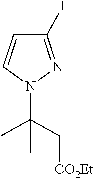

Ethyl 3-methylbut-2-enoate (2.28 mL, 16.4 mmol) was added to a mixture of 3-iodo-1H-pyrazole (1.06 g, 5.46 mmol) and DBU (1.63 mL, 10.9 mmol) in anhydrous MeCN (50.0 mL). The mixture was stirred at 60° C. for 16 h and diluted with sat. aq. NaHCO₃ (50.0 mL). The aqueous phase was extracted with EtOAc (100 mL), and the organic phase was washed with brine (100 mL), dried (Na₂SO₄), filtered, and concentrated to provide the title compound as an oil (1.72 g, 98%).

LCMS m/z 323.4 (M+H)⁺ (ES⁺).

¹H NMR (400 MHz, CDCl₃) δ 7.35 (d, J=2.3 Hz, 1H), 6.37 (d, J=2.3 Hz, 1H), 4.02 (q, J=7.1 Hz, 2H), 2.88 (s, 2H), 1.70 (s, 6H), 1.16 (t, J=7.1 Hz, 3H).

### Step B: 3-(3-iodopyrazol-1-yl)-3-methyl-butan-1-ol

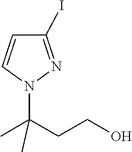

Borane tetrahydrofuran complex (1.00 M, 26.7 mL, 26.7 mmol) was added to a solution of ethyl 3-(3-iodopyrazol-1-yl)-3-methyl-butanoate (1.72 g, 5.34 mmol) in THF (50.0 mL) at 20° C. The mixture was stirred at 70° C. for 2 h and diluted with sat. aq. Rochelle's salt (50.0 mL). The mixture was stirred at 20° C. for 1 h. The aqueous phase was extracted with EtOAc (100 mL), and the organic phase was washed with water (50.0 mL) and brine (100 mL), dried (Na₂SO₄), filtered, and concentrated to provide the title compound as an oil (1.46 g, 98%).

LCMS m/z 281.6 (M+H)⁺ (ES⁺).

### Intermediate A13: methyl 2-methyl-2-(3-sulfanylphenyl)propanoate

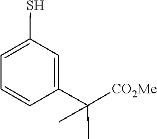

### Step A: methyl 2-[3-(3-methoxy-3-oxo-propyl)sulfanylphenyl]-2-methyl-propanoate

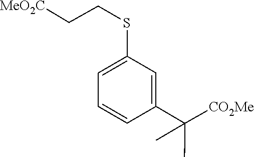 [SMILES: COC(=O)CCSc1cccc(C(C)(C)C(=O)OC)c1]

DIPEA (6.75 mL, 39.0 mmol) was added to a mixture of methyl 2-(3-bromophenyl)-methyl-propionate (5.00 g, 18.9 mmol), methyl 3-sulfanylpropanoate (4.14 mL, 37.7 mmol), Pd₂(dba)₃ (1.68 g, 2.83 mmol), and XantPhos (3.34 g, 5.66 mmol) in 1,4-dioxane (40.0 mL) at 22° C. under N₂. The mixture was degassed, stirred at 100° C. for 18 h, and diluted with aq. HCl (1M, 50.0 mL). The aqueous phase was extracted with DCM (3×75.0 mL), and the combined organic phases were washed with sat. aq. NaHCO₃ (75.0 mL), brine (75.0 ml), dried (Na₂SO₄), filtered and concentrated. The product was purified by FC (0-30% EtOAc/hexanes) to provide the title compound as an oil (3.00 g, 54%).

LCMS m/z 297.4 (M+H)⁺ (ES⁺).

¹H NMR (500 MHz, CDCl₃) δ 7.35-7.30 (m, 1H), 7.28-7.26 (m, 1H), 7.25-7.22 (m, 1H), 7.17 (dt, J=7.2, 1.9 Hz, 1H), 3.68 (s, 3H), 3.66 (s, 3H), 3.16 (t, J=7.4 Hz, 2H), 2.63 (t, J=7.4 Hz, 2H), 1.57 (s, 6H).

### Step B: methyl 2-methyl-2-(3-sulfanylphenyl)propanoate

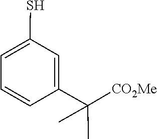

NaOMe (25 wt % in MeOH, 4.59 mL, 19.7 mmol) was added to a mixture of methyl 2-[3-(3-methoxy-3-oxo-propyl)sulfanylphenyl]-2-methyl-propanoate (3.00 g, 10.1 mmol) in a mixture of THF and MeOH (1:1 v/v, 30.0 mL) at 0° C. under N₂. The mixture was stirred at 22° C. for 6 h and diluted with aq. HCl (1 M, 50 mL). The aqueous phase was extracted with DCM (3×100 mL), and the organic phases were combined, washed with brine (100 mL), dried (Na₂SO₄), filtered and concentrated. The product was purified by FC (0-20% EtOAc/hexanes) to provide the title compound as an oil (1.78 g, 84%), which was stored in the freezer under an atmosphere of nitrogen.

¹H NMR (400 MHz, CDCl₃) δ 7.22-7.10 (m, 4H), 3.66 (s, 3H), 3.46 (s, 1H), 1.55 (s, 6H).

### Intermediate A14: 2-(methyl(piperidin-4-yl)amino)ethan-1-ol

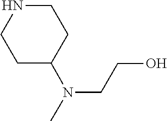

tert-Butyl 4-(methylamino)piperidine-1-carboxylate (1.00 g, 4.67 mmol) and K₂CO₃ (1.93 g, 14.0 mmol) were suspended in MeCN (20 mL). 2-bromoethan-1-ol (331 μL, 4.67 mmol) was added and the reaction stirred at 80° C. for 24 h. A further equivalent of 2-bromoethan-1-ol (331 μL, 4.67 mmol) was added and the reaction stirred for a further 24 h. The reaction was concentrated in vacuo and the resulting residue dissolved in DCM (100 mL), washed with brine (100 mL), dried using a phase separator and concentrated in vacuo. The crude product was purified by FC (0-100% (0.7 M ammonia/MeOH)/DCM) to afford the Boc-protected product. This was taken up in HCl (4 M in dioxane) (5 mL, 4.0 M, 20 mmol) and EtOH (3 mL) and stirred at RT for 18 h. The reaction was concentrated in vacuo and the resulting residue dissolved in 0.7 M NH₃ in MeOH (10 mL). The solution was concentrated in vacuo and the resulting residue dissolved in MeOH (30 mL) and stirred with SCX (10 g) for 45 min. The SCX resin was washed with methanol (100 mL), then the product was eluted with 0.7 M NH₃ in MeOH (150 mL). The resulting solution was concentrated in vacuo to afford the title compound (386 mg, 52%) as a straw-coloured oil.

¹H NMR (500 MHz, DMSO-d₆) δ 3.41 (t, J=6.6 Hz, 2H) 3.03-2.89 (m, 2H), 2.49-2.29 (m, 5H), 2.17 (s, 3H), 1.63-1.55 (m, 2H), 1.31-1.20 (m, 2H). 2 exchangeable protons not observed.

### Intermediate A15: 2-methyl-2-(2,6-diazaspiro[3.4]octan-6-yl)propan-1-ol, di-(2,2,2-trifluoroacetic acid)

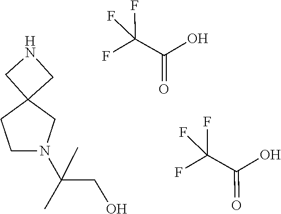

### Step A: tert-butyl 6-(1-methoxy-2-methyl-1-oxopropan-2-yl)-2,6-diazaspiro[3.4]-octane-2-carboxylate

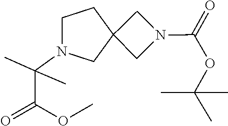 [SMILES: COC(=O)C(C)(C)N1CCC2(CN(C(=O)OC(C)(C)C)C2)C1]

tert-Butyl 2,6-diazaspiro[3.4]octane-2-carboxylate (1.00 g, 4.71 mmol) and K₂CO₃ (1.95 g, 14.1 mmol) were suspended in MeCN (30 mL). Methyl 2-bromo-2-methylpropanoate (610 μL, 4.71 mmol) was added and the reaction stirred at 80° C. for 24 h. The reaction was concentrated in vacuo and the resulting residue dissolved in DCM (100 mL), washed with brine (100 mL), dried using a phase separator and concentrated in vacuo. The crude product was purified by FC (0-10% (0.7 M ammonia/MeOH)/DCM) to afford the title compound (1.09 g, 73%) as a yellow oil.

¹H NMR (500 MHz, DMSO-d₆) δ 3.77-3.63 (m, 4H), 3.61 (s, 3H), 2.85 (s, 2H), 2.72 (t, J=6.9 Hz, 2H), 1.90 (t, J=6.9 Hz, 2H), 1.37 (s, 9H), 1.26 (s, 6H).

### Step B: 2-methyl-2-(2,6-diazaspiro[3.4]octan-6-yl)propan-1-ol, di-(2,2,2-trifluoroacetic

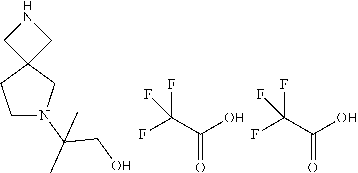

LiBH₄ (4 M in THF) (7.06 mL, 28.2 mmol) was added drop-wise to a stirred solution of tert-butyl 6-(1-methoxy-2-methyl-1-oxopropan-2-yl)-2,6-diazaspiro[3.4]octane-2-carboxylate (1.47 g, 4.71 mmol) in THF (15 mL) cooled to 0° C. The mixture was warmed to RT and stirred at 50° C. for a further 16 h. The mixture was partitioned between water (100 mL) and EtOAc (100 mL). The organic layer was collected and the aqueous layer was extracted with EtOAc (50 mL). The combined organic layers were dried using a phase separator and concentrated in vacuo. The resulting residue was purified by FC (0-10% (0.7 M ammonia/MeOH)/DCM) to afford the Boc-protected product. This was taken up in TFA (10 mL) and stirred at RT for 2 h. The reaction was concentrated in vacuo and the TFA residues were removed as an azeotrope with toluene (×3) to afford the title compound (2.01 g, 98%) as a light yellow oil, which began to form crystals on standing.

¹H NMR (500 MHz, DMSO-d₆) δ 9.71 (bs, 1H), 9.13 (bs, 1H), 8.87 (bs, 1H), 4.14-4.02 (m, 2H), 4.00-3.93 (m, 1H), 3.90-3.80 (m, 1H), 3.66-3.57 (m, 1H), 3.54-3.40 (m, 3H), 3.39-3.28 (m, 2H), 2.33-2.20 (m, 2H), 1.20 (s, 6H). One exchangeable proton not observed.

### Intermediate A16: 2-((azetidin-3-ylmethyl)(methyl)amino)ethan-1-ol

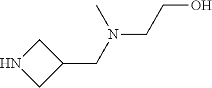

### Step A: tert-butyl 3-(((2-((tert-butyldimethylsilyl)oxy)ethyl)(methyl)amino)methyl)-azetidine-1-carboxylate

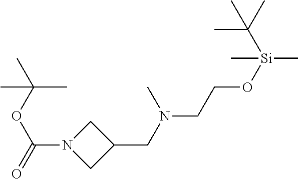 [SMILES: CN(CCO[Si](C)(C)C(C)(C)C)CC1CN(C(=O)OC(C)(C)C)C1]

2-((tert-butyldimethylsilyl)oxy)-N-methylethan-1-amine (1.33 g, 7.00 mmol) then sodium triacetoxyborohydride (1.72 g, 8.10 mmol) was added to a solution of tert-butyl 3-formylazetidine-1-carboxylate (1.00 g, 5.40 mmol) in THF (30 mL) at RT. The mixture was stirred for 24 h then partitioned between EtOAc (150 mL) and sat. aq. NaHCO₃ solution (50 mL). The organic layer was washed with water (50 mL), dried (MgSO₄), filtered and evaporated. The crude product was purified by FC (0-5% MeOH/DCM) to afford the title compound (1.71 g, 84%) as an oil.

¹H NMR (500 MHz, CDCl₃) δ 4.01 (t, J=8.2 Hz, 2H), 3.72 (t, J=6.3 Hz, 2H), 3.59 (dd, J=8.6, 5.1 Hz, 2H), 2.76-2.63 (m, 3H), 2.54 (t, J=6.3 Hz, 2H), 2.27 (s, 3H), 1.45 (s, 9H), 0.91 (s, 9H), 0.08 (s, 6H).

### Step B: 2-((azetidin-3-ylmethyl)(methyl)amino)ethan-1-ol

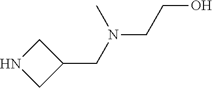

TFA (10.0 mL) was added to a solution of tert-butyl 3-(((2-((tert-butyldimethylsilyl)-oxy)ethyl)(methyl)amino)methyl)azetidine-1-carboxylate (1.70 g, 4.74 mmol) in DCM (20 mL) at RT. The mixture was stirred for 24 h, evaporated, and the residue loaded onto SCX column with MeOH, washed with MeOH then eluted with 0.7M NH₃/MeOH to afford the title compound (460 mg, 64%) as an oil.

¹H NMR (500 MHz, CDCl₃) δ 3.73 (t, J=7.9 Hz, 2H), 3.59 (t, J=5.6 Hz, 2H), 3.41 (t, J=7.4 Hz, 2H), 3.03-2.90 (m, 3H), 2.67 (d, J=7.2 Hz, 2H), 2.53 (t, J=5.4 Hz, 2H), 2.23 (s, 3H).

### Intermediate A17: (R)-2-(methyl(pyrrolidin-3-ylmethyl)amino)ethan-1-ol

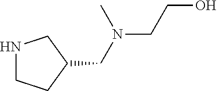

### Step A: tert-butyl (R)-3-((2-hydroxyethyl)(methyl)carbamoyl)pyrrolidine-1-carboxylate

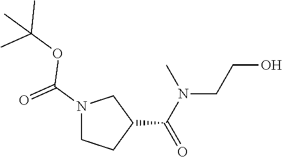

HATU (6.36 g, 16.7 mmol) was added portionwise to a solution of (R)-1-(tert-butoxy-carbonyl)pyrrolidine-3-carboxylic acid (3.00 g, 13.9 mmol), 2-(methylamino)ethan-1-ol (1.40 mL, 17.4 mmol) and DIPEA (4.00 mL, 23.0 mmol) in DMF (30 mL) cooled on an icebath. The mixture was warmed to RT, stirred overnight and partitioned between EtOAc (300 mL) and water (150 mL). The organic layer was washed with 1M HCl (2×50 mL), water (50 mL) and brine (50 mL), dried (MgSO₄), filtered and evaporated. The crude product was purified by FC (0-5% MeOH/DCM) to afford the title compound (996 mg, 24%) as an oil.

¹H NMR (500 MHz, CDCl₃, rotamers present) δ 3.81 (t, J=5.2 Hz, 2H), 3.69-3.52 (m, 4H), 3.52-3.44 (m, 1H), 3.41-3.22 (m, 2H), 3.16 and 2.99 (2×s, 3H), 2.64 (br s, 1H), 2.23-2.02 (m, 2H), 1.47 (s, 9H).

### Step B: tert-butyl (S)-3-(((2-hydroxyethyl)(methyl)amino)methyl)pyrrolidine-1-carboxylate

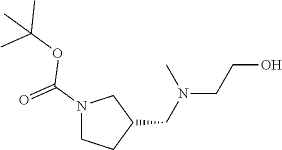

1 M Borane tetrahydrofuran complex in THF (14.5 mL, 14.5 mmol) was added to a solution of tert-butyl (R)-3-((2-hydroxyethyl)(methyl)carbamoyl)pyrrolidine-1-carboxylate (990 mg, 3.64 mmol) in THF (15 mL) and heated at 65° C. for 8 h. The mixture was cooled, MeOH (30 mL) added carefully, the mixture heated at 60° C. for 5 h then the solvent evaporated. The crude product was purified by FC (0-15% MeOH/DCM) to afford the title compound (485 mg, 46%) as an oil.

¹H NMR (500 MHz, CDCl3) δ 3.62 (t, J=5.3 Hz, 2H), 3.58-3.25 (m, 3H), 3.06-2.95 (m, 1H), 2.79 (br s, 1H), 2.59 (br s, 2H), 2.50-2.38 (m, 3H), 2.32 (s, 3H), 2.06-1.97 (m, 1H), 1.68-1.53 (m, 1H), 1.47 (s, 9H).

### Step C: (R)-2-(methyl(pyrrolidin-3-ylmethyl)amino)ethan-1-ol

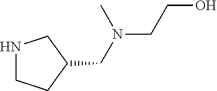

Synthesized according to the procedure outlined for 2-((azetidin-3-ylmethyl)(methyl)-amino)ethan-1-ol (Intermediate A16, Step B) from tert-butyl (S)-3-(((2-hydroxy-ethyl)(methyl)amino)methyl)pyrrolidine-1-carboxylate (470 mg, 1.82 mmol), TFA (3.0 mL) and DCM (5 mL) at RT to afford the title compound (300 mg, 83%) as an oil.

¹H NMR (500 MHz, DMSO-d₆) δ 4.25 (br s, 2H), 3.45 (t, J=6.4 Hz, 2H), 2.90-2.69 (m, 3H), 2.49-2.43 (m, 1H), 2.41-2.34 (m, 2H), 2.26-2.20 (m, 2H), 2.19-2.13 (m, 4H) 1.79-1.71 (m, 1H), 1.34-1.23 (m, 1H).

### Intermediate A18: 4-(methyl(piperidin-4-yl)amino)butan-1-ol, di-(2,2,2-trifluoroacetic acid)

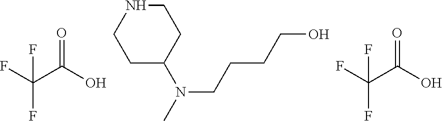

### Step A: tert-butyl 4-((4-ethoxy-4-oxobutyl)(methyl)amino)piperidine-1-carboxylate

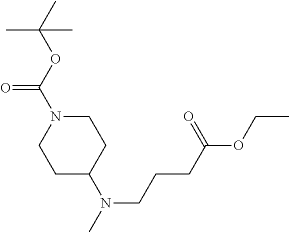 [SMILES: CCOC(=O)CCCN(C)C1CCN(C(=O)OC(C)(C)C)CC1]

tert-Butyl 4-(methylamino)piperidine-1-carboxylate (1.00 g, 4.67 mmol) and K₂CO₃ (1.93 g, 14.0 mmol) were suspended in MeCN (20 mL). Ethyl 4-bromobutanoate (668 μL, 4.67 mmol) was added and the reaction stirred at 80° C. for 48 h. The reaction was concentrated in vacuo and the resulting residue dissolved in DCM (100 mL), washed with brine (100 mL), dried using a phase separator and concentrated in vacuo. The crude product was purified by FC (0-10% (0.7 M ammonia/MeOH)/DCM)) to afford the title compound (1.023 g, 66%) as a colourless oil.

¹H NMR (500 MHz, DMSO-d₆) δ 4.03 (q, J=7.1 Hz, 2H), 4.00-3.38 (m, 2H), 2.77-2.57 (m, 2H), 2.47-2.39 (m, 1H), 2.36 (t, J=6.9 Hz, 2H), 2.27 (t, J=7.2 Hz, 2H), 2.12 (s, 3H), 1.68-1.57 (m, 4H), 1.39 (s, 9H), 1.28-1.15 (m, 5H).

### Step B: 4-(methyl(piperidin-4-yl)amino)butan-1-ol, di-(2,2,2-trifluoroacetic acid)

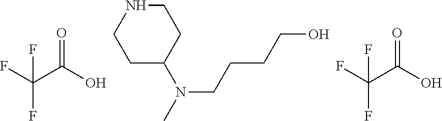

LiBH₄ (4 M in THF) (4.67 mL, 18.7 mmol) was added drop-wise to a stirred solution of tert-butyl 4-((4-ethoxy-4-oxobutyl)(methyl)amino)piperidine-1-carboxylate (1.02 g, 3.12 mmol) in THF (15 mL) cooled to 0° C. The mixture was warmed to RT and stirred for 48 h, before heating to 50° C. and stirring for a further 5 h. The mixture was then partitioned between water (100 mL) and EtOAc (100 mL). The organic layer was collected and the aqueous layer was extracted with EtOAc (50 mL). The combined organic layers were dried using a phase separator and concentrated in vacuo. The resulting residue was purified by FC (0-10% (0.7 M ammonia/MeOH)/DCM)) to afford the Boc-protected product. This was taken up in TFA (5 mL) and stirred at RT for 2 h. The reaction was concentrated in vacuo and the TFA residues were removed as an azeotrope with toluene (×3) to afford the title compound (612 mg, 47%) as a colourless oil.

¹H NMR (500 MHz, DMSO-d₆) δ 10.09-9.82 (m, 1H), 8.98-8.81 (m, 1H), 8.69-8.52 (m, 1H), 4.42 (t, J=5.8 Hz, 1H), 3.60-3.49 (m, 1H), 3.49-3.40 (m, 3H), 3.21-3.03 (m, 3H), 3.02-2.87 (m, 2H), 2.76-2.70 (m, 3H), 2.14 (d, J=13.2 Hz, 2H), 1.93-1.60 (m, 5H), 1.51-1.42 (m, 1H).

### Intermediate A19: (S)-2-(methyl(pyrrolidin-3-ylmethyl)amino)ethan-1-ol, 2,2,2-trifluoroacetic acid

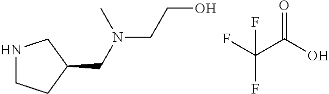

### Step A: tert-butyl (S)-3-((2-hydroxyethyl)(methyl)carbamoyl)pyrrolidine-1-carboxylate

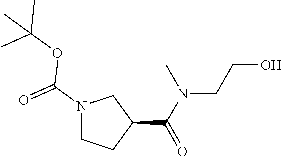 [SMILES: CN(CCO)C(=O)[C@H]1CCN(C(=O)OC(C)(C)C)C1]

HATU (10.6 g, 27.9 mmol) was added portionwise to a solution of (S)-1-(tert-butoxy-carbonyl)pyrrolidine-3-carboxylic acid (5.00 g, 23.2 mmol), 2-(methylamino)ethan-1-ol (2.50 mL, 31.1 mmol) and DIPEA (6.70 mL, 38.5 mmol) in DMF (60 mL) at 0° C. The mixture was warmed to RT, stirred overnight and partitioned between EtOAc (300 mL) and water (150 mL). The organic phase was washed with 1M HCl (75 mL), water (50 mL) and brine (50 mL), dried (MgSO₄), filtered and concentrated under reduced pressure. The crude product was purified by FC (0-5% MeOH/DCM) to afford the title compound (1.50 g, 12%) as an oil.

LCMS m/z 217.0 (M-^{t}Bu+H)⁺ (ES⁺).

### Step B: (S)-2-(methyl(pyrrolidin-3-ylmethyl)amino)ethan-1-ol, 2,2,2-trifluoroacetic acid

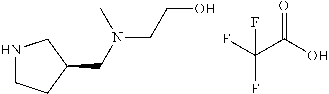

1M Borane tetrahydrofuran complex in THF (7.02 mL, 7.02 mmol) was added portionwise to a solution of tert-butyl (S)-3-((2-hydroxyethyl)(methyl)carbamoyl)pyrrolidine-1-carboxylate (0.956 g, 1.76 mmol) in THF (20 mL) and stirred at RT for 16 h. Additional 1M borane tetrahydrofuran complex in THF (7.02 mL, 7.02 mmol) was added and the reaction stirred at RT for 3 h. MeOH (30 mL) was carefully added and the reaction stirred at 60° C. for 3 h. The volatiles were removed under reduced pressure and the crude loaded onto SCX (10 g, pre-washed) and rinsed with MeOH (20 mL), then the product eluted with 0.7 M NH₃ in MeOH (3×20 mL). The ammoniacal solution was concentrated under reduced pressure to afford tert-butyl (R)-3-(((2-hydroxyethyl)(methyl)amino)methyl)pyrrolidine-1-carboxylate (661 mg, 1.7 mmol, 95%) as a yellow oil. The residue was taken up in DCM/TFA (1:1, 10 mL) and stirred at RT for 3 h. The volatiles were removed under reduced pressure azeotroping with MeOH (2×20 mL). The residue was loaded onto SCX (10 g, pre-washed with MeOH) and rinsed with MeOH (20 mL). The product was eluted with 0.7 M NH₃ in MeOH (2×30 mL). The ammoniacal solution was concentrated under reduced pressure to afford the title compound (226 mg, 45%) as a yellow oil.

¹H NMR (500 MHz, CDCl₃) δ 4.60 (br s, 2H), 3.63-3.52 (m, 2H), 3.17-3.11 (m, 1H), 3.11-3.04 (m, 1H), 3.03-2.97 (m, 1H), 2.82-2.70 (m, 1H), 2.58-2.45 (m, 2H), 2.43-2.33 (m, 3H), 2.25 (s, 3H), 2.03-1.90 (m, 1H), 1.49 (m, 1H). One exchangeable proton not observed.

### Intermediate A20: 3-(1,7-diazaspiro[3.5]nonan-1-yl)propan-1-ol, di-(2,2,2-trifluoroacetic acid)

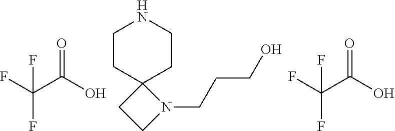

### Step A: tert-butyl 1-(3-hydroxypropyl)-1,7-diazaspiro[3.5]nonane-7-carboxylate

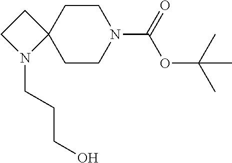 [SMILES: CC(C)(C)OC(=O)N1CCC2(CC1)CCN2CCCO]

A solution of tert-butyl 1,7-diazaspiro[3.5]nonane-7-carboxylate (500 mg, 2.21 mmol), 3-bromo-1-propanol (204 μL, 2.25 mmol) and Et₃N (0.70 mL, 5.0 mmol) in anhydrous THF (15 mL) was stirred at RT for 3 days. The reaction mixture was filtered and concentrated to dryness to give crude product. The crude product was purified by FC (0-100% EtOAc/(3:1 EtOAc:EtOH with 2% NH₄0H)) to afford the title compound (537 mg, 60%) as a pale yellow oil.

¹H NMR (500 MHz, DMSO-d₆) δ 3.88 (m, 2H), 3.40 (t, J=6.3 Hz, 2H), 3.13 (s, 2H), 2.74-2.60 (m, 2H), 2.46 (t, J=7.9 Hz, 2H), 1.87 (t, J=7.1 Hz, 2H), 1.71 (m, 2H), 1.39 (m, 13H). One exchangeable proton not observed.

### Step B: 3-(1,7-diazaspiro[3.5]nonan-1-yl)propan-1-ol, di-(2,2,2-trifluoroacetic acid)

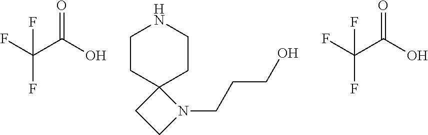

tert-Butyl 1-(3-hydroxypropyl)-1,7-diazaspiro[3-5]nonane-7-carboxylate (537 mg, 1.32 mmol) was dissolved in TFA (3 mL) and the mixture was stirred at RT for 24 h. The mixture was concentrated to dryness to afford the title compound (602 mg, 94%) as a thick orange oil.

LCMS m/z 185.4 (M-2TFA+H)⁺ (ES⁺).

¹H NMR (500 MHz, DMSO-d₆) δ 10.36 (br s, 1H), 8.97-8.79 (m, 1H), 8.66-8.56 (m, 1H), 4.58-4.33 (m, 2H), 4.14-3.82 (m, 2H), 3.53-3.31 (m, 3H), 3.26-2.94 (m, 2H), 2.94-2.79 (m, 1H), 2.47-2.27 (m, 5H), 2.14-2.02 (m, 2H), 2.00-1.84 (m, 2H).

¹⁹F NMR (471 MHz, DMSO-d₆) δ −74.62.

### Intermediate A21: 4-(3-((dimethylamino)methyl)azetidin-3-yl)butan-1-ol

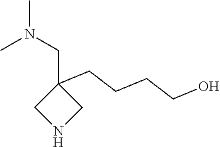

### Step A: 1-benzhydryl-3-(4-((tert-butyldimethylsilyl)oxy)butyl)azetidine-3-carbonitrile

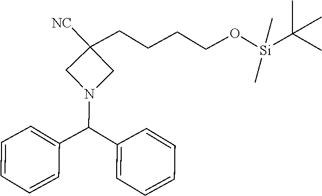 [SMILES: CC(C)(C)[Si](C)(C)OCCCCC1(C#N)CN(C(c2ccccc2)c2ccccc2)C1]

1M LiHMDS in THF (21.1 mL, 21.1 mmol) was added dropwise to 1-benzhydryl-azetidine-3-carbonitrile (5.00 g, 20.1 mmol) in THF (100 mL) cooled to −78° C. over 10 min. The mixture was left to stir at −78° C. for 30 min. tert-Butyl(4-iodobutoxy)-dimethylsilane (5.49 mL, 20.1 mmol) was then added in one portion and the mixture was left to warm to RT over 2.5 hours. The mixture was quenched with sat. aq. NH₄Cl (50 mL), water (20 mL) and EtOAc (100 mL). The organic layer was collected and the aqueous layer was extracted with EtOAc (2×50 mL). The combined organic layers were dried (MgSO₄), filtered and concentrated to dryness to give an orange oil. The crude product was purified by FC (0-50% EtOAc/heptane) to afford the title compound (5.31 g, 59%) as a pale yellow oil.

¹H NMR (500 MHz, DMSO-d₆) δ 7.43-7.38 (m, 4H), 7.29 (t, J=7.6 Hz, 4H), 7.22-7.17 (m, 2H), 4.53 (s, 1H), 3.59 (t, J=6.1 Hz, 2H), 3.35 (d, J=7.3 Hz, 2H), 3.08 (d, J=7.3 Hz, 2H), 1.90-1.83 (m, 2H), 1.49 (p, J=6.6 Hz, 2H), 1.43-1.34 (m, 2H), 0.85 (s, 9H), 0.03 (s, 6H).

LCMS m/z 435.5 (M+H)⁺ (ES⁺).

### Step B: 4-(3-(aminomethyl)-1-benzhydrylazetidin-3-yl)butan-1-ol

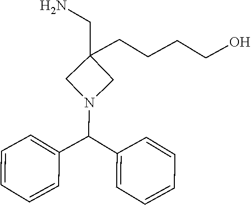 [SMILES: NCC1(CCCCO)CN(C(c2ccccc2)c2ccccc2)C1]

To a solution of 1-benzhydryl-3-(4-((tert-butyldimethylsilyl)oxy)butyl)azetidine-3-carbonitrile (5.31 g, 11.8 mmol) in THF (100 mL) at RT was added LiAlH₄ (1 M in THF) (41.5 mL, 41.5 mmol) drop-wise and the resultant reaction mixture refluxed for 3 h. The reaction was allowed to cool to RT and the reaction mixture was then treated dropwise with water (5 mL), NaOH (2M aq solution, 10 mL) and water (10 mL) and then stirred vigorously for 30 min. The mixture was then filtered and the filtrate concentrated in vacuo to afford the title compound (4.12 g, 96%) as a white solid.

¹H NMR (500 MHz, DMSO-d₆) δ 7.40 (d, J=7.3 Hz, 4H), 7.25 (t, J=7.6 Hz, 4H), 7.15 (t, J=7.3 Hz, 2H), 4.41 (s, 1H), 4.32 (s, 1H), 3.38 (t, J=6.5 Hz, 2H), 2.87 (d, J=7.1 Hz, 2H), 2.72-2.61 (m, 4H), 1.56-1.47 (m, 2H), 1.46-1.27 (m, 4H), 1.27-1.12 (m, 2H).

### Step C: 4-(1-benzhydryl-3-((dimethylamino)methyl)azetidin-3-yl)butan-1-ol

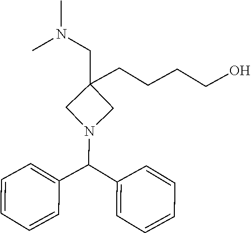 [SMILES: CN(C)CC1(CCCCO)CN(C(c2ccccc2)c2ccccc2)C1]

A mixture of 4-(3-(aminomethyl)-1-benzhydrylazetidin-3-yl)butan-1-ol (4.12 g, 12.7 mmol), formaldehyde (25 mL, 37% wt) and formic acid (25 mL) was refluxed for 3.5 h. The reaction mixture was then allowed to cool to RT before being concentrated in vacuo. The resulting residue was mixed with ice, basified with aq. NaOH (2 N) and extracted with DCM (150 mL). The organics were then washed with water and brine, dried (MgSO₄) and concentrated in vacuo to afford the title compound (4.77 g, 85%) as a yellow oil.

¹H NMR (500 MHz, DMSO-d₆) δ 7.43-7.37 (m, 4H), 7.26 (t, J=7.5 Hz, 4H), 7.19-7.12 (m, 2H), 4.40 (s, 1H), 4.12 (t, J=6.6 Hz, 1H), 3.43-3.36 (m, 1H), 2.94 (dd, J=7.3, 3.8 Hz, 2H), 2.69 (d, J=7.1 Hz, 2H), 2.30 (d, J=2.4 Hz, 2H), 2.02 (d, J=1.4 Hz, 6H), 1.76-1.57 (m, 3H), 1.43 (p, J=6.8 Hz, 1H), 1.36-1.23 (m, 2H). One exchangeable proton not observed.

### Step D: 4-(3-((dimethylamino)methyl)azetidin-3-yl)butan-1-ol

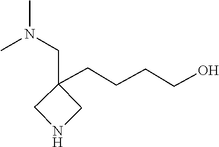

4-(1-Benzhydryl-3-((dimethylamino)methyl)azetidin-3-yl)butan-1-ol (4.77 g, 13.5 mmol) was dissolved in EtOH (80 mL) to which was added 20 wt % palladium hydroxide on carbon (452 mg, 644 μmol). The reaction mixture was then hydrogenated at 4 bar pressure for 24 h at 40° C. The catalyst was then filtered off and the filtrate concentrated under reduced pressure. ¹H NMR showed incomplete deprotection. The residue was dissolved in EtOH (80 mL) and AcOH (2 mL) and then 20 wt % palladium hydroxide on carbon (452 mg, 644 μmol) was added and the resultant mixture was hydrogenated at 4 bar pressure for 20 h at 40° C. The catalyst was then filtered off and the filtrate concentrated under reduced pressure. The crude was loaded onto SCX (20 g, pre-washed with MeOH), then rinsed with MeOH (50 mL) and the product was eluted with 7 M NH₃ in MeOH (100 mL). The ammoniacal solution was concentrated under reduced pressure to afford the title compound (1.14 g, 96%) as a yellow oil.

¹H NMR (500 MHz, CDCl₃) δ 3.69-3.61 (m, 2H), 3.44-3.38 (m, 2H), 3.32-3.26 (m, 2H), 2.43 (s, 2H), 2.12 (s, 6H), 1.81-1.74 (m, 2H), 1.61-1.52 (m, 2H), 1.39-1.30 (m, 2H). Two exchangeable protons not observed.

### Intermediate A22: (S)-3-(methyl(pyrrolidin-3-yl)amino)propan-1-ol

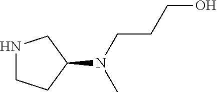

tert-Butyl (S)-3-(methylamino)pyrrolidine-1-carboxylate (1.00 g, 4.99 mmol) and 3-((tert-butyldimethylsilyl)oxy)propanal (1.41 g, 7.49 mmol) were dissolved in DCE (50 mL) and sodium triacetoxyborohydride (2.12 g, 9.99 mmol) was added slowly. The reaction was stirred at RT for 24 h. The reaction was quenched with sat. aq. NaHCO₃ (100 mL) and extracted with DCM (2×75 mL), dried using a phase separator and concentrated in vacuo. The crude product was purified by FC (0-50% (0.7 M ammonia/MeOH)/DCM)) to afford the protected product as a light yellow oil. This was taken up in HCl (4 M in dioxane) (5 mL) and stirred at RT for 20 h. The reaction was concentrated in vacuo and the resulting residue dissolved in 0.7 M NH₃ in MeOH. The solution was concentrated in vacuo and the resulting residue dissolved in methanol (30 mL) and stirred with SCX (10 g) for 45 min. The SCX resin was washed with methanol (100 mL), then the product was eluted with 0.7 M NH₃ in MeOH (150 mL). The resulting solution was concentrated in vacuo to afford the title compound (502 mg, 60%) as an orange oil.

¹H NMR (500 MHz, DMSO-d₆) δ 4.38-3.91 (m, 3H), 3.42 (t, J=6.3 Hz, 2H), 2.97-2.89 (m, 1H), 2.87-2.70 (m, 3H), 2.43-2.49 (m, 2H), 2.10 (s, 3H), 1.84-1.73 (m, 1H), 1.59-1.43 (m, 3H).

### Intermediate A23: (1-(piperidin-4-yl)azetidin-3-yl)methanol

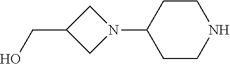 [SMILES: OCC1CN(C2CCNCC2)C1]

### Step A: tert-butyl 4-(3-(hydroxymethyl)azetidin-1-yl)piperidine-1-carboxylate

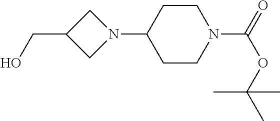 [SMILES: CC(C)(C)OC(=O)N1CCC(N2CC(CO)C2)CC1]

Sodium triacetoxyborohydride (2.80 g, 13.2 mmol) was added to a solution of tert-butyl 4-oxopiperidine-1-carboxylate (2.00 g, 10.0 mmol) and azetidin-3-ylmethanol (900 mg, 10.3 mmol) in DCE (25 mL) at RT. The mixture was stirred for 4 days then partitioned between DCM (150 mL) and sat. aq. NaHCO₃ (50 mL). The organic layer was washed with brine (20 mL), dried (MgSO₄), filtered, evaporated and the residue purified by FC (0-20% MeOH/DCM) to afford the title compound (973 mg, 34%) as a gum which solidified on standing.

¹H NMR (500 MHz, CDCl₃) δ 3.94 (br s, 2H), 3.77 (d, J=5.3 Hz, 2H), 3.38 (t, J=7.4 Hz, 2H), 3.13 (t, J=6.4 Hz, 2H), 2.85 (t, J=12.0 Hz, 2H), 2.70-2.61 (m, 1H), 2.27-2.20 (m, 1H), 1.67 (br d, J=12.9 Hz, 2H), 1.46 (s, 9H), 1.29-1.19 (m, 2H). One exchangeable proton not observed.

### Step B: (1-(piperidin-4-yl)azetidin-3-yl)methanol

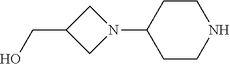 [SMILES: OCC1CN(C2CCNCC2)C1]

A solution of tert-butyl 4-(3-(hydroxymethyl)azetidin-1-yl)piperidine-1-carboxylate (963 mg, 3.56 mmol) and TFA (10 mL) in DCM (20 mL) was stirred at RT for 4 h then evaporated. The residue was purified on SCX resin (loading in MeOH, washing with MeOH then eluting with 0.7M NH₃/MeOH) to afford the title compound (562 mg, 88%) as a solid.

¹H NMR (500 MHz, DMSO-d₆) δ 4.50 (br s, 1H), 3.46 (d, J=6.7 Hz, 2H), 3.11 (t, J=7.2 Hz, 2H), 2.86 (dt, J=12.4, 3.9 Hz, 2H), 2.75 (t, J=6.7 Hz, 2H), 2.42-2.32 (m, 3H), 1.97-1.91 (m, 1H), 1.56-1.49 (m, 2H), 0.99-0.88 (m, 2H). One exchangeable proton not observed.

### Intermediate A24: 4-(2,6-diazaspiro[3.4]octan-6-yl)butan-1-ol, di-(2,2,2-trifluoroacetic acid)

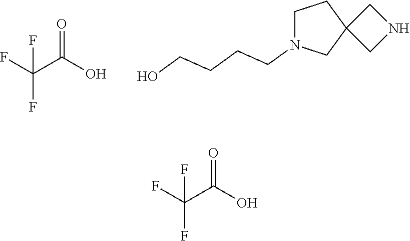 [SMILES: O=C(O)C(F)(F)F ; OCCCCN1CCC2(CNC2)C1 ; O=C(O)C(F)(F)F]

### Step A: tert-butyl 6-(4-ethoxy-4-oxobutyl)-2,6-diazaspiro[3.4]octane-2-carboxylate

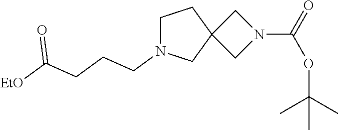 [SMILES: CCOC(=O)CCCN1CCC2(C1)CN(C(=O)OC(C)(C)C)C2]

tert-Butyl 2,6-diazaspiro[3.4]octane-2-carboxylate (0.800 g, 3.77 mmol) and K₂CO₃ (1.56 g, 11.3 mmol) were suspended in MeCN (20 mL). Ethyl 4-bromobutanoate (539 μL, 3.77 mmol) was added and the reaction stirred at 80° C. for 24 h. The reaction was concentrated in vacuo and the resulting residue dissolved in DCM (100 mL), washed with brine (100 mL), dried using a phase separator and concentrated in vacuo. The crude product was purified by FC (0-10% (0.7 M ammonia/MeOH)/DCM) to afford the title compound (690 mg, 53%) as a colourless oil.

¹H NMR (500 MHz, DMSO-d₆) δ 4.04 (q, J=7.1 Hz, 2H), 3.80-3.65 (m, 4H), 2.59 (s, 2H), 2.44 (t, J=7.1 Hz, 2H), 2.34 (t, J=7.3 Hz, 2H), 2.29 (t, J=7.3 Hz, 2H), 1.96-1.91 (m, 2H), 1.65 (p, J=7.2 Hz, 2H), 1.37 (s, 9H), 1.18 (t, J=7.1 Hz, 3H).

### Step B: 4-(2,6-diazaspiro[3.4]octan-6-yl)butan-1-ol, di-(2,2,2-trifluoroacetic acid)

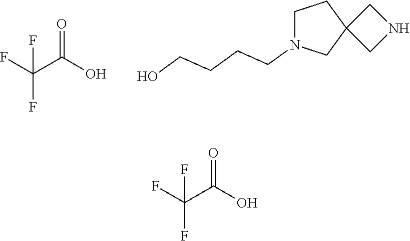 [SMILES: O=C(O)C(F)(F)F ; OCCCCN1CCC2(CNC2)C1 ; O=C(O)C(F)(F)F]

LiBH₄ (4M in THF) (3.17 mL, 12.7 mmol) was added drop-wise to a stirred solution of tert-butyl 6-(4-ethoxy-4-oxobutyl)-2,6-diazaspiro[3.4]octane-2-carboxylate (690 mg, 2.11 mmol) in THF (15 mL) cooled to 0° C. The mixture was warmed to RT and stirred for 48 h, before heating to 50° C. and stirring for a further 5 h. The mixture was then partitioned between water (100 mL) and EtOAc (100 mL). The organic layer was collected and the aqueous layer was extracted with EtOAc (50 mL). The combined organic layers were dried using a phase separator and concentrated in vacuo. The resulting residue was purified by FC (0-10% (0.7 M ammonia/MeOH)/DCM) to afford the Boc-protected product. This was taken up in TFA (5 mL) and stirred at RT for 2 h. The reaction was concentrated in vacuo and the TFA residues were removed as azeotrope with methanol (×3) to afford the title compound (242 mg, 28%) as a colourless oil.

LCMS m/z 185.4 (M-2TFA+H)⁺ (ES⁺).

### Intermediate A25: 3-(4-oxa-1,9-diazaspiro[5.5]undecan-1-yl)propan-1-ol-4-oxa-1,9-diazaspiro[5.5]undecane (1/1), di-(2,2,2-trifluoroacetic acid)

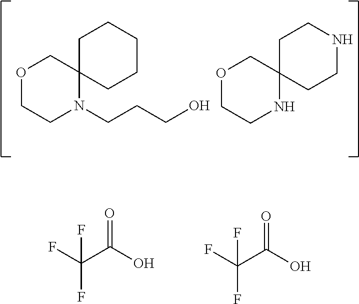

Acetic acid (0.5 mL, 9 mmol) was added to a solution of tert-butyl 4-oxa-1,9-diazaspiro-[5.5]undecane-9-carboxylate (1.00 g, 3.90 mmol) and 3-((tert-butyl-dimethylsilyl)oxy)-propanal (955 mg, 5.07 mmol) in anhydrous THF (35 mL) and the solution stirred at RT for 15 min before sodium triacetoxyborohydride (2.48 g, 11.7 mmol) was added portionwise. The reaction was stirred at RT for 18 h. The mixture was diluted with EtOAc (50 mL) and basified with 1 M aq. NaOH to pH˜9. The organic phase was separated, the aqueous further extracted with EtOAc (100 mL), the organic phases combined, dried (MgSO₄), filtered and concentrated under reduced pressure. The residue was loaded onto a pre-washed plug of SCX (15 g), the plug washed with MeOH (100 mL) and the product eluted with 0.7 N NH₃ in MeOH (150 mL). The ammoniacal solution was concentrated under reduced pressure to afford a yellow oil (889.4 mg). This oil was taken up in DCM/TFA (1:1, 20 mL) and stirred at RT for 3 h. The volatiles were removed under reduced pressure and the residue azeotroped with MeOH (2×30 mL) to afford the title compound (1.36 g, 58%) as the di-TFA salt.

LCMS m/z 215.3 (M+H)⁺ (ES⁺).

### Intermediate A26: 4-(3-((dimethylamino)methyl)pyrrolidin-3-yl)butan-1-ol

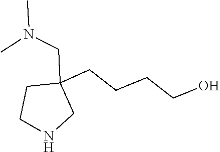

### Step A: 1-benzyl-3-(4-((tert-butyldimethylsilyl)oxy)butyl)pyrrolidine-3-carbonitrile

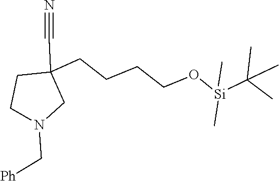 [SMILES: CC(C)(C)[Si](C)(C)OCCCCC1(C#N)CCN(Cc2ccccc2)C1]

1M LiHMDS in THF (14.6 mL, 14.6 mmol) was added dropwise to 1-benzylpyrrolidine-3-carbonitrile (2.59 g, 13.9 mmol) in THF (50 mL) cooled to −78° C. over 10 minutes. The mixture was left to stir at −78° C. for 30 minutes. tert-Butyl(4-iodobutoxy)dimethyl-silane (3.79 mL, 13.9 mmol) was then added in one portion and the mixture was left to warm to RT over 2.5 h. The mixture was quenched with sat. aq. NH₄Cl (50 mL), and the resulting mixture was diluted with water (20 mL) and EtOAc (100 mL). The organic layer was collected and the aqueous layer was extracted with EtOAc (2×50 mL). The combined organic layers were dried (MgSO₄), filtered and concentrated to dryness to give an orange oil. The crude product was purified by FC (0-50% EtOAc/heptane) to afford the title compound (2.60 g, 50%) as a pale yellow oil.

¹H NMR (500 MHz, DMSO-d₆) δ 7.35-7.28 (m, 4H), 7.27-7.22 (m, 1H), 3.64-3.51 (m, 4H), 2.94 (d, J=9.5 Hz, 1H), 2.81-2.69 (m, 1H), 2.47-2.39 (m, 1H), 2.35 (m, 1H), 2.25-2.15 (m, 1H), 1.89-1.80 (m, 1H), 1.67-1.59 (m, 2H), 1.53-1.33 (m, 4H), 0.85 (s, 9H), 0.02 (s, 6H).

### Step B: 4-(3-(aminomethyl)-1-benzylpyrrolidin-3-yl)butan-1-ol

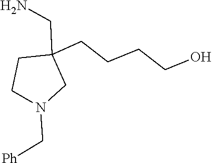 [SMILES: NCC1(CCCCO)CCN(Cc2ccccc2)C1]

To a solution of 1-benzyl-3-(4-((tert-butyldimethylsilyl)oxy)butyl)pyrrolidine-3-carbonitrile (2.60 g, 6.91 mmol) in THF (60 mL) at RT was added 1M LiAlH₄ in THF (20.7 mL, 20.7 mmol) drop-wise and the resultant reaction mixture refluxed for 3.5 h. The reaction was allowed to cool to RT and the reaction mixture was then treated dropwise with water (5 mL), NaOH (2M aq. soln, 10 mL) and water (15 mL) and then stirred vigorously for 30 min. The mixture was then filtered and the filtrate concentrated in vacuo to afford the title compound (1.93 g, 99%) as a pale yellow oil.

¹H NMR (500 MHz, DMSO-d₆) δ 7.33-7.25 (m, 4H), 7.24-7.18 (m, 1H), 3.53-3.44 (m, 2H), 3.38 (t, J=6.5 Hz, 2H), 2.44 (s, 2H), 2.42-2.32 (m, 2H), 1.57-1.45 (m, 1H), 1.45-1.29 (m, 6H), 1.28-1.13 (m, 3H). Three exchangeable protons not observed.

### Step C: 4-(1-benzyl-3-((dimethylamino)methyl)pyrrolidin-3-yl)butan-1-ol

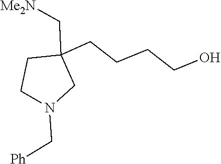 [SMILES: CN(C)CC1(CCCCO)CCN(Cc2ccccc2)C1]

A mixture of 4-(3-(aminomethyl)-1-benzylpyrrolidin-3-yl)butan-1-ol (1.93 g, 6.84 mmol), formaldehyde (20 mL, 37% wt) and formic acid (20 mL) was refluxed for 18 h. The reaction mixture was then allowed to cool to RT before being concentrated in vacuo. The resulting residue was mixed with ice, basified with aq. NaOH (2N) and extracted with DCM (100 mL). The organics were then washed with water and brine, dried (MgSO₄) and concentrated in vacuo to afford the title compound (1.75 g, 86%) as a thick yellow oil.

¹H NMR (500 MHz, DMSO-d₆) δ 7.32-7.26 (m, 4H), 7.24-7.18 (m, 1H), 4.08 (t, J=6.5 Hz, 1H), 3.56-3.44 (m, 2H), 3.38 (t, J=6.5 Hz, 1H), 2.49-2.41 (m, 2H), 2.32-2.18 (m, 4H), 2.15 (s, 6H), 1.61-1.47 (m, 3H), 1.46-1.33 (m, 4H), 1.33-1.18 (m, 2H).

### Step D: 4-(3-((dimethylamino)methyl)pyrrolidin-3-yl)butan-1-ol

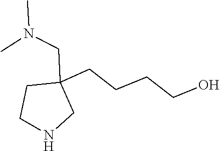

4-(1-benzyl-3-((dimethylamino)methyl)pyrrolidin-3-yl)butan-1-ol (1.75 g, 5.90 mmol) was dissolved in EtOH (35 mL) and acetic acid (676 μL, 11.8 mmol), to which was added palladium hydroxide on carbon (415 mg, 20% wt, 590 μmol). The reaction mixture was then hydrogenated at 4 bar pressure for ˜70 h at 40° C. The catalyst was then filtered off and the filtrate concentrated in vacuo, azeotroping with toluene, to afford the crude title compound (1.82 g) as a brown oil. The product used without further purification.

¹H NMR (500 MHz, DMSO-d₆) δ 3.41-3.35 (m, 2H), 3.10-3.02 (m, 1H), 2.80 (app s, 1H), 2.26-2.15 (m, 8H), 1.82 (app s, 5H), 1.64 (t, J=7.4 Hz, 1H), 1.43-1.32 (m, 4H), 1.29-1.19 (m, 2H).

### Intermediate A27: 3-((1S,4S)-2,5-diazabicyclo[2.2.1]heptan-2-yl)propan-1-ol, di-(2,2,2-trifluoroacetic acid)

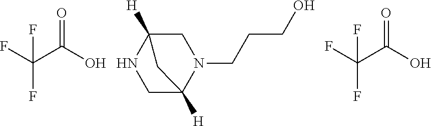

To a solution of tert-butyl (1S,4S)-2,5-diazabicyclo[2.2.1]heptane-2-carboxylate (1.10 g, 5.55 mmol) in MeCN (15 mL) was added K₂CO₃ (2.30 g, 16.6 mmol) and 3-bromo-propan-1-ol (502 μL, 5.55 mmol). The resulting suspension was stirred at 80° C. for 24 h, then concentrated in vacuo. The resulting residue was dissolved in EtOAc (50 mL), washed with brine (50 mL), dried using a phase separator and concentrated in vacuo. The crude product was purified by FC (0-10% (0.7 M ammonia/MeOH)/DCM) to afford the Boc-protected product as a colourless oil. This was taken up in TFA (5 mL) and stirred at RT for 2 h, before being concentrated in vacuo to afford the title compound (1.51 g, 70%) as a light brown oil.

¹H NMR (500 MHz, DMSO-d₆) δ 4.58-4.39 (m, 3H), 3.52-3.27 (m, 6H), 3.25-3.09 (m, 3H), 2.14-2.00 (m, 1H), 1.85-1.72 (m, 2H). Three exchangeable protons not observed.

### Intermediate A28: sodium 5-((5-(2-fluoropyridin-4-yl)-2,3-dihydro-1H-inden-4-yl)oxy)-1-((2-(trimethylsilyl)ethoxy)methyl)-1H-1,2,4-triazole-3-sulfinate

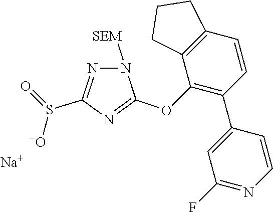

### Step A: 5-bromo-2,3-dihydro-1H-inden-4-ol

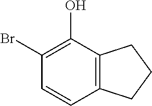 [SMILES: Oc1c(Br)ccc2c1CCC2]

NBS (5.3 g, 30 mmol) was added portionwise over 30 minutes to a stirred solution of 2,3-dihydro-1H-inden-4-ol (4.0 g, 30 mmol) and diisopropylamine (0.42 mL, 3.0 mmol) in DCM (80 mL) cooled in an ice bath. The mixture was then left to stir at RT for 18 h. The mixture was diluted with 1M aq. HCl (100 mL) and the organic layer was collected. The aqueous layer was extracted with DCM (2×50 mL) and the combined organic layers were concentrated to dryness to give a yellow oil. The crude product was purified by RP FC (C18, 15-100% (0.1% formic acid in MeCN)/(0.1% formic acid in water)) to afford the title compound (4.0 g, 62%) as a white solid.

¹H NMR (500 MHz, DMSO-d₆) δ 9.26 (s, 1H), 7.21 (d, J=7.9 Hz, 1H), 6.64 (d, J=7.9 Hz, 1H), 2.83 (t, J=7.4 Hz, 2H), 2.79 (t, J=7.5 Hz, 2H), 1.99 (p, J=7.5 Hz, 2H).

### Step B: 5-(2-fluoropyridin-4-yl)-2,3-dihydro-1H-inden-4-ol

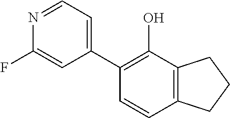 [SMILES: Oc1c(-c2ccnc(F)c2)ccc2c1CCC2]

(2-fluoropyridin-4-yl)boronic acid (2.6 g, 19 mmol) was added to a stirred mixture of 5-bromo-2,3-dihydro-1H-inden-4-ol (4.0 g, 19 mmol), Pd(dppf)Cl₂.CH₂Cl₂ (0.77 g, 0.94 mmol) and K₂CO₃ (7.8 g, 56 mmol) in 9:1 1,4-dioxane:water (11 mL). The mixture was degassed with a stream of nitrogen gas for 15 min then heated to 90° C. for 18 h. The mixture was left to cool to RT and filtered through a pad of Celite, washing through with EtOAc (2×15 mL). The filtrate was concentrated to dryness on silica (30 g). The crude product was purified by FC (0-30% EtOAc/isohexane (elution˜10%)) to afford a yellow/green solid. The material was triturated with 1:4 MTBE:heptane (100 mL) and filtered to afford the title compound (1.39 g, 23%) as an off-white solid.

LCMS m/z 230.3 (M+H)⁺ (ES⁺); 228.6 (M−H)⁻ (ES⁻).

¹H NMR (500 MHz, DMSO-d₆) δ 9.08 (s, 1H), 8.21 (d, J=5.3 Hz, 1H), 7.52 (ddd, J=5.3, 2.3, 1.4 Hz, 1H), 7.30 (d, J=1.3 Hz, 1H), 7.19 (d, J=7.7 Hz, 1H), 6.85 (d, J=7.7 Hz, 1H), 2.87 (td, J=7.3, 5.2 Hz, 4H), 2.03 (p, J=7.4 Hz, 2H).

¹⁹F NMR (471 MHz, DMSO-d⁶) δ −69.86.

### Step C: 4-(4-((3-bromo-1-((2-(trimethylsilyl)ethoxy)methyl)-1H-1,2,4-triazol-5-yl)oxy)-2,3-dihydro-1H-inden-5-yl)-2-fluoropyridine

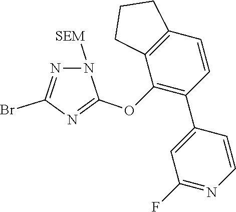 [SMILES: C[Si](C)(C)CCOCn1nc(Br)nc1Oc1c(-c2ccnc(F)c2)ccc2c1CCC2]

A stirred mixture of 2-[(3,5-dibromo-1,2,4-triazol-1-yl)methoxy]ethyl-trimethyl-silane (2.46 g, 6.88 mmol) (Intermediate A1), 5-(2-fluoropyridin-4-yl)-2,3-dihydro-1H-inden-4-ol (1.58 g, 6.88 mmol) and K₂CO₃ (1.90 g, 13.8 mmol) in NMP (30 mL) was heated to 100° C. for 24 h. The reaction was diluted with EtOAc (100 mL) and washed with water (50 mL). The aqueous layer was re-extracted with EtOAc (50 mL), and the combined organics washed with brine (75 mL), dried (MgSO₄) and concentrated in vacuo. The crude product was purified by FC (0-20% EtOAc/isohexane) to afford the title compound (3.08 g, 76%) as a thick colourless oil.

LCMS m/z 505.3/507.3 (M+H)⁺ (ES⁺).

¹H NMR (500 MHz, DMSO-d₆) δ 8.24 (d, J=5.2 Hz, 1H), 7.51-7.31 (m, 3H), 7.23 (d, J=1.5 Hz, 1H), 5.38 (s, 2H), 3.56 (t, J=8.0 Hz, 2H), 3.00 (t, J=7.5 Hz, 2H), 2.72-2.65 (m, 2H), 2.07 (p, J=7.5 Hz, 2H), 0.88-0.73 (m, 2H), −0.06 (s, 9H).

### Step D: methyl 3-((5-((5-(2-fluoropyridin-4-yl)-2,3-dihydro-1H-inden-4-yl)oxy)-1-((2-(trimethylsilyl)ethoxy)methyl)-1H-1,2,4-triazol-3-yl)thio)propanoate

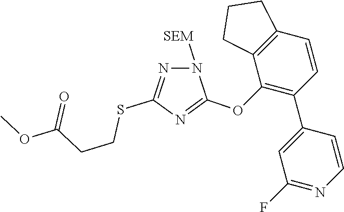 [SMILES: COC(=O)CCSc1nc(Oc2c(-c3ccnc(F)c3)ccc3c2CCC3)n(COCC[Si](C)(C)C)n1]

4-(4-((3-bromo-1-((2-(trimethylsilyl)ethoxy)methyl)-1H-1,2,4-triazol-5-yl)oxy)-2,3-dihydro-1H-inden-5-yl)-2-fluoropyridine (3.08 g, 5.25 mmol) was dissolved in 1,4-dioxane (70 mL) and the mixture was degassed with N₂ for 10 min. Methyl 3-mercaptopropanoate (1.16 mL, 10.5 mmol), XantPhos (455 mg, 787 μmol) and Pd₂(dba)₃ (360 mg, 393 μmol) were added followed by DIPEA (1.83 mL, 10.5 mmol) and the reaction mixture was degassed with N₂ (10 min), then evacuated and back-filled with N₂ (3 times). The reaction mixture was warmed up to 100° C. and stirred for 24 h. The reaction was cooled to RT and diluted with water (200 mL). The organics were then extracted with EtOAc (2×150 mL), and the combined organics dried using a phase separator and then concentrated in vacuo. The crude product was purified by FC (0-40% EtOAc/isohexane) to afford the title compound (3.22 g, 99%) as a yellow oil.

LCMS m/z 545-5 (M+H)⁺.

¹H NMR (500 MHz, DMSO-d⁶) δ 8.24 (d, J=5.2 Hz, 1H), 7.43-7.35 (m, 3H), 7.22 (s, 1H), 5-35 (s, 2H), 3.63-3.54 (m, 5H), 3.10 (t, J=7.0 Hz, 2H), 2.99 (t, J=7.4 Hz, 2H), 2.73-2.63 (m, 4H), 2.07 (p, J=7.4 Hz, 2H), 0.83 (t, J=8.0 Hz, 2H), −0.05 (s, 9H).

### Step E: methyl 3-((5-((5-(2-fluoropyridin-4-yl)-2,3-dihydro-1H-inden-4-yl)oxy)-1-((2-(trimethylsilyl)ethoxy)methyl)-1H-1,2,4-triazol-3-yl)sulfonyl)propanoate

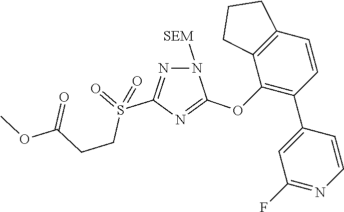 [SMILES: COC(=O)CCS(=O)(=O)c1nc(Oc2c(-c3ccnc(F)c3)ccc3c2CCC3)n(COCC[Si](C)(C)C)n1]

m-CPBA (3.06 g, 13.3 mmol) was added to solution of methyl 3-((5-((5-(2-fluoro-pyridin-4-yl)-2,3-dihydro-1H-inden-4-yl)oxy)-1-((2-(trimethylsilyl)ethoxy)methyl)-1H-1,2,4-triazol-3-yl)thio)propanoate (3.22 g, 5-32 mmol) in DCM (75 mL) at 0° C. The reaction was allowed to warm to RT and stirred for 18 h. The reaction was quenched with sat. aq. Na₂SO₃ (250 mL) and then extracted with DCM (3×150 mL). The combined organics were then washed with sat. aq. NaHCO₃ (400 mL), dried (MgSO₄) and concentrated in vacuo to afford the title compound (3.236 g, 100%) as a brown oil.

LCMS m/z 577.4 (M+H)⁺.

¹H NMR (500 MHz, DMSO-d₆) δ 8.23 (d, J=5.2 Hz, 1H), 7.46-7.40 (m, 3H), 7.24 (s, 1H), 5.55 (s, 2H), 3.65-3.54 (m, 7H), 3.02 (t, J=7.4 Hz, 2H), 2.73 (t, J=7.4 Hz, 2H), 2.63 (t, J=7.1 Hz, 2H), 2.08 (p, J=7.4 Hz, 2H), 0.85 (t, J=8.1 Hz, 2H), −0.05 (s, 9H).

### Step F: sodium 5-((5-(2-fluoropyridin-4-yl)-2,3-dihydro-1H-inden-4-yl)oxy)-1-((2-(trimethylsilyl)ethoxy)methyl)-1H-1,2,4-triazole-3-sulfinate

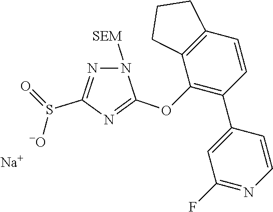

To a solution of methyl 3-((5-((5-(2-fluoropyridin-4-yl)-2,3-dihydro-1H-inden-4-yl)-oxy)-1-((2-(trimethylsilyl)ethoxy)methyl)-1H-1,2,4-triazol-3-yl)sulfonyl)propanoate (3.24 g, 5.22 mmol) in THF (70 mL) at 0° C. was added sodium 2-methylbutan-2-olate (2.5 M in THF) (3.13 mL, 7.83 mmol) and the reaction was then stirred at 0° C. for 1 h. The reaction was then concentrated in vacuo and the resulting residue was diluted with hexanes (75 mL) and concentrated in vacuo (×3), to afford the title compound (2.96 g, 99%) as a light brown solid.

LCMS m/z 491.7 (M-Na+2H)⁺ (ES⁺).

¹H NMR (500 MHz, DMSO-d₆) δ 8.24 (d, J=5.2 Hz, 1H), 7.46-7.39 (m, 2H), 7.35 (d, J=7.7 Hz, 1H), 7.22 (s, 1H), 5.31 (s, 2H), 3.55 (t, J=8.1 Hz, 2H), 3.00 (t, J=7.5 Hz, 2H), 2.62 (t, J=7.5 Hz, 2H), 2.05 (p, J=7.5 Hz, 2H), 0.84 (t, J=8.0 Hz, 2H), −0.05 (s, 9H).

### Intermediate A29: di-sodium (4-fluoro-2-(2-fluoropyridin-4-yl)-6-isopropyl-phenyl)(3-sulfinato-1-((2-(trimethylsilyl)ethoxy)methyl)-1H-1,2,4-triazol-5-yl)amide

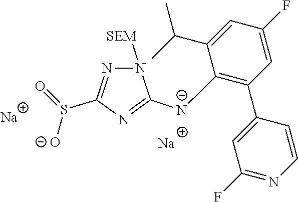 [SMILES: [Na+] ; CC(C)c1cc(F)cc(-c2ccnc(F)c2)c1[N-]c1nc(S(=O)[O-])nn1COCC[Si](C)(C)C ; [Na+]]

### Step A: 4-fluoro-2-(2-fluoropyridin-4-yl)-6-isopropylaniline

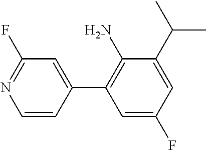 [SMILES: CC(C)c1cc(F)cc(-c2ccnc(F)c2)c1N]

A solution of 2-bromo-4-fluoro-6-isopropylaniline (0.750 g, 3.23 mmol) in anhydrous 1,4-dioxane (30 mL) was added to a mixture of (2-fluoropyridin-4-yl)boronic acid (0.460 g, 3.26 mmol) and Pd(dppf)Cl₂.DCM (0.130 g, 0.160 mmol) followed by a solution of K₂CO₃ (1.75 g, 12.7 mmol) in water (3 mL). The resulting suspension was evacuated and backfilled with N₂ twice before stirring at 95° C. for 18 h. The reaction was diluted with EtOAc (100 mL) and washed with water/brine (3:1, 100 mL). The crude was directly loaded onto silica for purification and the volatiles were removed. The crude product was purified by FC (0-50% EtOAc/isohexane) to afford the title compound (626 mg, 74%) as a purple gum.

LCMS m/z 249.0 (M+H)⁺ (ES⁺).

¹H NMR (500 MHz, CDCl₃) δ 8.30 (d, J=5.0 Hz, 1H), 7.31-7.28 (m, 1H), 7.04 (br s, 1H), 6.96 (dd, J=9.9, 2.9 Hz, 1H), 6.71 (dd, J=8.5, 2.9 Hz, 1H), 3.64 (br s, 2H), 2.97-2.87 (m, 1H), 1.29 (d, J=6.8 Hz, 6H).

### Step B: 3-bromo-N-(4-fluoro-2-(2-fluoropyridin-4-yl)-6-isopropylphenyl)-1-((2-(trimethylsilyl)ethoxy)methyl)-1H-1,2,4-triazol-5-amine

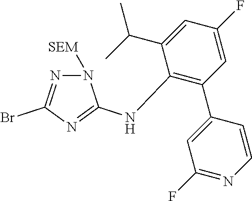 [SMILES: CC(C)c1cc(F)cc(-c2ccnc(F)c2)c1Nc1nc(Br)nn1COCC[Si](C)(C)C]

LiHMDS (1.0 M in THF) (40.0 mL, 40.0 mmol) was added over 15 min to a mixture of 2-[(3,5-dibromo-1,2,4-triazol-1-yl)methoxy]ethyl-trimethyl-silane (7.19 g, 20.1 mmol) (Intermediate A1) and 4-fluoro-2-(2-fluoropyridin-4-yl)-6-isopropylaniline (5.00 g, 20.1 mmol) in THF (100 mL) cooled to 0° C. The reaction was stirred at RT for 2 h. The reaction was quenched with sat. aq. NH₄Cl (60 mL) and extracted with EtOAc (2×100 mL). The combined organic phases were washed with brine (100 mL), dried (MgSO₄), filtered and concentrated under reduced pressure. The crude product was purified by FC (0-20% EtOAc/isohexane) to afford the title compound (5.89 g, 51%) as a thick orange oil.

LCMS m/z 524.7/526.3 (M+H)⁺ (ES⁺).

¹H NMR (500 MHz, DMSO-d₆) δ 8.78 (s, 1H), 8.19 (d, J=5.1 Hz, 1H), 7.36 (dd, J=9.9, 3.0 Hz, 1H), 7.31-7.28 (m, 1H), 7.19 (dd, J=8.7, 3.0 Hz, 1H), 7.13 (s, 1H), 5.23 (s, 2H), 3.50-3.41 (m, 2H), 3.19-3.09 (m, 1H), 1.15 (d, J=6.9 Hz, 6H), 0.83-0.78 (m, 2H), −0.03 (s, 9H).

### Step C: methyl 3-((5-((4-fluoro-2-(2-fluoropyridin-4-yl)-6-isopropylphenyl)amino)-1-((2-(trimethylsilyl)ethoxy)methyl)-1H-1,2,4-triazol-3-yl)thio)propanoate

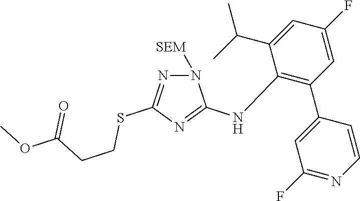 [SMILES: COC(=O)CCSc1nc(Nc2c(-c3ccnc(F)c3)cc(F)cc2C(C)C)n(COCC[Si](C)(C)C)n1]

Pd₂(dba)₃ (770 mg, 841 μmol) and XantPhos (975 mg, 1.68 mmol) were evacuated and backfilled with N₂ twice before a solution of 3-bromo-N-(4-fluoro-2-(2-fluoropyridin-4-yl)-6-isopropylphenyl)-1-((2-(trimethylsilyl)ethoxy)methyl)-1H-1,2,4-triazol-5-amine (5.89 g, 11.2 mmol) in anhydrous 1,4-dioxane (250 mL) was added followed by DIPEA (4.00 mL, 23.0 mmol) and methyl 3-mercaptopropanoate (2.50 mL, 22.6 mmol). The resulting solution was evacuated and backfilled twice at 60° C., then the reaction was stirred at 100° C. for 18 h. The reaction was diluted with water (300 mL), the organic phase separated, the aqueous further extracted with EtOAc (3×200 mL), the combined organic phases dried (MgSO₄), filtered and concentrated under reduced pressure. The crude product was purified by FC (0-40% EtOAc/isohexane) to afford the title compound (5.96 g, 85%) as a yellow oil, which solidified upon standing.

LCMS m/z 564.4 (M+H)⁺ (ES⁺); 562.5 (M−H)⁻ (ES⁻).

¹H NMR (500 MHz, DMSO-d₆) δ 8.52 (s, 1H), 8.17 (d, J=5.2 Hz, 1H), 7.34 (dd, J=10.0, 3.0 Hz, 1H), 7.32-7.27 (m, 1H), 7.17 (dd, J=8.7, 3.0 Hz, 1H), 7.14-7.11 (m, 1H), 5.21 (s, 2H), 3.58 (s, 3H), 3.49-3.40 (m, 2H), 3.20-3.10 (m, 1H), 3.02 (t, J=7.0 Hz, 2H), 2.61 (t, J=7.0 Hz, 2H), 1.13 (d, J=6.9 Hz, 6H), 0.84-0.76 (m, 2H), −0.03 (s, 9H).

### Step D: methyl 3-((5-((4-fluoro-2-(2-fluoropyridin-4-yl)-6-isopropylphenyl)amino)-1-((2-(trimethylsilyl)ethoxy)methyl)-1H-1,2,4-triazol-3-yl)sulfonyl)propanoate

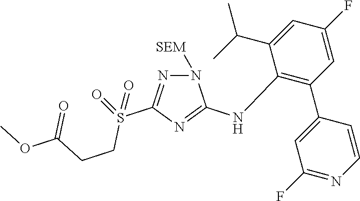 [SMILES: COC(=O)CCS(=O)(=O)c1nc(Nc2c(-c3ccnc(F)c3)cc(F)cc2C(C)C)n(COCC[Si](C)(C)C)n1]

m-CPBA (6.08 g, 75.0% wt, 26.4 mmol) was added to solution of methyl 3-((5-((4-fluoro-2-(2-fluoropyridin-4-yl)-6-isopropylphenyl)amino)-1-((2-(trimethylsilyl)-ethoxy)methyl)-1H-1,2,4-triazol-3-yl)thio)propanoate (5.96 g, 10.6 mmol) in DCM (200 mL) at 0° C. The reaction was allowed to warm to RT and stirred for 18 h. The reaction was quenched with sat. aq. Na₂SO₃ (200 mL) and then extracted with DCM (3×400 mL). The combined organics were then washed with sat. aq. NaHCO₃ (100 mL), dried (MgSO₄) and concentrated under reduced pressure to afford crude title compound (5-47 g, 85%) as an orange solid.

¹H NMR (500 MHz, DMSO-d₆) δ 9.05 (s, 1H), 8.16 (d, J=5.2 Hz, 1H), 7.39 (dd, J=9.9, 3.0 Hz, 1H), 7.30 (dt, J=5.2, 1.7 Hz, 1H), 7.21 (dd, J=8.7, 3.0 Hz, 1H), 7.13 (s, 1H), 5.39 (s, 2H), 3.59 (s, 3H), 3.49-3.41 (m, 4H), 3.20-3.13 (m, 1H), 2.53 (t, J=7.3 Hz, 2H), 1.14 (d, J=6.7 Hz, 6H), 0.85-0.77 (m, 2H), −0.03 (s, 9H).

### Step E: di-sodium (4-fluoro-2-(2-fluoropyridin-4-yl)-6-isopropylphenyl)(3-sulfinato-1-((2-(trimethylsilyl)ethoxy)methyl)-1H-1,2,4-triazol-5-yl)amide

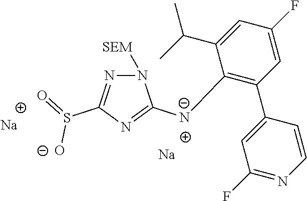 [SMILES: [Na+] ; CC(C)c1cc(F)cc(-c2ccnc(F)c2)c1[N-]c1nc(S(=O)[O-])nn1COCC[Si](C)(C)C ; [Na+]]

Sodium 2-methylbutan-2-olate (3.95 mL, 2.5 M, 9.88 mmol) was added to a solution of methyl 3-((5-((4-fluoro-2-(2-fluoropyridin-4-yl)-6-isopropylphenyl)amino)-1-((2-(trimethylsilyl)ethoxy)methyl)-1H-1,2,4-triazol-3-yl)sulfonyl)propanoate (5.47 g, 9.00 mmol) in anhydrous THF (150 mL) at 0° C. The reaction was stirred at this temperature for 2 h. Additional sodium 2-methylbutan-2-olate (3.95 mL, 2.5 M, 9.88 mmol) was added and the reaction stirred for 30 min before being concentrated under reduced pressure. The traces of solvent were azeotroped with hexanes (×4) to afford crude title compound (5.34 g, 91%) as a dark yellow solid.

¹H NMR (500 MHz, DMSO-d₆) δ 7.94 (s, 1H), 7.44 (s, 1H), 7.25 (s, 1H), 6.96-6.76 (m, 2H), 4.99 (s, 2H), 3.61-3.49 (m, 2H), 1.07 (d, J=6.9 Hz, 6H), 0.84-0.79 (m, 2H), −0.03 (s, 9H). One proton overlapped with water signal in DMSO-d₆.

### Intermediate A30: di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)amide

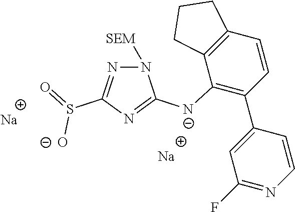 [SMILES: [Na+] ; C[Si](C)(C)CCOCn1nc(S(=O)[O-])nc1[N-]c1c(-c2ccnc(F)c2)ccc2c1CCC2 ; [Na+]]

To a solution of methyl 3-[[5-[[5-(2-fluoro-4-pyridyl)indan-4-yl]amino]-1-(2-trimethylsilylethoxymethyl)-1,2,4-triazol-3-yl]sulfonyl]propanoate (Intermediate A3, Step A) (12.2 g, 16.0 mmol) in THF (250 mL) at 0° C. was added sodium 2-methylbutan-2-olate (2.5 M in THF) (9.62 mL, 24.1 mmol) and the reaction was then stirred at 0° C. for 1 h. Additional sodium 2-methylbutan-2-olate (6.42 mL, 16.0 mmol) was added and the reaction stirred for a further 1 h at 0° C. The reaction was then concentrated in vacuo and the resulting residue was diluted with hexanes (75 mL) and concentrated in vacuo (×4), to afford crude title compound (10.8 g, 99.7%) as a brown solid.

¹H NMR (500 MHz, DMSO-d₆) δ 7.99 (d, J=5.3 Hz, 1H), 7.58-7.54 (m, 1H), 7.52 (d, J=1.3 Hz, 1H), 7.07 (d, J=7.7 Hz, 1H), 6.56 (d, J=7.7 Hz, 1H), 5.04 (s, 2H), 3.60-3.50 (m, 2H), 2.79 (t, J=7.4 Hz, 2H), 1.85 (p, J=7.4 Hz, 2H), 0.84-0.74 (m, 2H), −0.07 (s, 9H). Two aliphatic protons obscured.

### Intermediate A31: 3-(2,6-diazaspiro[3.4]octan-6-yl)propan-1-ol bis(2,2,2-trifluoroacetate)

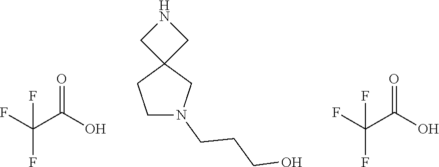 [SMILES: O=C(O)C(F)(F)F ; OCCCN1CCC2(CNC2)C1 ; O=C(O)C(F)(F)F]

### Step A: tert-butyl 6-(3-hydroxypropyl)-2,6-diazaspiro[3.4]octane-2-carboxylate

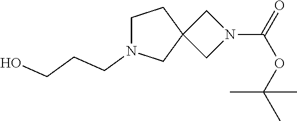 [SMILES: CC(C)(C)OC(=O)N1CC2(CCN(CCCO)C2)C1]

A solution of tert-butyl 2,6-diazaspiro[3.4]octane-2-carboxylate (2.40 g, 11.3 mmol), 3-bromo-1-propanol (1.15 mL, 12.7 mmol) and TEA (3.20 mL, 23.0 mmol) in anhydrous THF (50 mL) was stirred at RT for 18 h. The volatiles were removed under reduced pressure, the residue partitioned between EtOAc (20 mL) and water (20 mL) and the phases separated. The aqueous was further extracted with EtOAc (20 mL), the organic phases were combined, dried (MgSO4), filtered and concentrated under reduced pressure. The crude product was purified by FC, 0-10% ((0.7 M Ammonia/MeOH)/DCM) to afford the title compound (1.57 g, 40% yield, 77% purity) as an orange oil.

LCMS m/z 271.4 (M+H)⁺ (ES⁺), 215.4 (M-^{t}Bu+H)⁺ (ES⁺).

### Step B: 3-(2,6-diazaspiro[3.4]octan-6-yl)propan-1-ol bis(2,2,2-trifluoroacetate)

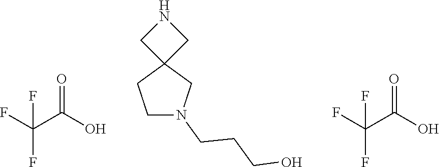 [SMILES: O=C(O)C(F)(F)F ; OCCCN1CCC2(CNC2)C1 ; O=C(O)C(F)(F)F]

To a solution of tert-butyl 6-(3-hydroxypropyl)-2,6-diazaspiro[3.4]octane-2-carboxylate (1.57 g, 77% wt, 4.47 mmol) in DCM (5 mL) was added TFA (5 mL, 0.06 mol). The reaction mixture was stirred at RT for 2 h after which the reaction mixture was concentrated in vacuo to give the title compound (4.4 g, 170% yield, 70% purity) as a clear yellow oil.

¹H NMR (500 MHz, DMSO-d₆) δ 10.56 (s, 1H), 9.22-8.71 (m, 2H), 4.44 (t, J=6.1 Hz, 2H), 4.12-4.04 (m, 2H), 4.01 (s, 2H), 3.91 (t, J=14.3 Hz, 2H), 3.63 (s, 1H), 3.38-3.29 (m, 1H), 3.24 (d, J=7.4 Hz, 2H), 3.10 (d, J=13.0 Hz, 1H), 2.49-2.38 (m, 1H), 2.23 (q, J=9.0, 8.5 Hz, 1H), 2.08 (qd, J=8.9, 6.0 Hz, 2H).

### Preparation of Examples

### Example 1: 26-methyl-22-oxa-2λ⁶-thia-1,4,5,7,20,26,33-heptazahexacyclo-[25.2.2.1^{3,6}.1^{17,21}.0^{8,16}.0^{9,13}]tritriaconta-3,6(33),8,13,15,17,19,21(32)-octaene 2,2-dioxide

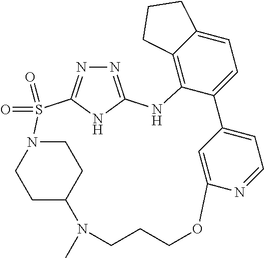 [SMILES: CN1CCCOc2cc(ccn2)-c2ccc3c(c2Nc2nnc([nH]2)S(=O)(=O)N2CCC1CC2)CCC3]

### Step A: 3-[[1-[[5-[[5-(2-fluoro-4-pyridyl)indan-4-yl]amino]-1-(2-trimethylsilyl-ethoxymethyl)-1,2,4-triazol-3-yl]sulfonyl]-4-piperidyl]-methyl-amino]propan-1-ol

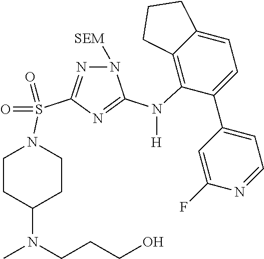 [SMILES: CN(CCCO)C1CCN(S(=O)(=O)c2nc(Nc3c(-c4ccnc(F)c4)ccc4c3CCC4)n(COCC[Si](C)(C)C)n2)CC1]

NCS (160 mg, 1.20 mmol) was added to a mixture of ammonium 5-[[5-(2-fluoro-4-pyridyl)indan-4-yl]amino]-1-(2-trimethylsilylethoxymethyl)-1,2,4-triazole-3-sulfinate (Intermediate A4) (489 mg, 1.00 mmol) in DCM (9.00 mL) at 0° C. under N₂. The mixture was stirred at 0° C. for 1 h and a mixture of 3-hydroxypropyl-methyl-piperidin-1-ium-4-yl-ammonium dichloride (Intermediate A5) (262 mg, 1.52 mmol) and DIPEA (0.520 mL, 3.00 mmol) in DCM (3.00 mL) was added. The mixture and stirred for 1 h at 0° C. and diluted with water (7.00 mL). The aqueous phase was extracted with EtOAc (2×100 mL), and the combined organic phases were washed with brine (20.0 mL), dried (MgSO₄), filtered, and concentrated. The product was purified by FC (0-20% MeOH/DCM) to provide the title compound as a solid (419 mg, 63%).

LCMS m/z 660.2 (M+H)⁺ (ES⁺).

¹H NMR (400 MHz, CDCl₃) δ 8.18 (d, J=5.1 Hz, 1H), 7.25-7.18 (m, 2H), 7.14 (d, J=7.7 Hz, 1H), 6.97 (s, 1H), 6.42-6.30 (m, 1H), 5.37 (s, 2H), 3.96-3.87 (m, 2H), 3.83-3.74 (m, 2H), 3.58-3.42 (m, 2H), 3.02 (t, J=7.4 Hz, 2H), 2.79 (t, J=7.4 Hz, 2H), 2.73-2.68 (m, 2H), 2.59-2.48 (m, 3H), 2.29 (s, 3H), 2.17-2.06 (m, 2H), 1.84-1.76 (m, 2H), 1.76-1.58 (m, 5H), 0.82-0.73 (m, 2H), −0.00 (s, 9H).

¹⁹F NMR (376 MHz, CDCl₃) δ −67.37 (s).

### Step B: trimethyl-[2-[(26-methyl-2,2-dioxo-22-oxa-2λ⁶-thia-1,4,5,7,20,26,33-heptazahexacyclo[25.2.2.1^{3,6}.1^{17,21}.0^{8,16}.0^{9,13}]tritriaconta-3,6(33),8,13,15,17,19,21(32)-octaen-5-yl)methoxy]ethyl]silane

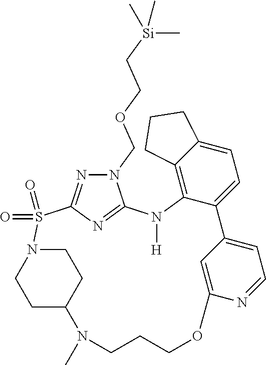 [SMILES: CN1CCCOc2cc(ccn2)-c2ccc3c(c2Nc2nc(nn2COCC[Si](C)(C)C)S(=O)(=O)N2CCC1CC2)CCC3]

tBuOK (1 M in THF, 5.89 mL, 5.89 mmol) was added to a mixture of 3-[[1-[[5-[[5-(2-fluoro-4-pyridyl)indan-4-yl]amino]-1-(2-trimethylsilylethoxymethyl)-1,2,4-triazol-3-yl]sulfonyl]-4-piperidyl]-methyl-amino]propan-1-ol (389 mg, 0.589 mmol) in THF (260 mL) at 0° C. The mixture was stirred at 0° C. for 2.5 h, diluted with EtOH (10.0 mL) and concentrated. The product was purified by FC (0-20% MeOH/DCM) to provide the title compound (139 mg, 37%).

LCMS m/z 640.6 (M+H)⁺ (ES⁺).

¹H NMR (500 MHz, CDCl₃) δ 8.07 (d, J=5.3 Hz, 1H), 7.22 (d, J=7.7 Hz, 1H), 7.18 (d, J=7.7 Hz, 1H), 7.08-7.01 (m, 1H), 6.80-6.75 (m, 1H), 6.22 (s, 1H), 5.41 (s, 2H), 4.32-4.24 (m, 2H), 3.63-3.45 (m, 4H), 3.01 (t, J=7.5 Hz, 2H), 2.81 (t, J=7.3 Hz, 2H), 2.66-2.50 (m, 2H), 2.33-2.25 (m, 1H), 2.21 (s, 3H), 2.19-2.06 (m, 3H), 1.96-1.88 (m, 2H), 1.78-1.66 (m, 2H), 1.64-1.53 (m, 3H), 0.91-0.83 (m, 2H), 0.01 (s, 9H).

### Step C: 26-methyl-22-oxa-2λ⁶-thia-1,4,5,7,20,26,33-heptazahexacyclo-[25.2.2.1^{3,6}.1^{17,21}.0^{8,16}.0^{9,13}]tritriaconta-3,6(33),8,13,15,17,19,21(32)-octaene 2,2-dioxide

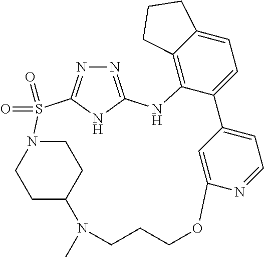 [SMILES: CN1CCCOc2cc(ccn2)-c2ccc3c(c2Nc2nnc([nH]2)S(=O)(=O)N2CCC1CC2)CCC3]

A mixture of trimethyl-[2-[(26-methyl-2,2-dioxo-22-oxa-2λ⁶-thia-1,4,5,7,20,26,33-heptazahexacyclo[25.2.2.1^{3,6}.1^{17,21}.0^{8,16}.0^{9,13}]tritriaconta-3,6(33),8,13,15,17,19,21(32)-octaen-5-yl)methoxy]ethyl]silane (17.0 mg, 0.0266 mmol) in DCM (1.00 mL) and TFA (1.00 mL) was stirred at 22° C. for 2 h and concentrated. The product was purified by basic prep HPLC (0-100% MeCN in water) to provide the title compound as a solid (6.85 mg, 51%).

LCMS m/z 509.9 (M+H)⁺ (ES⁺).

¹H NMR (500 MHz, DMSO-d₆) δ 12.99 (s, 1H), 8.95 (s, 1H), 8.12 (d, J=5.4 Hz, 1H), 7.27 (d, J=7.6 Hz, 1H), 7.23 (d, J=7.7 Hz, 1H), 7.09-7.05 (m, 1H), 6.70 (s, 1H), 4.20 (t, J=5.4 Hz, 2H), 3.45-3.37 (m, 2H), 2.96 (t, J=7.5 Hz, 2H), 2.76 (t, J=7.4 Hz, 2H), 2.65-2.61 (m, 1H), 2.53-2.51 (m, 1H), 2.37-2.35 (m, 2H), 2.28-2.22 (m, 1H), 2.17 (s, 3H), 2.07-1.99 (m, 2H), 1.80-1.71 (m, 2H), 1.60-1.54 (m, 2H), 1.44-1.34 (m, 2H).

### Example 2: 26-oxa-16λ⁶-thia-11,13,14,17,22,28,34-heptazaheptacyclo-[25.3.1.1^{12,15}.1^{17,19}.1^{19,22}.0^{2,10}.0^{5,9}]tetratriaconta-1(30),2,4,9,12,14,27(31),28-octaene 16,16-dioxide

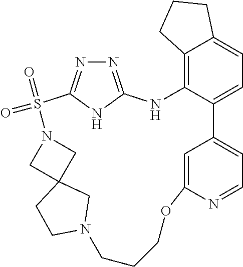 [SMILES: O=S1(=O)c2nnc([nH]2)Nc2c(ccc3c2CCC3)-c2ccnc(c2)OCCCN2CCC3(C2)CN1C3]

### Step A: 3-[2-[[5-[[5-(2-fluoro-4-pyridyl)indan-4-yl]amino]-1-(2-trimethylsilyl-ethoxymethyl)-1,2,4-triazol-3-yl]sulfonyl]-2,7-diazaspiro[3.4]octan-7-yl]propan-1-ol

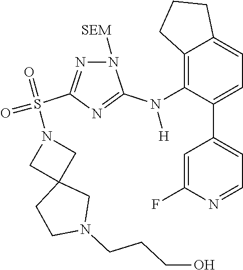 [SMILES: C[Si](C)(C)CCOCn1nc(S(=O)(=O)N2CC3(CCN(CCCO)C3)C2)nc1Nc1c(-c2ccnc(F)c2)ccc2c1CCC2]

Synthesized according to the procedure outlined for 3-[[1-[[5-[[5-(2-fluoro-4-pyridyl)indan-4-yl]amino]-1-(2-trimethylsilylethoxymethyl)-1,2,4-triazol-3-yl]sulfonyl]-4-piperidyl]-methyl-amino]propan-1-ol (Example 1, Step A), from NCS (45.1 mg, 0.338 mmol) and ammonium 5-[[5-(2-fluoro-4-pyridyl)indan-4-yl]amino]-1-(2-trimethylsilylethoxymethyl)-1,2,4-triazole-3-sulfinate (Intermediate A4) (80% purity, 180 mg, 0.281 mmol) in DCM (3.00 mL) at 0° C. for 30 min, and 3-(2,6-diazaspiro[3.4]octan-6-yl)propan-1-ol (Intermediate A7) (91.0 mg, 0.535 mmol) and DIPEA (0.072 ml, 0.422 mmol) in DCM (5.00 ml) at 22° C. for 30 min, to provide the title compound as a solid (128 mg, 69%).

LCMS m/z 658.5 (M+H)⁺ (ES⁺).

¹H NMR (500 MHz, CDCl₃) δ 8.20 (d, J=5.1 Hz, 1H), 7.24 (d, J=7.7 Hz, 1H), 7.19 (d, J=5.1 Hz, 1H), 7.14 (d, J=7.7 Hz, 1H), 6.93 (s, 1H), 6.40 (s, 1H), 5.40 (s, 2H), 3.91 (dd, J=19.6, 8.1 Hz, 4H), 3.79-3.69 (m, 2H), 3.57-3.48 (m, 2H), 3.01 (t, J=7.4 Hz, 2H), 2.78 (t, J=7.4 Hz, 2H), 2.67 (dd, J=14.4, 8.5 Hz, 4H), 2.60 (t, J=7.0 Hz, 2H), 2.18-2.04 (m, 2H), 1.88 (t, J=7.1 Hz, 2H), 1.72-1.62 (m, 2H), 1.25 (s, 1H), 0.77-0.72 (m, 2H), −0.00 (s, 9H).

¹⁹F NMR (471 MHz, CDCl₃) δ −67.24 (s).

### Step B: 2-[(16,16-dioxo-26-oxa-16λ⁶-thia-11,13,14,17,22,28,34-heptazaheptacyclo-[25.3.1.1^{12,15}.1^{17,19}.1^{19,22}.0^{2,10}.0^{5,9}]tetratriaconta-1(30),2,4,9,12(34),14,27(31),28-octaen-13-yl)methoxy]ethyl-trimethyl-silane, formic acid

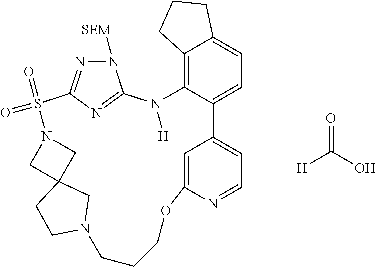 [SMILES: C[Si](C)(C)CCOCn1nc2nc1Nc1c(ccc3c1CCC3)-c1ccnc(c1)OCCCN1CCC3(C1)CN(C3)S2(=O)=O ; O=CO]

Synthesized according to the procedure outlined for trimethyl-[2-[(26-methyl-2,2-dioxo-22-oxa-2λ⁶-thia-1,4,5,7,20,26,33-heptazahexacyclo[25.2.2.1^{3,6}.1^{17,21}.0^{8,16}.0^{9,13}]-tritriaconta-3,6(33),8,13,15,17,19,21(32)-octaen-5-yl)methoxy]ethyl]silane (Example 1, Step B), from tBuOK (1M in THF, 1.52 mL, 1.52 mmol) and 3-[2-[[5-[[5-(2-fluoro-4-pyridyl)indan-4-yl]amino]-1-(2-trimethylsilylethoxymethyl)-1,2,4-triazol-3-yl]-sulfonyl]-2,7-diazaspiro[3.4]octan-7-yl]propan-1-ol (100 mg, 0.152 mmol) in THF (74.0 mL) at 0° C. for 1 h. The product was purified by FC (0-10% MeOH/DCM) followed by basic prep HPLC (0-60% MeCN in water) provided the title compound as a solid (58 mg, 56%).

LCMS m/z 638.8 (M−HCO₂H+H)⁺ (ES⁺).

¹H NMR (400 MHz, CDCl₃) δ 8.18 (dd, J=5.3, 0.7 Hz, 1H), 8.17 (s, 1H), 7.22 (s, 1H), 7.14 (d, J=7.7 Hz, 1H), 6.98 (dd, J=5.3, 1.4 Hz, 1H), 6.66 (dd, J=1.4, 0.7 Hz, 1H), 6.29 (s, 1H), 5.43 (s, 2H), 4.25 (s, 2H), 3.95-3.86 (m, 4H), 3.61-3.56 (m, 2H), 2.99 (dd, J=17.6, 10.2 Hz, 8H), 2.69 (t, J=7.4 Hz, 2H), 2.29 (t, J=7.1 Hz, 2H), 2.08 (dd, J=12.6, 5.3 Hz, 4H), 0.76-0.67 (m, 2H), −0.06 (s, 9H). One exchangeable proton not observed.

### Step C: 26-oxa-16λ⁶-thia-11,13,14,17,22,28,34-heptazaheptacyclo-[25.3.1.1^{12,15}.1^{17,19}.1^{19,22}.0^{2,10}.0^{5,9}]tetratriaconta-1(30),2,4,9,12,14,27(31),28-octaene 16,16-dioxide, formic acid

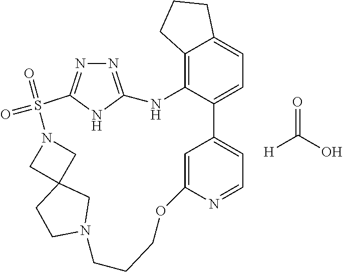

TFA (2.00 mL, 26.9 mmol) was added to a mixture of 2-[(16,16-dioxo-26-oxa-16λ⁶-thia-11,13,14,17,22,28,34-heptazaheptacyclo[25.3.1.1^{12,15}.1^{17,19}.1^{19,22}.0^{2,10}.0^{5,9}]tetratriaconta-1(30),2,4,9,12(34),14,27(31),28-octaen-13-yl)methoxy]ethyl-trimethyl-silane, formic acid (58 mg, 0.091 mmol) in DCM (2.00 mL) at 0° C. The mixture was stirred at 22° C. for 1.5 h and concentrated. The residue was diluted with MeOH (2.00 mL) and passed through a column of Amberlite IRA-402(OH) resin (3.10 g), eluting with MeOH (50.0 mL), followed by HCO₂H in MeOH (7M, 100 ml). The mixture was concentrated to provide the title compound as a solid (12.0 mg, 26%).

LCMS m/z 508.6 (M−HCO₂H+H)⁺ (ES⁺).

¹H NMR (500 MHz, CD₃OD) δ 8.21 (s, 1H), 8.20 (d, J=5.3 Hz, 1H), 7.32 (d, J=7.6 Hz, 1H), 7.27 (d, J=7.8 Hz, 1H), 7.13 (d, J=4.5 Hz, 1H), 6.78 (s, 1H), 4.43 (s, 2H), 3.93 (dd, J=22.9, 9.0 Hz, 4H), 3.41 (d, J=24.3 Hz, 4H), 3.26 (s, 2H), 3.03 (t, J=7.4 Hz, 2H), 2.80 (t, J=7.4 Hz, 2H), 2.41 (t, J=6.9 Hz, 2H), 2.13 (dt, J=14.8, 7.6 Hz, 4H).

Three exchangeable protons not observed.

### Example 3: 25-oxa-16λ⁶-thia-11,13,14,17,22,27,33-heptazaheptacyclo-[24.3.1.1^{12,15}.1^{17,19}.1^{19,22}.0^{2,10}.0^{5,9}]tritriaconta-1(29),2,4,9,12,14,26(30),27-octaene 16,16-dioxide

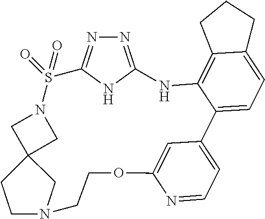 [SMILES: O=S1(=O)c2nnc([nH]2)Nc2c(ccc3c2CCC3)-c2ccnc(c2)OCCN2CCC3(C2)CN1C3]

### Step A: 2-[2-[[5-[[5-(2-fluoro-4-pyridyl)indan-4-yl]amino]-1-(2-trimethylsilyl-ethoxymethyl)-1,2,4-triazol-3-yl]sulfonyl]-2,7-diazaspiro[3.4]octan-7-yl]ethanol

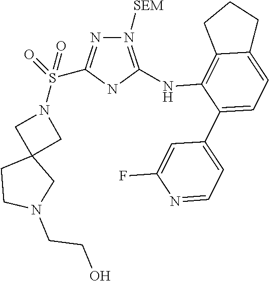

Synthesized according to the procedure outlined for 3-[[1-[[5-[[5-(2-fluoro-4-pyridyl)-indan-4-yl]amino]-1-(2-trimethylsilylethoxymethyl)-1,2,4-triazol-3-yl]sulfonyl]-4-piperidyl]-methyl-amino]propan-1-ol (Example 1, Step A), from NCS (95.5 mg, 0.715 mmol) and sodium 5-[[5-(2-fluoro-4-pyridyl)indan-4-yl]amino]-1-(2-trimethylsilyl-ethoxymethyl)-1,2,4-triazole-3-sulfinate (Intermediate A3) (305 mg, 0.596 mmol) in DMF (5.00 mL) at 0° C. under N₂ for 45 min, and 2-(2,6-diazaspiro-[3.4]octan-6-yl)-ethanol di-(2,2,2-trifluoroacetic acid) (Intermediate A9) (458 mg, 1.19 mmol) and DIPEA (0.51 mL, 2.98 mmol) in DMF (2.00 mL) for 1 h at 0° C., to provide the title compound as a solid (61.0 mg, 16%).

LCMS m/z 644.2 (M+H)⁺ (ES⁺).

¹H NMR (400 MHz, CDCl₃) δ 8.19 (d, J=5.0 Hz, 1H), 7.24 (d, J=7.7 Hz, 1H), 7.21-7.19 (m, 1H), 7.14 (d, J=7.7 Hz, 1H), 6.95-6.93 (m, 1H), 6.44 (s, 1H), 5.40 (s, 2H), 3.92 (d, J=8.4 Hz, 2H), 3.90 (d, J=8.5 Hz, 2H), 3.64-3.57 (m, 2H), 3.57-3.50 (m, 2H), 3.01 (t, J=7.4 Hz, 2H), 2.82-2.72 (m, 5H), 2.68-2.61 (m, 4H), 2.17-2.07 (m, 2H), 1.92 (t, J=7.0 Hz, 2H), 0.81-0.73 (m, 2H), −0.00 (s, 9H).

¹⁹F NMR (376 MHz, CDCl₃) δ −67.41 (s).

### Step B: 2-[(16,16-dioxo-25-oxa-16λ⁶-thia-11,13,14,17,22,27,33-heptazaheptacyclo-[24.3.1.1^{12,15}.1^{17,19}.1^{19,22}.0^{2,10}.0^{5,9}]tritriaconta-1(29),2,4,9,12(33),14,26(30),27-octaen-13-yl)methoxy]ethyl-trimethyl-silane, formic acid

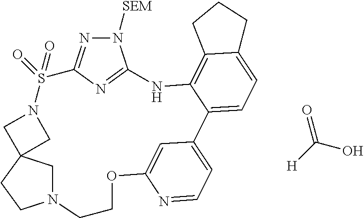 [SMILES: C[Si](C)(C)CCOCn1nc2nc1Nc1c(ccc3c1CCC3)-c1ccnc(c1)OCCN1CCC3(C1)CN(C3)S2(=O)=O ; O=CO]

Synthesized according to the procedure outlined for trimethyl-[2-[(26-methyl-2,2-dioxo-22-oxa-2λ⁶-thia-1,4,5,7,20,26,33-heptazahexacyclo[25.2.2.1^{3,6}.1^{17,21}.0^{8,16}.0^{9,13}]-tritriaconta-3,6(33),8,13,15,17,19,21(32)-octaen-5-yl)methoxy]ethyl]silane (Example 1, Step B), from tBuOK (1M in THF, 0.947 mL, 0.947) and 2-[2-[[5-[[5-(2-fluoro-4-pyridyl)indan-4-yl]amino]-1-(2-trimethylsilylethoxymethyl)-1,2,4-triazol-3-yl]-sulfonyl]-2,7-diazaspiro[3.4]octan-7-yl]-ethanol (61.0 mg, 0.0947 mmol) in THF (45.0 mL) at 0° C. for 3 h to provide the title compound as a solid (11.0 mg, 1900).

LCMS m/z 624.2 (M−HCO₂H+H)⁺ (ES⁺).

¹H NMR (400 MHz, CDCl₃) δ 8.18 (d, J=5.9 Hz, 1H), 8.12 (s, 1H), 7.28 (d, J=7.8 Hz, 1H), 7.20 (d, J=7.8 Hz, 1H), 7.05-6.94 (m, 2H), 6.76 (s, 1H), 5.27 (s, 2H), 4.63-4.56 (m, 2H), 3.99 (d, J=9.2 Hz, 2H), 3.95 (d, J=9.1 Hz, 2H), 3.58-3.51 (m, 2H), 3.23-3.18 (m, 2H), 3.14 (t, J=7.1 Hz, 2H), 3.09-3.06 (m, 2H), 3.02 (t, J=7.5 Hz, 2H), 2.78 (t, J=7.5 Hz, 2H), 2.31 (t, J=7.1 Hz, 2H), 2.19-2.08 (m, 2H), 0.87-0.80 (m, 2H), −0.03 (s, 9H). One exchangeable proton not observed.

### Step C: 25-oxa-16λ⁶-thia-11,13,14,17,22,27,33-heptazaheptacyclo-[24.3.1.1^{12,15}.1^{17,19}.1^{19,22}.0^{2,10}.0^{5,9}]tritriaconta-1(29),2,4,9,12,14,26(30),27-octaene 16,16-dioxide

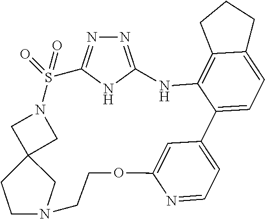 [SMILES: O=S1(=O)c2nnc([nH]2)Nc2c(ccc3c2CCC3)-c2ccnc(c2)OCCN2CCC3(C2)CN1C3]

A mixture of 2-[(16,16-dioxo-25-oxa-16λ⁶-thia-11,13,14,17,22,27,33-heptazaheptacyclo-[24.3.1.1^{12,15}.1^{17,19}.1^{19,22}.0^{2,10}.0^{5,9}]tritriaconta-1(29),2,4,9,12(33),14,26(30),27-octaen-13-yl)methoxy]ethyl-trimethyl-silane, formic acid (12.0 mg, 0.0192 mmol) in DCM (2.00 mL) and TFA (1.00 mL) was stirred at 22° C. for 4 h and concentrated. The product was purified by basic prep HPLC (0-100% MeCN in water) to provide the title compound as a solid (7.84 mg, 83%).

LCMS m/z 494.8 (M+H)⁺ (ES⁺).

¹H NMR (400 MHz, DMSO-d₆) δ 13.22 (br, 1H), 9.22 (s, 1H), 8.21 (d, J=5.2 Hz, 1H), 7.33 (d, J=7.6 Hz, 1H), 7.27 (d, J=7.7 Hz, 1H), 7.02 (d, J=5.3 Hz, 1H), 6.62 (s, 1H), 4.40-4.30 (m, 2H), 3.86-3.80 (m, 4H), 2.98 (t, J=7.3 Hz, 2H), 2.73 (t, J=7.1 Hz, 2H), 2.68-2.62 (m, 2H), 2.59-2.54 (m, 2H), 2.43-2.31 (m, 2H), 2.11-2.01 (m, 2H), 2.01-1.91 (m, 2H).

### Example 4: 3-methyl-26-oxa-16λ⁶-thia-11,13,14,17,22,28,34-heptazaheptacyclo-[25.3.1.1^{12,15}.1^{17,19}.1^{19,22}.0^{2,10}.0^{5,9}]tetratriaconta-1(30),2,4,9,12,14,27(31),28-octaene 16,16-dioxide

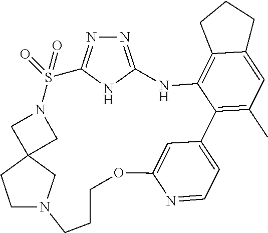 [SMILES: Cc1cc2c(c3c1-c1ccnc(c1)OCCCN1CCC4(C1)CN(C4)S(=O)(=O)c1nnc([nH]1)N3)CCC2]

### Step A: 3-[2-[[5-[[5-(2-fluoro-4-pyridyl)-6-methyl-indan-4-yl]amino]-1-(2-trimethylsilylethoxymethyl)-1,2,4-triazol-3-yl]sulfonyl]-2,7-diazaspiro[3.4]octan-7-yl]propan-1-ol

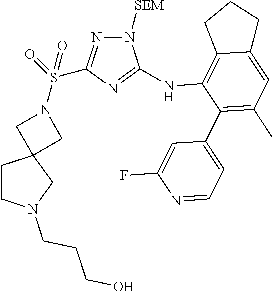 [SMILES: Cc1cc2c(c(Nc3nc(S(=O)(=O)N4CC5(CCN(CCCO)C5)C4)nn3COCC[Si](C)(C)C)c1-c1ccnc(F)c1)CCC2]

Synthesized according to the procedure outlined for 3-[[1-[[5-[[5-(2-fluoro-4-pyridyl)-indan-4-yl]amino]-1-(2-trimethylsilylethoxymethyl)-1,2,4-triazol-3-yl]sulfonyl]-4-piperidyl]-methyl-amino]propan-1-ol (Example 1, Step A), from NCS (107 mg, 0.804 mmol) and sodium 5-[[5-(2-fluoro-4-pyridyl)-6-methyl-indan-4-yl]amino]-1-(2-trimethylsilylethoxymethyl)-1,2,4-triazole-3-sulfinate (Intermediate A10) (352 mg, 0.670 mmol) in DCM (7.50 mL) at 0° C. for 1 h, and 3-(2,6-diazaspiro[3.4]octan-6-yl)-propan-1-ol (Intermediate A7) (148 mg, 0.871 mmol) and DIPEA (0.348 mL, 2.01 mmol) in DCM (2.50 mL) for 1 h at 0° C., to provide the title compound as a solid (235 mg, 52%).

LCMS m/z 672.2 (M+H)⁺ (ES⁺).

¹H NMR (400 MHz, CDCl₃) δ 8.26 (d, J=5.1 Hz, 1H), 7.11 (s, 1H), 7.07-7.05 (m, 1H), 6.82-6.80 (m, 1H), 6.06 (s, 1H), 5.30 (d, J=3.3 Hz, 1H), 5.28 (d, J=4.1 Hz, 1H), 3.98-3.87 (m, 6H), 3.76 (t, J=5.3 Hz, 2H), 3.42-3.32 (m, 2H), 2.97 (t, J=7.4 Hz, 2H), 2.77-2.64 (m, 5H), 2.59 (t, J=7.0 Hz, 2H), 2.12-2.07 (m, 2H), 2.06 (s, 3H), 1.89 (t, J=7.1 Hz, 2H), 1.72-1.65 (m, 2H), 0.72-0.65 (m, 2H), 0.00 (s, 9H).

¹⁹F NMR (376 MHz, CDCl₃) δ −66.66 (s).

### Step B: trimethyl-[2-[(3-methyl-16,16-dioxo-26-oxa-16λ⁶-thia-11,13,14,17,22,28,34-heptazaheptacyclo[25.3.1.1^{12,15}1^{17,19}.1^{19,22}.0^{2,10}.0^{5,9}]tetratriaconta-1(30),2,4,9,12(34),14,27(31),28-octaen-13-yl)methoxy]ethyl]silane

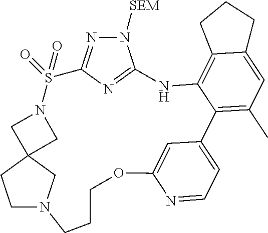 [SMILES: Cc1cc2c(c3c1-c1ccnc(c1)OCCCN1CCC4(C1)CN(C4)S(=O)(=O)c1nc(n(COCC[Si](C)(C)C)n1)N3)CCC2]

Synthesized according to the procedure outlined for trimethyl-[2-[(26-methyl-2,2-dioxo-22-oxa-2λ⁶-thia-1,4,5,7,20,26,33-heptazahexacyclo[25.2.2.1^{3,6}.1^{17,21}.0^{8,16}.0^{9,13}]-tritriaconta-3,6(33),8,13,15,17,19,21(32)-octaen-5-yl)methoxy]ethyl]silane (Example 1, Step B), from tBuOK (1M in THF, 2.81 mL, 2.81 mmol) and 3-[2-[[5-[[5-(2-fluoro-4-pyridyl)-6-methyl-indan-4-yl]amino]-1-(2-trimethylsilylethoxymethyl)-1,2,4-triazol-3-yl]sulfonyl]-2,7-diazaspiro[3.4]octan-7-yl]propan-1-ol (189 mg, 0.281 mmol) in THF (145 mL) at 0° C. for 2 h to provide the title compound as a solid (123 mg, 67%).

LCMS m/z 652.3 (M+H)⁺ (ES⁺).

¹H NMR (400 MHz, CDCl₃) δ 8.20 (dd, J=5.2, 0.6 Hz, 1H), 7.07 (s, 1H), 6.78 (dd, J=5.2, 1.4 Hz, 1H), 6.54-6.52 (m, 1H), 5.94 (s, 1H), 5.31 (s, 2H), 4.71-4.63 (m, 1H), 4.06-3.99 (m, 1H), 3.97-3.92 (m, 1H), 3.88-3.80 (m, 2H), 3.73-3.69 (m, 1H), 3.45-3.31 (m, 2H), 3.05-2.87 (m, 2H), 2.79-2.60 (m, 4H), 2.59-2.45 (m, 4H), 2.23-2.08 (m, 4H), 2.05 (s, 3H), 2.03-1.87 (m, 2H), 0.72 (t, J=7.9 Hz, 2H), −0.03 (s, 9H).

### Step C: 3-methyl-26-oxa-16λ⁶-thia-11,13,14,17,22,28,34-heptazaheptacyclo-[25.3.1.1^{12,15}.1^{17,19}.1^{19,22}.0^{2,10}.0^{5,9}]tetratriaconta-1(30),2,4,9,12,14,27(31),28-octaene 16,16-dioxide

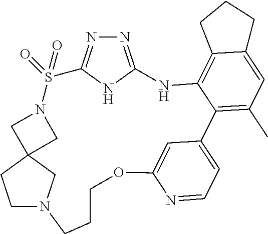 [SMILES: Cc1cc2c(c3c1-c1ccnc(c1)OCCCN1CCC4(C1)CN(C4)S(=O)(=O)c1nnc([nH]1)N3)CCC2]

A mixture of trimethyl-[2-[(3-methyl-16,16-dioxo-26-oxa-1A-thia-11,13,14,17,22,28,34-heptazaheptacyclo[25.3.1.1^{12,15}.1^{17,19}.1^{19,22}.0^{2,10}.0^{5,9}]tetratriaconta-1(30),2,4,9,12(34),14,27(31),28-octaen-13-yl)methoxy]ethyl]silane (141 mg, 0.216 mmol) in DCM (2.00 mL) and TFA (2.00 mL) was stirred at 22° C. for 4 h and concentrated. The product was purified by basic prep HPLC (0-1000% MeCN in water) to provide the title compound as a solid (66.0 mg, 59%).

LCMS m/z 522.0 (M+H)⁺ (ES⁺).

¹H NMR (400 MHz, DMSO-d₆) δ 12.98 (br, 1H), 8.74 (s, 1H), 8.15 (dd, J=5.2, 0.6 Hz, 1H), 7.17 (s, 1H), 6.85 (dd, J=5.2, 1.3 Hz, 1H), 6.48 (s, 1H), 4.69-4.54 (m, 1H), 4.04-3.92 (m, 1H), 3.91-3.84 (m, 1H), 3.78-3.66 (m, 2H), 3.48-3.40 (m, 1H), 2.97-2.88 (m, 2H), 2.66 (t, J=6.9 Hz, 2H), 2.63-2.60 (m, 1H), 2.60-2.53 (m, 2H), 2.46-2.36 (m, 1H), 2.27-2.19 (m, 1H), 2.17-2.08 (m, 1H), 2.04 (s, 3H), 2.02-1.91 (m, 4H), 1.77-1.69 (m, 2H).

### Example 5: 8-oxa-28λ⁶-thia-5,10,23,25,26,29,34-heptazaheptacyclo-[27.2.2.1^{9,13}.1^{24,27}.0^{1,5}.0^{14,22}.0^{17,21}]pentatriaconta-9(35),10,12,14,16,21,24,26-octaene 28,28-dioxide

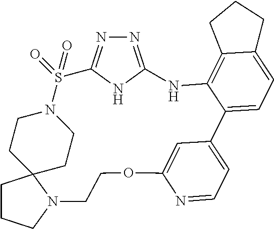 [SMILES: O=S1(=O)c2nnc([nH]2)Nc2c(ccc3c2CCC3)-c2ccnc(c2)OCCN2CCCC23CCN1CC3]

### Step A: 2-[8-[[5-[[5-(2-fluoro-4-pyridyl)indan-4-yl]amino]-1-(2-trimethylsilyl-ethoxymethyl)-1,2,4-triazol-3-yl]sulfonyl]-1,8-diazaspiro[4.5]decan-1-yl]ethanol

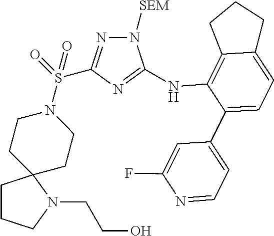 [SMILES: C[Si](C)(C)CCOCn1nc(S(=O)(=O)N2CCC3(CCCN3CCO)CC2)nc1Nc1c(-c2ccnc(F)c2)ccc2c1CCC2]

Synthesized according to the procedure outlined for 3-[[1-[[5-[[5-(2-fluoro-4-pyridyl)indan-4-yl]amino]-1-(2-trimethylsilylethoxymethyl)-1,2,4-triazol-3-yl]sulfonyl]-4-piperidyl]-methyl-amino]propan-1-ol (Example 1, Step A), from NCS (144 mg, 1.08 mmol) and sodium 5-[[5-(2-fluoro-4-pyridyl)indan-4-yl]amino]-1-(2-trimethylsilylethoxymethyl)-1,2,4-triazole-3-sulfinate (Intermediate A3) (500 mg, 0.977 mmol) in DCM:DMF (4:1 v/v, 20 mL) at 0° C. for 30 min, and 2-(1,8-diazoniaspiro[4.5]decan-1-yl)ethanol di-(2,2,2-trifluoroacetate) (Intermediate A8) (270 mg, 1.47 mmol) and DIPEA (0.836 mL, 4.89 mmol) in DMF (4 mL) at 22° C. for 15 min, to provide the title compound as a solid (264 mg, 40%).

LCMS m/z 672.1 (M+H)⁺ (ES⁺).

¹H NMR (400 MHz, DMSO-d₆) δ 9.05 (s, 1H), 8.18 (d, J=5.2 Hz, 2H), 7.30 (t, J=6.9 Hz, 3H), 7.09 (s, 2H), 5.42 (s, 2H), 4.27 (s, 1H), 3.63-3.46 (m, 3H), 3.40-3.36 (m, 2H), 2.98 (t, J=7.6 Hz, 2H), 2.75-2.70 (m, 3H), 2.48-2.41 (m, 3H), 2.12-1.93 (m, 3H), 1.69-1.65 (m, 2H), 1.57-1.53 (m, 2H), 1.48-1.44 (m, 2H), 1.27 (d, J=11.9 Hz, 2H), 0.85 (dd, J=10.4, 6.0 Hz, 2H), −0.03 (s, 9H).

¹⁹F NMR (376 MHz, DMSO-d₆) δ −69.07 (s).

### Step B: 2-[(28,28-dioxo-8-oxa-28λ⁶-thia-5,10,23,25,26,29,34-heptazaheptacyclo-[27.2.2.1^{9,13}.1^{24,27}.0^{1,5}.0^{14,22}.0^{17,21}]pentatriaconta-9(35),10,12,14,16,21,24(34),26-octaen-25-yl)methoxy]ethyl-trimethyl-silane

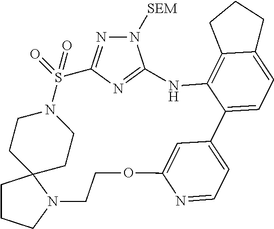 [SMILES: C[Si](C)(C)CCOCn1nc2nc1Nc1c(ccc3c1CCC3)-c1ccnc(c1)OCCN1CCCC13CCN(CC3)S2(=O)=O]

Synthesized according to the procedure outlined for trimethyl-[2-[(26-methyl-2,2-dioxo-22-oxa-2λ⁶-thia-1,4,5,7,20,26,33-heptazahexacyclo[25.2.2.1^{3,6}.1^{17,21}.0^{8,16}.0^{9,13}]-tritriaconta-3,6(33),8,13,15,17,19,21(32)-octaen-5-yl)methoxy]ethyl]silane (Example 1, Step B), from tBuOK (1M in THF, 2.98 mL, 2.98 mmol) and 2-[8-[[5-[[5-(2-fluoro-4-pyridyl)indan-4-yl]amino]-1-(2-trimethylsilyl-ethoxymethyl)-1,2,4-triazol-3-yl]sulfonyl]-1,8-diazaspiro[4.5]decan-1-yl]ethanol (200 mg, 0.298 mmol) in THF (110 mL) at 0° C. for 2 h, to provide the title compound as a solid (50.0 mg, 25%).

LCMS m/z 650.7 (M−H)⁻ (ES⁻).

¹H NMR (400 MHz, CDCl₃) δ 8.18 (d, J=5.1 Hz, 1H), 7.22 (d, J=7.7 Hz, 1H), 7.12 (d, J=7.6 Hz, 1H), 6.91 (dd, J=5.2, 1.2 Hz, 1H), 6.81 (s, 1H), 6.19 (s, 1H), 5.30 (s, 2H), 4.22-4.17 (m, 2H), 3.69 (dt, J=13.4, 6.7 Hz, 2H), 3.48-3.37 (m, 2H), 3.28 (t, J=9.6 Hz, 2H), 3.00 (t, J=7.5 Hz, 2H), 2.84-2.78 (m, 2H), 2.78 (t, J=7.3 Hz, 2H), 2.68-2.62 (m, 2H), 2.11 (p, J=7.5 Hz, 2H), 1.91-1.69 (m, 2H), 1.69-1.60 (m, 2H), 1.56-1.52 (m, 2H), 1.39-1.31 (m, 2H), 0.83-0.63 (m, 2H), −0.03 (s, 9H).

### Step C: 8-oxa-28λ⁶-thia-5,10,23,25,26,29,34-heptazaheptacyclo-[27.2.2.1^{9,13}.1^{24,27}.0^{1,5}.0^{14,22}.0^{17,21}]pentatriaconta-9(35),10,12,14,16,21,24,26-octaene 28,28-dioxide, formic acid

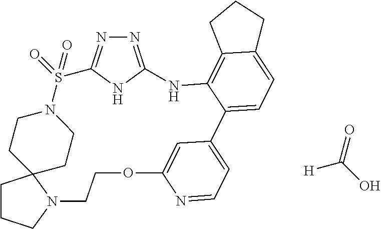 [SMILES: O=S1(=O)c2nnc([nH]2)Nc2c(ccc3c2CCC3)-c2ccnc(c2)OCCN2CCCC23CCN1CC3 ; O=CO]

Synthesized according to the procedure outlined for 26-oxa-16λ⁶-thia-11,13,14,17,22,28,34-heptazaheptacyclo[25.3.1.1^{12,15}.1^{17,19}.1^{19,22}.0^{2,10}.0^{0,9}]tetratriaconta-1(30),2,4,9,12,14,27(31),28-octaene 16,16-dioxide, formic acid (Example 2, Step C), from TFA (0.481 mL, 6.29 mmol) and 2-[(28,28-dioxo-8-oxa-28λ⁶-thia-5,10,23,25,26,29,34-heptazaheptacyclo[27.2.2.1^{9,13}.1^{24,27}.0^{1,5}.0^{14,22}.0^{17,21}]pentatriaconta-9(35),10,12,14,16,21,24(34),26-octaen-25-yl)methoxy]ethyl-trimethyl-silane (50.0 mg, 0.0767 mmol in DCM (2.50 mL) at 22° C. for 1 h to provide the title compound as a solid (30 mg, 48%).

LCMS m/z 522.3 (M−HCO₂H+H)⁺ (ES⁺).

¹H NMR (400 MHz, DMSO-d₆) δ 12.85 (bs, 1H), 9.14 (s, 1H), 8.15 (d, J=5.1 Hz, 1H), 7.94 (dd, J=8.3, 1.3 Hz, 1H), 7.54 (dt, J=45.9, 7.4 Hz, 1H), 7.30 (d, J=7.7 Hz, 1H), 7.23 (d, J=7.7 Hz, 1H), 6.98 (dd, J=5.2, 1.4 Hz, 1H), 6.62 (s, 1H), 4.22-4.18 (m, 2H), 3.28-3.21 (m, 2H), 3.20-3.13 (m, 2H), 2.97 (t, J=7.5 Hz, 2H), 2.83 (t, J=7.1 Hz, 4H), 2.58-2.52 (m, 2H), 2.06 (p, J=7.5 Hz, 2H), 1.73-1.67 (m, 2H), 1.60-1.35 (m, 4H), 1.35-1.16 (m, 2H).

### Example 6: 21,21-dimethyl-23-oxa-16λ⁶-thia-11,13,14,20,25,29,30-heptazahexacyclo-[22.3.1.1^{12,15}.1^{17,20}.0^{2,10}.0^{5,9}]triaconta-1(27),2,4,9,12(30),14,17(29),18,24(28),25-decaene 16,16-dioxide

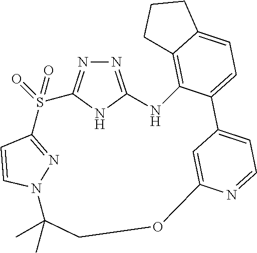 [SMILES: CC1(C)COc2cc(ccn2)-c2ccc3c(c2Nc2nnc([nH]2)S(=O)(=O)c2ccn1n2)CCC3]

### Step A

### (a) 5-[[5-(2-fluoro-4-pyridyl)indan-4-yl]amino]-1-(2-trimethylsilylethoxymethyl)-1,2,4-triazole-3-thiol; and

(b) 5-[[5-(2-methoxy-4-pyridyl)indan-4-yl]amino]-1-(2-trimethylsilyl-ethoxymethyl-1,2,4-triazole-3-thiol

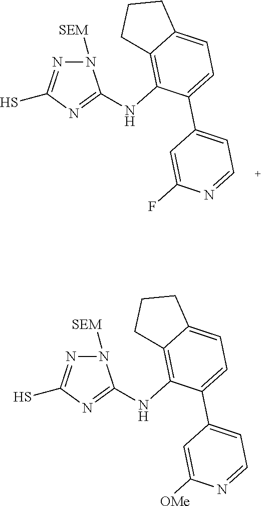 [SMILES: C[Si](C)(C)CCOCn1nc(S)nc1Nc1c(-c2ccnc(F)c2)ccc2c1CCC2 ; COc1cc(-c2ccc3c(c2Nc2nc(S)nn2COCC[Si](C)(C)C)CCC3)ccn1]

tBuOK (1.0 M in THF, 2.57 mL, 2.57 mmol) was added to a mixture of methyl 3-[[5-[[5-(2-fluoro-4-pyridyl)indan-4-yl]amino]-1-(2-trimethylsilylethoxymethyl)-1,2,4-triazol-3-yl]sulfanyl]propanoate (Intermediate A2) (700.0 mg, 1.29 mmol) in THF (10.0 mL) at 22° C. under N₂. The mixture was stirred at 22° C. for 5 min and diluted with aq. HCl (1.0 M, 5.00 mL). The aqueous phase was extracted with EtOAc (3×50.0 mL), and the combined organic phases were dried (Na₂SO₄), filtered, and concentrated to provide a mixture of the title compounds as a solid (1:1, 400 mg, 61%).

(a) LCMS m/z 458.3 (M+H)⁺ (ES⁺).

¹⁹F NMR (376 MHz, CDCl₃) δ −67.46.

(b) LCMS m/z 470.3 (M+H)⁺ (ES⁺).

### Step B: 2-[3-[[5-[[5-(2-fluoro-4-pyridyl)indan-4-yl]amino]-1-(2-trimethylsilyl-ethoxymethyl)-1,2,4-triazol-3-yl]sulfanyl]pyrazol-1-yl]-2-methyl-propan-1-ol

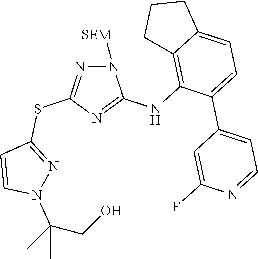 [SMILES: CC(C)(CO)n1ccc(Sc2nc(Nc3c(-c4ccnc(F)c4)ccc4c3CCC4)n(COCC[Si](C)(C)C)n2)n1]

DMEDA (3.52 mL, 32.7 mmol) was added to a degassed mixture of the product of step A (1:1 mixture, 749 mg, 1.64 mmol), CuI (623 mg, 3.27 mmol), and 2-(3-iodopyrazol-1-yl)-2-methyl-propan-1-ol (Intermediate A11) (435 mg, 1.64 mmol) in dioxane (10.0 mL) at 22° C. under N₂. The mixture was stirred at 70° C. for 1 h and diluted with water (20.0 mL). The aqueous phase was extracted with EtOAc (3×20.0 mL), and the combined organic phases were washed with brine (20.0 mL), dried (Na₂SO₄), filtered, and concentrated. The product was purified by FC (0-100% EtOAc/hexanes), and by basic prep HPLC (29-39% MeCN in water) to provide the title compound as a solid (125 mg, 13%).

LCMS m/z 596.4 (M+H)⁺ (ES⁺).

¹H NMR (500 MHz, CDCl₃) δ 8.15 (d, J=5.1 Hz, 1H), 7.51 (d, J=2.4 Hz, 1H), 7.22-7.15 (m, 2H), 7.09 (d, J=7.7 Hz, 1H), 6.92 (s, 1H), 6.37 (d, J=2.4 Hz, 1H), 6.14 (s, 1H), 5.20 (s, 2H), 3.76 (s, 2H), 3.52-3.41 (m, 2H), 2.98 (t, J=7.5 Hz, 2H), 2.72 (t, J=7.4 Hz, 2H), 2.08 (p, J=7.5 Hz, 2H), 1.54 (s, 6H), 0.78-0.66 (m, 2H), −0.02 (s, 9H). One exchangeable proton not observed.

¹⁹F NMR (471 MHz, CDCl₃) δ −67.47 (s).

### Step C: 2-[(21,21-dimethyl-23-oxa-16-thia-11,13,14,20,25,29,30-heptazahexacyclo-[22.3.1.1^{12,15}.1^{17,20}.0^{2,10}.0^{5,9}]triaconta-1(27),2,4,9,12(30),14,17(29),18,24(28),25-decaen-13-yl)methoxy]ethyl-trimethyl-silane

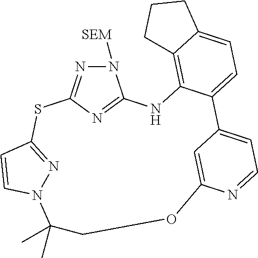 [SMILES: CC1(C)COc2cc(ccn2)-c2ccc3c(c2Nc2nc(nn2COCC[Si](C)(C)C)Sc2ccn1n2)CCC3]

A mixture of 2-[3-[[5-[[5-(2-fluoro-4-pyridyl)indan-4-yl]amino]-1-(2-trimethylsilyl-ethoxymethyl)-1,2,4-triazol-3-yl]sulfanyl]pyrazol-1-yl]-2-methyl-propan-1-ol (300 mg, 0.504 mmol) in THF (250 mL) was added to a mixture of tBuOK (1M in THF, 5.04 mL, 5.04 mmol) in THF (50.0 mL) over the course of 4 h at 22° C. The mixture was stirred at 22° C. for 1 h, diluted with EtOH (20.0 mL), and concentrated. The product was purified by FC (0-10% MeOH/DCM) to provide the title compound as a solid (78%, 190 mg, 51%).

LCMS m/z 576.2 (M+H)⁺ (ES⁺).

¹H NMR (400 MHz, CDCl₃) δ 7.78 (dd, J=5.3, 0.5 Hz, 1H), 7.56 (d, J=2.3 Hz, 1H), 7.10 (d, J=7.7 Hz, 1H), 7.03 (d, J=7.7 Hz, 1H), 6.76 (dd, J=5.3, 1.4 Hz, 1H), 6.45 (d, J=2.3 Hz, 1H), 6.30 (d, J=0.8 Hz, 1H), 6.10 (s, 1H), 5.24 (s, 2H), 4.57 (s, 2H), 3.59-3.51 (m, 2H), 2.99 (t, J=7.5 Hz, 2H), 2.88 (t, J=7.4 Hz, 2H), 2.20-2.11 (m, 2H), 1.72 (s, 6H), 0.98-0.88 (m, 2H), 0.02 (s, 9H).

### Step D: 2-[(21,21-dimethyl-16,16-dioxo-23-oxa-16λ⁶-thia-11,13,14,20,25,29,30-heptazahexacyclo[22.3.1.1^{12,15}.1^{17,20}.0^{2,10}.0^{5,9}]triaconta-1(27),2,4,9,12(30),14,17(29),18,24(28),25-decaen-13-yl)methoxy]ethyl-trimethyl-silane

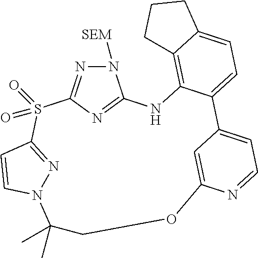 [SMILES: CC1(C)COc2cc(ccn2)-c2ccc3c(c2Nc2nc(nn2COCC[Si](C)(C)C)S(=O)(=O)c2ccn1n2)CCC3]

m-CPBA (162 mg, 0.722 mmol) was added to a mixture of 2-[(21,21-dimethyl-23-oxa-16-thia-11,13,14,20,25,29,30-heptazahexacyclo[22.3.1.1^{12,15}.1^{17,20}.0^{2,10}.0^{5,9}]triaconta-1(27),2,4,9,12(30),14,17(29),18,24(28),25-decaen-13-yl)methoxy]ethyl-trimethyl-silane (78%, 180 mg, 0.241 mmol) in DCM (15.0 mL) at 0° C. The mixture was stirred at 22° C. for 1 h and diluted with sat. aq. Na₂S₂O₄ (10.0 mL). The aqueous phase was extracted with DCM (3×15.0 mL), and the combined organic phases were dried (Na₂SO₄), filtered, and concentrated. The product was purified by FC (0-35% MeOH/DCM) to provide the title compound as a solid (50.0 mg, 27%).

LCMS m/z 608.2 (M+H)⁺ (ES⁺).

¹H NMR (400 MHz, CDCl₃) δ 7.75 (d, J=4.8 Hz, 1H), 7.65 (d, J=2.5 Hz, 1H), 7.11 (d, J=7.7 Hz, 1H), 6.97 (d, J=7.6 Hz, 1H), 6.84 (d, J=2.5 Hz, 1H), 6.74 (dd, J=5.3, 1.4 Hz, 1H), 6.39 (s, 1H), 6.24 (s, 1H), 5.27 (s, 2H), 4.49 (s, 2H), 3.58 (dd, J=9.2, 7.9 Hz, 2H), 2.97 (t, J=7.4 Hz, 2H), 2.87 (t, J=7.4 Hz, 2H), 2.17-2.09 (m, 2H), 1.74 (s, 6H), 0.97-0.87 (m, 2H), 0.00 (s, 9H).

### Step E: 21,21-dimethyl-23-oxa-16λ⁶-thia-11,13,14,20,25,29,30-heptazahexacyclo-[22.3.1.1^{12,15}.1^{17,20}.0^{2,10}.0^{5,9}]triaconta-1(27),2,4,9,12(30),14,17(29),18,24(28),25-decaene 16,16-dioxide

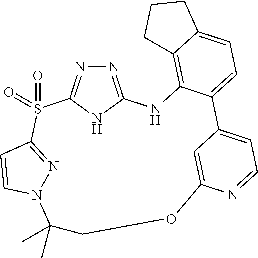 [SMILES: CC1(C)COc2cc(ccn2)-c2ccc3c(c2Nc2nnc([nH]2)S(=O)(=O)c2ccn1n2)CCC3]

TFA (2.00 mL) was added to a mixture of 2-[(21,21-dimethyl-16,16-dioxo-23-oxa-16λ⁶-thia-11,13,14,20,25,29,30-heptazahexacyclo[22.3.1.1^{12,15}.1^{17,20}.0^{2,10}.0^{5,9}]triaconta-1(27),2,4,9,12(30),14,17(29),18,24(28),25-decaen-13-yl)methoxy]ethyl-trimethyl-silane (50.0 mg, 0.0823 mmol) in DCM (5.00 mL) at 22° C. under N₂. The mixture was stirred at 22° C. for 1 h and concentrated. The product was purified by basic prep HPLC (45-65% MeCN in water) to provide the title compound as a solid (12.0 mg, 24%).

LCMS m/z 478.3 (M+H)⁺ (ES⁺).

¹H NMR (400 MHz, DMSO-d₆) δ 9.06 (s, 1H), 8.37 (s, 1H), 8.11 (d, J=2.5 Hz, 1H), 7.84 (d, J=5.3 Hz, 1H), 7.14 (d, J=7.7 Hz, 1H), 7.08 (d, J=7.7 Hz, 1H), 6.86 (dd, J=5.3, 1.5 Hz, 1H), 6.74 (d, J=2.5 Hz, 1H), 6.14 (d, J=0.8 Hz, 1H), 4.49 (s, 2H), 3.08-2.77 (m, 4H), 2.16-1.92 (m, 2H), 1.67 (s, 6H).

### Example 7: 21,21-dimethyl-24-oxa-16λ⁶-thia-11,13,14,20,26,30,31-heptazahexacyclo-[23.3.1.1^{12,15}.1^{17,20}.0^{2,10}.0^{5,9}]hentriaconta-1(28),2,4,9,12,14,17(30),18,25(29),26-decaene 16,16-dioxide

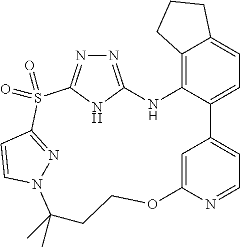 [SMILES: CC1(C)CCOc2cc(ccn2)-c2ccc3c(c2Nc2nnc([nH]2)S(=O)(=O)c2ccn1n2)CCC3]

### Step A: 5-[[5-(2-fluoro-4-pyridyl)indan-4-yl]amino]-1-(2-trimethylsilylethoxymethyl)-1,2,4-triazole-3-thiol

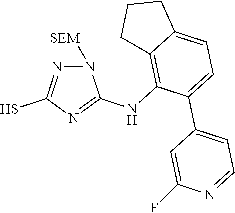 [SMILES: C[Si](C)(C)CCOCn1nc(S)nc1Nc1c(-c2ccnc(F)c2)ccc2c1CCC2]

tBuOK (1.00 M, 4.71 mL, 4.71 mmol) was added to a mixture of methyl 3-[[5-[[5-(2-fluoro-4-pyridyl)indan-4-yl]amino]-1-(2-trimethylsilylethoxymethyl)-1,2,4-triazol-3-yl]sulfanyl]propanoate (Intermediate A2) (1.28 g, 2.35 mmol) in degassed THF (20 mL) at 22° C. The mixture was stirred at 22° C. for 30 min and Dowex MAC-3 Hydrogen form (3.00 g, 9.66 mmol) was added. The mixture was stirred at 20° C. for 30 min and filtered, washing with THF (20.0 mL). The filtrate was concentrated to provide the title compound as a solid which was directly engaged in the next step.

LCMS m/z 458.6 (M+H)⁺ (ES⁺).

### Step B: 3-[3-[[5-[[5-(2-fluoro-4-pyridyl)indan-4-yl]amino]-1-(2-trimethylsilyl-ethoxymethyl)-1,2,4-triazol-3-yl]sulfanyl]pyrazol-1-yl]-3-methyl-butan-1-ol

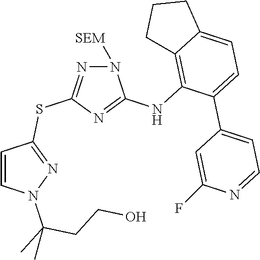 [SMILES: CC(C)(CCO)n1ccc(Sc2nc(Nc3c(-c4ccnc(F)c4)ccc4c3CCC4)n(COCC[Si](C)(C)C)n2)n1]

A mixture of the product of step A (1.08 g, 2.35 mmol) in degassed dioxane (15.0 mL) was added to a mixture of CuI (896 mg, 4.71 mmol) and DMEDA (5.07 mL, 47.1 mmol) in degassed dioxane (5.00 mL). 3-(3-Iodopyrazol-1-yl)-3-methyl-butan-1-ol (Intermediate A12) (659 mg, 2.35 mmol) was added to the mixture. The mixture was stirred at 70° C. for 18 h and filtered on Celite, washing with EtOAc (80.0 mL). The filtrate was concentrated and purified by FC (0-50% EtOAc/hexanes) to provide the title compound as a solid (643 mg, 45% over 2 steps).

LCMS m/z 608.7 (M−H)⁻ (ES⁻).

¹H NMR (400 MHz, CDCl₃) δ 8.17 (d, J=5.3 Hz, 1H), 7.56 (d, J=2.3 Hz, 1H), 7.22-7.15 (m, 2H), 7.09 (d, J=7.6 Hz, 1H), 6.93 (s, 1H), 6.43 (d, J=2.3 Hz, 1H), 6.10 (s, 1H), 5.17 (s, 2H), 3.51 (t, J=5.5 Hz, 2H), 3.48-3.42 (m, 2H), 2.99 (t, J=7.5 Hz, 2H), 2.73 (t, J=7.5 Hz, 2H), 2.12-2.07 (m, 2H), 2.07-2.02 (m, 2H), 1.64 (s, 6H), 0.77-0.63 (m, 2H), −0.02 (s, 9H). One exchangeable proton not observed.

¹⁹F NMR (376 MHz, CDCl₃) δ −67.46 (s).

### Step C: 2-[(21,21-dimethyl-24-oxa-16-thia-11,13,14,20,26,30,31-heptazahexacyclo-[23.3.1.1^{12,15}.1^{17,20}.0^{2,10}.0^{5,9}]hentriaconta-1(28),2,4,9,12(31),14,17(30),18,25(29),26-decaen-13-yl)methoxy]ethyl-trimethyl-silane

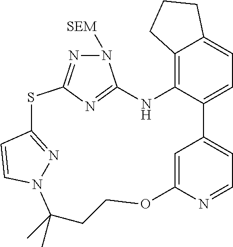 [SMILES: CC1(C)CCOc2cc(ccn2)-c2ccc3c(c2Nc2nc(nn2COCC[Si](C)(C)C)Sc2ccn1n2)CCC3]

A mixture of 3-[3-[[5-[[5-(2-fluoro-4-pyridyl)indan-4-yl]amino]-1-(2-trimethylsilyl-ethoxymethyl)-1,2,4-triazol-3-yl]sulfanyl]pyrazol-1-yl]-3-methyl-butan-1-ol (320 mg, 0.525 mmol) in THF (400 mL) was added to a mixture of tBuOK (1.00 M, 5.25 mL, 5.25 mmol) in THF (100.0 mL) over 90 min at 0° C. The mixture was stirred at 0° C. for 90 min and Dowex MAC-3 Hydrogen form (2500 mg, 8.05 mmol) was added. The mixture was stirred at 20° C. for 10 min, filtered and concentrated to provide the title compound as a solid which was directly engaged in the next step.

LCMS m/z 588.8 (M−H)⁻ (ES⁻).

### Step D: 2-[(21,21-dimethyl-16,16-dioxo-24-oxa-16λ⁶-thia-11,13,14,20,26,30,31-heptazahexacyclo[23.3.1.1^{12,15}.1^{17,20}.0^{2,10}.0^{5,9}]hentriaconta-1(28),2,4,9,12(31),14,17(30),18,25(29),26-decaen-13-yl)methoxy]ethyl-trimethyl-silane

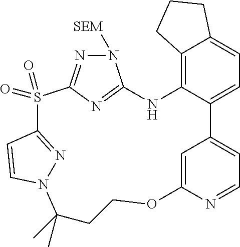 [SMILES: CC1(C)CCOc2cc(ccn2)-c2ccc3c(c2Nc2nc(nn2COCC[Si](C)(C)C)S(=O)(=O)c2ccn1n2)CCC3]

m-CPBA (77.0%, 353 mg, 1.58 mmol) was added to a solution of 2-[(21,21-dimethyl-24-oxa-16-thia-11,13,14,20,26,30,31-heptazahexacyclo[23.3.1.1^{12,15}.1^{17,20}.0^{2,10}.0^{5,9}]-hentriaconta-1(28),2,4,9,12(31),14,17(30),18,25(29),26-decaen-13-yl)methoxy]ethyl-trimethyl-silane from step C (310 mg, 0.526 mmol) in DCM (100 mL) at 20° C. The mixture was stirred at 20° C. for 1 h and diluted with sat. aq. NaS₂O₃ (100 mL) and sat. aq. NaHCO₃ (100 mL). The aqueous phase was extracted with EtOAc (100 mL), and the combined organic phases were washed with brine (100 mL), dried (Na₂SO₄), filtered, and concentrated. The residue was diluted with MeCN (15.0 mL), and m-CPBA (77.0%. 353 mg, 1.58 mmol) was added. The mixture was stirred at 20° C. for 4 h, and m-CPBA (77.0%, 100 mg, 0.447 mmol) was added. The mixture was stirred at 20° C. for 2 h and diluted with sat. aq. NaS₂O₃ (100 mL) and sat. aq. NaHCO₃ (100 mL). The aqueous phase was extracted with EtOAc (100 mL), and the combined organic phases were washed with brine (100 mL), dried (Na₂SO₄), filtered, and concentrated. The product was purified by FC (0-100% MeOH/DCM) to provide the title compound as a solid (179 mg, 43% over 2 steps).

LCMS m/z 620.8 (M−H)⁻ (ES⁻).

### Step E: 21,21-dimethyl-24-oxa-16λ⁶-thia-11,13,14,20,26,30,31-heptazahexacyclo-[23.3.1.1^{12,15}.1^{17,20}.0^{2,10}.0^{5,9}]hentriaconta-1(28),2,4,9,12,14,17(30),18,25(29),26-decaene 16,16-dioxide

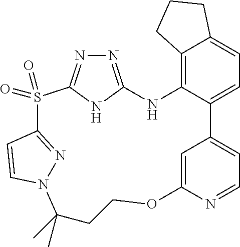 [SMILES: CC1(C)CCOc2cc(ccn2)-c2ccc3c(c2Nc2nnc([nH]2)S(=O)(=O)c2ccn1n2)CCC3]

A mixture of 2-[(21,21-dimethyl-16,16-dioxo-24-oxa-16λ⁶-thia-11,13,14,20,26,30,31-heptazahexacyclo[23.3.1.1^{12,15}.1^{17,20}.0^{2,10}.0^{5,9}]hentriaconta-1(28),2,4,9,12(31),14,17(30),18,25(29),26-decaen-13-yl)methoxy]ethyl-trimethyl-silane (100 mg, 0.161 mmol) in HCl (4 M in dioxane, 0.804 mL, 3.22 mmol) was stirred at 20° C. for 18 h and concentrated. The residue was diluted with MeOH (2.00 ml) and ethylenediamine (0.100 mL). The mixture was stirred at 22° C. for 20 min and concentrated. The product was purified by basic prep HPLC (0-70% MeCN in water) and triturated with DMSO and water (0.500 and 5.00 mL) to provide the title compound as a solid (8.30 mg, 10%).

LCMS m/z 492.3 (M+H)⁺ (ES⁺).

¹H NMR (500 MHz, DMSO-d₆) δ 12.50 (s, 1H), 8.75 (s, 1H), 8.09 (s, 1H), 7.90 (d, J=4.8 Hz, 1H), 7.18 (d, J=7.6 Hz, 1H), 7.00 (d, J=7.1 Hz, 1H), 6.79 (d, J=4.8 Hz, 1H), 6.75 (s, 1H), 6.01 (s, 1H), 4.15 (s, 2H), 2.95 (t, J=6.7 Hz, 2H), 2.85 (t, J=6.9 Hz, 2H), 2.25 (s, 2H), 2.10-2.02 (m, 2H), 1.63 (s, 6H).

### Example 8: 22,22-dimethyl-24-oxa-16 λ⁶-thia-11,13,14,26,31-pentazahexacyclo-[23.3.1.1^{12,15}.1^{17,21}.0^{2,10}.0^{5,9}] hentriaconta-1(28),2,4,9,12,14,17,19,21(30),25(29),26-undecaene 16,16-dioxide

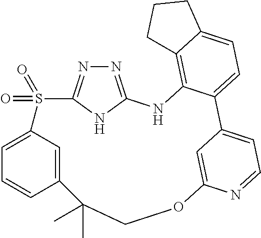 [SMILES: CC1(C)COc2cc(ccn2)-c2ccc3c(c2Nc2nnc([nH]2)S(=O)(=O)c2cccc1c2)CCC3]

### Step A: methyl 2-[3-[[5-[[5-(2-fluoro-4-pyridyl)indan-4-yl]amino]-1-(2-trimethylsilyl-ethoxymethyl)-1,2,4-triazol-3-yl]sulfanyl]phenyl]-2-methyl-propanoate

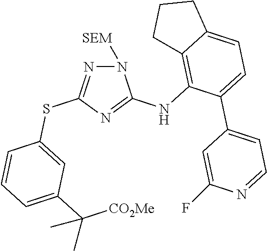 [SMILES: COC(=O)C(C)(C)c1cccc(Sc2nc(Nc3c(-c4ccnc(F)c4)ccc4c3CCC4)n(COCC[Si](C)(C)C)n2)c1]

DMEDA (0.44 mL, 3.96 mmol) was added to a mixture of 5-bromo-N-[5-(2-fluoro-4-pyridyl)indan-4-yl]-2-(2-trimethylsilylethoxymethyl)-1,2,4-triazol-3-amine (Intermediate A2, Step E) (100 mg, 198 μmol), methyl 2-methyl-2-(3-sulfanylphenyl)propanoate (Intermediate A13) (80%, 73.0 mg, 278 μmol), and CuI (75-9 mg, 396 μmol) in 1,4-dioxane (1.60 mL) at 22° C. under N₂. The mixture was degassed by bubbling N₂ through the mixture for 20 min, stirred at 100° C. for 6 h, and diluted with water (10.0 mL). The aqueous phase was extracted with EtOAc (3×10.0 mL), and the combined organic phases were washed with brine (10.0 mL), dried (Na₂SO₄), filtered and concentrated. The product was purified by FC (0-600% EtOAc/hexanes) to provide the title compound as a solid (47.2 mg, 38%).

LCMS m/z 632.8 (M−H)⁻ (ES⁻).

¹H NMR (500 MHz, CDCl₃) δ 8.16 (d, J=5.1 Hz, 1H), 7.45 (s, 1H), 7.25-7.06 (m, 6H), 6.93 (s, 1H), 6.15 (s, 1H), 5.28 (s, 2H), 3.63 (s, 3H), 3.53-3.46 (m, 2H), 2.96 (t, J=7.4 Hz, 2H), 2.70 (t, J=7.5 Hz, 2H), 2.06 (p, J=7.3 Hz, 2H), 1.53 (s, 6H), 0.77-0.70 (m, 2H), −0.01 (s, 9H).

### Step B: 2-[3-[[5-[[5-(2-fluoro-4-pyridyl)indan-4-yl]amino]-1-(2-trimethylsilylethoxy-methyl)-1,2,4-triazol-3-yl]sulfanyl]phenyl]-2-methyl-propan-1-ol

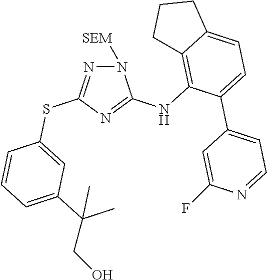 [SMILES: CC(C)(CO)c1cccc(Sc2nc(Nc3c(-c4ccnc(F)c4)ccc4c3CCC4)n(COCC[Si](C)(C)C)n2)c1]

LiAlH₄ (2M in THF, 684 uL, 1.37 mmol) was added to a mixture of methyl 2-[3-[[5-[[5-(2-fluoro-4-pyridyl)indan-4-yl]amino]-1-(2-trimethylsilyl-ethoxymethyl)-1,2,4-triazol-3-yl]sulfanyl]phenyl]-2-methyl-propanoate (431 mg, 652 μmol) in THF (9.66 mL) at 0° C. under N₂. The mixture was stirred at 22° C. for 90 min and slowly diluted with water (60.0 μL), aq. NaOH (15 w %, 60.0 uL), and water (180 μL). The mixture was stirred at 22° C. for 15 min, dried (MgSO₄, 250 mg), and filtered, washing with DCM (120 mL). The filtrate was concentrated to provide the title compound as a solid (385 mg, 97%).

LCMS m/z 606.4 (M+H)⁺ (ES⁺).

¹H NMR (400 MHz, CDCl₃) δ 8.12 (d, J=5.2 Hz, 1H), 7.54 (d, J=1.1 Hz, 1H), 7.31-7.26 (m, 3H), 7.22-7.04 (m, 3H), 6.92 (s, 1H), 6.15 (s, 1H), 5.26 (s, 2H), 3.54 (d, J=6.7 Hz, 2H), 3.52-3.43 (m, 2H), 2.97 (t, J=7.5 Hz, 2H), 2.71 (t, J=7.4 Hz, 2H), 2.07 (p, J=7.5 Hz, 2H), 1.69 (t, J=6.8 Hz, 1H), 1.30 (s, 6H), 0.78-0.69 (m, 2H), −0.01 (s, 9H).

### Step C: 2-[(22,22-dimethyl-24-oxa-16-thia-11,13,14,26,31-pentazahexacyclo-[23.3.1.1^{12,15}.1^{17,21}.0^{2,10}.0^{5,9}] hentriaconta-1(28),2,4,9,12(31),14,17,19,21(30),25 (29),26-undecaen-13-yl)methoxy]ethyl-trimethyl-silane

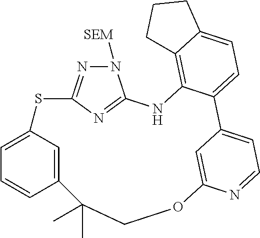 [SMILES: CC1(C)COc2cc(ccn2)-c2ccc3c(c2Nc2nc(nn2COCC[Si](C)(C)C)Sc2cccc1c2)CCC3]

A mixture of 2-[3-[[5-[[5-(2-fluoro-4-pyridyl)indan-4-yl]amino]-1-(2-trimethylsilyl-ethoxymethyl)-1,2,4-triazol-3-yl]sulfanyl]phenyl]-2-methyl-propan-1-ol (423 mg, 0.635 mmol) in THF (500 mL) was added over 5 h to a mixture of tBuOK (1M in THF, 6.35 mL, 6.35 mmol) in THF (100 mL) at 0° C. The mixture was stirred at 0° C. for 1 h, diluted with EtOH (20.0 mL), and concentrated. The product was purified by FC (0-50% EtOAc/hexanes) to provide the title compound as a solid (79.9 mg, 18%).

LCMS m/z 586.3 (M+H)⁺ (ES⁺).

¹H NMR (500 MHz, CDCl₃) δ 7.99 (d, J=5.3 Hz, 1H), 7.28-7.04 (m, 7H), 6.79 (dd, J=5.3, 1.5 Hz, 1H), 6.57 (s, 1H), 4.88 (s, 2H), 4.35 (s, 2H), 3.45-3.30 (m, 2H), 3.06 (t, J=7.5 Hz, 2H), 2.96 (t, J=7.4 Hz, 2H), 2.19 (p, J=7.5 Hz, 2H), 1.40 (s, 6H), 0.83-0.77 (m, 2H), −0.02 (s, 9H).

### Step D: 2-[(22,22-dimethyl-16,16-dioxo-24-oxa-16λ⁶-thia-11,13,14,26,31-pentazahexacyclo[23.3.1.1^{12,15}.1^{17,21}.0^{2,10}.0^{5,9}]hentriaconta-1(28),2,4,9,12(31),14,17,19,21(30),25(29),26-undecaen-13-yl)methoxy]ethyl-trimethyl-silane

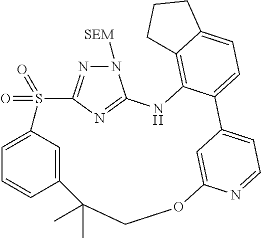 [SMILES: CC1(C)COc2cc(ccn2)-c2ccc3c(c2Nc2nc(nn2COCC[Si](C)(C)C)S(=O)(=O)c2cccc1c2)CCC3]

m-CPBA (84.9 mg, 379 μmol) was added to a mixture of 2-[(22,22-dimethyl-24-oxa-16-thia-11,13,14,26,31-pentazahexacyclo[23.3.1.1^{12,15}.1^{17,21}.0^{2,10}.0^{5,9}]hentriaconta-1(28),2,4,9,12(31),14,17,19,21(30),25(29),26-undecaen-13-yl)methoxy]ethyl-trimethyl-silane (88.8 mg, 152 μmol) in DCM (3.50 mL) at 0° C. under N₂. The mixture was stirred at 22° C. for 3 h and diluted with sat. aq. Na₂S₂O₃ (5.00 mL). The aqueous phase was extracted with DCM (3×10.0 mL) and the combined organic phases were washed with brine (10.0 mL), dried (Na₂SO₄), filtered, and concentrated. The residue was purified by FC (0-60% EtOAc/hexanes) to provide the title compound as a solid (44.5 mg, 47.5%).

LCMS m/z 618.3 (M+H)⁺ (ES⁺).

¹H NMR (500 MHz, CDCl₃) δ 7.95 (t, J=1.8 Hz, 1H), 7.86-7.76 (m, 1H), 7.71 (dd, J=6.9, 1.7 Hz, 1H), 7.51 (d, J=5.3 Hz, 1H), 7.44 (t, J=7.9 Hz, 1H), 7.16 (d, J=7.7 Hz, 1H), 7.08 (d, J=7.7 Hz, 1H), 6.79 (d, J=0.8 Hz, 1H), 6.61 (dd, J=5.3, 1.4 Hz, 1H), 6.51 (s, 1H), 5.28 (s, 2H), 4.38 (s, 2H), 3.61-3.42 (m, 2H), 3.00 (t, J=7.5 Hz, 2H), 2.89 (t, J=7.4 Hz, 2H), 2.17 (dq, J=14.9, 7.5 Hz, 2H), 1.53 (s, 6H), 0.94-0.87 (m, 2H), 0.00 (s, 9H).

### Step E: 22,22-dimethyl-24-oxa-16 λ⁶-thia-11,13,14,26,31-pentazahexacyclo-[23.3.1.1^{12,15}.1^{17,21}.0^{2,10}.0^{5,9}] hentriaconta-1(28),2,4,9,12,14,17,19,21(30),25(29),26-undecaene 16,16-dioxide

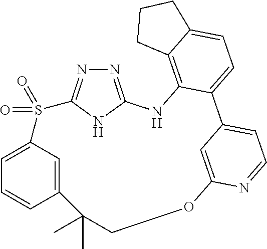 [SMILES: CC1(C)COc2cc(ccn2)-c2ccc3c(c2Nc2nnc([nH]2)S(=O)(=O)c2cccc1c2)CCC3]

TFA (1.50 ml, 20.2 mmol) was added to a mixture of 2-[(22,22-dimethyl-16,16-dioxo-24-oxa-16λ⁶-thia-11,13,14,26,31-pentazahexacyclo[23.3.1.1^{12,15}.1^{17,21}.0^{2,10}.0^{5,9}]-hentriaconta-1(28),2,4,9,12(31),14,17,19,21(30),25(29),26-undecaen-13-yl)methoxy]-ethyl-trimethyl-silane (44.5 mg, 72.0 μmol) in DCM (1.5 ml) at 0° C., and the solution was stirred at 22° C. for 2 h and concentrated. The residue was diluted with DCM (2.00 ml) and ethylenediamine (0.100 ml), and the mixture was stirred at 22° C. for 40 min and concentrated. The product was purified by basic prep HPLC (0-70% MeCN in water) and triturated with DMSO and water to provide the title compound as a solid (21.5 mg, 61%).

LCMS m/z 488.3 (M+H)⁺ (ES⁺).

¹H NMR (400 MHz, DMSO-d₆) δ 12.95 (s, 1H), 9.06 (s, 1H), 7.88 (d, J=5.2 Hz, 1H), 7.77 (dd, J=12.2, 8.0 Hz, 2H), 7.55 (t, J=7.8 Hz, 1H), 7.36 (s, 1H), 7.19 (d, J=7.7 Hz, 1H), 7.14 (d, J=7.7 Hz, 1H), 6.81 (dd, J=5.3, 1.2 Hz, 1H), 6.27 (s, 1H), 4.22 (s, 2H), 2.91 (t, J=7.4 Hz, 2H), 2.83 (t, J=7.4 Hz, 2H), 2.01 (p, J=7.4 Hz, 2H), 1.37 (s, 6H).

### Example 9: 25-methyl-22-oxa-2λ⁶-thia-1,4,5,7,20,25,32-heptaazahexacyclo-[24.2.2.1^{3,6}.1^{17,21}.0^{8,16}.0^{9,13}]dotriaconta-3,5,8,13,15,17,19,21(31)-octaene-2,2-dioxide

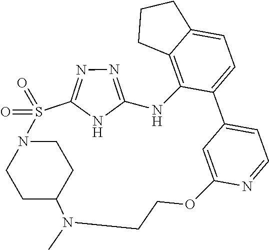 [SMILES: CN1CCOc2cc(ccn2)-c2ccc3c(c2Nc2nnc([nH]2)S(=O)(=O)N2CCC1CC2)CCC3]

### Step A: 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)-amino)ethan-1-ol

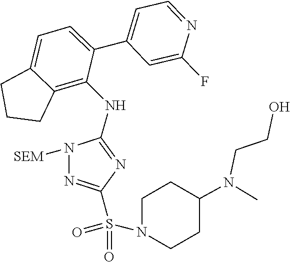 [SMILES: CN(CCO)C1CCN(S(=O)(=O)c2nc(Nc3c(-c4ccnc(F)c4)ccc4c3CCC4)n(COCC[Si](C)(C)C)n2)CC1]

NCS (125 mg, 938 μmol) was added to a solution of sodium 5-[[5-(2-fluoro-4-pyridyl)-indan-4-yl]amino]-1-(2-trimethylsilylethoxymethyl)-1,2,4-triazole-3-sulfinate (Intermediate A3) (0.500 g, 782 μmol) in DCM (9 mL) and stirred at 0° C. for 1 h. 2-(methyl(piperidin-4-yl)amino)ethan-1-ol (Intermediate A14) (186 mg, 1.17 mmol) and DIPEA (204 μL, 1.17 mmol) were then added in DCM (3 mL) at 0° C. and the reaction stirred at RT for 16 h. The reaction was washed with water (10 mL) and extracted with EtOAc (3×50 mL). The combined organics were then washed with brine (50 mL), dried (MgSO₄) and concentrated in vacuo. The crude product was purified by FC (0-20% MeOH/DCM) to afford the title compound (0.170 g, 30%) as a yellow oil.

LCMS m/z 646.5 (M+H)⁺ (ES⁺).

¹H NMR (500 MHz, DMSO-d₆) δ 9.04 (s, 1H), 8.17 (d, J=5.2 Hz, 1H), 7.35-7.32 (m, 1H), 7.31 (d, J=5.0 Hz, 2H), 7.12 (s, 1H), 5.42 (s, 2H), 4.30 (s, 1H), 3.82 (s, 1H), 3.62-3.54 (m, 2H), 3.54-3.48 (m, 2H), 3.45-3.38 (m, 2H), 2.98 (t, J=7.4 Hz, 2H), 2.75 (t, J=7.7 Hz, 2H), 2.48-2.40 (m, 2H), 2.34 (t, J=12.0 Hz, 2H), 2.16 (s, 3H), 2.03 (p, J=7.3 Hz, 2H), 1.75-1.68 (m, 2H), 1.47-1.34 (m, 2H), 0.89-0.81 (m, 2H), −0.03 (s, 9H).

### Step B: 25-methyl-22-oxa-2λ⁶-thia-1,4,5,7,20,25,32-heptaazahexacyclo-[24.2.2.1^{3,6}.1^{17,21}.0^{8,16}.0^{9,13}]dotriaconta-3,5,8,13,15,17,19,21(31)-octaene-2,2-dioxide

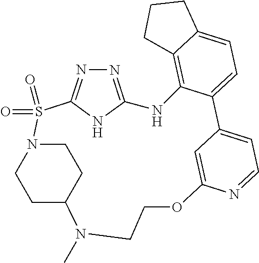 [SMILES: CN1CCOc2cc(ccn2)-c2ccc3c(c2Nc2nnc([nH]2)S(=O)(=O)N2CCC1CC2)CCC3]

To a solution of 2-((1-((5-((5-(2-fluoropyridin-4-yl)-2,3-dihydro-1H-inden-4-yl)-amino)-1-((2-(trimethylsilyl)ethoxy)methyl)-1H-1,2,4-triazol-3-yl)sulfonyl)piperidin-4-yl)(methyl)amino)ethan-1-ol (0.170 g, 232 μmol) in THF (20 mL) at 0° C. was added potassium tert-butoxide (260 mg, 2.32 mmol). The reaction was warmed to RT and stirred for 18 h. The reaction was diluted with EtOH (10 mL) and concentrated in vacuo. The resulting residue was purified by FC (0-20% MeOH/DCM). The isolated material was then taken up in DCM (3 mL) and TFA (3 mL) and stirred at RT for 90 min. The reaction was concentrated and the resulting residue purified by acidic prep HPLC (15-40% MeOH in water) to provide the title compound (8.00 mg, 7%) as a flocculent white solid.

LCMS m/z 496.4 (M+H)⁺ (ES⁺).

¹H NMR (500 MHz, DMSO-d₆) δ 13.39-12.78 (m, 1H), 9.10-8.71 (m, 1H), 8.10 (br s, 1H), 7.28 (d, J=7.7 Hz, 1H), 7.21 (br s, 1H), 7.02-6.43 (m, 2H), 4.65 (br s, 1H), 4.33 (br s, 1H), 3.46 (br s, 1H), 3.23-3.08 (m, 2H), 2.97 (t, J=7.5 Hz, 2H), 2.91-2.71 (m, 3H), 2.67-2.57 (m, 1H), 2.44-2.30 (m, 1H), 2.29 (br s, 1H), 2.16 (br s, 1H), 2.06 (p, J=7.5 Hz, 2H), 1.84 (br s, 2H), 1.65-1.40 (m, 2H). Two aliphatic protons obscured by water peak.

### Example 10: 23,23-dimethyl-25-oxa-16λ⁶-thia-11,13,14,17,22,27,33-heptaazaheptacyclo[24.3.1.1^{12,15}.1^{17,19}.1^{19,22}.0^{2,10}.0^{5,9}]tritriaconta-1(29),2,4,9,12,14,26(30),27-octaene-16,16-dioxide

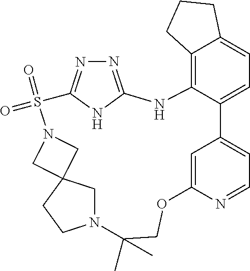 [SMILES: CC1(C)COc2cc(ccn2)-c2ccc3c(c2Nc2nnc([nH]2)S(=O)(=O)N2CC4(CCN1C4)C2)CCC3]

### Step A: 2-(2-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-2,6-diazaspiro[3.4]-octan-6-yl)-2-methylpropan-1-ol

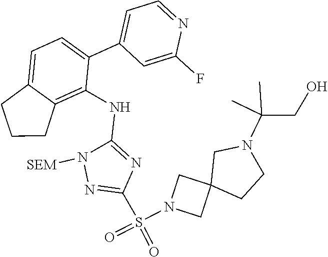 [SMILES: CC(C)(CO)N1CCC2(C1)CN(S(=O)(=O)c1nc(Nc3c(-c4ccnc(F)c4)ccc4c3CCC4)n(COCC[Si](C)(C)C)n1)C2]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (122 mg, 915 μmol), sodium 5-[[5-(2-fluoro-4-pyridyl)indan-4-yl]amino]-1-(2-trimethylsilylethoxymethyl)-1,2,4-triazole-3-sulfinate (Intermediate A3) (0.500 g, 762 μmol), DIPEA (531 μL, 3.05 mmol) and 2-methyl-2-(2,6-diazaspiro[3.4]octan-6-yl)propan-1-ol, di-(2,2,2-trifluoroacetic acid) (Intermediate A15) (469 mg, 1.14 mmol) in DCM (13 mL) at 0° C. to afford the title compound (214 mg, 33%) as a colourless oil.

LCMS m/z 672.5 (M+H)⁺ (ES⁺); 670.5 (M−H)⁻ (ES⁻).

¹H NMR (500 MHz, DMSO-d₆) δ 9.10 (s, 1H), 8.19 (d, J=5.2 Hz, 1H), 7.36-7.28 (m, 3H), 7.11 (s, 1H), 5.46 (s, 2H), 4.28 (br s, 1H), 3.79-3.66 (m, 4H), 3.58 (t, J=8.1 Hz, 2H), 3.20-3.16 (m, 2H), 2.99 (t, J=7.4 Hz, 2H), 2.73 (t, J=7.4 Hz, 2H), 2.68-2.58 (m, 2H), 2.57-2.50 (m, 2H), 2.03 (p, J=7.3 Hz, 2H), 1.63 (t, J=6.9 Hz, 2H), 0.93-0.80 (m, 8H), −0.02 (s, 9H).

### Step B: 23,23-dimethyl-25-oxa-16λ⁶-thia-11,13,14,17,22,27,33-heptaazaheptacyclo-[24.3.1.1^{12,15}.1^{17,19}.1^{19,22}.0^{2,10}.0^{5,9}]tritriaconta-1(29),2,4,9,12,14,26(30),27-octaene-16,16-dioxide

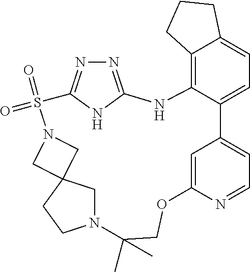 [SMILES: CC1(C)COc2cc(ccn2)-c2ccc3c(c2Nc2nnc([nH]2)S(=O)(=O)N2CC4(CCN1C4)C2)CCC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (3.31 mL, 3.31 mmol) and 2-(2-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-2,6-diazaspiro[3.4]octan-6-yl)-2-methylpropan-1-ol (214 mg, 248 μmol) in THF (150 mL) at 0° C. followed by TFA (3 mL), DCM (3 mL) and basic prep HPLC (20-50% MeCN in water) to afford the title compound (39 mg, 29%) as a white solid.

LCMS m/z 522.5 (M+H)⁺ (ES⁺); 520.3 (M−H)⁻ (ES⁻).

¹H NMR (500 MHz, DMSO-d₆) δ 9.21 (br s, 1H), 8.19 (d, J=5.2 Hz, 1H), 7.32 (d, J=7.8 Hz, 1H), 7.26 (d, J=7.8 Hz, 1H), 7.04 (dd, J=5.3, 1.4 Hz, 1H), 6.67 (s, 1H), 4.11 (s, 2H), 3.81-3.65 (m, 4H), 2.98 (t, J=7.5 Hz, 2H), 2.78 (t, J=7.5 Hz, 2H), 2.63 (t, J=7.0 Hz, 2H), 2.55 (s, 2H), 2.06 (p, J=7.5 Hz, 2H), 1.95 (t, J=6.9 Hz, 2H), 1.03 (s, 6H).

One exchangeable proton not observed.

### Example 11: 21-methyl-24-oxa-16λ⁶-thia-11,13,14,17,21,26,31-heptaazahexacyclo-[23.3.1.1^{12,15}.1^{17,19}.0^{2,10}.0^{5,9}]hentriaconta-1(28),2,4,9,12,14,25(29),26-octaene-16,16-dioxide

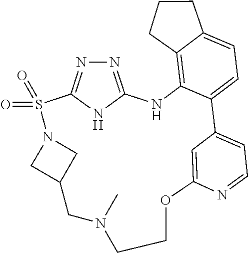 [SMILES: CN1CCOc2cc(ccn2)-c2ccc3c(c2Nc2nnc([nH]2)S(=O)(=O)N2CC(C1)C2)CCC3]

### Step A: 2-(((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)azetidin-3-yl)methyl)-(methyl)amino)ethan-1-ol

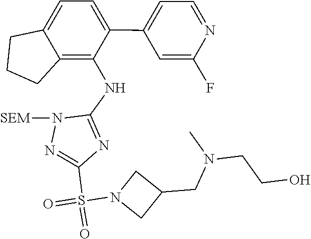 [SMILES: CN(CCO)CC1CN(S(=O)(=O)c2nc(Nc3c(-c4ccnc(F)c4)ccc4c3CCC4)n(COCC[Si](C)(C)C)n2)C1]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (122 mg, 915 μmol), sodium 5-[[5-(2-fluoro-4-pyridyl)indan-4-yl]amino]-1-(2-trimethylsilylethoxymethyl)-1,2,4-triazole-3-sulfinate (Intermediate A3) (0.500 g, 762 μmol), DIPEA (204 μL, 1.17 mmol) and 2-((azetidin-3-ylmethyl)(methyl)amino)-ethan-1-ol (Intermediate A16) (113 mg, 782 μmol) in DCM (12 mL) at 0° C. to afford the title compound (122.6 mg, 15%) as a yellow oil.

LCMS m/z 632.4 (M+H)⁺ (ES⁺).

¹H NMR (500 MHz, CDCl₃) δ 8.12 (d, J=5.2 Hz, 1H), 7.23-7.16 (m, 2H), 7.11-7.08 (m, 2H), 6.96-6.90 (m, 1H), 5.38 (s, 2H), 4.00-3.94 (m, 2H), 3.68-3.63 (m, 2H), 3.54-3.46 (m, 4H), 3.00-2.93 (m, 2H), 2.79-2.73 (m, 2H), 2.66 (s, 1H), 2.65-2.58 (m, 1H), 2.58-2.50 (m, 2H), 2.47-2.40 (m, 2H), 2.15 (s, 3H), 2.10-2.02 (m, 2H), 0.82-0.73 (m, 2H), −0.05 (s, 9H).

### Step B: 21-methyl-24-oxa-16λ⁶-thia-11,13,14,17,21,26,31-heptaazahexacyclo-[23.3.1.1^{12,15}.1^{17,19}.0^{2,10}.0^{5,9}]hentriaconta-1(28),2,4,9,12,14,25(29),26-octaene-16,16-dioxide

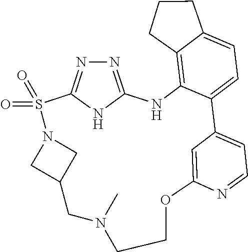 [SMILES: CN1CCOc2cc(ccn2)-c2ccc3c(c2Nc2nnc([nH]2)S(=O)(=O)N2CC(C1)C2)CCC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (1.25 mL, 1.25 mmol) and 2-(((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-azetidin-3-yl)methyl)(methyl)amino)ethan-1-ol (22.6 mg, 124.2 μmol) in 1,4-dioxane (60 mL) at 0° C. followed by TFA (3 mL), DCM (3 mL) and basic prep HPLC (20-50% MeCN in water) to afford the title compound (6 mg, 10%) as a white solid.

LCMS m/z 482.4 (M+H)⁺ (ES⁺); 480.4 (M−H)⁻ (ES⁻).

¹H NMR (500 MHz, DMSO-d₆) δ 8.96 (br s, 1H), 8.15 (d, J=5.3 Hz, 1H), 7.28 (d, J=7.7 Hz, 1H), 7.24 (d, J=7.7 Hz, 1H), 7.09-7.04 (m, 1H), 6.67 (s, 1H), 4.26-4.20 (m, 2H), 3.78 (t, J=8.0 Hz, 2H), 3.52-3.45 (m, 2H), 2.97 (t, J=7.5 Hz, 2H), 2.80 (t, J=7.4 Hz, 2H), 2.66-2.60 (m, 2H), 2.21 (s, 3H), 2.11-2.00 (m, 3H). Two aliphatic protons overlapped with DMSO-d₆ signal. One exchangeable proton not observed.

### Example 12: (20S)-22-methyl-25-oxa-16λ⁶-thia-11,13,14,17,22,27,32-heptaazahexacyclo[24.3.1.1^{12,15}.1^{17,20}.0^{2,10}.0^{5,9}]dotriaconta-1(29),2,4,9,12,14,26(30),27-octaene-16,16-dioxide

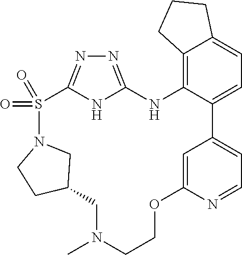 [SMILES: CN1CCOc2cc(ccn2)-c2ccc3c(c2Nc2nnc([nH]2)S(=O)(=O)N2CC[C@@H](C1)C2)CCC3]

### Step A: (S)-2-(((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)pyrrolidin-3-yl)-methyl)(methyl)amino)ethan-1-ol

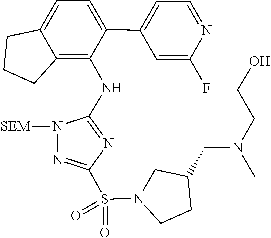

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (257 mg, 1.92 mmol), sodium 5-[[5-(2-fluoro-4-pyridyl)indan-4-yl]amino]-1-(2-trimethylsilylethoxymethyl)-1,2,4-triazole-3-sulfinate (Intermediate A3) (1.02 g, 1.59 mmol), DIPEA (0.4 mL, 2 mmol) and (R)-2-(methyl(pyrrolidin-3-ylmethyl)-amino)ethan-1-ol (300 mg, 1.54 mmol) (Intermediate A17) (469 mg, 1.14 mmol) in DCM (25 mL) at 0° C. to afford the title compound (421 mg, 34%) as a orange oil.

LCMS m/z 646.6 (M+H)⁺ (ES⁺); 644.2 (M−H)⁻ (ES⁻).

### Step B: (20S)-22-methyl-25-oxa-16λ⁶-thia-11,13,14,17,22,27,32-heptaazahexacyclo-[24.3.1.1^{12,15}.1^{17,20}.0^{2,10}.0^{5,9}]dotriaconta-1(29),2,4,9,12,14,26(30),27-octaene-16,16-dioxide

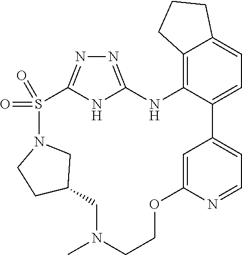 [SMILES: CN1CCOc2cc(ccn2)-c2ccc3c(c2Nc2nnc([nH]2)S(=O)(=O)N2CC[C@@H](C1)C2)CCC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (6.5 mL, 6.5 mmol) and (S)-2-(((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-pyrrolidin-3-yl)methyl)(methyl)amino)ethan-1-ol (421 mg, 521 μmol) in 1,4-dioxane (350 mL) at 0° C. followed by TFA (5 mL), DCM (5 mL) and basic prep HPLC (20-50% MeCN in water) to afford the title compound (60 mg, 21%) as a white solid.

LCMS m/z 496.4 (M+H)⁺ (ES⁺); 494.4 (M−H)⁻ (ES⁻).

¹H NMR (500 MHz, DMSO-d₆) δ 12.92 (s, 1H), 8.95 (s, 1H), 8.14 (d, J=5.3 Hz, 1H), 7.29 (d, J=7.7 Hz, 1H), 7.22 (d, J=7.7 Hz, 1H), 7.01 (dd, J=5.3, 1.5 Hz, 1H), 6.59 (s, 1H), 4.33-4.28 (m, 1H), 4.26-4.18 (m, 1H), 3.42-3.35 (m, 1H), 3.26-3.19 (m, 1H), 2.97 (t, J=7.5 Hz, 2H), 2.81-2.76 (m, 2H), 2.75-2.69 (m, 2H), 2.65 (t, J=9.6 Hz, 1H), 2.60-2.54 (m, 1H), 2.41-2.34 (m, 1H), 2.33-2.26 (m, 1H), 2.24 (s, 3H), 2.22-2.14 (m, 1H), 2.09-2.01 (m, 2H), 1.93-1.85 (m, 1H), 1.46-1.35 (m, 1H).

### Example 13: 27-methyl-22-oxa-2λ⁶-thia-1,4,5,7,20,27,34-heptaazahexacyclo-[26.2.2.1^{3,6}.1^{17,21}.0^{8,16}.0^{9,13}]tetratriaconta-3,5,8,13,15,17,19,21(33)-octaene-2,2-dioxide

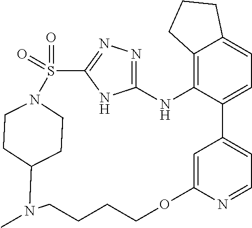 [SMILES: CN1CCCCOc2cc(ccn2)-c2ccc3c(c2Nc2nnc([nH]2)S(=O)(=O)N2CCC1CC2)CCC3]

### Step A: 4-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)-amino)butan-1-ol

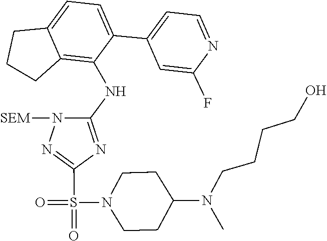 [SMILES: CN(CCCCO)C1CCN(S(=O)(=O)c2nc(Nc3c(-c4ccnc(F)c4)ccc4c3CCC4)n(COCC[Si](C)(C)C)n2)CC1]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (149 mg, 1.11 mmol), sodium 5-[[5-(2-fluoro-4-pyridyl)indan-4-yl]amino]-1-(2-trimethylsilylethoxymethyl)-1,2,4-triazole-3-sulfinate (Intermediate A3) (620 mg, 928 μmol), DIPEA (646 μL, 3.71 mmol) and 4-(methyl(piperidin-4-yl)amino)butan-1-ol, di-(2,2,2-trifluoroacetic acid) (Intermediate A18) (585 mg, 1.42 mmol) in DCM (12 mL) at 0° C. to afford the title compound (93 mg, 11%) as a colourless oil.

LCMS m/z 674.5 (M+H)⁺ (ES⁺); 672.4 (M−H)⁻ (ES⁻).

¹H NMR (500 MHz, DMSO-d₆) δ 9.06 (s, 1H), 8.17 (d, J=5.2 Hz, 1H), 7.39-7.27 (m, 3H), 7.12 (s, 1H), 5.43 (s, 2H), 4.48 (br s, 1H), 3.63-3.48 (m, 4H), 3.43-3.35 (m, 2H), 2.99 (t, J=7.5 Hz, 2H), 2.75 (t, J=7.4 Hz, 2H), 2.40-2.24 (m, 5H), 2.12 (s, 3H), 2.04 (p, J=7.4 Hz, 2H), 1.74-1.65 (m, 2H), 1.44-1.35 (m, 6H), 0.87 (t, J=8.3 Hz, 2H), −0.02 (s, 9H).

### Step B: 27-methyl-22-oxa-2λ⁶-thia-1,4,5,7,20,27,34-heptaazahexacyclo-[26.2.2.1^{3,6}.1^{17,21}.0^{8,16}.0^{9,13}]tetratriaconta-3,5,8,13,15,17,19,21(33)-octaene-2,2-dioxide

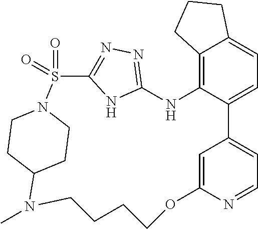 [SMILES: CN1CCCCOc2cc(ccn2)-c2ccc3c(c2Nc2nnc([nH]2)S(=O)(=O)N2CCC1CC2)CCC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (1.14 mL, 1.14 mmol) and 4-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-piperidin-4-yl)(methyl)amino)butan-1-ol (90 mg, 114 μmol) in THF (200 mL) at 0° C. followed by TFA (3 mL), DCM (3 mL) and basic prep HPLC (20-50% MeCN in water) to afford the title compound (9 mg, 15%) as a white solid.

LCMS m/z 524.4 (M+H)⁺ (ES⁺); 522.7 (M−H)⁻ (ES⁻).

¹H NMR (500 MHz, DMSO-d₆) δ 8.93 (br s, 1H), 8.14 (d, J=5.3 Hz, 1H), 7.27 (d, J=7.7 Hz, 1H). 7.19 (d, J=7.6 Hz, 1H), 7.01 (d, J=5.3 Hz, 1H), 6.65 (s, 1H), 4.23 (t, J=5.3 Hz, 2H), 3.53-3.46 (m, 2H), 2.97 (t, J=7.4 Hz, 2H), 2.71 (t, J=7.5 Hz, 2H), 2.44 (t, J=6.3 Hz, 2H), 2.39-2.30 (m, 2H), 2.11 (s, 3H), 2.04 (p, J=7.4 Hz, 2H), 1.74-1.67 (m, 2H), 1.60-1.42 (m, 7H). One exchangeable proton not observed.

### Example 14: (20R)-22-methyl-25-oxa-16λ⁶-thia-11,13,14,17,22,27,32-heptaazahexacyclo[24.3.1.1^{12,15}.1^{17,20}.0^{2,10}.0^{5,9}]dotriaconta-1(29),2,4,9,12,14,26(30),27-octaene-16,16-dioxide

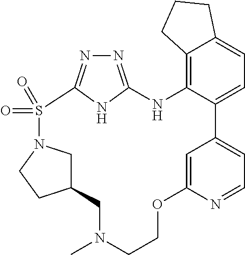 [SMILES: CN1CCOc2cc(ccn2)-c2ccc3c(c2Nc2nnc([nH]2)S(=O)(=O)N2CC[C@H](C1)C2)CCC3]

### Step A: (R)-2-(((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)pyrrolidin-3-yl)-methyl)(methyl)amino)ethan-1-ol

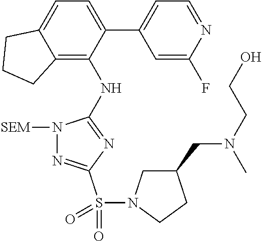 [SMILES: CN(CCO)C[C@H]1CCN(S(=O)(=O)c2nc(Nc3c(-c4ccnc(F)c4)ccc4c3CCC4)n(COCC[Si](C)(C)C)n2)C1]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (240 mg, 1.80 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.940 g, 1.50 mmol), DIPEA (391 μL, 2.25 mmol) and (S)-2-(methyl(pyrrolidin-3-ylmethyl)amino)ethan-1-ol, 2,2,2-trifluoroacetic acid (226 mg, 0.83 mmol) (Intermediate A19) in DCM (12 mL) at 0° C. to afford the title compound (201 mg, 16%) as a yellow oil.

LCMS m/z 646.5 (M+H)⁺ (ES⁺); 644.4 (M−H)⁻ (ES⁻).

¹H NMR (500 MHz, CDCl₃) δ 8.19 (d, J=5.2 Hz, 1H), 7.25-7.20 (m, 2H), 7.13 (d, J=7.7 Hz, 1H), 6.99-6.94 (m, 1H), 6.49 (s, 1H), 5.36 (s, 2H), 3.93 (s, 1H), 3.65-3.56 (m, 2H), 3.53-3.47 (m, 2H), 3.39-3.30 (m, 2H), 3.16-3.09 (m, 2H), 3.01 (t, J=7.6 Hz, 2H), 2.77 (t, J=7.5 Hz, 2H), 2.63-2.56 (m, 2H), 2.52-2.42 (m, 2H), 2.36-2.25 (m, 4H), 2.16-2.07 (m, 2H), 1.69-1.56 (m, 2H), 0.81-0.72 (m, 2H), −0.01 (s, 9H).

### Step B: (20R)-22-methyl-25-oxa-16λ⁶-thia-11,13,14,17,22,27,32-heptaazahexacyclo-[24.3.1.1^{12,15}.1^{17,20}.0^{2,10}.0^{5,9}]dotriaconta-1(29),2,4,9,12,14,26(30),27-octaene-16,16-dioxide

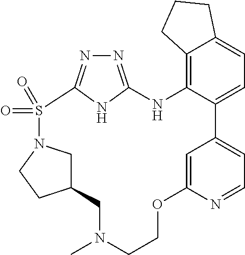 [SMILES: CN1CCOc2cc(ccn2)-c2ccc3c(c2Nc2nnc([nH]2)S(=O)(=O)N2CC[C@H](C1)C2)CCC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KOtBu in THF (2.5 mL, 2.5 mmol) and (R)-2-(((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-pyrrolidin-3-yl)methyl)(methyl)amino)ethan-1-ol (201 mg, 249 μmol) in THF (180 mL) at 0° C. followed by TFA (6 mL), DCM (6 mL) and basic prep HPLC (20-50% MeCN in water) to afford the title compound (12 mg, 9%) as a white solid.

LCMS m/z 496.4 (M+H)⁺ (ES⁺); 494.4 (M−H)⁻ (ES⁻).

¹H NMR (500 MHz, DMSO-d₆) δ 8.93 (s, 1H), 8.14 (d, J=5.3 Hz, 1H), 7.29 (d, J=7.7 Hz, 1H), 7.22 (d, J=7.7 Hz, 1H), 7.01 (dd, J=5.2, 1.5 Hz, 1H), 6.59 (s, 1H), 4.34-4.28 (m, 1H), 4.25-4.18 (m, 1H), 3.26-3.19 (m, 1H), 2.97 (t, J=7.4 Hz, 2H), 2.82-2.76 (m, 2H), 2.76-2.68 (m, 1H), 2.65 (t, J=9.5 Hz, 1H), 2.60-2.53 (m, 1H), 2.41-2.34 (m, 1H), 2.33-2.26 (m, 1H), 2.24 (s, 3H), 2.22-2.14 (m, 1H), 2.10-2.01 (m, 2H), 1.94-1.85 (m, 1H), 1.46-1.34 (m, 2H). One exchangeable proton not observed. One aliphatic proton overlapped with water in DMSO-d⁶ peak.

### Example 15: 8-oxa-28λ⁶-thia-4,10,23,25,26,29,34-heptaazaheptacyclo-[27.2.2.1^{9,13}.1^{24,27}.0^{1,4}.0^{14,22}.0^{17,21}]pentatriaconta-9(35),10,12,14,16,21,24,26-octaene-28,28-dioxide

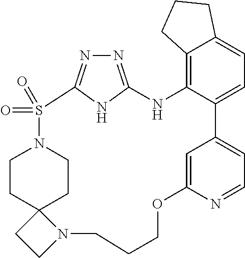 [SMILES: O=S1(=O)c2nnc([nH]2)Nc2c(ccc3c2CCC3)-c2ccnc(c2)OCCCN2CCC23CCN1CC3]

### Step A: 3-(7-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-1,7-diazaspiro[3-5]-nonan-1-yl)propan-1-ol

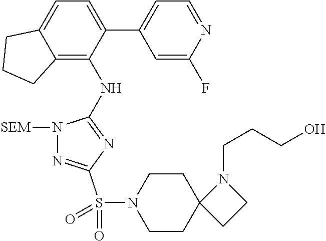 [SMILES: C[Si](C)(C)CCOCn1nc(S(=O)(=O)N2CCC3(CCN3CCCO)CC2)nc1Nc1c(-c2ccnc(F)c2)ccc2c1CCC2]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (128 mg, 956 μmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (500 mg, 796 μmol), DIPEA (555 μL, 3.19 mmol) and 3-(1,7-diazaspiro[3.5]nonan-1-yl)propan-1-ol, di-(2,2,2-trifluoroacetic acid) (Intermediate A20) (0.60 g, 1.2 mmol) in DCM (15 mL) at 0° C. to afford the title compound (0.44 g, 41%) as a sticky brown gum.

LCMS m/z 672.5 (M+H)⁺ (ES⁺); 670.5 (M−H)⁻ (ES⁻).

¹H NMR (500 MHz, DMSO-d₆) δ 9.11 (s, 1H), 8.20 (d, J=5.2 Hz, 1H), 7.35-7.27 (m, 3H), 7.09 (s, 1H), 5.41 (s, 2H), 3.70-3.57 (m, 9H), 3.55-3.48 (m, 2H), 3.43 (t, J=6.1 Hz, 2H), 3.20-3.07 (m, 7H), 2.99 (t, J=7.4 Hz, 2H), 2.71 (t, J=7.4 Hz, 2H), 2.04-1.98 (m, 2H), 0.87-0.80 (m, 2H), −0.03 (s, 9H). One exchangeable proton not observed.

¹⁹F NMR (471 MHz, DMSO-d₆) δ −73.45.

### Step B: 8-oxa-28λ⁶-thia-4,10,23,25,26,29,34-heptaazaheptacyclo-[27.2.2.1^{9,13}.1^{24,27}.0^{1,4}.0^{14,22}.0^{17,21}]pentatriaconta-9(35),10,12,14,16,21,24,26-octaene-28,28-dioxide

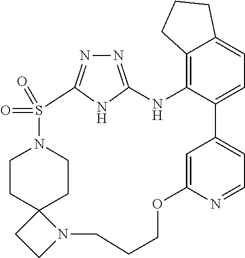 [SMILES: O=S1(=O)c2nnc([nH]2)Nc2c(ccc3c2CCC3)-c2ccnc(c2)OCCCN2CCC23CCN1CC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (4.6 mL, 4.6 mmol) and 3-(7-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-1,7-diaza-spiro[3.5]-nonan-1-yl)propan-1-ol (0.44 g, 0.46 mmol) in THF (170 mL) at 0° C. followed by TFA (5 mL), DCM (5 mL) and basic prep HPLC (20-50% MeCN in water) to afford the title compound (16 mg, 6%) as a white solid.

LCMS m/z 522.5 (M+H)⁺ (ES⁺); 520.4 (M−H)⁻ (ES⁻).

¹H NMR (500 MHz, DMSO-d⁶) δ 12.97 (s, 1H), 9.06 (s, 1H), 8.15 (d, J=5.2 Hz, 1H), 7.30 (d, J=7.7 Hz, 1H), 7.25 (d, J=7.6 Hz, 1H), 6.97 (dd, J=5.3, 1.5 Hz, 1H), 6.61 (d, J=1.4 Hz, 1H), 4.35 (t, J=5.3 Hz, 2H), 3.17-3.01 (m, 3H), 2.95 (t, J=7.2 Hz, 2H), 2.69 (t, J=7.4 Hz, 2H), 2.45-2.37 (m, 2H), 2.08-1.96 (m, 2H), 1.82 (t, J=7.1 Hz, 2H), 1.78-1.66 (m, 2H), 1.57-1.47 (m, 3H). 4 protons obscured by water signal.

### Example 16: 19-[(dimethylamino)methyl]-24-oxa-16λ⁶-thia-11,13,14,17,26,31-hexaazahexacyclo[23.3.1.1^{12,15}.1^{17,19}.0^{2,10}.0^{5,9}]hentriaconta-1(28),2,4,9,12,14,25(29),26-octaene-16,16-dioxide

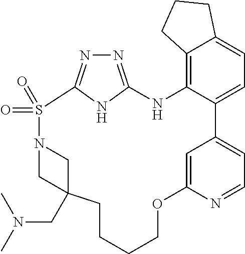 [SMILES: CN(C)CC12CCCCOc3cc(ccn3)-c3ccc4c(c3Nc3nnc([nH]3)S(=O)(=O)N(C1)C2)CCC4]

### Step A: 4-(3-((dimethylamino)methyl)-1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)-sulfonyl)azetidin-3-yl)butan-1-ol

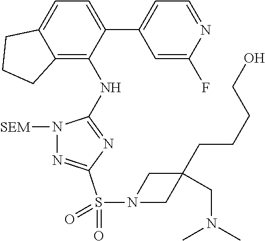 [SMILES: CN(C)CC1(CCCCO)CN(S(=O)(=O)c2nc(Nc3c(-c4ccnc(F)c4)ccc4c3CCC4)n(COCC[Si](C)(C)C)n2)C1]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (236 mg, 1.77 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.875 g, 1.39 mmol), DIPEA (383 μL, 3.20 mmol) and 4-(3-((dimethylamino)methyl)azetidin-3-yl)butan-1-ol (Intermediate A21) (0.60 g, 1.2 mmol) in DCM (15 mL) at 0° C. to afford the title compound (0.44 g, 41%) as a sticky brown gum.

LCMS m/z 674.5 (M+H)⁺ (ES⁺); 672.0 (M−H)⁻ (ES⁻).

¹H NMR (500 MHz, CDCl₃) δ 8.17 (d, J=5.2 Hz, 1H), 7.22 (d, J=7.7 Hz, 1H), 7.20-7.16 (m, 1H), 7.11 (d, J=7.7 Hz, 1H), 6.92 (d, J=1.7 Hz, 1H), 6.53 (s, 1H), 5.38 (s, 2H), 3.90 (s, 1H), 3.72-3.66 (m, 4H), 3.63-3.57 (m, 2H), 3.55-3.49 (m, 2H), 2.99 (t, J=7.6 Hz, 2H), 2.77 (t, J=7.4 Hz, 2H), 2.13-2.02 (m, 10H), 1.68-1.59 (m, 2H), 1.54-1.46 (m, 2H), 1.31-1.22 (m, 2H), 0.78-0.72 (m, 2H), −0.02 (s, 9H).

### Step B: 19-[(dimethylamino)methyl]-24-oxa-16λ⁶-thia-11,13,14,17,26,31-hexaazahexacyclo[23.3.1.1^{12,15}.1^{17,19}.0^{2,10}.0^{5,9}]hentriaconta-1(28),2,4,9,12,14,25(29),26-octaene-16,16-dioxide

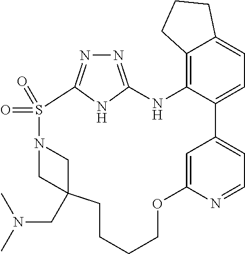 [SMILES: CN(C)CC12CCCCOc3cc(ccn3)-c3ccc4c(c3Nc3nnc([nH]3)S(=O)(=O)N(C1)C2)CCC4]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (3.00 mL, 3.00 mmol) and 4-(3-((dimethylamino)methyl)-1-((5-((5-(2-fluoropyridin-4-yl)-2,3-dihydro-1H-inden-4-yl)amino)-1-((2-(trimethylsilyl)ethoxy)-methyl)-1H-1,2,4-triazol-3-yl)sulfonyl)azetidin-3-yl)butan-1-ol (376 mg, 447 μmol) in THF (250 mL) at 0° C. followed by TFA (7 mL), DCM (7 mL) and basic prep HPLC (20-50% MeCN in water) to afford the title compound (110 mg, 29%) as a white solid.

LCMS m/z 524.4 (M+H)⁺ (ES⁺); 522.3 (M−H)⁻ (ES⁻).

¹H NMR (500 MHz, DMSO-d₆) δ 13.13 (s, 1H), 9.07 (s, 1H), 8.19 (d, J=5.2 Hz, 1H), 7.30 (d, J=7.7 Hz, 1H), 7.26 (d, J=7.7 Hz, 1H), 7.01 (dd, J=5.2, 1.4 Hz, 1H), 6.65 (s, 1H), 4.21 (t, J=5.6 Hz, 2H), 3.52 (s, 4H), 2.97 (t, J=7.5 Hz, 2H), 2.75 (t, J=7.5 Hz, 2H), 2.15 (s, 6H), 2.09-2.00 (m, 2H), 1.68-1.61 (m, 2H), 1.47-1.37 (m, 2H), 1.27-1.17 (m, 2H). Two exchangeable protons not observed.

### Example 17: (20S)-21-methyl-25-oxa-16λ⁶-thia-11,13,14,17,21,27,32-heptaazahexacyclo[24.3.1.1^{12,15}.1^{17,20}.0^{2,10}.0^{0,9}]dotriaconta-1(29),2,4,9,12,14,26(30),27-octaene-16,16-dioxide

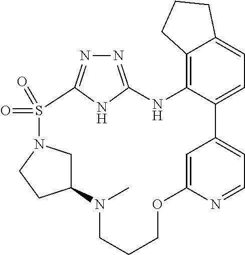 [SMILES: CN1CCCOc2cc(ccn2)-c2ccc3c(c2Nc2nnc([nH]2)S(=O)(=O)N2CC[C@H]1C2)CCC3]

### Step A: (S)-3-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)pyrrolidin-3-yl)-(methyl)amino)propan-1-ol

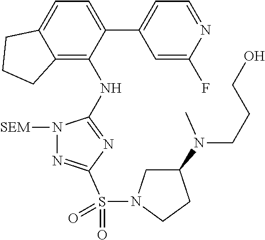 [SMILES: CN(CCCO)[C@H]1CCN(S(=O)(=O)c2nc(Nc3c(-c4ccnc(F)c4)ccc4c3CCC4)n(COCC[Si](C)(C)C)n2)C1]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (192 mg, 1.44 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.640 g, 1.20 mmol), DIPEA (836 μL, 4.80 mmol) and (S)-3-(methyl(pyrrolidin-3-yl)amino)propan-1-ol (Intermediate A22) (285 mg, 1.80 mmol) in DCM (12 mL) at 0° C. to afford the title compound (225 mg, 23%) as a colourless oil.

LCMS m/z 646.5 (M+H)⁺ (ES⁺); 644.5 (M−H)⁻ (ES⁻).

¹H NMR (500 MHz, DMSO-d₆) δ 9.11-8.99 (m, 1H), 8.19 (d, J=5.2 Hz, 1H), 7.35-7.27 (m, 3H), 7.10 (s, 1H), 5.42 (s. 2H), 4.36 (t, J=5.0 Hz, 1H), 3.53 (t, J=8.1 Hz, 2H), 3.46-3.34 (m, 3H), 3.17-3.09 (m, 1H), 3.02-2.91 (m, 3H), 2.82-2.64 (m, 3H), 2.34-2.23 (m, 2H), 2.09-1.98 (m, 5H), 1.97-1.89 (m, 1H), 1.63-1.52 (m, 1H), 1.48 (p, J=6.8 Hz, 2H), 0.85 (t, J=8.1 Hz, 2H), −0.03 (s, 9H). One proton obscured by water peak.

### Step B: (20S)-21-methyl-25-oxa-16λ⁶-thia-11,13,14,17,21,27,32-heptaazahexacyclo-[24.3.1.1^{12,15}.1^{17,20}.0^{2,10}.0^{5,9}]dotriaconta-1(29),2,4,9,12,14,26(30),27-octaene-16,16-dioxide

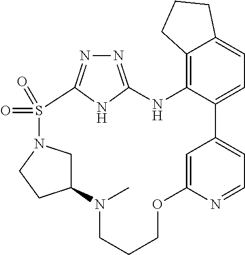 [SMILES: CN1CCCOc2cc(ccn2)-c2ccc3c(c2Nc2nnc([nH]2)S(=O)(=O)N2CC[C@H]1C2)CCC3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (2.90 mL, 2.90 mmol) and (S)-3-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-pyrrolidin-3-yl)(methyl)amino)propan-1-ol (220 mg, 290 μmol) in THF (100 mL) at 0° C. followed by TFA (3 mL), DCM (3 mL) and basic prep HPLC (20-50% MeCN in water) to afford the title compound (44 mg, 29%) as a white solid.

LCMS m/z 496.5 (M+H)⁺ (ES⁺); 494.2 (M−H)⁻ (ES⁻).

¹H NMR (500 MHz, DMSO-d₆) δ 12.83 (br s, 1H), 8.97 (br s, 1H), 8.12 (d, J=5.2 Hz, 1H), 7.28 (d, J=7.7 Hz, 1H), 7.21 (d, J=7.7 Hz, 1H), 6.99 (dd, J=5.2, 1.5 Hz, 1H), 6.63 (s, 1H), 4.29-4.17 (m, 2H), 3.48-3.41 (m, 1H), 3.31-3.27 (m, 2H), 3.08-2.93 (m, 3H), 2.89-2.75 (m, 2H), 2.66-2.58 (m, 1H), 2.45-2.28 (m, 2H), 2.16 (s, 3H), 2.06 (p, J=7.5 Hz, 2H), 1.95-1.86 (m, 1H), 1.82-1.67 (m, 2H), 1.56-1.44 (m, 1H).

### Example 18: 6-oxa-26λ⁶-thia-2,8,21,23,24,27,32-heptaazaheptacyclo-[25.2.2.1^{2,4}.1^{7,11}.1^{22,25}.0^{12,20}.1^{15,19}]tetratriaconta-7(33),8,10,12,14,19,22,24-octaene-26,26-dioxide

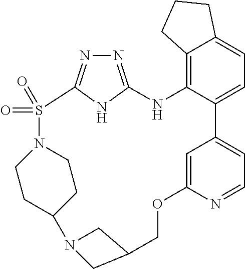 [SMILES: O=S1(=O)c2nnc([nH]2)Nc2c(ccc3c2CCC3)-c2ccnc(c2)OCC2CN(C2)C2CCN1CC2]

### Step A: (1-(1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)azetidin-3-yl)methanol

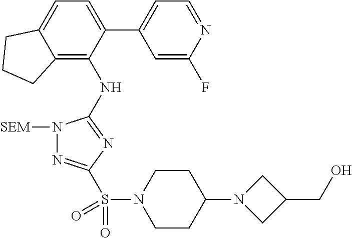 [SMILES: C[Si](C)(C)CCOCn1nc(S(=O)(=O)N2CCC(N3CC(CO)C3)CC2)nc1Nc1c(-c2ccnc(F)c2)ccc2c1CCC2]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (140 mg, 1.05 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.550 g, 876 μmol), DIPEA (230 μL, 1.32 mmol) and (1-(piperidin-4-yl)azetidin-3-yl)methanol (Intermediate A23) (150 mg, 881 μmol) in DCM (12 mL) at 0° C. to afford the title compound (195 mg, 20%) as a colourless oil.

LCMS m/z 658-5 (M+H)⁺ (ES⁺); 656.3 (M−H)⁻ (ES⁻).

### Step B: 6-oxa-26λ⁶-thia-2,8,21,23,24,27,32-heptaazaheptacyclo-[25.2.2.1^{2,4}.1^{7,11}.1^{22,25}.0^{12,20}.1^{15,19}]tetratriaconta-7(33),8,10,12,14,19,22,24-octaene-26,26-dioxide

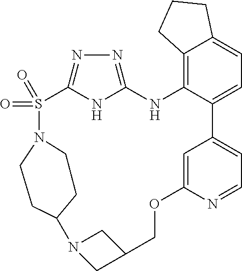 [SMILES: O=S1(=O)c2nnc([nH]2)Nc2c(ccc3c2CCC3)-c2ccnc(c2)OCC2CN(C2)C2CCN1CC2]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (3.00 mL, 3.00 mmol) and (1-(1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-piperidin-4-yl)azetidin-3-yl)methanol (195 mg, 297 μmol) in THF (250 mL) at 0° C. followed by TFA (5 mL), DCM (5 mL) and basic prep HPLC (20-50% MeCN in water) to afford the title compound (5 mg, 3%) as a white solid.

LCMS m/z 508.5 (M+H)⁺ (ES⁺); 506.5 (M−H)⁻ (ES⁻).

¹H NMR (500 MHz, DMSO-d₆) δ 13.12 (s, 1H), 9.04 (s, 1H), 8.20 (d, J=5.3 Hz, 1H), 7.35-7.21 (m, 2H), 7.02 (dd, J=5.3, 1.5 Hz, 1H), 6.67 (s, 1H), 4.31 (s, 2H), 3.25-3.10 (m, 4H), 3.04-2.90 (m, 4H), 2.68 (t, J=7.4 Hz, 2H), 2.59-2.55 (m, 1H), 2.39-2.29 (m, 1H), 2.08-2.00 (m, 2H), 1.58-1.43 (m, 4H). Two aliphatic protons overlapped with water peak.

### Example 10: 27-oxa-16λ⁶-thia-11,13,14,17,22,29,35-heptaazaheptacyclo-[26.3.1.1^{12,15}.1^{17,19}.1^{19,22}.0^{2,10}.0^{5,9}]pentatriaconta-1(31),2,4,9,12,14,28(32),29-octaene-16,16-dioxide

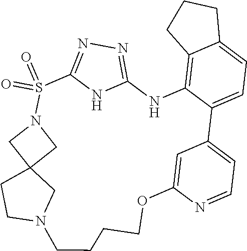 [SMILES: O=S1(=O)c2nnc([nH]2)Nc2c(ccc3c2CCC3)-c2ccnc(c2)OCCCCN2CCC3(C2)CN1C3]

### Step A: 4-(2-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-2,6-diazaspiro[3.4]-octan-6-yl)butan-1-ol

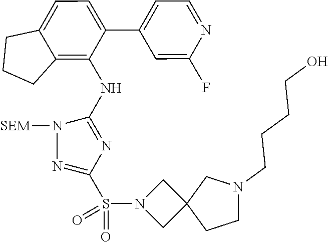 [SMILES: C[Si](C)(C)CCOCn1nc(S(=O)(=O)N2CC3(CCN(CCCCO)C3)C2)nc1Nc1c(-c2ccnc(F)c2)ccc2c1CCC2]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (192 mg, 1.44 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.550 g, 876 μmol), DIPEA (836 μL, 4.80 mmol) and 4-(2,6-diazaspiro[3.4]octan-6-yl)butan-1-ol, di-(2,2,2-trifluoroacetic acid) (Intermediate A24) (667 mg, 1.63 mmol) in DCM (12 mL) at 0° C. to afford the title compound (158 mg, 16%) as a colourless oil.

LCMS m/z 672.5 (M+H)⁺ (ES⁺); 670.5 (M−H)⁻ (ES⁻).

¹H NMR (500 MHz, DMSO-d₆) δ 9.10 (s, 1H), 8.25 (d, J=5.2 Hz, 1H), 7.50-7.34 (m, 3H), 7.27 (s, 1H), 5.60 (s, 2H), 4.38 (br s, 1H), 3.80-3.59 (m, 6H), 3.40 (t, J=6.4 Hz, 2H), 3.02 (t, J=7.4 Hz, 2H), 2.74 (t, J=7.4 Hz, 2H), 2.45-2.25 (m, 6H), 2.09 (p, J=7.4 Hz, 2H), 1.63 (t, J=8.0 Hz, 2H), 1.56-1.45 (m, 4H), 0.87 (t, J=8.1 Hz, 2H), −0.03 (s, 9H).

### Step B: 27-oxa-16λ⁶-thia-11,13,14,17,22,29,35-heptaazaheptacyclo-[26.3.1.1^{12,15}.1^{17,19}.1^{19,22}.0^{2,10}.0^{5,9}]pentatriaconta-1(31),2,4,9,12,14,28(32),29-octaene-16,16-dioxide

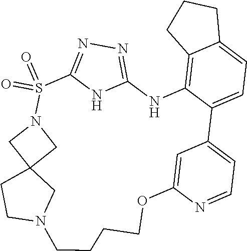 [SMILES: O=S1(=O)c2nnc([nH]2)Nc2c(ccc3c2CCC3)-c2ccnc(c2)OCCCCN2CCC3(C2)CN1C3]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (2.00 mL, 2.00 mmol) and 4-(2-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-2,6-diazaspiro[3.4]-octan-6-yl)butan-1-ol (158 mg, 200 μmol) in THF (100 mL) at 0° C. followed by TFA (5 mL), DCM (5 mL) and basic prep HPLC (20-50% MeCN in water) to afford the title compound (26 mg, 24%) as a white solid.

LCMS m/z 522.5 (M+H)⁺ (ES⁺); 520.5 (M−H)⁻ (ES⁻).

¹H NMR (500 MHz, DMSO-d₆) δ 13.26 (br s, 1H), 9.04 (br s, 1H), 8.15 (d, J=5.3 Hz, 1H), 7.28 (d, J=7.7 Hz, 1H), 7.14 (d, J=7.7 Hz, 1H), 7.00 (d, J=5.2 Hz, 1H), 6.72 (s, 1H), 4.38-4.25 (m, 2H), 3.75 (s, 4H), 2.96 (t, J=7.5 Hz, 2H), 2.65 (t, J=7.4 Hz, 2H), 2.44 (t, J=7.1 Hz, 2H), 2.34 (t, J=6.3 Hz, 2H), 2.15 (s, 2H), 2.09-1.97 (m, 4H), 1.76-1.67 (m, 2H), 1.47 (p, J=7.0 Hz, 2H).

### Example 20: 27-oxa-16λ⁶-thia-11,13,14,17,22,29,35-heptaazaheptacyclo-[26.3.1.1^{12,15}.1^{17,19}.1^{19,22}.0^{2,10}.0^{5,9}]pentatriaconta-1(31),2,4,9,12,14,28(32),29-octaene-16,16-dioxide

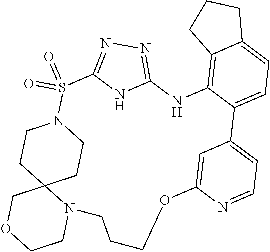 [SMILES: O=S1(=O)c2nnc([nH]2)Nc2c(ccc3c2CCC3)-c2ccnc(c2)OCCCN2CCOCC23CCN1CC3]

### Step A: 3-(9-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-4-oxa-1,9-diazaspiro-[5.5]undecan-1-yl)propan-1-ol and 3-((4-oxa-1,9-diazaspiro[5.5]undecan-9-yl)-sulfonyl)-N-(5-(2-fluoropyridin-4-yl)-2,3-dihydro-1H-inden-4-yl)-1-((2-(trimethyl-silyl)ethoxy)methyl)-1H-1,2,4-triazol-5-amine (1/1)

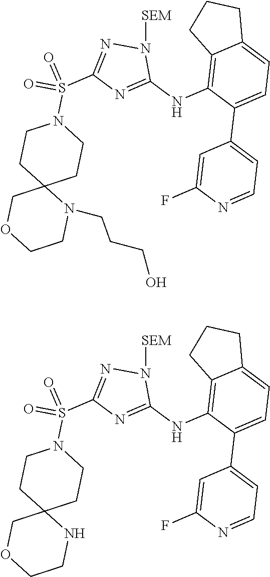 [SMILES: C[Si](C)(C)CCOCn1nc(S(=O)(=O)N2CCC3(CC2)COCCN3CCCO)nc1Nc1c(-c2ccnc(F)c2)ccc2c1CCC2 ; C[Si](C)(C)CCOCn1nc(S(=O)(=O)N2CCC3(CC2)COCCN3)nc1Nc1c(-c2ccnc(F)c2)ccc2c1CCC2]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (451 mg, 1.44 mmol), di-sodium (s-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (1.77 g, 2.82 mmol), DIPEA (1.50 mL, 8.61 mmol) and 3-(4-oxa-1,9-diazaspiro[5.5]undecan-1-yl)propan-1-ol-4-oxa-1,9-diazaspiro[5-5]-undecane (1/1), di-(2,2,2-trifluoroacetic acid) (1.36 g, 2.28 mmol) (Intermediate A25) (667 mg, 1.63 mmol) in DCM (140 mL) at 0° C. to afford the title compound (1.04 g, 34%) as a pale yellow solid.

LCMS m/z 702.4 (M+H)⁺ (ES⁺); 700.5 (M−H)⁻ (ES⁻).

### Step B: 27-oxa-16λ⁶-thia-11,13,14,17,22,29,35-heptaazaheptacyclo-[26.3.1.1^{12,15}.1^{17,19}.1^{19,22}.0^{2,10}.0^{5,9}]pentatriaconta-1(31),2,4,9,12,14,28(32),29-octaene-16,16-dioxide

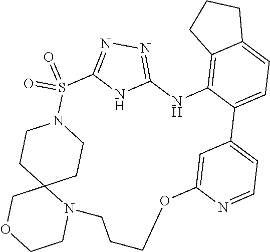 [SMILES: O=S1(=O)c2nnc([nH]2)Nc2c(ccc3c2CCC3)-c2ccnc(c2)OCCCN2CCOCC23CCN1CC3]

1M potassium 2-methylpropan-2-olate in THF (7.73 mL, 7.73 mmol) was added to a solution of 3-(9-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-4-oxa-1,9-diazaspiro-[5.5]undecan-1-yl)propan-1-ol and 3-((4-oxa-1,9-diazaspiro[5.5]undecan-9-yl)-sulfonyl)-N-(5-(2-fluoropyridin-4-yl)-2,3-dihydro-1H-inden-4-yl)-1-((2-(trimethyl-silyl)ethoxy)methyl)-1H-1,2,4-triazol-5-amine (1/1) (1.04 g, 773 μmol) in anhydrous THF (800 mL) at 0° C. The reaction was stirred at this temperature for 45 min before EtOH (25 mL) was added and the reaction mixture concentrated under reduced pressure. The crude product was purified by FC (0-8% (0.7 M ammonia/MeOH)/DCM) to afford protected triazole as an orange oil. The product was taken up in DCM/TFA (1:1, 20 mL) and the reaction stirred at RT for 1.5 h. The volatiles were removed under reduced pressure and traces of TFA were azeotroped with MeOH (3×10 mL). The crude was taken up in DMSO (3 mL) and purified by basic prep HPLC (10-40% MeCN in water) to afford the title compound (23 mg, 4.9%) as a white solid.

LCMS m/z 552.5 (M+H)⁺ (ES⁺); 550.5 (M−H)⁻ (ES⁻).

¹H NMR (500 MHz, DMSO-d₆) δ 8.91 (s, 1H), 8.15 (d, J=5.2 Hz, 1H), 7.27 (d, J=7.8 Hz, 1H), 7.22 (d, J=7.7 Hz, 1H), 6.98 (dd, J=5.2, 1.4 Hz, 1H), 6.65 (s, 1H), 4.21 (t, J=5.4 Hz, 2H), 3.58-3.52 (m, 2H), 3.44 (s, 2H), 3.23-3.13 (m, 2H), 2.96 (t, J=7.5 Hz, 2H), 2.73 (t, J=7.5 Hz, 2H), 2.67-2.58 (m, 2H), 2.46-2.39 (m, 2H), 2.07-1.99 (m, 2H), 1.81-1.65 (m, 4H), 1.41-1.33 (m, 2H). Two aliphatic protons overlapped with water signal in DMSO-d₆. One exchangeable proton not observed.

### Example 21: 20-[(dimethylamino)methyl]-25-oxa-16λ⁶-thia-11,13,14,17,27,32-hexaazahexacyclo[24.3.1.1^{12,15}.1^{17,20}.0^{2,10}.0^{5,9}]dotriaconta-1(29),2,4,9,12,14,26(30),27-octaene-16,16-dioxide

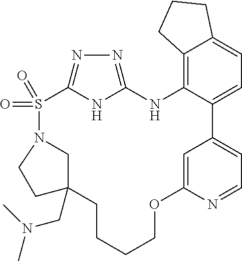 [SMILES: CN(C)CC12CCCCOc3cc(ccn3)-c3ccc4c(c3Nc3nnc([nH]3)S(=O)(=O)N(CC1)C2)CCC4]

### Step A: 4-(3-((dimethylamino)methyl)-1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)-sulfonyl)pyrrolidin-3-yl)butan-1-ol

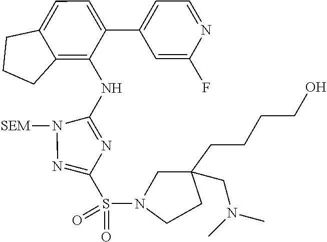 [SMILES: CN(C)CC1(CCCCO)CCN(S(=O)(=O)c2nc(Nc3c(-c4ccnc(F)c4)ccc4c3CCC4)n(COCC[Si](C)(C)C)n2)C1]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (180 mg, 1.35 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.750 g, 1.12 mmol), DIPEA (294 μL, 1.69 mmol) and 4-(3-((dimethylamino)methyl)pyrrolidin-3-yl)butan-1-ol (Intermediate A26) (282 mg, 1.12 mmol) in DCM (20 mL) at 0° C. to afford the title compound (0.222 g, 15%) as a yellow oil.

LCMS m/z 688.5 (M+H)⁺ (ES⁺).

¹H NMR (500 MHz, DMSO-d₆) δ 9.02 (s, 1H), 8.19 (d, J=5.2 Hz, 1H), 7.34-7.26 (m, 3H), 7.08 (s, 1H), 5.40 (s, 2H), 4.34 (t, J=5.1 Hz, 1H), 3.82 (s, 1H), 3.58-3.49 (m, 2H), 3.38-3.33 (m, 2H), 3.28-3.21 (m, 2H), 3.02-2.92 (m, 4H), 2.74-2.64 (m, 2H), 2.11 (d, J=2.1 Hz, 6H), 2.06-1.98 (m, 2H), 1.55 (t, J=7.1 Hz, 2H), 1.32 (p, J=6.7 Hz, 3H), 1.27-1.12 (m, 4H), 0.88-0.80 (m, 2H), −0.03 (s, 9H).

### Step B: 20-[(dimethylamino)methyl]-25-oxa-16λ⁶-thia-11,13,14,17,27,32-hexaazahexacyclo[24.3.1.1^{12,15}.1^{17,20}.0^{2,10}.0^{5,9}]dotriaconta-1(29),2,4,9,12,14,26(30),27-octaene-16,16-dioxide

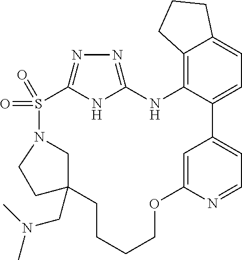 [SMILES: CN(C)CC12CCCCOc3cc(ccn3)-c3ccc4c(c3Nc3nnc([nH]3)S(=O)(=O)N(CC1)C2)CCC4]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1 M KO^{t}Bu in THF (1.09 mL, 1.09 mmol) and 4-(3-((dimethylamino)methyl)-1-((5-((5-(2-fluoro-pyridin-4-yl)-2,3-dihydro-1H-inden-4-yl)amino)-1-((2-(trimethylsilyl)ethoxy)methyl)-1H-1,2,4-triazol-3-yl)sulfonyl)pyrrolidin-3-yl)butan-1-ol (0.222 g, 174 μmol) in THF (250 mL) at 0° C. followed by TFA (3 mL), DCM (3 mL) and basic prep HPLC (10-40% MeCN in water) to afford the title compound (11 mg, 12%) as a white solid.

LCMS m/z 538.5 (M+H)⁺ (ES⁺).

¹H NMR (500 MHz, DMSO-d₆) δ 8.16 (d, J=5.2 Hz, 1H), 7.29 (d, J=7.6 Hz, 1H), 7.24 (d, J=7.7 Hz, 1H), 6.99 (dd, J=5.2, 1.5 Hz, 1H), 6.61 (s, 1H), 4.24 (t, J=5.7 Hz, 2H), 3.10 (d, J=9.7 Hz, 1H), 2.96 (t, J=7.5 Hz, 2H), 2.79-2.67 (m, 3H), 2.28 (m, 1H), 2.21 (s, 6H), 2.10-2.00 (m, 2H), 1.70-1.53 (m, 4H), 1.38-1.21 (m, 4H). Two exchangeable protons not observed, 3 aliphatic protons obscured by water peak.

### Example 22: (19S,21S)-27-oxa-16λ⁶-thia-11,13,14,17,23,29,33-heptaazaheptacyclo-[26.3.1.1^{12,15}.0^{2,10}.0^{5,9}.0^{17,21}.0^{19,23}]tritriaconta-1(31),2,4,9,12,14,28(32),29-octaene-16,16-dioxide

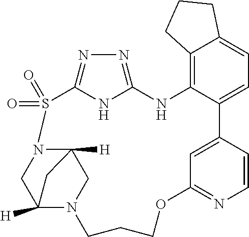 [SMILES: O=S1(=O)c2nnc([nH]2)Nc2c(ccc3c2CCC3)-c2ccnc(c2)OCCCN2C[C@@H]3C[C@H]2CN31]

### Step A: 3-((1S,4S)-5-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-2,5-diazabicyclo-[2.2.1]heptan-2-yl)propan-1-ol

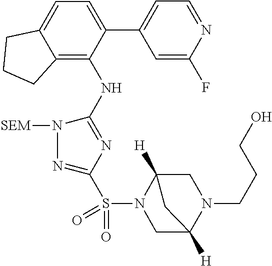 [SMILES: C[Si](C)(C)CCOCn1nc(S(=O)(=O)N2C[C@@H]3C[C@H]2CN3CCCO)nc1Nc1c(-c2ccnc(F)c2)ccc2c1CCC2]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (150 mg, 1.12 mmol), di-sodium (5-(2-fluoropyridin-4-yl)-2,3-dihydro-1H-inden-4-yl)(3-sulfinato-1-((2-(trimethylsilyl)ethoxy)methyl)-1H-1,2,4-triazol-5-yl)-amide (Intermediate A30) (0.500 g, 937 μmol), DIPEA (653 μL, 3.75 mmol) and 3-((1S,4S)-2,5-diazabicyclo[2.2.1]heptan-2-yl)propan-1-ol, di-(2,2,2-trifluoroacetic acid) (Intermediate A27) (282 mg, 1.12 mmol) in DCM (9 mL) at 0° C. to afford the title compound (348 mg, 38%) as a colourless solid.

LCMS m/z 644.5 (M+H)⁺ (ES⁺); 642.4 (M−H)⁻ (ES⁻).

¹H NMR (500 MHz, DMSO-d₆) δ 9.03 (br s, 1H), 8.19 (d, J=5.3 Hz, 1H), 7.36-7.27 (m, 3H), 7.11 (s, 1H), 5.42 (s, 2H), 4.39 (br s, 1H), 4.04 (s, 1H), 3.52 (t, J=8.3 Hz, 2H), 3.45-3.36 (m, 3H), 3.08-2.95 (m, 3H), 2.75-2.66 (m, 3H), 2.48-2.39 (m, 2H), 2.03 (p, J=7.3 Hz, 2H), 1.55-1.41 (m, 3H), 0.91-0.78 (m, 3H), −0.02 (s, 9H). Two aliphatic protons obscured by solvent peak.

### Step B: (19S,21S)-27-oxa-16λ⁶-thia-11,13,14,17,23,29,33-heptaazaheptacyclo-[26.3.1.1^{12,15}.0^{2,10}.0^{5,9}.0^{17,21}.0^{19,23}]tritriaconta-1(31),2,4,9,12,14,28(32),29-octaene-16,16-dioxide

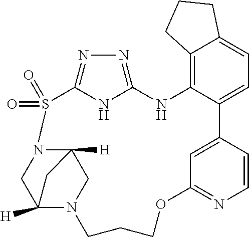 [SMILES: O=S1(=O)c2nnc([nH]2)Nc2c(ccc3c2CCC3)-c2ccnc(c2)OCCCN2C[C@@H]3C[C@H]2CN31]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1.6 M KO^{t}Bu in THF (1.35 mL, 2.17 mmol) and 3-((1S,4S)-5-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)-2,5-diazabicyclo[2.2.1]heptan-2-yl)propan-1-ol (164 mg, 217 μmol) in THF (200 mL) at 0° C. followed by TFA (2.5 mL), DCM (2.5 mL) and basic prep HPLC (20-50% MeCN in water) to afford the title compound (6 mg, 5%) as a white solid.

LCMS m/z 494.3 (M+H)⁺ (ES⁺); 492.3 (M−H)⁻ (ES⁻).

¹H NMR (500 MHz, DMSO-d₆) δ 12.86 (br s, 1H), 9.08 (br s, 1H), 8.20 (d, J=5.2 Hz, 1H), 7.31 (d, J=7.7 Hz, 1H), 7.27 (d, J=7.7 Hz, 1H), 7.01 (dd, J=5.2, 1.4 Hz, 1H), 6.69 (s, 1H), 4.36-4.28 (m, 1H), 4.22 (s, 1H), 4.14-4.03 (m, 1H), 3.50 (s, 1H), 3.30 (s, 1H), 3.24-3.16 (m, 1H), 2.98 (t, J=7.5 Hz, 2H), 2.76 (t, J=7.6 Hz, 2H), 2.66-2.59 (m, 1H), 2.48-2.29 (m, 3H), 2.06 (p, J=7.4 Hz, 2H), 1.85-1.58 (m, 4H).

### Example 23: 11,26-dioxa-16λ⁶-thia-13,14,17,22,28,34-hexaazaheptacyclo-[25.3.1.1^{12,15}.1^{17,19}.1^{19,22}.0^{2,10}.0^{5,9}]tetratriaconta-1(30),2,4,9,12,14,27(31),28-octaene-16,16-dioxide

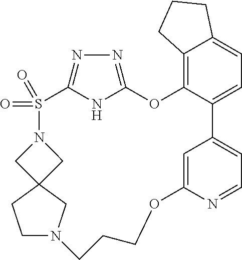 [SMILES: O=S1(=O)c2nnc([nH]2)Oc2c(ccc3c2CCC3)-c2ccnc(c2)OCCCN2CCC3(C2)CN1C3]

### Step A: 3-(2-((5-((5-(2-fluoropyridin-4-yl)-2,3-dihydro-1H-inden-4-yl)oxy)-1-((2-(trimethylsilyl)ethoxy)methyl)-1H-1,2,4-triazol-3-yl)sulfonyl)-2,6-diazaspiro[3.4]-octan-6-yl)propan-1-ol

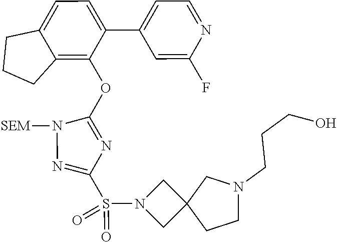 [SMILES: C[Si](C)(C)CCOCn1nc(S(=O)(=O)N2CC3(CCN(CCCO)C3)C2)nc1Oc1c(-c2ccnc(F)c2)ccc2c1CCC2]

NCS (165 mg, 1.24 mmol) was added to a solution of sodium 5-((5-(2-fluoropyridin-4-yl)-2,3-dihydro-1H-inden-4-yl)oxy)-1-((2-(trimethylsilyl)ethoxy)methyl)-1H-1,2,4-triazole-3-sulfinate (600 mg, 1.03 mmol) (Intermediate A28) in DCM (9 mL) and stirred at 0° C. for 30 min. 3-(2,6-Diazaspiro[3.4]octan-6-yl)propan-1-ol bis(2,2,2-trifluoroacetate) (1.03 g, 1.55 mmol) (Intermediate A31) was dissolved in DCM (3 mL) and DIPEA (0.74 mL, 4.12 mmol) was added. The resulting solution was added to the reaction and stirred at 0° C. for 1 h. The reaction mixture was diluted with water (20 mL) and extracted with EtOAc (3×50 mL). The combined organics were washed with brine (50 mL), dried using a phase separator, then concentrated in vacuo. The crude product was purified by FC (0-100% MeOH/DCM) to afford the title compound (245 mg, 29%) as a colourless solid.

LCMS m/z 659.5 (M+H)⁺ (ES⁺); 657.4 (M−H)⁻ (ES⁻).

¹H NMR (500 MHz, DMSO-d₆) δ 8.24 (d, J=5.2 Hz, 1H), 4.17-4.04 (m, 3H), 7.27 (s, 1H), 5.59 (s, 2H), 4.38 (br s, 1H), 3.77-3.67 (m, 4H), 3.64 (t, J=8.1 Hz, 2H), 3.44-3.36 (m, 2H), 3.02 (t, J=7.5 Hz, 2H), 2.74 (t, J=7.5 Hz, 2H), 2.43-2.29 (m, 6H), 2.09 (p, J=7.4 Hz, 2H), 1.63 (t, J=7.0 Hz, 2H), 1.51 (p, J=7.0 Hz, 2H), 0.87 (t, J=8.0 Hz, 2H), −0.03 (s, 9H).

### Step B: 11,26-dioxa-16λ⁶-thia-13,14,17,22,28,34-hexaazaheptacyclo-[25.3.1.1^{12,15}.1^{17,19}.1^{19,22}.0^{2,10}.0^{5,9}]tetratriaconta-1(30),2,4,9,12,14,27(31),28-octaene-16,16-dioxide

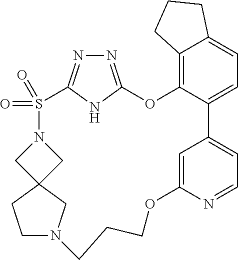 [SMILES: O=S1(=O)c2nnc([nH]2)Oc2c(ccc3c2CCC3)-c2ccnc(c2)OCCCN2CCC3(C2)CN1C3]

KO^{t}Bu 1.6M in THF (1.96 mL, 3.13 mmol) was added to a solution of 3-(2-((5-((5-(2-fluoropyridin-4-yl)-2,3-dihydro-1H-inden-4-yl)oxy)-1-((2-(trimethylsilyl)ethoxy)-methyl)-1H-1,2,4-triazol-3-yl)sulfonyl)-2,6-diazaspiro[3.4]-octan-6-yl)propan-1-ol (243 mg, 313 μmol) in anhydrous THF (200 mL) at 0° C. and the reaction was stirred at this temperature for 30 min. To the reaction mixture was added water (150 mL) and the reaction was extracted with EtOAc (2×100 mL), dried using a phase separator and concentrated in vacuo. The resulting crude product was purified by FC (0-100% (0.7 M ammonia/MeOH)/DCM) to afford the SEM-protected product. This was taken up in 1:1 DCM:TFA (5 mL) and stirred at RT for 2 h, before being concentrated in vacuo and purified by basic prep HPLC (10-40% MeCN in water) to afford the title compound (13 mg, 7%) as a light yellow solid LCMS m/z 509.4 (M+H)⁺ (ES⁺); 507.3 (M−H)⁻ (ES⁻).

¹H NMR (500 MHz, DMSO-d₆) δ 8.23 (d, J=5.3 Hz, 1H), 7.33 (d, J=7.6 Hz, 1H), 7.22 (d, J=7.5 Hz, 1H), 7.13 (d, J=5.3 Hz, 1H), 6.91 (s, 1H), 4.32-4.21 (m, 2H), 3.87-3.62 (m, 6H), 3.04-2.88 (m, 4H), 2.47 (t, J=7.0 Hz, 2H), 2.23 (t, J=7.3 Hz, 2H), 2.05-1.93 (m, 4H). One exchangeable proton not observed. Two protons obscured by DMSO peak.

### Example 24: 4-fluoro-6-(propan-2-yl)-23-oxa-13λ⁶-thia-8,10,11,14,19,25,31-heptaazahexacyclo[22.3.1.1^{9,12}.1^{14,16}.1^{16,19}.0^{2,7}]hentriaconta-1(27),2(7),3,5,9,11,24(28),25-octaene-13,13-dioxide

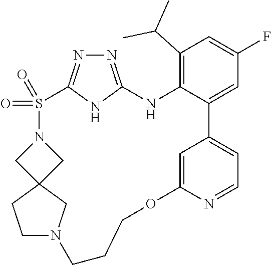 [SMILES: CC(C)c1cc(F)cc2c1Nc1nnc([nH]1)S(=O)(=O)N1CC3(CCN(CCCOc4cc-2ccn4)C3)C1]

### Step A: 3-(2-((5-((4-fluoro-2-(2-fluoropyridin-4-yl)-6-isopropylphenyl)amino)-1-((2-(trimethylsilyl)ethoxy)methyl)-1H-1,2,4-triazol-3-yl)sulfonyl)-2,6-diazaspiro[3.4]-octan-6-yl)propan-1-ol

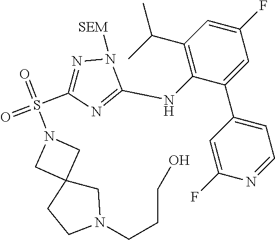 [SMILES: CC(C)c1cc(F)cc(-c2ccnc(F)c2)c1Nc1nc(S(=O)(=O)N2CC3(CCN(CCCO)C3)C2)nn1COCC[Si](C)(C)C]

Synthesized according to the procedure outlined for 2-((1-((5-((5-(2-fluoropyridin-4-yl)-2,3-dihydro-1H-inden-4-yl)amino)-1-((2-(trimethylsilyl)ethoxy)methyl)-1H-1,2,4-triazol-3-yl)sulfonyl)piperidin-4-yl)(methyl)amino)ethan-1-ol (Example 9, Step A), from NCS (148 mg, 1.11 mmol), di-sodium (4-fluoro-2-(2-fluoropyridin-4-yl)-6-isopropylphenyl)(3-sulfinato-1-((2-(trimethylsilyl)ethoxy)methyl)-1H-1,2,4-triazol-5-yl)amide (Intermediate A29) (0.600 g, 921 μmol), DIPEA (700 μL, 4.02 mmol) and 3-(2,6-diazaspiro[3.4]octan-6-yl)propan-1-ol (261 mg, 921 μmol) (Intermediate A7) in DCM (35 mL) at 0° C. to afford the title compound (0.120 g, 11%) as a thick yellow oil.

LCMS m/z 678.5 (M+H)⁺ (ES⁺).

### Step B: 4-fluoro-6-(propan-2-yl)-23-oxa-13λ⁶-thia-8,10,11,14,19,25,31-heptaazahexacyclo[22.3.1.1^{9,12}.1^{14,16}.1^{16,19}.0^{2,7}]hentriaconta-1(27),2(7),3,5,9,11,24(28),25-octaene-13,13-dioxide

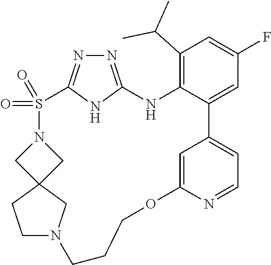 [SMILES: CC(C)c1cc(F)cc2c1Nc1nnc([nH]1)S(=O)(=O)N1CC3(CCN(CCCOc4cc-2ccn4)C3)C1]

Synthesized according to the procedure outlined for 25-methyl-22-oxa-2λ⁶-thia-1,4,5,7,20,25,32-heptaazahexacyclo[24.2.2.1^{3,6}.1^{17,21}.0^{8,16}.0^{9,13}]dotriaconta-3,5,8,13,15,17,19,21(31)-octaene-2,2-dioxide (Example 9, Step B) from 1.6 M KO^{t}Bu in THF (1.11 mL, 1.77 mmol) and 3-(2-((5-((4-fluoro-2-(2-fluoropyridin-4-yl)-6-isopropylphenyl)amino)-1-((2-(trimethylsilyl)ethoxy)methyl)-1H-1,2,4-triazol-3-yl)-sulfonyl)-2,6-diazaspiro[3.4]octan-6-yl)propan-1-ol (120 mg, 117 μmol) in THF (145 mL) at 0° C. followed by TFA (3 mL), DCM (3 mL) and basic prep HPLC (3-35% MeCN in water) to afford the title compound (1.1 mg, 1%) as a white solid.

LCMS m/z 528.1 (M+H)⁺ (ES⁺).

¹H NMR (500 MHz, DMSO-d₆) δ 8.17-8.11 (m, 1H), 7.32 (dd, J=10.0, 3.0 Hz, 1H), 7.13 (dd, J=8.8, 3.0 Hz, 1H), 7.03 (dd, J=5.3, 1.5 Hz, 1H), 6.76 (s, 1H), 6.53 (s, 1H), 4.33-4.24 (m, 2H), 3.70-3.55 (m, 4H), 3.19-3.10 (m, 2H), 2.41-2.32 (m, 1H), 2.22 (s, 2H), 1.99 (t, J=7.1 Hz, 2H), 1.81-1.72 (m, 2H), 1.20-1.07 (m, 6H). Two aliphatic protons overlapped with DMSO-d₆ signal; one exchangeable proton not observed.

### Examples —Biological Studies

NLRP and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1R) from the cell.

THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 g/ml Final Assay Concentration (FAC). 40 μl of the final preparation was aliquoted into each well of a 96-well plate. The plate thus prepared was used for compound screening.

THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hrs at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters) 96-well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

| High | MCC950 (10 uM) | Compound 8-point half-log dilution |
|---|---|---|
| Low | Drug free control | |

The results of the pyroptosis assay are summarised in Table 1 below as THP IC₅₀.

Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 1 below as HWB IC₅₀.

TABLE 1
NLRP3 inhibitory activity (≤0.1 μM = ‘+++++’, ≤0.5
μM = ‘++++’, ≤1 μM = ‘+++’, ≤5
μM = ‘++’, ≤10 μM = not determined = ‘ND’).

| Example | THP | HWB |
|---|---|---|
| No | IC₅₀ | IC₅₀ |
| 1 | ++ | ++ |
| 2† | +++++ | +++++ |
| 3 | +++++ | ++++ |
| 4 | +++++ | ++++ |
| 5† | ++++ | ++++ |
| 6 | ++ | ND |
| 7 | + | ND |
| 8 | ++ | + |
| 9 | ++++ | ++++ |
| 10 | +++++ | ++++ |
| 11 | +++++ | ++++ |
| 12 | ++++ | ++++ |
| 13 | +++ | ND |
| 14 | ++++ | ND |
| 15 | +++++ | ND |
| 16 | +++ | ND |
| 17 | ++++ | ND |
| 18 | ++ | ND |
| 19 | ++++ | ND |
| 20 | ++++ | ND |
| 21 | +++ | ND |
| 22 | ++ | ND |
| 23 | ++ | ND |
| 24 | +++++ | ND |

† = tested as the formic acid salt.

As is evident from the results presented in Table 1, surprisingly in spite of the structural differences versus the prior art compounds, the compounds of the invention show high levels of NLRP3 inhibitory activity in the pyroptosis assay and in the human whole blood assay.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 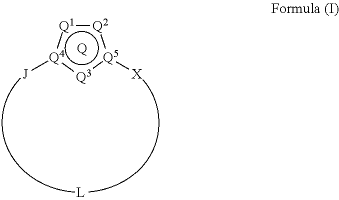
or a pharmaceutically acceptable salt or solvate thereof, wherein:
J is —SO—, —SO₂—, —SO(═NH)— or —SO(═NR^{j})—;
Q¹ and Q² are each independently selected from O, S, N, NH, NR^{q}, CH, CHal or CR^{qq}, provided that at least one of Q¹ and Q² is selected from N, NH and NR^{q};
Q³ is selected from O, S, N, NH and NR^{q}; and
Q⁴ and Q⁵ are each independently selected from C and N, provided that at least one of Q⁴ and Q⁵ is C;
such that ring Q is a 5-membered heteroaryl ring;
X is —O—, —NH—, —NR^{x}—, —CH₂—, —CH(Hal)-, —C(Hal)₂-, —CH(R^{xx})—, —C(Hal)(R^{xx})— or —C(R^{xx})₂—;
L is a saturated or unsaturated hydrocarbylene group, wherein the hydrocarbylene group may be straight-chained or branched, or be or include one or more cyclic groups, wherein the hydrocarbylene group may optionally be substituted, and wherein the hydrocarbylene group may optionally include one or more heteroatoms independently selected from N, O and S in its carbon skeleton;
-J-, ring Q, —X— and -L- together form a ring, such that the minimum single ring size that encompasses all or part of each of -J-, ring Q, —X— and -L- is from 8 to 30 atoms;
each R^{j}, R^{q} and R^{x} is independently selected from a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton;
each R^{qq} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton;
each R^{xx} is independently selected from —OH, —NO₂, —NH₂, —N₃, —SH, —SO₂H, —SO₂NH₂, or a saturated or unsaturated hydrocarbyl group, wherein the hydrocarbyl group may be straight-chained or branched, or be or include one or more cyclic groups, wherein the hydrocarbyl group may optionally be substituted, and wherein the hydrocarbyl group may optionally include one or more heteroatoms independently selected from N, O and S in its carbon skeleton, or any two R^{x} may, together with the carbon atom to which they are attached, form a saturated or unsaturated cyclic group, wherein the cyclic group may optionally be substituted; and
each Hal is independently selected from F, Cl, Br or I.

2. The compound or pharmaceutically acceptable salt or solvate thereof, as claimed in claim 1, wherein:
(i) J is —SO₂—; and/or
(ii) Q¹ and Q² are each independently selected from N, NH and NR^{q}, optionally wherein Q¹ and Q² are both N; and/or
(iii) Q³ is selected from O, S, N and NH, optionally wherein Q³ is NH; and/or
(iv) Q⁴ and Q⁵ are both C.

3-7. (canceled)

8. The compound or pharmaceutically acceptable salt or solvate thereof, as claimed in claim 1, wherein:
(i) X is —O—, —NH—, —NR^{x}—, —CH₂—, —CH(F)—, —CH(Cl)— or —CH(R^{xx})—; or
(ii) X is —O— or —NH—; or
(iii) X is —NH—.

9-10. (canceled)

11. The compound or pharmaceutically acceptable salt or solvate thereof, as claimed in claim 1, wherein the compound has the formula (Ia): 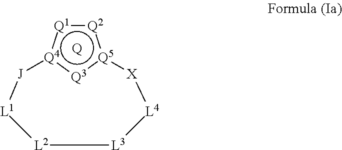
wherein:
J, Q¹, Q², Q³, Q⁴, Q⁵, ring Q and X are as previously defined;
-J-, ring Q, —X—, -L¹-, -L²-, -L³- and -L⁴- together form a ring, such that the minimum single ring size that encompasses all or part of each of -J-, ring Q, —X—, -L¹-, -L²-, -L³- and -L⁴- is from 8 to 30 atoms;
L¹ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents;
L² is an alkylene, alkenylene or alkynylene group, wherein the alkylene, alkenylene or alkynylene group may be straight-chained or branched, or be or include one or more cyclic groups, wherein one or more carbon atoms in the backbone of the alkylene, alkenylene or alkynylene group may optionally be replaced by one or more heteroatoms independently selected from N, O and S, and wherein the alkylene, alkenylene or alkynylene group may optionally be substituted with one or more monovalent substituents, and/or one or more π-bonded substituents;
L³ is a bond, a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents; and
L⁴ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents.

12. The compound or pharmaceutically acceptable salt or solvate thereof, as claimed in claim 11, wherein:
(i) L¹ is a divalent 3- to 7-membered monocyclic group, a divalent 5- to 12-membered bicyclic group, or a divalent 7- to 18-membered tricyclic group, any of which may optionally be substituted with one or more monovalent substituents and/or π-bonded substituents; or
(ii) L¹ is a divalent 5- to 12-membered spiro bicyclic group, which may optionally be substituted with one or more monovalent substituents and/or π-bonded substituents.

13. (canceled)

14. The compound or pharmaceutically acceptable salt or solvate thereof, as claimed in claim 11, wherein the ring of the divalent monocyclic, bicyclic or tricyclic group of L⁴ that is directly attached to X is aromatic.

15. The compound or pharmaceutically acceptable salt or solvate thereof, as claimed in claim 1, wherein the compound has the formula (Ic) or (Ic′): 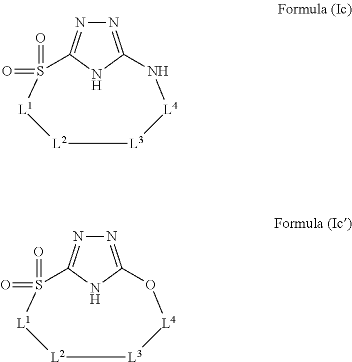
wherein:
L¹ is a divalent 3- to 7-membered monocyclic group, or a divalent 7- to 11-membered bicyclic group, wherein the divalent 3- to 7-membered monocyclic group or divalent 7- to 11-membered bicyclic group may optionally be substituted with one or more halo groups and/or one or more oxo (═O) groups and/or one or more substituents R^{L};
L² is an alkylene or alkenylene group, wherein the alkylene or alkenylene group may be straight-chained or branched, or be or include one or more cyclic groups, wherein one or more carbon atoms in the backbone of the alkylene or alkenylene group may optionally be replaced by one or more heteroatoms independently selected from N and O, and wherein the alkylene or alkenylene group may optionally be substituted with one or more halo groups and/or one or more oxo (═O) groups;
L³ is a divalent phenyl or 5- or 6-membered heteroaryl group, wherein the divalent phenyl or 5- or 6-membered heteroaryl group may optionally be substituted with one or more halo groups and/or one or more substituents R^{L};
L⁴ is a divalent phenyl or 5- or 6-membered heteroaryl group, wherein the divalent phenyl or 5- or 6-membered heteroaryl group may optionally be substituted with one or more halo groups and/or one or more substituents R^{L};
for a compound having the formula (Ic), the ring atom of L⁴ that is directly attached to L³ is at the α-position relative to the ring atom of L⁴ that is directly attached to the nitrogen atom of the —NH— group;
for a compound having the formula (Ic′), the ring atom of L⁴ that is directly attached to L³ is at the α-position relative to the ring atom of L⁴ that is directly attached to the oxygen atom of the —O— group;
each R^{L} is independently selected from a C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R^{II}—SO₂R¹³ or —R^{II}—SO₂N(R¹³)₂ group, and/or any two R^{L} attached to the same divalent phenyl or 5- or 6-membered heteroaryl group of L³ or L⁴ may, together with the atoms of the divalent phenyl or 5- or 6-membered heteroaryl group to which they are attached, form a fused 5- or 6-membered cyclic group, wherein the fused 5- or 6-membered cyclic group may optionally be substituted with one or more halo groups and/or one or two oxo (═O) groups and/or one, two or three substituents independently selected from a C₁-C₆ alkyl, C₁-C₆ haloalkyl, C₂-C₆ alkenyl, C₂-C₆ haloalkenyl, —R¹¹—R¹², —R¹¹—CN, —R¹¹—N(R¹³)₂, —R¹¹—OR¹³, —R¹¹—COR¹³, —R¹¹—COOR¹³, —R¹¹—CON(R¹³)₂, —R¹¹—C(═NR¹³)R¹³, —R¹¹—C(═NR¹³)N(R¹³)₂, —R¹¹—C(═NOR¹³)R¹³, —R¹¹—SO₂R¹³ or —R¹¹—SO₂N(R¹³)₂ group;
each R¹¹ is independently selected from a bond, or a C₁-C₄ alkylene group, wherein the C₁-C₄ alkylene group may be straight-chained or branched, or be or include a C₃-C₄ cycloalkylene group, and wherein the C₁-C₄ alkylene group may optionally be substituted with one or more halo groups;
each R¹² is independently selected from a 3- to 6-membered cyclic group, wherein the 3- to 6-membered cyclic group may optionally be substituted with one or more halo groups and/or one, two or three substituents independently selected from —CN, —R¹⁴, —OH, —OR¹⁴, —NH₂, —NHR¹⁴ and —N(R¹⁴)₂;
each R¹³ is independently selected from hydrogen or a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups and/or one, two or three substituents independently selected from —CN, —R¹⁴, —OH, —OR¹⁴, —NH₂, —NHR¹⁴ and —N(R¹⁴)₂, or any two R¹³ attached to the same nitrogen atom may together form a C₂-C₅ alkylene or C₂-C₅ haloalkylene group;
each R¹⁴ is independently selected from a C₁-C₄ alkyl or C₁-C₄ haloalkyl group; for a compound having the formula (Ic), the minimum single ring size that encompasses all or part of each of -L¹-, -L²-, -L³-, -L⁴- and 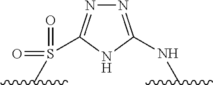 [SMILES: *Nc1nnc(S(*)(=O)=O)[nH]1]
is from 8 to 30 atoms; and
for a compound having the formula (Ic′), the minimum single ring size that encompasses all or part of each of -L¹-, -L²-, -L³-, -L⁴- and 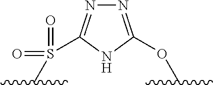 [SMILES: *Oc1nnc(S(*)(=O)=O)[nH]1]
is from 8 to 30 atoms.

16. The compound or pharmaceutically acceptable salt or solvate thereof, as claimed in claim 15, wherein:
(i) the divalent phenyl or 5- or 6-membered heteroaryl group of L⁴ is substituted at the α′-position, relative to the ring atom of L⁴ that is directly attached to the nitrogen atom of the —NH— group where the compound has the formula (Ic), or relative to the ring atom of L⁴ that is directly attached to the oxygen atom of the —O— group where the compound has the formula (Ic′), with a C₁-C₆ alkyl, C₁-C₆ haloalkyl, C₂-C₆ alkenyl, C₂-C₆ haloalkenyl, or 3- to 6-membered cyclic group, wherein the 3- to 6-membered cyclic group may optionally be substituted with one or more halo groups; or
(ii) the divalent phenyl or 5- or 6-membered heteroaryl group of L⁴ is ortho-fused to a 5- or 6-membered cyclic group across the α′,β′-positions, relative to the ring atom of L⁴ that is directly attached to the nitrogen atom of the —NH— group where the compound has the formula (Ic), or relative to the ring atom of L⁴ that is directly attached to the oxygen atom of the —O— group where the compound has the formula (Ic′), wherein the ortho-fused 5-or 6-membered cyclic group is optionally substituted with one or more halo groups.

17. The compound or pharmaceutically acceptable salt or solvate thereof, as claimed in claim 1, wherein the minimum single ring size that encompasses:
all or part of each of -J-, ring Q, —X— and -L-, is from 12 to 24 atoms, or from 14 to 20 atoms.

18. (canceled)

19. The compound or pharmaceutically acceptable salt or solvate thereof, as claimed in claim 1, wherein the compound:
(i) has the formula (Id): 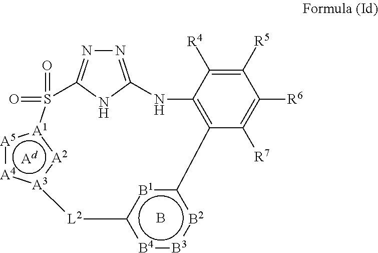
wherein:
A¹ and A³ are each independently selected from C and N, and A², A⁴ and A⁵ are each independently selected from N, CH, CY¹, CR^{A}, NH and NR^{A}, such that ring A^{d} is a 5-membered heteroaryl ring containing one, two or three nitrogen atoms in its ring structure;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₅ alkylene or C₁-C₅ fluoroalkylene group may optionally be replaced by a single oxygen atom;
R⁴ is selected from a C₁-C₄ alkyl, C₁-C₄ fluoroalkyl, C₃-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group, or
(ii) has the formula (e): 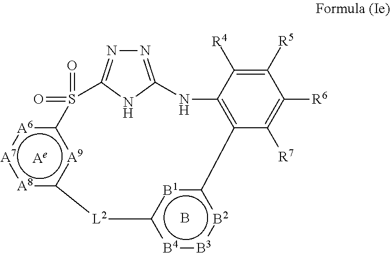
wherein:
A⁶, A⁷, A⁸ and A⁹ are each independently selected from N, CH, CY¹ and CR^{A}, such that ring A^{e} is a 6-membered aryl ring or a 6-membered heteroaryl ring containing one, two or three nitrogen atoms in its ring structure;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{A} is independently selected from —OH, —NH₂, —CN or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{A} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₄ alkylene or C₁-C₅ fluoroalkylene group may optionally be replaced by a single oxygen atom;
R⁴ is selected from a C₁-C₄ alkyl, C₁-C₄ fluoroalkyl, C₁-C₆ cycloalkyl or C₁-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group; or
(iii) has the formula (If): 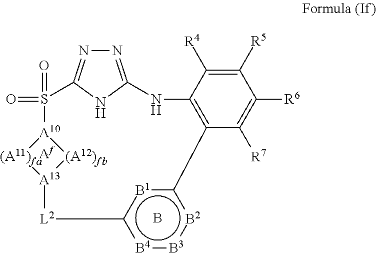
wherein:
A¹⁰ and A¹³ are each independently selected from N, CH, CY² and CR^{AA}, and each A¹ and A¹² is independently selected from O, NH, NR^{AAA}, C═O, CH₂, CH(Y²), CH(R^{AA}), C(Y²)₂, C(Y²)(R^{AA}) and C(R^{AA})₂, such that ring A^{f} contains one or two atoms independently selected from oxygen and nitrogen in its ring structure;
fa is 1, 2 or 3, and fb is 1, 2 or 3, provided that fa+fb≤5;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₄ alkylene or C₁-C₅ fluoroalkylene group may optionally be replaced by a single oxygen atom;
R⁴ is selected from a C₃-C₄ alkyl, C₁-C₄ fluoroalkyl, C₁-C₆ cycloalkyl or C₆-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group; or
(iv) has the formula (Ig): 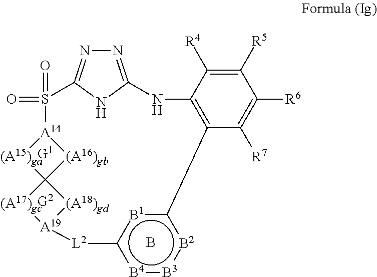
wherein:
A¹⁴ and A¹⁹ are each independently selected from N, CH, CY² and CR^{AA}, and each A¹⁵, A¹⁶, A¹⁷ and A¹⁸ is independently selected from O, NH, NR^{AAA}, C═O, CH₂, CH(Y²), CH(R^{AA}), C(Y²)₂, C(Y²)(R^{AA}) and C(R^{AA})₂, such that ring G¹ contains zero, one or two atoms independently selected from oxygen and nitrogen in its ring structure, and ring G² contains zero, one or two atoms independently selected from oxygen and nitrogen in its ring structure;
ga is 0, 1, 2, 3 or 4 and gb is 0, 1, 2, 3 or 4 provided that 1≤ga+gb≤5;
gc is 0, 1, 2, 3, or 4 and gd is 0, 1, 2, 3, or 4 provided that 1≤gc+gd≤5;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl, C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₄ alkylene or C₁-C₅ fluoroalkylene group may optionally be replaced by a single oxygen atom;
R⁴ is selected from a C₁-C₄ alkyl, C₁-C₄ fluoroalkyl, C₁-C₆ cycloalkyl or C₃-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group; or
(v) has the formula (Ig′): 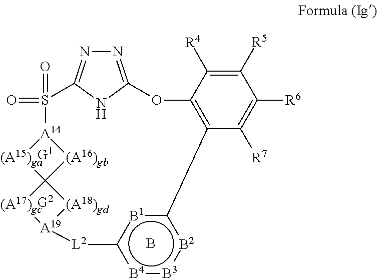
wherein:
A¹⁴ and A¹⁹ are each independently selected from N, CH, CY² and CR^{AA}, and each A¹⁵, A¹⁶, A¹⁷ and A¹⁸ is independently selected from O, NH, NR^{AAA}, C═O, CH₂, CH(Y²), CH(R^{AA}), C(Y²)₂, C(Y²)(R^{AA}) and C(R^{AA})₂, such that ring G¹ contains zero, one or two atoms independently selected from oxygen and nitrogen in its ring structure, and ring G² contains zero, one or two atoms independently selected from oxygen and nitrogen in its ring structure;
ga is 0, 1, 2, 3, or 4 and gb is 0, 1, 2, 3, or 4 provided that 1≤ga+gb≤5;
gc is 0, 1, 2, 3, or 4 and gd is 0, 1, 2, 3, or 4 provided that 1≤gc+gd≤5;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₄ alkylene or C₁-C₅ fluoroalkylene group may optionally be replaced by a single oxygen atom;
R⁴ is selected from a C₁-C₄ alkyl, C₁-C₄ fluoroalkyl, C₁-C₆ cycloalkyl or C₁-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group; or
(vi) has the formula (Ih): 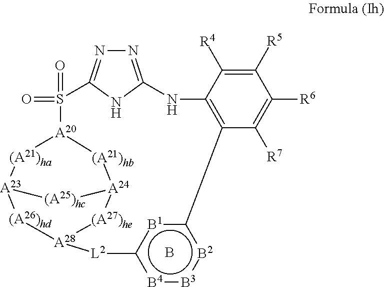
wherein:
each A²⁰, A²³, A²⁴ and A²⁸ is independently selected from N, CH, CY² and CR^{AA}, and each A²¹, A²², A²⁵, A²⁶ and A²⁷ is independently selected from O, NH, NR^{AAA}, C═O, CH₂, CH(Y²), CH(R^{AA}), C(Y²)₂, C(Y²)(R^{AA}) and C(R^{AA})₂, such that the divalent bridged bicyclic group defined by A²⁰, A²¹, A²², A²³, A²⁴, A²⁵, A²⁶, A²⁷ and A²⁸ contains zero, one, two or three atoms independently selected from oxygen and nitrogen in its ring structure;
ha is 0, 1 or 2;
hb is 0, 1 or 2;
he is 1, 2 or;
hd is 0, 1 or 2;
he is 0, 1 or 2;
1≤ha+hb+he+hd+he≤7;
B¹, B², B³ and B⁴ are each independently selected from N, CH, CY¹ and CR^{B}, such that ring B is a 6-membered aryl ring or a 6-membered heteroaryl ring containing one, two or three nitrogen atoms in its ring structure;
each R^{AA} is independently selected from —OH, —NH₂, —CN, or a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AA} contains, in total, from 1 to 6 carbon, nitrogen and oxygen atoms;
each R^{AAA} is independently selected from a saturated hydrocarbyl group, wherein the saturated hydrocarbyl group is straight-chained or branched, or is or includes a cyclic group, wherein the saturated hydrocarbyl group optionally includes one or two heteroatoms independently selected from O and N in its carbon skeleton, wherein the saturated hydrocarbyl group is optionally substituted with one or more fluoro groups and/or one or two oxo (═O) groups, and wherein each R^{AAA} contains, in total, from 1 to 6 carbon, nitrogen and oxygen atoms;
each R^{B} is independently selected from a —CN, —R^{B1}, —OH, —OR^{B1}, —NH₂, —NHR^{B1} or —N(R^{B1})₂ group, wherein each R^{B1} is independently selected from a C₁-C₄ alkyl or C₁-C₄ fluoroalkyl group;
each Y¹ and Y² is independently selected from F, Cl or Br;
L² is a straight-chained alkylene group, wherein the straight-chained alkylene group optionally includes one or two heteroatoms independently selected from O and N in its carbon skeleton, wherein L² has a chain length of from 2 to 8 atoms, and wherein L² may optionally be substituted with one or more fluoro groups and/or one or two oxo (═O) groups and/or one or more groups R^{L2}, wherein each R^{L2} is independently selected from a C₁-C₄ alkyl, —O—C₁-C₄ alkyl C₁-C₄ fluoroalkyl or —O—C₁-C₄ fluoroalkyl group, or wherein any two R^{L2} may together form a C₁-C₅ alkylene or C₁-C₅ fluoroalkylene group, wherein one carbon atom in the backbone of the C₁-C₄ alkylene or C₁-C₅ fluoroalkylene group may optionally be replaced by a single oxygen atom;
R⁴ is selected from a C₁-C₄ alkyl, C₁-C₄ fluoroalkyl, C₁-C₆ cycloalkyl or C₁-C₆ fluorocycloalkyl group, and R⁵ is selected from hydrogen, F, Cl, Br or a methyl or fluoromethyl group, or R⁴ and R⁵ together form a divalent group selected from —CH₂CH₂CH₂—, —CH═CHCH₂—, —CH₂CH═CH—, —CH₂CH₂O— and —OCH₂CH₂—, wherein the divalent group formed by R⁴ and R⁵ may optionally be fluoro-substituted; and
R⁶ and R⁷ are each independently selected from hydrogen, F, Cl, Br or a methyl or fluoromethyl group.

20-24. (canceled)

25. The compound or pharmaceutically acceptable salt or solvate thereof, as claimed in claim 1, which is (a) a compound selected from the group consisting of: 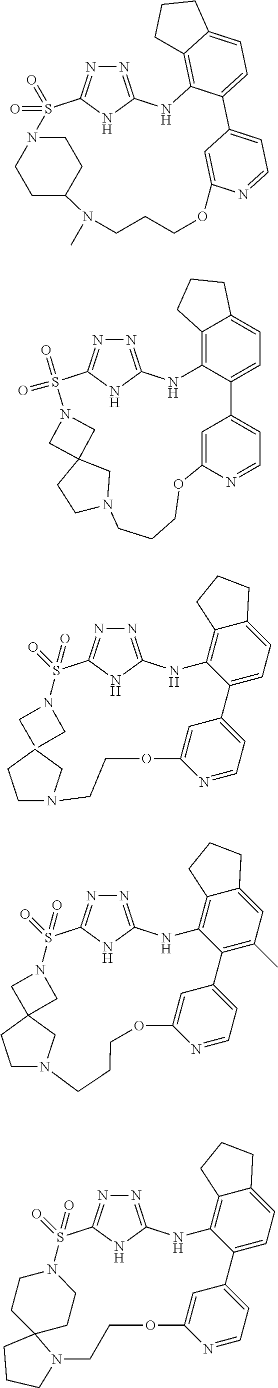 [SMILES: CN1CCCOc2cc(ccn2)-c2ccc3c(c2Nc2nnc([nH]2)S(=O)(=O)N2CCC1CC2)CCC3 ; O=S1(=O)c2nnc([nH]2)Nc2c(ccc3c2CCC3)-c2ccnc(c2)OCCCN2CCC3(C2)CN1C3 ; O=S(=O)(c1nnc(Nc2c(-c3ccnc(OCCN4CCCC4)c3)ccc3c2CCC3)[nH]1)N1CCC1 ; Cc1cc2c(c3c1-c1ccnc(c1)OCCCN1CCC4(C1)CN(C4)S(=O)(=O)c1nnc([nH]1)N3)CCC2 ; O=S1(=O)c2nnc([nH]2)Nc2c(ccc3c2CCC3)-c2ccnc(c2)OCCN2CCCC23CCN1CC3] 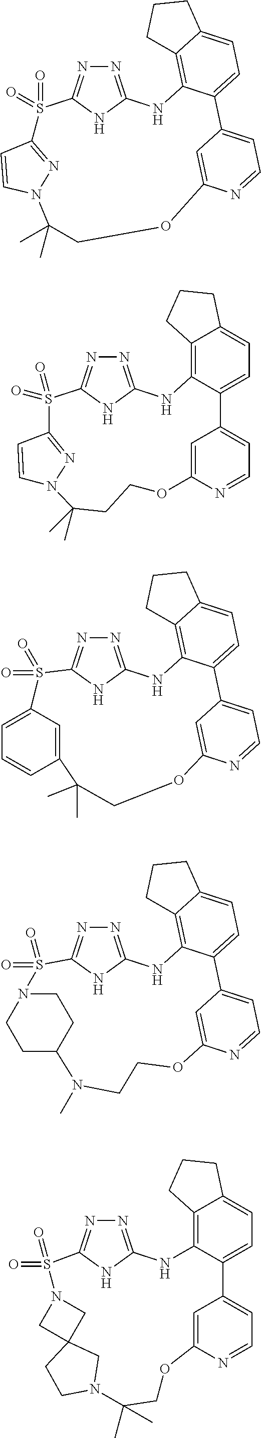 [SMILES: CC1(C)COc2cc(ccn2)-c2ccc3c(c2Nc2nnc([nH]2)S(=O)(=O)c2ccn1n2)CCC3 ; CC1(C)CCOc2cc(ccn2)-c2ccc3c(c2Nc2nnc([nH]2)S(=O)(=O)c2ccn1n2)CCC3 ; CC1(C)COc2cc(ccn2)-c2ccc3c(c2Nc2nnc([nH]2)S(=O)(=O)c2cccc1c2)CCC3 ; CN1CCOc2cc(ccn2)-c2ccc3c(c2Nc2nnc([nH]2)S(=O)(=O)N2CCC1CC2)CCC3 ; CC1(C)COc2cc(ccn2)-c2ccc3c(c2Nc2nnc([nH]2)S(=O)(=O)N2CC4(CCN1C4)C2)CCC3] 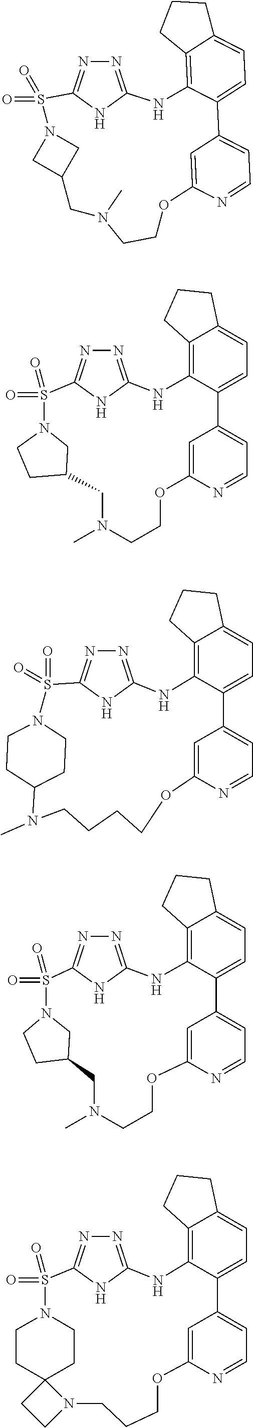 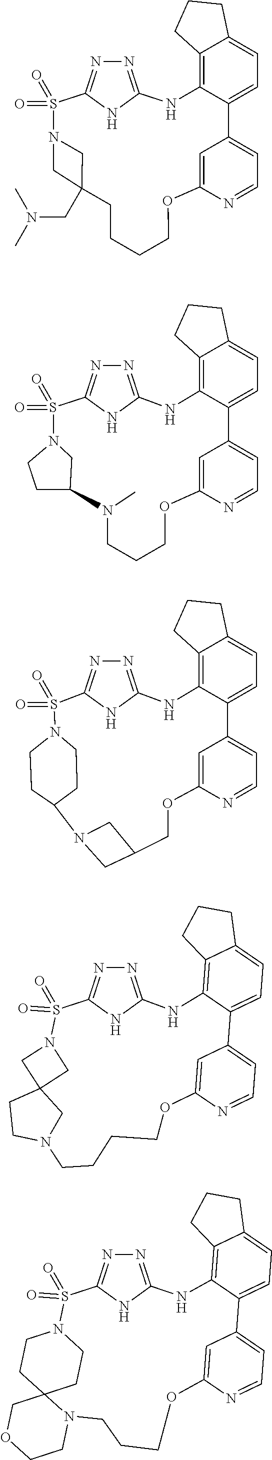 [SMILES: CN(C)CC12CCCCOc3cc(ccn3)-c3ccc4c(c3Nc3nnc([nH]3)S(=O)(=O)N(C1)C2)CCC4 ; CN1CCCOc2cc(ccn2)-c2ccc3c(c2Nc2nnc([nH]2)S(=O)(=O)N2CC[C@H]1C2)CCC3 ; O=S1(=O)c2nnc([nH]2)Nc2c(ccc3c2CCC3)-c2ccnc(c2)OCC2CN(C2)C2CCN1CC2 ; O=S1(=O)c2nnc([nH]2)Nc2c(ccc3c2CCC3)-c2ccnc(c2)OCCCCN2CCC3(C2)CN1C3 ; O=S1(=O)c2nnc([nH]2)Nc2c(ccc3c2CCC3)-c2ccnc(c2)OCCCN2CCOCC23CCN1CC3] 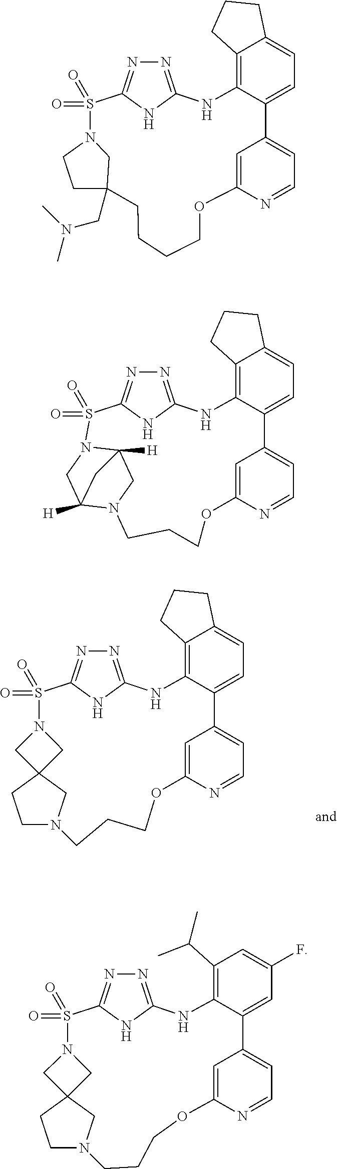 [SMILES: CN(C)CC12CCCCOc3cc(ccn3)-c3ccc4c(c3Nc3nnc([nH]3)S(=O)(=O)N(CC1)C2)CCC4 ; O=S1(=O)c2nnc([nH]2)Nc2c(ccc3c2CCC3)-c2ccnc(c2)OCCCN2C[C@@H]3C[C@H]2CN31 ; O=S1(=O)c2nnc([nH]2)Nc2c(ccc3c2CCC3)-c2ccnc(c2)OCCCN2CCC3(C2)CN1C3 ; CC(C)c1cc(F)cc2c1Nc1nnc([nH]1)S(=O)(=O)N1CC3(CCN(CCCOc4cc-2ccn4)C3)C1]
or (b) a pharmaceutically acceptable salt or solvate of the selected compound.

26. A prodrug of the compound as claimed in claim 1, or a pharmaceutically acceptable salt or solvate thereof.

27. A pharmaceutical composition comprising the compound or the pharmaceutically acceptable salt or solvate thereof, as claimed in claim 1, and a pharmaceutically acceptable excipient.

28. A method of treating or preventing a disease, disorder or condition in a subject, the method comprising the step of administering an effective amount of the compound or the pharmaceutically acceptable salt or solvate thereof, as claimed in claim 1, to the subject, thereby treating or preventing the disease, disorder or condition, optionally wherein the disease, disorder or condition is responsive to NLRP3 inhibition.

29. (canceled)

30. The method as claimed in claim 28, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) pain;
(xvii) a condition associated with diabetes;
(xviii) a condition associated with arthritis;
(xix) a headache;
(xx) a wound or burn; and
(xxi) any disease where an individual has been determined to carry a germline or somatic non-silent mutation in NLRP3.

31. The method as claimed in claim 28, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinaemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

32. A method of inhibiting NLRP3 in a subject, the method comprising administering the compound or the pharmaceutically acceptable salt or solvate thereof, as claimed in claim 1, to the subject thereby inhibiting NLRP3.

33. A method of analysing inhibition of NLRP3 or an effect of inhibition of NLRP3 by a compound, comprising contacting a cell or non-human animal with the compound or the pharmaceutically acceptable salt or solvate thereof, as claimed in claim 1, and analysing inhibition of NLRP3 or an effect of inhibition of NLRP3 in the cell or non-human animal by the compound.

34. The method as claimed in claim 28, wherein the compound or the pharmaceutically acceptable salt or solvate thereof is administered as a pharmaceutical composition further comprising a pharmaceutically acceptable excipient.

35. A method of treating or preventing a disease, disorder or condition in a subject, the method comprising the step of administering an effective amount of the prodrug or the pharmaceutically acceptable salt or solvate thereof, as claimed in claim 26, to the subject, thereby treating or preventing the disease, disorder or condition, optionally wherein the disease, disorder or condition is responsive to NLRP3 inhibition.
